(19) United States
(12) Patent Application Publication
(10) Pub. No.: US 2026/0159527 A1
(43) Pub. Date: Jun. 11, 2026

(54) MACROCYCLIC BCL6 DEGRADERS

(71) Applicant: DANA-FARBER CANCER INSTITUTE, INC., Boston, MA (US)

(72) Inventors: Lyn Howard JONES, Boston, MA (US); Huang HUANG, Boston, MA (US); Yingpeng LIU, Boston, MA (US); Jianwei CHE, Boston, MA (US); Nikki KONG, Boston, MA (US)

(73) Assignee: DANA-FARBER CANCER INSTITUTE, INC., Boston, MA (US)

(21) Appl. No.: 19/099,963

(22) PCT Filed: Aug. 8, 2023
(86) PCT No.: PCT/US2023/029714

§ 371 Date: Jan. 30, 2025

Related U.S. Application Data
(60) Provisional application No. 63/396,831, filed on Aug. 10, 2022.
(60) Provisional application No. 63/432,580, filed on Dec. 14, 2022.
(60) Provisional application No. 63/465,584, filed on May 11, 2023.

(51) Int. Cl. C07D 498/22; A61K 31/529; A61K 31/553; A61K 45/06

(52) U.S. Cl. CPC C07D 498/22; A61K 31/529; A61K 31/553; A61K 45/06

(57) ABSTRACT

Described are the compounds, compositions and methods of treating diseases and disorders that involve aberrant B-cell lymphoma 6 (BCL6) activity.

### RELATED APPLICATIONS

This application claims the benefit of priority under 35 U.S.C. § 119 (e) to U.S. Provisional Application No. 63/396,381, filed Aug. 9, 2022, U.S. Provisional Application No. 63/432,580, filed Dec. 14, 2022, and U.S. Provisional Application No. 63/465,584, filed May 11, 2023, each of which are incorporated herein by reference in their entireties.

### BACKGROUND OF THE DISCLOSURE

As demonstrated by the clinical efficacy of thalidomide analogs for the treatment of hematologic malignancies, small molecule-induced protein degradation has emerged as a powerful therapeutic strategy. Thalidomide analogs, including lenalidomide and pomalidomide, modulate the activity of the Cullin Really Interesting New Gene (RING) ligase 4-cereblon (CRBN) (CRL4^{CRBN}) E3 ubiquitin ligase to recruit and ubiquitinate neo-substrates including Ikaros family zinc finger 1 (IKZF1), IKZF3, and casein kinase 1-alpha (CK1α), which leads to their proteasomal degradation (Kronke et al., Science 343:301-305 (2014); Lu et al., Science 343:305-309 (2014); Kronke et al., Nature 523:183-188 (2015)). Other small molecules that induce protein degradation include aryl sulfonamides, which promote the destruction of RNA binding motif protein 39 (RBM39) in a CRL4-DNA damage binding protein 1 (DDBI) and CUL4 associated factor 15 (DCAF15) (CRL4^{DCAF15})-dependent manner (Han et al., Science 356: eaal3755 (2017).

Other types of small molecules that include hetero-bifunctional degraders (also known as PROTACs) (Toure et al., Angew. Chem. Int. Ed. Engl. 55:1966-1973 (2016)) have been developed for a wide range of targets including kinases (Huang et al., Cell Chem. Biol. 25:88-99 (2018)), nuclear receptors (Bondeson et al., Nat. Chem. Biol. 17:611-617 (2015)), and epigenetic enzymes (Winter et al., Science 348:1376-1381 (2015)). These small molecule degraders engage both the E3 ligase and the target protein substrate, promoting formation of a substrate-drug-ligase ternary complex (Nowak et al., Nat. Chem. Biol. 14:706-714 (2018); Petzold et al., Nature 532:127-130 (2016); Sievers et al., Science 362: aat0572 (2018)).

While degraders have shown remarkable efficacy and sustained depletion for some target proteins, other proteins have proven recalcitrant to this approach. One such example is the B cell lymphoma 6 (BCL6) protein, for which hetero-bifunctional degraders have shown insufficient target modulation to induce growth inhibition (McCoull et al., ACS Chem. Biol. 13:3131-3141 (2018)).

BCL6 was first identified as a locus affected by chromosomal translocations in diffuse large B-cell lymphomas (DLBCL). It is now known to be broadly expressed in many lymphomas. Its role in lymphomagenesis stems from its function in the humoral immune system, where upregulation of BCL6 is required for the formation of germinal centers (GC) during the humoral immune response (Ye et al., Nat. Genet. 16:161-170 (1997); Dent et al., Science 276:89-92 (1997)). GCs are transient structures that form in response to antigen stimulation. Within GCs, B cells tolerate massive proliferation and the mutagenic effect of the DNA-editing enzyme AICDA to undergo immunoglobulin affinity maturation (Klein et al., Nat. Rev. Immunol. 8:22-33 (2008)). These activities are orchestrated by and dependent on BCL6, a powerful transcriptional repressor that silences hundreds of genes. Some of these target genes control DNA damage sensing (i.e., ATR, CHEK1, TP53, ARF) and proliferation checkpoints (i.e., CDKN₁A, CDKN₁B, CDKN₂A, CDKN₂B, PTEN) (Hatzi et al., Trends Mol. Med. 20:343-352 (2014)). BCL6 also represses genes required for exit from the GC reaction and plasma cell differentiation (e.g., IRF4, PRDM1). This ensures that GC B cells have sufficient time to acquire somatic hyper-mutation of their immunoglobulin genes. Thus, deregulated suppression of these target genes could result in malignant transformation of B cells.

BCL6 also represses numerous oncogenes in GC B cells, including MYC, BCL2, BMI1, and CCND1 (Ci et al., Blood 173:5536-5548 (2009)). Through this function, BCL6 may mitigate its own pro-oncogenic checkpoint repression effect and thus reduce the potential for malignant transformation of GC B cells. This effect is abrogated in the presence of BCL2 or MYC translocations, which drive expression of these oncogenes through aberrant regulatory elements. The presence of both MYC and/or BCL2 together with BCL6 (regardless of translocations) is clearly deleterious because it provides B cells with simultaneous suppression of checkpoints through BCL6, along with the pro-growth and survival effects of MYC and BCL6 (Cardenas et al., Clin. Cancer Res. 23:885-893 (2017)). In the normal immune response, BCL6 function is terminated by the disruption of BCL6 transcriptional complexes through CD40-induced ERK signaling and downregulation of BCL6 mRNA by IRF4 and PRDM1 (Polo et al., Blood 172:644-651 (2008)). Termination of BCL6 function is required for B cells to exit the GC reaction.

BCL6 is a promising drug target for non-Hodgkin lymphomas such as diffuse large B cell lymphoma (DLBCL) (Cerchietti et al., Cancer Cell 17:400-411 (2010); Cardenas et al., J. Clin. Invest. 126:3351-3362 (2016)) and follicular lymphoma (Bosga-Bouwer et al., Genes Chromosomes Cancer 44:301-304 (2005)). Pathologically increased BCL6 expression, as a result of somatic BCL6 translocation, exonic mutation, promoter mutation, or mutations in regulatory pathways, is a common driver of B cell malignancies (Hatzi et al., Trends Mol. Med. 20:343-352 (2014)). In genetically engineered mice, overexpression of BCL6 is sufficient to drive lymphoma development (Cattoretti et al., Cancer Cell 7:445-455 (2005)). BCL6 acts as a master transcriptional repressor enabling rapid expression of germinal center (GC) B cells and tolerance to genomic instability caused by hypermutation of the immunoglobulin genes and class switch recombination (Hatzi et al., Trends Mol. Med. 20:343-352 (2014)). BCL6 represses a broad range of genes involved in the DNA damage response (Ranuncolo et al., Blood Cells Mol. Dis. 41:95-99 (2008)), cell cycle checkpoints (Tunyaplin et al., J. Immunol. 173:1158-1165 (2004)), and differentiation (Phan et al., Nat. Immunol. 6:1054-1060 (2005)). As expected, knock-out of BCL6 in lymphoma cells results in tumor stasis (Schlager et al., Oncotarget//: 875-890 (2020)). Several peptide and small molecule inhibitors targeting BCL6 have shown efficacy in vivo, but only at high concentrations, which has limited their translation into clinical therapeutic agents (Cerchietti et al., Cancer Cell 17:400-411 (2010); Cardenas et al., J. Clin. Invest. 126:3351-3362 (2016)).

Broad complex/Tramtrack/Bric-a-brac (BTB) proteins are a diverse family of proteins that are characterized by the presence of a common protein-protein interaction domain, known as the BTB domain. BTB proteins have diverse functions ranging from transcriptional regulation and chromatin remodeling to protein degradation and cytoskeletal regulation. Specificity of function is determined in part by additional domains present in a given BTB protein, as well as by interaction partners. Studies of BTB proteins in Drosophila and mammalian systems have revealed the importance of these proteins in multiple developmental contexts, as well as in cancer and neurological and musculoskeletal diseases. BTB proteins play critical roles in transcriptional regulation and chromatin remodeling (Chaharbakhshi et al., Genesis 54:505-518 (2016)).

The BTB domain mediates various functions of BCL6, such as homodimerization and interaction with co-repressor proteins (Ghetu et al., Mol. Cell 29:384-391 (2008); Ahmad et al., Mol. Cell 12:1551-1564 (2003)). Techniques that disrupt the protein-protein interaction between the BTB domain of BCL6 and its co-repressors may be useful to combat BCL6-related diseases.

### SUMMARY OF DISCLOSURE

A first aspect of the present disclosure is directed to a compound having a structure represented by formula (I):

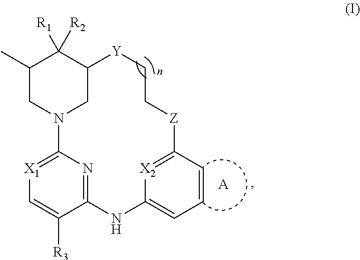

or a pharmaceutically acceptable salt or stereoisomer thereof,
wherein:
X₁ is N, CH, CCl, CF, or CCN;
X₂ is N or CR₄;
   R₄ is H, (C₁-C₄)alkyl, halo, OH, NH₂, (C₁-C₄)alkoxy, (C₁-C₄)haloalkyl, (C₁-C₄)haloalkoxy, (C₂-C₄)alkenyl, (C₂-C₄)alkynyl, NO₂, CN, NH(C₁-C₄)alkyl, or N(C₁-C₄ alkyl)₂;
R₁ is H, OH, or halo;
R₂ is H, OH, or halo;
R₃ is H, halo, or CN;
Y is CH₂, NH, or O,
Z is CH₂, NH, S, or O;
n is 0, 1, or 2;
A is

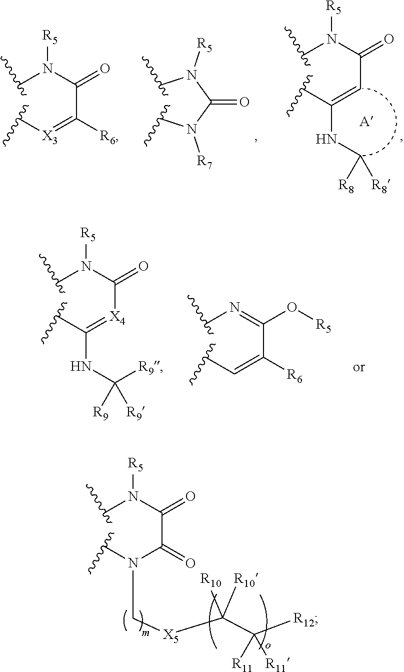

X₃ is CH or N;
R₅ is H, (C₁-C₆)alkyl, (C₁-C₆) hydroxyalkyl, (C₁-C₆)aminoalkyl, (C₃-C₆)carbocyclyl, 4- to 7-membered heterocyclyl, (C₁-C₅)alkyl-(C₃-C₇)carbocyclyl, or (C₃-C₇)carbocyclyl-4- to 7-membered heterocyclyl; wherein said alkyl, carbocyclyl, or heterocyclyl is further optionally substituted by one or more, identical or different R₁₃ groups, wherein R15 is (C₁-C₆)alkyl, (C₁-C₆)alkoxy, halo, NH₂, OH, (C₁-C₆)haloalkyl, NH(C₁-C₆)alkyl, or N((C₁-C₆)alkyl)₂, (C₃-C₆)carbocyclyl, 4- to 7-membered heterocyclyl, or
   R₅ is —L-Y₁-Z₁, wherein
L is absent or (C₁-C₅)alkylene optionally substituted by one or more substituents selected from (C₁-C₂)alkyl and oxo;
Y₁is absent, O, S, S(O), S(O)₂, NR″, C(O), C(O)O, OC(O), C(O)N(R^{r}), N(R^{r})C(O), N(R^{r})C(O)N(R^{r}), N(R^{r})C(O)O, OC(O)N(R^{r}), S(O)₂N(R^{r}), or N(R^{r})S(O)₂;
   each R^{r} is independently H or (C₁-C₄)alkyl;
Z₁is H, (C₁-C₆)alkyl, (C₂-C₆)alkenyl, (C₂-C₆)alkynyl, (C₃-C₁₀)carbocyclyl, or 3- to 10-membered heterocyclyl, wherein Zi is optionally substituted by one or more substituents independently selected from (C₁-C₄)alkyl, halo, (C₁-C₄)haloalkyl, (C₁-C₄)haloalkoxy, NH₂, (C₁-C₄)aminoalkyl, CN, OH, carboxy, carbamoyl, sulphamoyl, mercapto, ureido, NR^{r}R^{s}, OR^{r}, C(O)R″, C(O)OR″, OC(O)R″, C(O)NR^{r}R^{s}, N(R^{r})C(O)R″, S(O)₀₋₂R″, S(O)₂NR^{r}R^{s}, N(R^{r})SO₂R″, Si(R^{r})(R^{s})R^{t} and (CH₂)₁₋₃NR^{r}R^{s}; wherein R^{t}, R^{s}, and R^{r} are each independently H, (C₁-C₆)alkyl, or (C₃-C₆)cycloalkyl; or R^{r} and R^{s} together with the nitrogen atom to which they are attached form a 4-to 9-membered heterocyclyl which is optionally substituted by one or more substituents selected from (C₁-C₄)alkyl, halo, (C₁-C₄)haloalkyl, (C₁-C₄)haloalkoxy, (C₁-C₄)alkoxy, (C₁-C₄)alkylamino, NH₂, CN, and OH;
R₆ is —L₁CR₁₄R₁₅R₁₆, or —CH═CH-R₁₆, wherein
   L₁ is absent, O, S, (C₁-C₄)alkylene, —O-(C₁-C₄)alkylene, or -5-(C₁-C₄)alkylene;
   R₁₄ is H or (C₁-C₄)alkyl;
   R₁₅ is H or (C₁-C₄)alkyl, or
R₁₄ and R₁₅ together with the carbon atom to which they are attached form a (C₃-C₅)carbocyclyl, 4- to 7-membered heterocyclyl, or C═O;
   R₁₆ is (C₁-C₆)alkyl, —NR₁₇R₁₈, —OR₁₇, —C(O)R₁₇, —C(O)OR₁₇, —N(R₁₈)C(O)R₁₇, —C(O)NR₁₄R₁₅, —S(O)-(C₁-C₆)alkyl, —S(O)₂—(C₁-C₆)alkyl, —P(O)-(C₁-C₆ alkyl)₂, —C(NH)NH₂, -(C₁-C₄)alkyl-NR₁₈C(O)R₁₇, or 4- to 7-membered heterocyclyl, wherein
   R₁₇ is H, 3- to 6-membered heterocyclyl, or (C₁-C₄)alkyl optionally substituted by one or more, identical or different groups selected from OH, Cl, F, CF₃, N(C₁-C₄ alkyl)₂, (C₃-C₆)carbocyclyl, 3- to 6-membered heterocyclyl, (C₂-C₄)alkenyl, and (C₂-C₄)alkynyl;
   R₁₈ is H or (C₁-C₄)alkyl;
R₇ is H, methyl, —(CH₂)₁₋₃W₁W₂, or

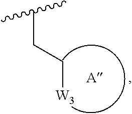

wherein
W₁ is CR₁₉R₁₉ or C(O);
   R₁₉ and R₁₀ are independently H, (C₁-C₂)alkyl, F, OH, CN, NO₂, (C₁-C₂)alkoxy, (C₁-C₂)haloalkyl, (C₁-C₂)haloalkoxy, NH₂, NH(C₁-C₂)alkyl, or N(C₁-C₂ alkyl)₂, or
R₁₉ and R₁₉′ together with the carbon atom to which they are attached form C(O), (C₃-C₆)carbocyclyl or 3- to 6-membered heterocyclyl, which is optionally substituted by one or more substituents independently selected from (C₁-C₂)alkyl, halo, (C₁-C₂)haloalkyl, (C₁-C₂)haloalkoxy, (C₁-C₂)alkoxy, (C₁-C₂)alkylamino, NH₂, CN, and OH;
W₂ is CN, OH, 5- or 6-membered heteroaryl, phenyl, C(O)-(C₁-C₂)alkyl, S(O)₂-(C₁-C₂)alkyl, S(O)(NH)-(C₁-C₂)alkyl, C(O)OCH₃, C(O)NHCH₃, CR₂₀R₂₁R₂₂, NH₂, NH(C₁-C₂)alkyl, or N(C₁-C₂ alkyl)₂, wherein
R₂₀ is H, (C₁-C₂)alkyl, F, Cl, Br, OH, NH₂, CN, NO₂, (C₁-C₂)alkoxy, (C₁-C₂)haloalkyl, or (C₁-C₂)haloalkoxy;
R₂₁ is H, (C₁-C₂)alkyl, F, Cl, Br, OH, CN, NO₂, (C₁-C₂)alkoxy, (C₁-C₂)haloalkyl, (C₁-C₂)haloalkoxy, or -Y₂-L₂-Z₂, wherein
   Y₂ is absent, O, S, S(O), S(O)₂, NR″, C(O), C(O)O, OC(O), C(O)N(R^{r}), N(R^{r})C(O), S(O)₂N(R^{r}), or N(R^{r})SO₂;
L₂ is absent or (C₁-C₂)alkylene;
Z₂ is H, (C₁-C₆)alkyl, (C₂-C₄)alkenyl, (C₂-C₄)alkynyl, phenyl, (C₃-C₆)carbocyclyl, or 4- to 6-membered heterocyclyl, wherein Z₂ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, NH₂, CN, OH, C(O)R″, C(O)OR″, OC(O)R″, C(O)NR^{r}R″, and N(R^{r})C(O)R″, wherein each R^{r} is independently H or (C₁-C₄)alkyl;
or R₂₀ and R₂₁ together with the carbon atom to which they are attached form (C₃-C₆)carbocyclyl or 3- to 6-membered heterocyclyl, optionally substituted by one or more substituents selected from (C₁-C₂)alkyl, halo, (C₁-C₂)haloalkyl, (C₁-C₂)haloalkoxy, (C₁-C₂)alkoxy, (C₁-C₂)alkylamino, NH₂, CN, and OH;
R₂₂ is (C₁-C₂)alkyl, —C(O)OR″, OR″, —C(O)N₂R″, NR″R″, phenyl, or 5-membered heteroaryl, wherein each R″ is independently H or (C₁-C₂)alkyl;
A″ is (C₄-C₆)carbocyclyl or 4- to 6-membered heterocyclyl, optionally substituted with one or more substituents independently selected from (C₁-C₂)alkyl, halo, OH, oxo, CN, and (C₁-C₂)alkoxy;
W₃ is NR₂₃ or CR₂₄R₂₄′, wherein
   R₂₃ is H, (C₁-C₂)alkyl, (C₁-C₄)haloalkyl, (C₁-C₄) hydroxyalkyl, —C(O)CH₃, or —C(O)O-(C₁-C₄)alkyl;
   R₂₄ and R₂₄ are independently H, (C₁-C₂)alkyl, cyclopropyl, F, Cl, Br, OH, NH₂, CN, NO₂, (C₁-C₂)haloalkyl, (C₁-C₂)haloalkoxy, (C₁-C₂)alkoxy, —C(O)OR″, NR″R″, phenyl, or 5-membered heteroaryl;
R₈ is H, (C₁-C₄)alkyl, (C₃-C₆)cycloalkyl, (C₁-C₄)haloalkyl, or CN, wherein said alkyl or cycloalkyl is optionally substituted by one or more substituents selected from (C₁-C₄)alkyl, (C₃-C₆)cycloalkyl, OH, (C₁-C₂)alkoxy, NH₂, NH(C₁-C₂)alkyl, N((C₁-C₂)alkyl)₂, (C₁-C₂)aminoalkyl, and halo;
R₈' is H, (C₁-C₄)alkyl, CN, (C₁-C₄)haloalkyl, or -Y₃-L₃-Z₃, wherein
   Y₃ is absent, C(O)O, or C(O)N(R″);
   L₃ is absent or (C₁-C₂)alkylene;
   Z₃ is H, (C₁-C₆)alkyl, phenyl, (C₃-C₆)cycloalkyl, or 4- to 6-membered heterocyclyl,
wherein Z₃ is optionally substituted by one or more substituents independently selected from (C₁-C₂)alkyl, halo, (C₁-C₂)haloalkyl, (C₁-C₂)haloalkoxy, (C₁-C₂)alkoxy, NH₂, NO₂, CN, and OH, or
R₈ and R₈′, together with the carbon atom to which they are attached, form a (C₄-C₆)carbocyclyl, or 4- to 6-membered heterocyclyl;
A′ is a 6- or 7-membered heterocyclyl, which in addition to R₅ and R₅′, is optionally further substituted by one more substituents independently selected from oxo, (C₁-C₂)alkyl, cyclopropyl, spiro-cyclopropyl, halo, (C₁-C₂)haloalkyl, (C₁-C₂)haloalkoxy, (C₁-C₂)alkoxy, NH₂, CN, and OH;
X₄ is CR₂₅ or N, wherein
R₂₅ is H, F, C₁, or methyl;
R₆ is H, (C₁-C₂)alkyl, (C₃-C₄)cycloalkyl, (C₁-C₂)haloalkyl, CN, (C₂-C₄)alkenyl, or (C₂-C₄)alkynyl;
R₉′ is (C₁-C₄)alkyl, CN, (C₁-C₄)haloalkyl, or -Y₄-L₄-Z₄, wherein
   Y₄ is absent, C(O), C(O)O, OC(O), C(O)N(R″), or S(O)₂N(R″);
   L₄ is absent or (C₁-C₂)alkylene optionally substituted by one or more substituents selected from (C₁-C₂)alkyl and oxo;
   Z₄ is H, (C₁-C₆)alkyl, (C₂-C₄)alkenyl, (C₂-C₄)alkynyl, phenyl, (C₃-C₆)carbocyclyl, (C₃-C₆)cycloalkenyl, or 4- to 6-membered heterocyclyl, wherein Z₄ is optionally substituted by one or more substituents independently selected from oxo, (C₁-C₄)alkyl, (C₃-C₆)cycloalkyl, halo, (C₁-C₄)haloalkyl, (C₁-C₄)haloalkoxy, (C₁-C₄)alkoxy, (C₁-C₄)alkylamino, NH₂, NO₂, CN, OH, C(O)R″, C(O)OR″, OC(O)R″, C(O)NR″R″, and N(R″)C(O)R″, wherein each R^{u} is independently H, (C₁-C₄)alkyl, (C₃-C₆)cycloalkyl, or Z₄ is —Q-L₅-W₄, wherein
   Q is absent, O, NH, or N(C₁-C₂)alkyl;
   L₅ is absent or (C₁-C₂)alkylene optionally substituted by one or more substituents selected from oxo and (C₁-C₂)alkyl;
   W₄ is (C₁-C₄)alkyl, phenyl, (C₃-C₆)cycloalkyl, (C₃-C₆)cycloalkenyl, or 5- or 6-membered heterocyclyl, wherein W₄ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, NH₂, NO₂, CN, or OH; or
R₉ and R₉′, together with the carbon atom to which they are attached, for a (C₃-C₁₀)carbocyclyl or a 4- to 10-membered heterocyclyl, which is optionally substituted by one or more substituents independently selected from oxo, (C₁-C₂)alkyl, halo, (C₁-C₂)haloalkyl, (C₁-C₂)haloalkoxy, (C₁-C₂)alkoxy, (C₁-C₂)alkylamino, NH₂, NO₂, CN, or OH; or the (C₃-C₁₀)carbocyclyl or 4- to 10-membered heterocyclyl is optionally fused to a 5- or 6-membered heteroaryl or phenyl ring, and the 5- or 6-membered heteroaryl or phenyl ring is optionally substituted by (C₁-C₂)alkyl, halo, (C₁-C₂)haloalkyl, (C₁-C₂)haloalkoxy, (C₁-C₂)alkoxy, (C₁-C₂)alkylamino, NH₂, NO₂, CN, or OH;
R₉″ is H, (C₁-C₄)alkyl, (C₁-C₂)haloalkyl, (C₁-C₂)alkoxy, (C₁-C₂)haloalkoxy, CN, NO₂, acetylenyl, phenyl, or S- or 6-membered heteroaryl, wherein said alkyl, phenyl, or heteroaryl is optionally substituted by one or more substituents independently selected from halo, OH, and NH₂;
X₅ is a absent, (C₃-C₆)carbocyclyl, (C₃-C₆)carbocyclyl (C═O), or SO₂;
R₁₀ and R₁₀′ are each independently H, (C₁-C₃)alkyl, (C₁-C₄) hydroxyalkyl, (C₁-C₃)aminoalkyl, or
R₁₀ and R₁₀′, together with the same carbon atom to which they are attached, form (C₃-C₆)carbocyclyl or 4- to 6-membered heterocyclyl, wherein said alkyl, hydroxyalkyl, aminoalkyl, carbocyclyl, or heterocyclyl is optionally substituted by one or more, identical or different R₁₀ₐ groups, wherein R₁₀ₐ is (C₁-C₆)alkyl, (C₁-C₆)alkoxy, (C₁-C₆)alkyl-(C₁-C₃)alkoxy, halo, NH₂, OH, (C₁-C₆)haloalkyl, NH-(C₁-C₆)alkyl, N((C₁-C₆)alkyl)₂, (C₃-C₆)carbocyclyl, 4- to 6-membered heterocyclyl, or
R₁₀′ and R₁₀′, together with the carbon atoms to which they are attached, form (C₃-C₆)carbocyclyl or 4- to 6-membered heterocyclyl;
R₁₁ and R₁₁′ are each independently H, (C₁-C₃)alkyl, (C₁-C₃) hydroxyalkyl, (C₁-C₃)aminoalkyl, or
R₁₁ and R₁₁′, together with the same carbon atom to which they are attached, form C—O, (C₃-C₆)carbocyclyl, or 4- to 6-membered heterocyclyl, wherein said carbocyclyl or heterocyclyl is optionally substituted by one or more, identical or different Rita groups, wherein each R₁₁ₐ is independently (C₁-C₄)alkyl, (C₁-C₄)alkoxy, (C₁-C₆)alkyl-(C₁-C₃)alkoxy, halo, NH₂, OH, (C₁-C₆)haloalkyl, NH-(C₁-C₆)alkyl, N((C₁-C₆)alkyl)₂, (C₃-C₆)carbocyclyl, or 4- to 6-membered heterocyclyl, or wherein two R₁₁ₐ groups, together with the same carbon atom to which they are attached, form C═O;
R₁₂ is H, OH, NH-(C₁-C₆)alkyl, NH-(C₁-C₆) hydroxyalkyl, NH-(C₁-C₆)aminoalkyl, NH-(C₃-C₆)carbocyclyl, NH-4- to 6-membered heterocyclyl, NH-(C₁-C₆)alkyl-(C₃-C₆)carbocyclyl, or NH-(C₁-C₆)alkyl-4- to 6-membered heterocyclyl, wherein said alkyl, hydroxyalkyl, aminoalkyl, carbocyclyl, or heterocyclyl is further optionally substituted by one or more, identical or different R₁₂ₐ groups, wherein each Riza is independently (C₁-C₆)alkyl, (C₁-C₃)alkoxy, (C₁-C₆)alkyl-(C₁-C₄)alkoxy, halo, NH₂, OH, (C₁-C₆)haloalkyl, NH-(C₁-C₆)alkyl, N((C₁-C₆)alkyl)₂, (C₃-C₆)carbocyclyl, or 4- to 6-membered heterocyclyl;
m is 0, 1, or 2; and
o is 0 or 1.

Another aspect of the present disclosure is directed to a pharmaceutical composition that includes a therapeutically effective amount of a compound of formula (I) or a pharmaceutically acceptable salt or stereoisomer thereof, and a pharmaceutically acceptable carrier.

A further aspect of the present disclosure is directed to a method of treating a disease or disorder that involves aberrant BCL6 activity that entails administering to a subject in need thereof a therapeutically effective amount of a compound of formula (I) or a pharmaceutically acceptable salt or stereoisomer thereof. In some embodiments, the disease or disorder is cancer. In some embodiments, the disease or disorder is an inflammatory disease.

In some embodiments, the cancer is a lymphoid malignancy. In some embodiments, the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), cutaneous T-cell lymphoma, chronic myeloid leukemia, or B-cell non-Hodgkin's lymphoma.

### DETAILED DESCRIPTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Therefore, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising,” which is synonymous with “including,” “containing,” or “characterized by,” is inclusive or open-ended and does not exclude additional, unrecited elements or method steps. When used in the context of the number of heteroatoms in a heterocyclic structure, it means that the heterocyclic group that has that minimum number of heteroatoms. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the disclosure.

With respect to compounds of the present disclosure, and to the extent the following terms are used herein to further describe them, the following definitions apply.

As used herein, the term “alkyl” refers to a saturated linear or branched-chain monovalent hydrocarbon radical. In some embodiments, the alkyl radical is a C₁-C₆ group. In some embodiments, and to the extent not disclosed otherwise for any one or more groups of the compounds of formula (I), the alkyl radical is a C₀-C₆, C₀-C₅, C₀-C₃, C₁-C₆, C₁-C₅, C₁-C₄ or C₁-C₃ group (wherein Co alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. In some embodiments, an alkyl group is a C₁-C₃ alkyl group. In some embodiments, an alkyl group is a C₁-C₂ alkyl group. In some embodiments, an alkyl group is a methyl group.

As used herein, the term “alkylene” refers to a straight or branched divalent hydrocarbon chain linking the rest of the molecule to a radical group, consisting solely of carbon and hydrogen, containing no unsaturation and having from one to six carbon atoms, for example, methylene, ethylene, propylene, n-butylene, and the like. The alkylene chain may be attached to the rest of the molecule through a single bond and to the radical group through a single bond. In some embodiments,, and to the extent not disclosed otherwise for any one or more groups of the compounds of formula (I), an alkylene group contains one to four carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In some embodiments, the alkenyl radical is a C₂-Cis group. In some embodiments, and to the extent not disclosed otherwise for any one or more groups of the compounds of formula (I), the alkenyl radical is a C₂-C₁₂, C₂-C₁₀, C₂-C₅, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In some embodiments, the alkynyl radical is a C₂-Cis group. In some embodiments, and to the extent not disclosed otherwise for any one or more groups of the compounds of formula (I), the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. In some embodiments, the alkoxyl group is methoxy, ethoxy, propyloxy, or tert-butoxy. An “ether” is two hydrocarbyl groups covalently linked by an oxygen. Accordingly, the substituent of an alkyl that renders that alkyl an ether is or resembles an alkoxyl, such as can be represented by one of—O-alkyl, —O-alkenyl, and-O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Therefore, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 12 carbon atoms, that is alone or part of a larger moiety (e.g., an alkylcarbocyclic group). The term carbocyclyl includes mono-, bi-, tri-, fused, bridged, and spiro-ring systems, and combinations thereof. In one embodiment, carbocyclyl includes 3 to 10 carbon atoms (C₃-C₁₀). In one embodiment, carbocyclyl includes 3 to 6 carbon atoms (C₃-C₆). In one embodiment, carbocyclyl includes 5 to 6 carbon atoms (C₅-C₆). In some embodiments, carbocyclyl, as a bicycle, includes C₆-C₁₀. In another embodiment, carbocyclyl, as a spiro system, includes C₅-C₁₁. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and phenyl; bicyclic carbocyclyls having 7 to 11 ring atoms include [4,3], [4,4], [4,5], [5,5], [5,6] or [6,6]ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Therefore, the term carbocyclic also embraces carbocyclylalkyl groups which as used herein refer to a group of the formula-R^{c}-carbocyclyl where R^{c} is an alkylene chain. The term carbocyclic also embraces carbocyclylalkoxy groups which as used herein refer to a group bonded through an oxygen atom of the formula--O-Re-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-12 carbon atoms. In another embodiment, aryl includes groups having 6-10 carbon atoms. Examples of aryl groups include phenyl, naphthyl, biphenyl, 1,2,3,4-tetrahydronaphthalenyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Therefore, the term aryl embraces aralkyl groups (e.g., benzyl) which as disclosed above refer to a group of the formula--Re-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula--O-Re--aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, 4, or 5)carbon atoms have been replaced with a heteroatom or heteroatom-containing group (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3- to 12-membered heterocyclyl ring system. In some embodiments, a heterocyclyl refers to a saturated ring system, such as a 3- to 12-membered saturated heterocyclyl ring system. In some embodiments, a heterocyclyl refers to a heteroaryl ring system, such as a 5-to 12-membered heteroaryl ring system. The term heterocyclyl also includes C₂-C₈ heterocycloalkyl, which is a saturated or partially unsaturated mono-, bi-, or spiro-ring system containing 2-8 carbons and one or more (e.g., 1, 2, or 3) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from O, N, and S. In some embodiments, heterocyclyl includes 4- to 6-membered monocycles having one or more heteroatoms selected from O, N, and S. In some embodiments, heterocyclyl includes 3-membered monocycles. In some embodiments, heterocyclyl includes 4-membered monocycles. In some embodiments, heterocyclyl includes 5- to 6-membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be substituted (e.g., methyl, isopropyl) and/or quaternized (e.g., [NR₄]⁺CI; [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrotbienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol [4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl (e.g., thiazol-2-yl), thiadiazolyl (e.g., 1,3,4-thiadiazol-5-yl and 1,2,4-thiadiazol-5-yl), oxazolyl (e.g., oxazol-2-yl), and oxadiazolyl (e.g., 1,3,4-oxadiazol-5-yl and 1,2,4-oxadiazol-5-yl). Example of 5-membered heterocyclyls containing 2 to 4 nitrogen atoms include imidazolyl (e.g., imidazol-2-yl), triazolyl (e.g., 1,3,4-triazol-5-yl, 1,2,3-triazol-5-yl, and 1,2,4-triazol-5-yl), and tetrazolyl (e.g., 1H-tetrazol-5-yl). Representative examples of benzo-fused S-membered heterocyclyls include benzoxazol-2-yl, benzthiazol-2-yl and benzimidazol-2-yl. Example of 6-membered heterocyclyls containing one to three nitrogen atoms and optionally a sulfur or oxygen atom are pyridyl (e.g., pyrid-2-yl, pyrid-3-yl, and pyrid-4-yl), pyrimidyl (e.g., pyrimid-2-yl and pyrimid-4-yl), triazinyl (e.g., 1,3,4-triazin-2-yl and 1,3,5-triazin-4-yl), pyridazinyl (e.g., pyridazin-3-yl), and pyrazinyl. In some embodiments, a heterocyclic group includes a heterocyclic ring fused to one or more (e.g., 1 or 2) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Therefore, the term heterocyclic embraces N-heterocyclyl groups which as used herein refer to a heterocyclyl group containing at least one nitrogen atom and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, 1-pyrazolidinyl, 1-imidazolinyl and 1-imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2— or 3-morpholinyl, 2— or 3- or 4-piperidinyl, 2-piperazinyl, and 2— or 3-pyrrolidinyl. The term heterocyclic also embraces heterocyclylalkyl groups which as disclosed above refer to a group of the formula--Re-heterocyclyl where is an chain, alkylene

The term heterocyclic also embraces heterocyclylalkoxy groups which as used herein refer to a radical bonded through an oxygen atom of the formula--O-Re-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”)) refers to a monocyclic, bicyclic or tricyclic ring system having 5 to 12 ring atoms, wherein at least one ring is aromatic and contains at least one heteroatom. In one embodiment, heteroaryl includes 5- to 6-membered monocyclic aromatic groups where one or more ring atoms is O, N, or S. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, and 1,2,3-triazol-5-yl. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3 (4H)-one. A heteroaryl group may be mono-, bi- or tri-cyclic. In some embodiments, a heteroaryl group includes a heteroaryl ring fused to one or more (e.g., 1 or 2) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Therefore, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula--R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula--O--R^{c}-heteroaryl, where R^{c} is an alkylene group as defined above.

Unless stated otherwise, and to the extent not further defined for any particular group(s) in the compounds of formula (I), any of the groups described herein may be substituted or unsubstituted. To the extent not disclosed otherwise for any particular group(s), representative examples of substituents may include alkyl (e.g., C₁-C₆, C₁-C₅, C₁-C₄, C₁-C₃, C₁-C₂, C₁), substituted alkyl (e.g., substituted C₁-C₆, C₁-C₅, C₁-C₄, C₁-C₃, C₁-C₂, C₁), alkoxy (e.g., C₁-C₆, C₁-Cs, C₁-C₄, C₁-C₃, C₁-C₂, C₁), substituted alkoxy (e.g., substituted C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., substituted C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., substituted C₂-C₆, C₂-C₅, C₂-C₄, C₂-C₃, C₂), cyclic (e.g., C₃-C₁₂, C₅-C₆), substituted cyclic (e.g., substituted C₃-C₁₂, C₅-C₆), carbocyclic (e.g., C₃-C₁₂, C₅-C₆), substituted carbocyclic (e.g., substituted C₃-C₁₂, C₅-C₆), heterocyclic (e.g., 3- to 12-membered, 5- to 6-membered), substituted heterocyclic (e.g., substituted 3- to 12-membered, 5- to 6-membered), aryl (e.g., benzyl and phenyl), substituted aryl (e.g., substituted benzyl or substituted phenyl), heteroaryl (e.g., pyridyl or pyrimidyl), substituted heteroaryl (e.g., substituted pyridyl or substituted pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., substituted C₆-C₁₂, C₆), alkylthio (e.g., C₁-C₆), substituted alkylthio (e.g., substituted C₁-C₆), arylthio (e.g., C₆-C₁₂, C₆), substituted arylthio (e.g., substituted C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

Broadly, the compounds of the disclosure are represented by formula (I):

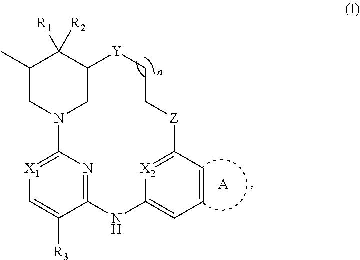

or a pharmaceutically acceptable salt or stereoisomer thereof,
wherein:
X₁ is N, CH, CCl, CF, or CCN;
X₂ is N or CR₄;
   R₄ is H, (C₁-C₄)alkyl, halo, OH, NH₂, (C₁-C₄)alkoxy, (C₁-C₄)haloalkyl, (C₁-C₄)haloalkoxy, (C₂-C₄)alkenyl, (C₂-C₄)alkynyl, NO₂, CN, NH(C₁-C₄)alkyl, or N(C₁-C₄ alkyl)₂;
R₁ is H, OH, or halo;
R₂ is H, OH, or halo;
R₃ is H, halo, or CN;
Y is CH₂, NH, or O;
Z is CH₂, NH, S, or O;
n is 0, 1, or 2;
A is

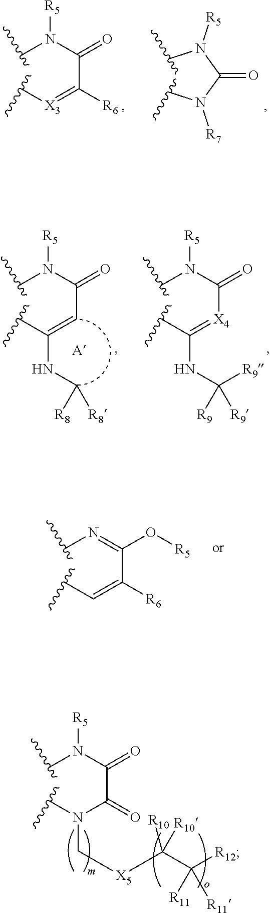

X₃ is CH or N;
R₅ is H, (C₁-C₆)alkyl, (C₁-C₆) hydroxyalkyl, (C₁-C₆)aminoalkyl, (C₃-C₆)carbocyclyl, 4- to 7-membered heterocyclyl, (C₁-C₆)alkyl-(C₃-C₇)carbocyclyl, or (C₃-C₇)carbocyclyl-4- to 7-membered heterocyclyl; wherein said alkyl, carbocyclyl, or heterocyclyl is further optionally substituted by one or more, identical or different R₁₅ groups, wherein R₁₅ is (C₁-C₆)alkyl, (C₁-C₆)alkoxy, halo, NH₂, OH, (C₁-C₆)haloalkyl, NH(C₁-C₆)alkyl, or N((C₁-C₆)alkyl)₂, (C₃-C₆)carbocyclyl, 4- to 7-membered heterocyclyl, or R₅ is —L-Y₁-Z₁, wherein
L is absent or (C₁-C₅)alkylene optionally substituted by one or more substituents selected from (C₁-C₂)alkyl and oxo;
Y₁is absent, O, S, S(O), S(O)₂, NR″, C(O), C(O)O, OC(O), C(O)N(R″), N(R″)C(O), N(R″)C(O)N(R″), N(R″)C(O)O, OC(O)N(R″), S(O)₂N(R″), or N(R″)S(O)₂;
each R′ is independently H or (C₁-C₄)alkyl;
Z₃ is H, (C₁-C₆)alkyl, (C₂-C₆)alkenyl, (C₂-C₆)alkynyl, (C₃-C₁₀)carbocyclyl, or 3- to 10-membered heterocyclyl, wherein Zi is optionally substituted by one or more substituents independently selected from (C₁-C₄)alkyl, halo, (C₁-C₄)haloalkyl, (C₁-C₄)haloalkoxy, NH₂, (C₁-C₄)aminoalkyl, CN, OH, carboxy, carbamoyl, sulphamoyl, mercapto, ureido, NR^{r}R^{s}, OR^{r}, C(O)R″, C(O)OR″, OC(O)R″, C(O)NR^{r}R^{s}, N(R″)C(O)R″, S(O)₀₋₂R″, S(O)₂NR^{r}R₅, N(RO)SO₂R″, Si(R^{r})(R^{s})R^{t} and (CH₂)₁₋₃NR^{r}R^{s}; wherein R″, R₅, and R^{t} are each independently H, (C₁-C₆)alkyl, or (C₃-C₆)cycloalkyl; or R′ and R^{s} together with the nitrogen atom to which they are attached form a 4- to 9-membered heterocyclyl which is optionally substituted by one or more substituents selected from (C₁-C₄)alkyl, halo, (C₁-C₄)haloalkyl, (C₁-C₄)haloalkoxy, (C₁-C₄)alkoxy, (C₁-C₄)alkylamino, NH₂, CN, and OH;
R₆ is —L₁CR₁₄R₁₅R₁₆, or —CH═CH-R₁₆, wherein
L₁ is absent, O, S, (C₁-C₄)alkylene, —O-(C₁-C₄)alkylene, or -5-(C₁-C₄)alkylene;
R₁₄ is H or (C₁-C₄)alkyl;
R₁₅ is H or (C₁-C₄)alkyl, or
R₁₄ and R₁₅ together with the carbon atom to which they are attached form a (C₃-C₅)carbocyclyl, 4- to 7-membered heterocyclyl, or C═O;
R₁₆ is (C₁-C₆)alkyl, —NR₁₇R₁₈, —OR₁₇, —C(O)R₁₇, —C(O)OR₁₇, —N(R₁₈)C(O)R₁₇, —C(O)NR₁₄R₁₅, —S(O)-(C₁-C₆)alkyl, —S(O)₂—(C₁-C₆)alkyl, —P(O)-(C₁-C₆ alkyl)₂, —C(NH)NH₂, -(C₁-C₄)alkyl-NR₁&C(O)R₁₇, or 4- to 7-membered heterocyclyl, wherein
   R₁₇ is H, 3- to 6-membered heterocyclyl, or (C₁-C₄)alkyl optionally substituted by one or more, identical or different groups selected from OH, Cl, F, CF₃, N(C₁-C₄ alkyl)₂, (C₃-C₆)carbocyclyl, 3- to 6-membered heterocyclyl, (C₂-C₄)alkenyl, and (C₂-C₄)alkynyl;
   R₁₈ is H or (C₁-C₄)alkyl;
R₇ is H, methyl, —(CH₂)13W₁W₂, or

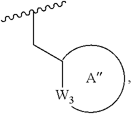

wherein
W₁ is CR₁₉R₁₉ or C(O);
   R₁₉ and R₁₉′ are independently H, (C₁-C₂)alkyl, F, OH, CN, NO₂, (C₁-C₂)alkoxy, (C₁-C₂)haloalkyl, (C₁-C₂)haloalkoxy, NH₂, NH(C₁-C₂)alkyl, or N(C₁-C₂ alkyl)₂, or
   R₁₉ and R₁₉′ together with the carbon atom to which they are attached form C(O), (C₃-C₆)carbocyclyl or 3- to 6-membered heterocyclyl, which is optionally substituted by one or more substituents independently selected from (C₁-C₂)alkyl, halo, (C₁-C₂)haloalkyl, (C₁-C₂)haloalkoxy, (C₁-C₂)alkoxy, (C₁-C₂)alkylamino, NH₂, CN, and OH;
W₂ is CN, OH, 5- or 6-membered heteroaryl, phenyl, C(O)-(C₁-C₂)alkyl, S(O)₂-(C₁-C₂)alkyl, S(O)(NH)-(C₁-C₂)alkyl, C(O)OCH₃, C(O)NHCH₃, CR₂₀R₂₁R₂₂, NH₂, NH(C₁-C₂)alkyl, or N(C₁-C₂ alkyl)₂, wherein
R₂₀ is H, (C₁-C₂)alkyl, F, Cl, Br, OH, NH₂, CN, NO₂, (C₁-C₂)alkoxy, (C₁-C₂)haloalkyl, or (C₁-C₂)haloalkoxy;
R₂₁ is H, (C₁-C₂)alkyl, F, Cl, Br, OH, CN, NO₂, (C₁-C₂)alkoxy, (C₁-C₂)haloalkyl, (C₁-C₂)haloalkoxy, or -Y₂-L₂-Z₂, wherein
   Y₂ is absent, O, S, S(O), S(O)₂, NR″, C(O), C(O)O, OC(O), C(O)N(R″), N(R″)C(O), S(O)₂N(R″), or N(R″)SO₂;
   L₂ is absent or (C₁-C₂)alkylene;
   Z₂ is H, (C₁-C₆)alkyl, (C₂-C₄)alkenyl, (C₂-C₄)alkynyl, phenyl, (C₃-C₆)carbocyclyl, or 4- to 6-membered heterocyclyl, wherein Z₂ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, NH₂, CN, OH, C(O)R″, C(O)OR″, OC(O)R″, C(O)NR^{r}R″, and N(R″)C(O)R″, wherein each R′ is independently H or (C₁-C₄)alkyl;
or R₂₀ and R₂₁ together with the carbon atom to which they are attached form (C₃-C₆)carbocyclyl or 3- to 6-membered heterocyclyl, optionally substituted by one or more substituents selected from (C₁-C₂)alkyl, halo, (C₁-C₂)haloalkyl, (C₁-C₂)haloalkoxy, (C₁-C₂)alkoxy, (C₁-C₂)alkylamino, NH₂, CN, and OH;
R₂₂ is (C₁-C₂)alkyl, —C(O)OR″, OR″, —C(O)NR″, NR″R″, phenyl, or 5-membered heteroaryl, wherein each R″ is independently H or (C₁-C₂)alkyl;
A″ is (C₄-C₆)carbocyclyl or 4- to 6-membered heterocyclyl, optionally substituted with one or more substituents independently selected from (C₁-C₂)alkyl, halo, OH, oxo, CN, and (C₁-C₂)alkoxy;
W₃ is NR₂₃ or CR₂₄R₂₄′, wherein
   R₂₃ is H, (C₁-C₂)alkyl, (C₁-C₄)haloalkyl, (C₁-C₄) hydroxyalkyl, —C(O)CH₃, or —C(O)O-(C₁-C₄)alkyl;
   R₂₄ and R₂₄′ are independently H, (C₁-C₂)alkyl, cyclopropyl, F, Cl, Br, OH, NH₂, CN, NO₂, (C₁-C₂)haloalkyl, (C₁-C₂)haloalkoxy, (C₁-C₂)alkoxy, —C(O)OR″, NR″R″, phenyl, or 5-membered heteroaryl;
R₈ is H, (C₁-C₄)alkyl, (C₃-C₆)cycloalkyl, (C₁-C₄)haloalkyl, or CN, wherein said alkyl or cycloalkyl is optionally substituted by one or more substituents selected from (C₁-C₄)alkyl, (C₃-C₆)cycloalkyl, OH, (C₁-C₂)alkoxy, NH₂, NH(C₁-C₂)alkyl, N((C₁-C₂)alkyl)₂, (C₁-C₂)aminoalkyl, and halo;
R₈' is H, (C₁-C₄)alkyl, CN, (C₁-C₄)haloalkyl, or -Y₃-L₃-Z₃, wherein
   Y₃ is absent, C(O)O, or C(O)N(R″);
   L₃ is absent or (C₁-C₂)alkylene;
   Z₃ is H, (C₁-C₆)alkyl, phenyl, (C₃-C₆)cycloalkyl, or 4- to 6-membered heterocyclyl, wherein Z₃ is optionally substituted by one or more substituents independently selected from (C₁-C₂)alkyl, halo, (C₁-C₂)haloalkyl, (C₁-C₂)haloalkoxy, (C₁-C₂)alkoxy, NH₂, NO₂, CN, and OH, or
R₈ and R₈′, together with the carbon atom to which they are attached, form a (C₄-C₆)carbocyclyl, or 4- to 6-membered heterocyclyl;
A′ is a 6- or 7-membered heterocyclyl, which in addition to R₈ and R₈′, is optionally further substituted by one more substituents independently selected from oxo, (C₁-C₂)alkyl, cyclopropyl, spiro-cyclopropyl, halo, (C₁-C₂)haloalkyl, (C₁-C₂)haloalkoxy, (C₁-C₂)alkoxy, NH₂, CN, and OH;
X₄ is CR₂₅ or N, wherein
   R₉ is H, F, C₁, or methyl; R₆ is H, (C₁-C₂)alkyl, (C₃-C₄)cycloalkyl, (C₁-C₂)haloalkyl, CN, (C₂-C₄)alkenyl, or (C₂-C₄)alkynyl;
R₉′ is (C₁-C₄)alkyl, CN, (C₁-C₄)haloalkyl, or -Y₄-L₄-Z₄, wherein
   Y₄ is absent, C(O), C(O)O, OC(O), C(O)N(R″), or S(O),N(R″);
   L₄ is absent or (C₁-C₂)alkylene optionally substituted by one or more substituents selected from (C₁-C₂)alkyl and oxo;
   Z₄ is H, (C₁-C₆)alkyl, (C₂-C₄)alkenyl, (C₂-C₄)alkynyl, phenyl, (C₃-C₆)carbocyclyl, (C₃-C₆)cycloalkenyl, or 4- to 6-membered heterocyclyl, wherein Z₄ is optionally substituted by one or more substituents independently selected from oxo, (C₁-C₄)alkyl, (C₃-C₆)cycloalkyl, halo, (C₁-C₄)haloalkyl, (C₁-C₄)haloalkoxy, (C₁-C₄)alkoxy, (C₁-C₄)alkylamino, NH₂, NO₂, CN, OH, C(O)R″, C(O)OR″, OC(O)R″, C(O)NR″R″, and N(R″)C(O)R″, wherein each R″ is independently H, (C₁-C₄)alkyl, (C₃-C₆)cycloalkyl, or
Z₄ is —Q-L₅-W₄, wherein
Q is absent, O, NH, or N(C₁-C₂)alkyl;
L₅ is absent or (C₁-C₂)alkylene optionally substituted by one or more substituents selected from oxo and (C₁-C₂)alkyl;
W₄ is (C₁-C₄)alkyl, phenyl, (C₃-C₆)cycloalkyl, (C₃-C₆)cycloalkenyl, or 5- or 6-membered heterocyclyl, wherein W₄ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, NH₂, NO₂, CN, or OH; or
R₉ and R₉′, together with the carbon atom to which they are attached, for a (C₃-C₁₀)carbocyclyl or a 4- to 10-membered heterocyclyl, which is optionally substituted by one or more substituents independently selected from oxo, (C₁-C₂)alkyl, halo, (C₁-C₂)haloalkyl, (C₁-C₂)haloalkoxy, (C₁-C₂)alkoxy, (C₁-C₂)alkylamino, NH₂, NO₂, CN, or OH; or the (C₃-C₁₀)carbocyclyl or 4- to 10-membered heterocyclyl is optionally fused to a 5- or 6-membered heteroaryl or phenyl ring, and the 5- or 6-membered heteroaryl or phenyl ring is optionally substituted by (C₁-C₂)alkyl, halo, (C₁-C₂)haloalkyl, (C₁-C₂)haloalkoxy, (C₁-C₂)alkoxy, (C₁-C₂)alkylamino, NH₂, NO₂, CN, or OH;
R₉″ is H, (C₁-C₄)alkyl, (C₁-C₂)haloalkyl, (C₁-C₂)alkoxy, (C₁-C₂)haloalkoxy, CN, NO₂, acetylenyl, phenyl, or 5- or 6-membered heteroaryl, wherein said alkyl, phenyl, or heteroaryl is optionally substituted by one or more substituents independently selected from halo, OH, and NH₂,
X₅ is a absent, (C₃-C₆)carbocyclyl, (C₃-C₆)carbocyclyl (C═O), or SO₂;
R₁₀ and R₁₀′ are each independently H, (C₁-C₃)alkyl, (C₁-C₄) hydroxyalkyl, (C₁-C₃)aminoalkyl, or
R₁₀ and R₁₀′, together with the same carbon atom to which they are attached, form (C₃-C₆)carbocyclyl or 4- to 6-membered heterocyclyl, wherein said alkyl, hydroxyalkyl, aminoalkyl, carbocyclyl, or heterocyclyl is optionally substituted by one or more, identical or different R₁₀ₐ groups, wherein R₁₀ₐ is (C₁-C₆)alkyl, (C₁-C₆)alkoxy, (C₁-C₆)alkyl-(C₁-C₃)alkoxy, halo, NH₂, OH, (C₁-C₆)haloalkyl, NH-(C₁-C₆)alkyl, N((C₁-C₆)alkyl)₂, (C₃-C₆)carbocyclyl, 4- to 6-membered heterocyclyl, or
R₁₀′ and R₁₁′, together with the carbon atoms to which they are attached, form (C₃-C₆)carbocyclyl or 4- to 6-membered heterocyclyl;
R₁₁ and R₁₀′ are each independently H, (C₁-C₄)alkyl, (C₁-C₄) hydroxyalkyl, (C₁-C₃)aminoalkyl, or
R₁₁ and R₁₁′, together with the same carbon atom to which they are attached, form C═O, (C₃-C₆)carbocyclyl, or 4- to 6-membered heterocyclyl, wherein said carbocyclyl or heterocyclyl is optionally substituted by one or more, identical or different R₁₁ₐ groups, wherein each Rita is independently (C₁-C₆)alkyl, (C₁-C₆)alkoxy, (C₁-C₆)alkyl-(C₁-C₃)alkoxy, halo, NH₂, OH, (C₁-C₆)haloalkyl, NH-(C₁-C₆)alkyl, N((C₁-C₆)alkyl)₂, (C₃-C₆)carbocyclyl, or 4- to 6-membered heterocyclyl, or wherein two Rita groups, together with the same carbon atom to which they are attached, form C═O;
R₁₂ is H, OH, NH-(C₁-C₆)alkyl, NH-(C₁-C₆) hydroxyalkyl, NH-(C₁-C₆)aminoalkyl, NH-(C₃-C₆)carbocyclyl, NH-4- to 6-membered heterocyclyl, NH-(C₁-C₆)alkyl-(C₃-C₆)carbocyclyl, or NH-(C₁-C₆)alkyl-4- to 6-membered heterocyclyl, wherein said alkyl, hydroxyalkyl, aminoalkyl, carbocyclyl, or heterocyclyl is further optionally substituted by one or more, identical or different R₁₂ₐ groups, wherein each R₁₂ₐ is independently (C₁-C₆)alkyl, (C₁-C₆)alkoxy, (C₁-C₆)alkyl-(C₁-C₂)alkoxy, halo, NH₂, OH, (C₁-C₆)haloalkyl, NH-(C₁-C₆)alkyl, N((C₁-C₆)alkyl)₂, (C₃-C₆)carbocyclyl, or 4- to 6-membered heterocyclyl;
m is 0, 1, or 2; and
o is 0 or 1.

In some embodiments, Z is O). In some embodiments, Z is CH₂.

In some embodiments, Y is CH₂. In some embodiments, Y is O. In some embodiments, Y is NH.

In some embodiments, n is 0. In some embodiments, n is 1. In some embodiments, n is 2.

In some embodiments, R₁ and R₂ are each H. In some embodiments, R₁ and R₂ are each halo. In some embodiments, R₁ and R₂ are each F.

In some embodiments, R₃ is halo. In some embodiments, R₃ is C₁.

In some embodiments, X₁ is N.

In some embodiments, X₂ is CH.

In some embodiments, Z is O and Y is CH₂. In some embodiments, Z is O and Y is O. In some embodiments, Z is O and Y is NH. In some embodiments, Z is CH₂ and Y is CH₂. In some embodiments, Z is CH₂ and Y is O. In some embodiments, Z is CH₂ and Y is NH.

In some embodiments, Z is O, Y is CH₂, and n is 1. In some embodiments, Z is O, Y is O, and n is 1. In some embodiments, Z is O, Y is NH, and n is 1. In some embodiments, Z is CH₂, Y is CH₂, and n is 1. In some embodiments, Z is CH₂, Y is O, and nis 1. In some embodiments, Z is CH₂, Y is NH, and n is 1.

In some embodiments, Z is O, Y is CH₂, n is 1, and R₁ and R₂ are each H or F. In some embodiments, Z is O, Y is O, nis 1, and R₁ and R₂ are each H or F. In some embodiments, Z is O, Y is NH, nis 1, and R^{t} and R₂ are each H or F. In some embodiments, Z is CH₂, Y is CH₂, n is 1, and R₁ and R₂ are each H or F. In some embodiments, Z is CH₂, Y is O, nis 1, and R₁ and R₂ are each H or F. In some embodiments, Z is CH₂, Y is NH, nis 1, and R₁ and R₂ are each H or F.

In some embodiments, Z is O, Y is CH₂, n is 1, R₁ and R₂ are each H or F, and R₃ is C₁. In some embodiments, Z is O, Y is O, n is 1, R₁ and R₂ are each H or F, and R₃ is C₁. In some embodiments, Z is O, Y is NH, n is 1, R₁ and R₂ are each H or F, and R₃ is C₁. In some embodiments, Z is CH₂, Y is CH₂, n is 1, R₁ and R₂ are each H or F, and R₃ is C₁. In some embodiments, Z is CH₂, Y is O, n is 1, R₁ and R₂ are each H or F, and R₃ is C₁. In some embodiments, Z is CH₂, Y is NH, n is 1, R₁ and R₂ are each H or F, and R₃ is C₁.

In some embodiments, Z is O, Y is CH₂, n is 1, R₁ and R₂ are each H or F, R₅ is C₁, and X₁ and X₂ are independently N or CH. In some embodiments, Z is O, Y is O, nis 1, R₁ and R₂ are each H or F, R₅ is C₁, and X₁ and X₂ are independently N or CH. In some embodiments, Z is O, Y is NH, n is 1, R₁ and R₂ are each H or F, R₃ is C₁, and X₁ and X₂ are independently N or CH. In some embodiments, Z is CH₂, Y is CH₂, n is 1, R₁ and R₂ are each H or F, Ra is C₁, and X₁ and X₂ are independently N or CH. In some embodiments, Z is CH₂, Y is O, nis 1, R₁ and R₂ are each H or F, R₃ is C₁, and X₁ and X₂ are independently N or CH. In some embodiments, Z is CH₂, Y is NH, nis 1, R₁ and R₂ are each H or F, R₃ is C₁, and X₁ and X₂ are independently N or CH.

In some embodiments, the compounds of the disclosure are represented any of formulas (Ia-If):

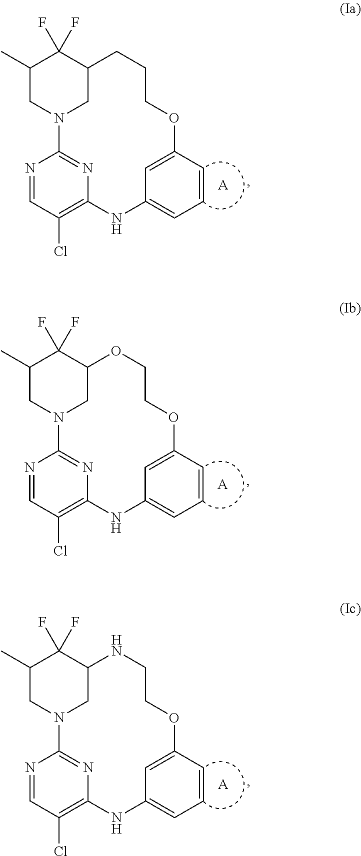

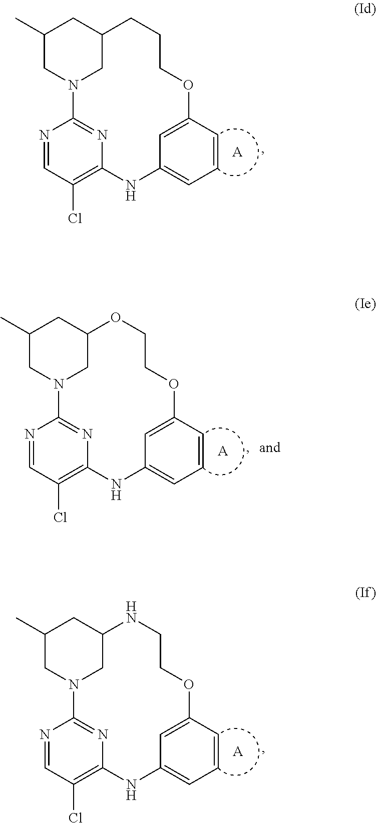

or a pharmaceutically acceptable salt or stereoisomer thereof.

In some embodiments, the compounds of the disclosure are represented any of formulas:

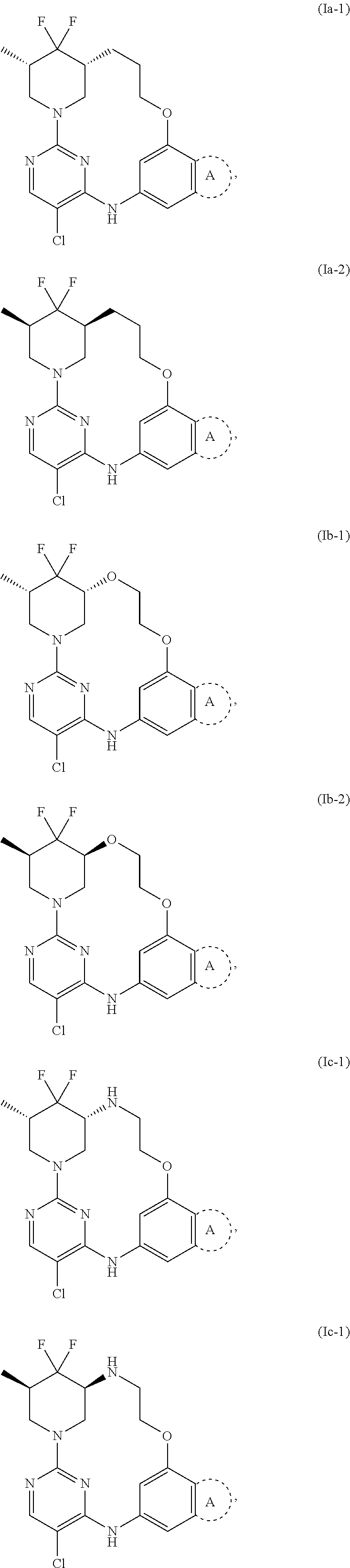

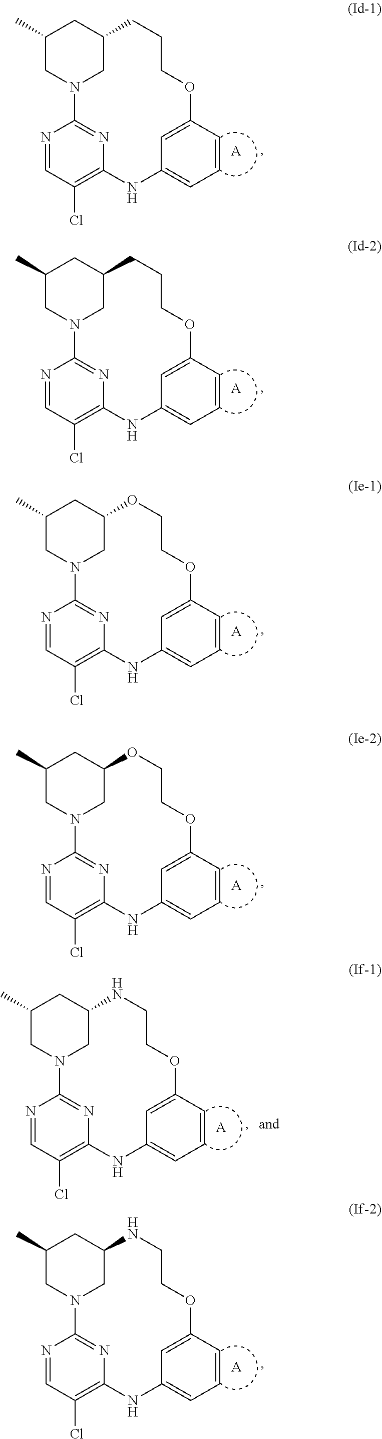

or a pharmaceutically acceptable salt or stereoisomer thereof.

In some embodiments, A is

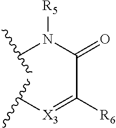

and the compound of formula (I) has the structure of formula I-1,

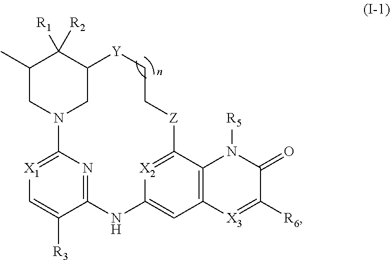

or a pharmaceutically acceptable salt or stereoisomer thereof.

In some embodiments of formula I-1, R₅ is optionally substituted (C₁-C₆)alkyl. In some embodiments of formula I-1, R₅ is methyl.

In some embodiments of formula I-1, X₃ is CH.

In some embodiments of formula I-1, R₆ is —L₁CR₁₄R₁₅R₁₆. In some embodiments of formula I-1, L₁ is —O-(C₁-C₄)alkylene. In some embodiments of formula I-1, L₁ is-O-(C₁)alkylene. In some embodiments of formula I-1, R₁₄ and R₁₅ together with the same carbon atom to which they are attached form C═O. In some embodiments of formula I-1, R₁₆ is methyl, OH, NH₂, or NHMe. In some embodiments of formula I-1, R₁₆ is NHMe.

In some embodiments of formula I-1, R₅ is (C₁-C₆)alkyl, X₅ is CH, and R₆ is —L₁CR₁₄R₁₅R₁₆, wherein L₁ is —O-(C₁-C₄)alkylene, R₁₄ and R₁₅ together with the same carbon atom to which they are attached form C═O, and R₁₅ is methyl, OH, NH₂, or NHMe.

In some embodiments, the compound of formula I-1 is of formula I-1a,

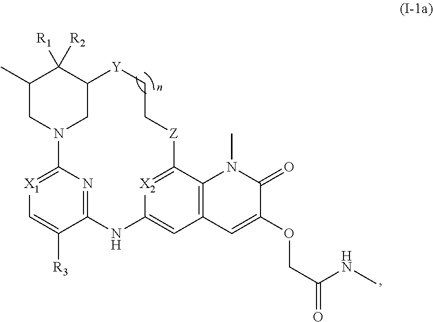

or a pharmaceutically acceptable salt or stereoisomer thereof.

In some embodiments, the compound of formula I-1a is of formula I-1al or I-1ab,

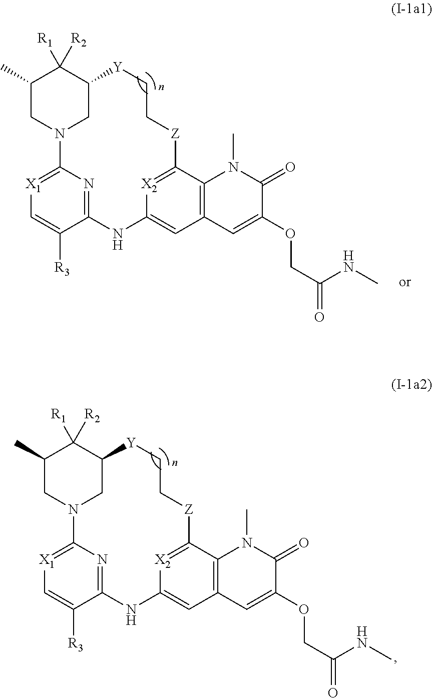

or a pharmaceutically acceptable salt or stereoisomer thereof.

In some embodiments, A is

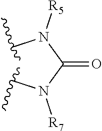

and the compound of formula (I) has the structure of formula I-2,

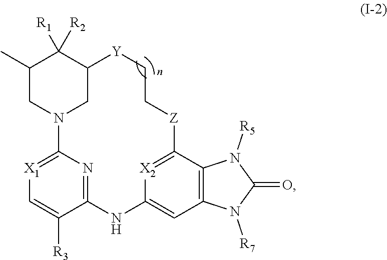

or a pharmaceutically acceptable salt or stereoisomer thereof.

In some embodiments of formula I-2, R₅ is optionally substituted (C₁-C₆)alkyl. In some embodiments of formula I-2, R₅ is methyl.

In some embodiments of formula I-2, R₇ is —(CH₂)₁₋₃W₁W₂. In some embodiments of formula I-2, R₇ is —(CH₂)₂ W: W₂. In some embodiments of formula I-2, W₁ is CR₁₉R₁₉′. In some embodiments of formula I-2, R₁₉ and R₁₉′ are both methyl. In some embodiments of formula I-2, W₂ is CN, OH, or NH₂. In some embodiments of formula I-2, W₂ is OH. In some embodiments of formula I-2, R₇ is H. In some embodiments of formula I-2, R₇ is methyl.

In some embodiments of formula 1-2, R₇ is —(CH₂)₁₋₂W₁W₂. In some embodiments of formula I-2, W₁ is CR₁₉R₁₉: In some embodiments of formula I-2, R₁₉ and R₁₉ are independently H, OH, or methyl. In some embodiments of formula 1-2, W₂ is CR₂oR₂₁R₂₂. In some embodiments of formula I-2, R₂₀ and Rat are both H and Rex is OH. In some embodiments of formula I-2, R₂₀ and R₂₁ are both methyl and R₂₂ is OH. In some embodiments of formula I-2, R₂₀ and R₂₁ are both H and R₂₂ is S(O)(NH)-(C₁-C₂)alkyl. In some embodiments of formula I-2, R₂₀ and Rai together with the carbon atom to which they are attached form 4- to 6-membered heterocyclyl, optionally substituted by one or more substituents selected from (C₁-C₂)alkyl, halo, (C₁-C₂)haloalkyl, (C₁-C₂)haloalkoxy, (C₁-C₂)alkoxy, (C₁-C₂)alkylamino, NH₂, CN, and OH, and R₂₂ is OH.

In some embodiments of formula I-2, R₅ is (C₁-C₆)alkyl and R₇ is —(CH₂)₁₋₃W₁W₂. In some embodiments of formula I-2, R₅ is methyl and R₇ is —(CH₂)₂W₁W₂, wherein W₁ is CRORDD′, wherein R₁₅ and R₁₉ are both methyl, and W₂ is CN, OH, or NH₂.

In some embodiments of formula I-2, R₅ is (C₁-C₆)alkyl and R₇ is H. In some embodiments of formula I-2, R₅ is methyl and R₇ is H.

In some embodiments of formula I-2, R₅ is (C₁-C₃)alkyl and R₇ is methyl. In some embodiments of formula I-2, R₅ is methyl and R₇ is methyl.

In some embodiments of formula 1-2, R₅ is (C₁-C₆)alkyl and Ry is —(CH₂)₁₋₂W₁W₂, wherein W₁ is CR₁₉R₁₉′ and W₂ is CR₂₀R₂₁R₂₂. In some embodiments of formula I-2, R₁₉ and R₁₉′ are independently H, OH, or methyl. In some embodiments of formula I-2, R₂₀ and R₁₁ are both H and R₂₂ is OH. In some embodiments of formula I-2, R₂₀ and R₂₁ are both methyl and R₂₂ is OH. In some embodiments of formula I-2, R₂₀ and R₂₁ are both H and R₂₂ is S(O)(NH)-(C₁-C₂)alkyl. In some embodiments of formula I-2, R₂₀ and Rat together with the carbon atom to which they are attached form 4- to 6-membered heterocyclyl, optionally substituted by one or more substituents selected from (C₁-C₂)alkyl, halo, (C₁-C₂)haloalkyl, (C₁-C₃)haloalkoxy, (C₁-C₂)alkoxy, (C₁-C₂)alkylamino, NH₂, CN, and OH, and R₂₂ is OH.

In some embodiments, the compound of formula 1-2 is of formula I-2a,

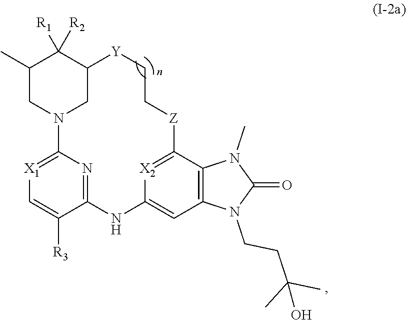

or a pharmaceutically acceptable salt or stereoisomer thereof.

In some embodiments, the compound of formula I-2a is of formula 1-2al or I-2a2,

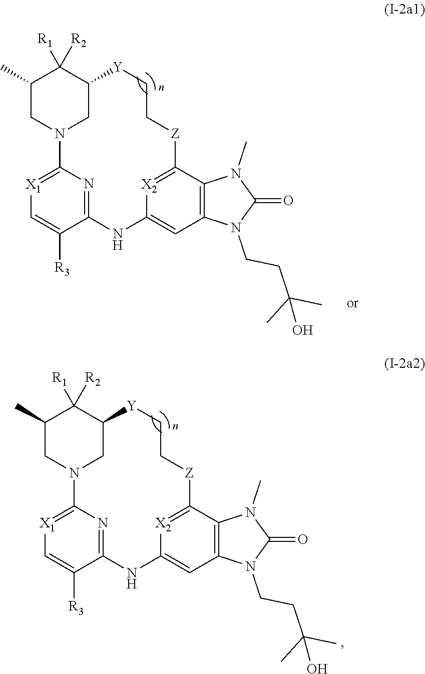

or a pharmaceutically acceptable salt or stereoisomer thereof.

In some embodiments, the compound of formula I-2 is of formula I-2b,

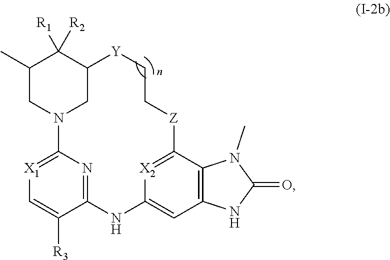

or a pharmaceutically acceptable salt or stereoisomer thereof.

In some embodiments, the compound of formula I-2b is of formula I-2b1 or I-2b2,

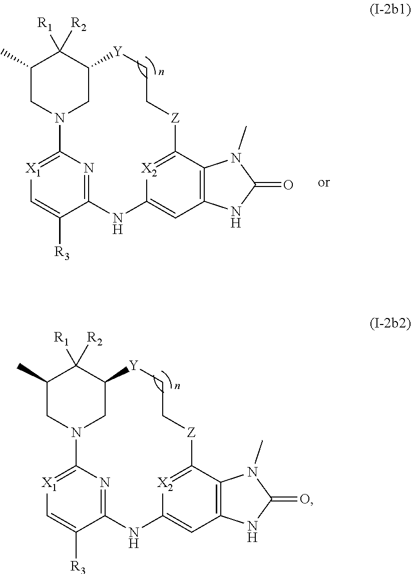

or a pharmaceutically acceptable salt or stereoisomer thereof.

In some embodiments, the compound of formula I-2 is of formula I-2c,

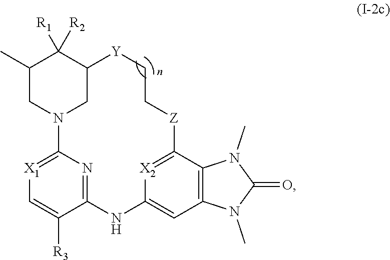

or a pharmaceutically acceptable salt or stereoisomer thereof.

In some embodiments, the compound of formula I-2c is of formula I-2cl or I-2c2,

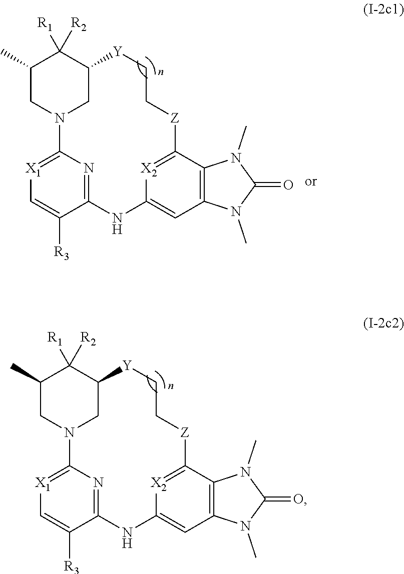

or a pharmaceutically acceptable salt or stereoisomer thereof.

In some embodiments, A is

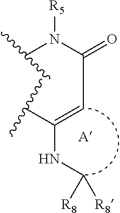

and the compound of formula (I) has the structure of formula I-3,

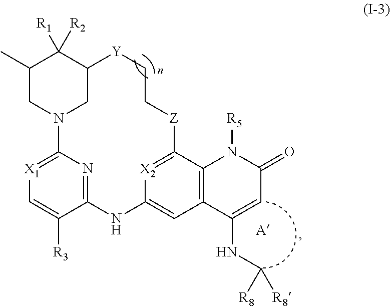

or a pharmaceutically acceptable salt or stereoisomer thereof.

In some embodiments of formula I-3, R₅ is optionally substituted (C₁-C₆)alkyl. In some embodiments of formula 1-3, R₅ is methyl.

In some embodiments of formula I-3, R₅ is (C₃-C₆)cycloalkyl and R₅' is H. In some embodiments of formula I-3, R₅ is cyclopropyl and R₅' is H.

In some embodiments of formula I-3, A′ is a 7-membered heterocyclyl, wherein the heterocyclyl contains 2 heteroatoms selected from N and O, and which in addition to R₅ and R₅′, is optionally further substituted by one more substituents independently selected from oxo, (C₁-C₂)alkyl, cyclopropyl, spiro-cyclopropyl, halo, (C₁-C₂)haloalkyl, (C₁-C₂)alkoxy, NH₂, CN, and OH.

In some embodiments of formula 1-3, R₅ is (C₁-C₆)alkyl, R₈ is (C₃-C₆)cycloalkyl, R₈′ is H, and A′ is a 7-membered heterocyclyl, wherein the heterocyclyl contains 2 heteroatoms selected from N and O, and which in addition to R₅ and R₅′, is optionally further substituted by one more substituents independently selected from oxo, (C₁-C₂)alkyl, cyclopropyl, spiro-cyclopropyl, halo, (C₁-C₂)haloalkyl, (C₁-C₂)alkoxy, NH₂, CN, and OH.

In some embodiments, the compound of formula I-3 is of formula I-3a,

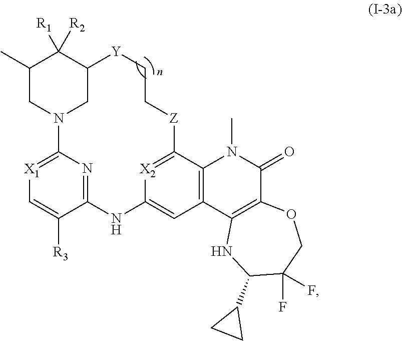

or a pharmaceutically acceptable salt or stereoisomer thereof.

In some embodiments, the compound of formula I-3a is of formula I-3al or I-3a2,

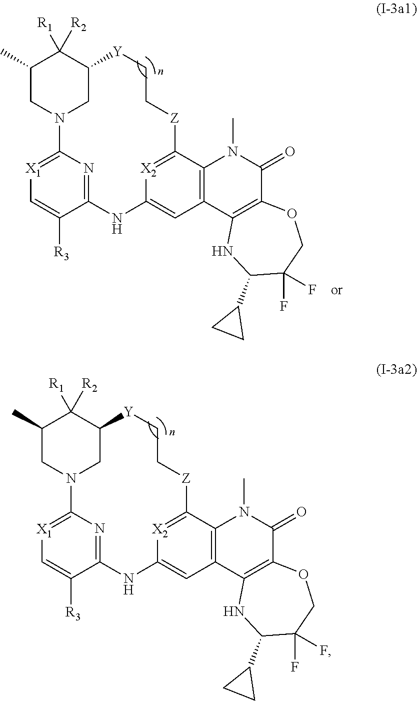

or a pharmaceutically acceptable salt or stereoisomer thereof.

In some embodiments, A is

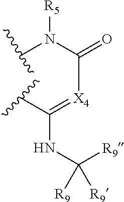

and the compound of formula (I) has the structure of formula I-4,

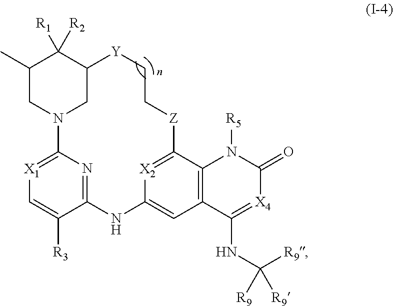

or a pharmaceutically acceptable salt or stereoisomer thereof.

In some embodiments of formula I-4, R₅ is optionally substituted (C₁-C₆)alkyl. In some embodiments of formula 1-4, R₅ is methyl.

In some embodiments of formula I-4, X₄ is CH. In some embodiments of formula I-4, X₄ is NH.

In some embodiments, A is

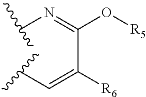

and the compound of formula (I) has the structure of formula I-5,

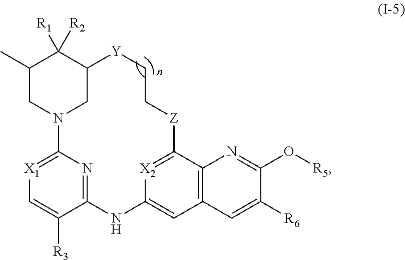

or a pharmaceutically acceptable salt or stereoisomer thereof.

In some embodiments of formula I-5, R₅ is 4-membered heterocyclyl or 4-membered heterocyclyl (C₂)alkyl; wherein said alkyl, carbocyclyl, or heterocyclyl is further optionally substituted by one or more, identical or different R₁₅ groups. In some embodiments of formula I-5, R₂ is 4-membered heterocyclyl or 4-membered heterocyclyl (C₂)alkyl, wherein the heterocyclyl contains 1 heteroatom selected from N and O.

In some embodiments of formula I-5, R₂ is (C₁-C₆)alkyl, (C₁-C₆)alkyl-OH, (C₁-C₆)alkyl-NH(C₁-C₆)alkyl, or (C₁-C₆)alkyl-N((C₁-C₆)alkyl)₂.

In some embodiments of formula I-5, Re is -L₁CR₁₄R₁₅R₁₆. In some embodiments of formula I-5, L₁ is —O-(C₁-C₄)alkylene. In some embodiments of formula I-5, L₁ is —O-(C₁)alkylene. In some embodiments of formula I-5, R₁₄ and R₁₅ together with the same carbon atom to which they are attached form C═O. In some embodiments of formula I-5, R₁₆ is methyl, OH, NH₂, or NHMe. In some embodiments of formula I-5, R₁₆ is NHMe.

In some embodiments, A is

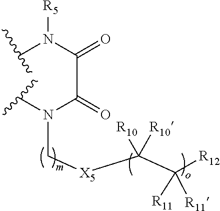

and the compound of formula (I) has the structure of formula I-6,

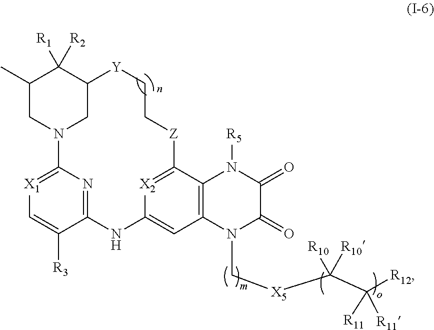

or a pharmaceutically acceptable salt or stereoisomer thereof.

In some embodiments of formula I-6, R₅ is optionally substituted (C₁-C₆)alkyl. In some embodiments of formula I-6, R₅ is methyl.

In some embodiments of formula I-6, m is 0.

In some embodiments of formula I-6, mis 1.

In some embodiments of formula I-6, X₅ is absent.

In some embodiments of formula I-6, o is 0.

In some embodiments of formula I-6, o is 1.

In some embodiments of formula I-6, R₁₀ and R₁₁ are each H and R₁₀′ and R₁₁′, together with the carbon atoms to which they are attached, form cyclobutyl.

In some embodiments of formula I-6, R₁₂ is OH.

In some embodiments of formula I-6, R₅ is (C₁-C₆)alkyl, m is 1, X₅ is absent, o is 1, R₁₀ and R₁₁ are each H and R₁₀′ and Run′, together with the carbon atoms to which they are attached, form cyclobutyl, and R₁₂ is OH.

In some embodiments of formula I-6, R₅ is (C₁-C₆)alkyl, m is 0, X₅ is absent, o is 0, and R₁₂ is H.

In some embodiments, the compound of formula I-6 is of formula I-6a,

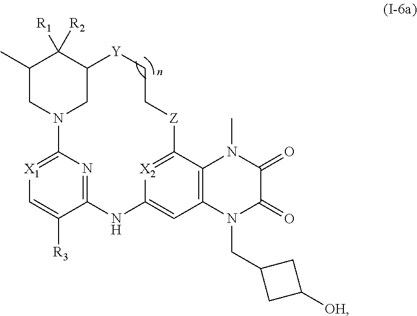

or a pharmaceutically acceptable salt or stereoisomer thereof.

In some embodiments, the compound of formula I-6a is of formula I-6al or I-6a2,

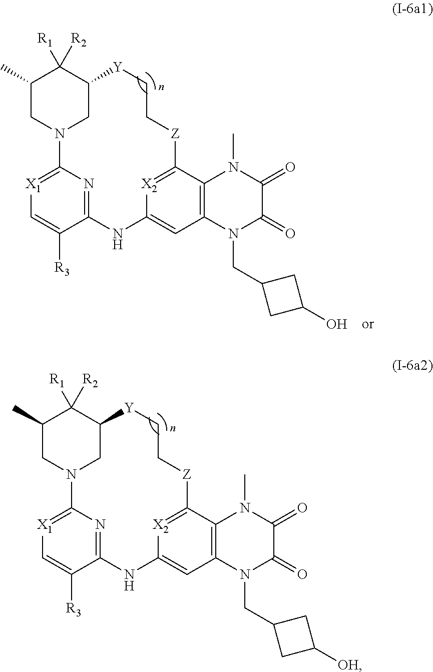

or a pharmaceutically acceptable salt or stereoisomer thereof.

In some embodiments, the compound of formula I-6 is of formula I-6b,

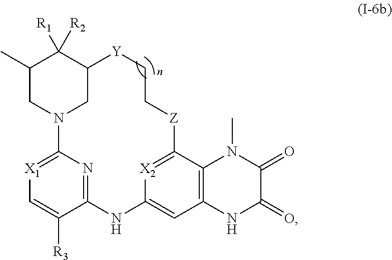

or a pharmaceutically acceptable salt or stereoisomer thereof.

In some embodiments, the compound of formula I-6b is of formula I-6b1 or I-6b2,

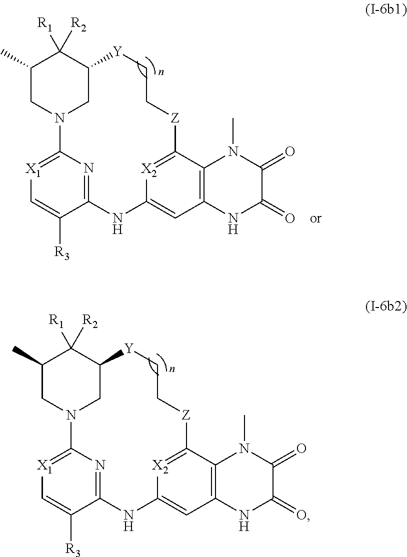

or a pharmaceutically acceptable salt or stereoisomer thereof.

In some embodiments of formulas I-1a to I-6b, Z is O. In some embodiments of formulas I-1a to I-6b, Z is CH₂.

In some embodiments of formulas I-1a to I-6b, Y is CH₂. In some embodiments of formulas I-1a to I-6b, Y is O. In some embodiments, Y is NH.

In some embodiments of formulas I-1a to I-6b, nis 0. In some embodiments of formulas I-1a to I-6b, n is 1. In some embodiments of formulas I-1a to I-6b, n is 2.

In some embodiments of formulas I-1a to I-6b, R₁ and R₂ are each H. In some embodiments of formulas I-1a to I-6b, R₁ and R₂ are each halo. In some embodiments of formulas I-1a to I-6b, R₁ and R₂ are each F.

In some embodiments of formulas I-1a to I-6b, R₅ is halo. In some embodiments of formulas I-1a to I-6b, R₃ is C₁. In some embodiments of formulas I-1a to I-6b, R₅ is F.

In some embodiments of formulas I-1a to I-6b, X₁ is N.

In some embodiments of formulas I-1a to I-6b, X₂ is CH.

In some embodiments of formulas I-1a to I-6b, Z is O and Y is CH₂. In some embodiments of formulas I-1a to I-6b, Z is O and Y is O. In some embodiments of formulas I-1a to I-6b, Z is O and Y is NH. In some embodiments of formulas I-1a to I-6b, Z is CH₂ and Y is CH₂. In some embodiments of formulas I-1a to I-6b, Z is CH₂ and Y is O. In some embodiments of formulas I-1a to I-6b, Z is CH₂ and Y is NH.

In some embodiments of formulas I-1a to I-6b, Z is O, Y is CH₂, and n is 1. In some embodiments of formulas I-1a to I-6b, Z is O, Y is O, and nis 1. In some embodiments of formulas I-1a to I-6b, Z is O, Y is NH, and nis 1. In some embodiments of formulas I-1a to I-6b, Z is CH₂, Y is CH₂, and n is 1. In some embodiments of formulas I-1a to I-6b, Z is CH₂, Y is O, and nis 1. In some embodiments of formulas I-1a to I-6b, Z is CH₂, Y is NH, and nis 1.

In some embodiments of formulas I-1a to I-6b, Z is O, Y is CH₂, n is 1, and R₁ and R₂ are each H or F. In some embodiments of formulas I-1a to I-6b, Z is O, Y is O, nis 1, and R₁₁ and R₂ are each H or F. In some embodiments of formulas I-1a to I-6b, Z is O, Y is NH, n is 1, and Ri and R₂ are each H or F. In some embodiments of formulas I-1a to I-6b, Z is CH₂, Y is CH₂, nis 1, and R₁ and R₂ are each H or F. In some embodiments of formulas I-1a to I-6b, Z is CH₂, Y is O, n is 1, and R₁ and R₂ are each H or F. In some embodiments of formulas I-1a to I-6b, Z is CH₂, Y is NH, nis 1, and R₁ and R₂ are each H or F.

In some embodiments of formulas I-1a to I-6b, Z is O, Y is CH₂, n is 1, R₁ and R₂ are each H or F, and R₅ is C₁ or F. In some embodiments of formulas I-1a to I-6b, Z is O, Y is O, nis 1, R₁ and R₂ are each H or F, and R₃ is C₁ or F. In some embodiments of formulas I-1a to I-6b, Z is O, Y is NH, n is 1, R₁ and R₂ are each H or F, and R₃ is CI or F. In some embodiments of formulas I-1a to I-6b, Z is CH₂, Y is CH₂, n is 1, R₁ and R₂ are each H or F, and R₃ is C₁ or F. In some embodiments of formulas I-1a to I-6b, Z is CH₂, Y is O, n is 1, R₁ and R₂ are each H or F, and R₃ is C₁ or F. In some embodiments of formulas I-1a to I-6b, Z is CH₂, Y is NH, nis 1, R₁ and R₂ are each H or F, and R₃ is C₁ or F.

In some embodiments of formulas I-1a to I-6b, Z is O, Y is CH₂, n is 1, R₁₁ and Re are each H or F, R₃ is C₁ or F, and X₁ and X₂ are independently N or CH. In some embodiments of formulas I-1a to I-6b, Z is O, Y is O, n is 1, R₁₁ and Rₐ are each H or F, Ra is C₁ or F, and X₁ and X₂ are independently N or CH. In some embodiments of formulas I-1a to I-6b, Z is O, Y is NH, n is 1, R₁ and R₂ are each H or F, R₃ is C₁ or F, and X₁ and X₂ are independently N or CH. In some embodiments of formulas I-1a to I-6b, Z is CH₂, Y is CH₂, n is 1, R₁ and R₂ are each H or F, R₃ is C₁ or F, and X₁ and X₂ are independently N or CH. In some embodiments of formulas I-1a to I-6b, Z is CH₂, Y is O, nis 1, R₁ and R₂ are each H or F, R₃ is CI or F, and X₁ and X₂ are independently N or CH. In some embodiments of formulas I-1a to I-6b, Z is CH₂, Y is NH, nis 1, R₁ and R₂ are each H or F, R₃ is C₁ or F, and X₁ and X₂ are independently N or CH.

In some embodiments, compounds of the present disclosure are represented by any one of the following structures:

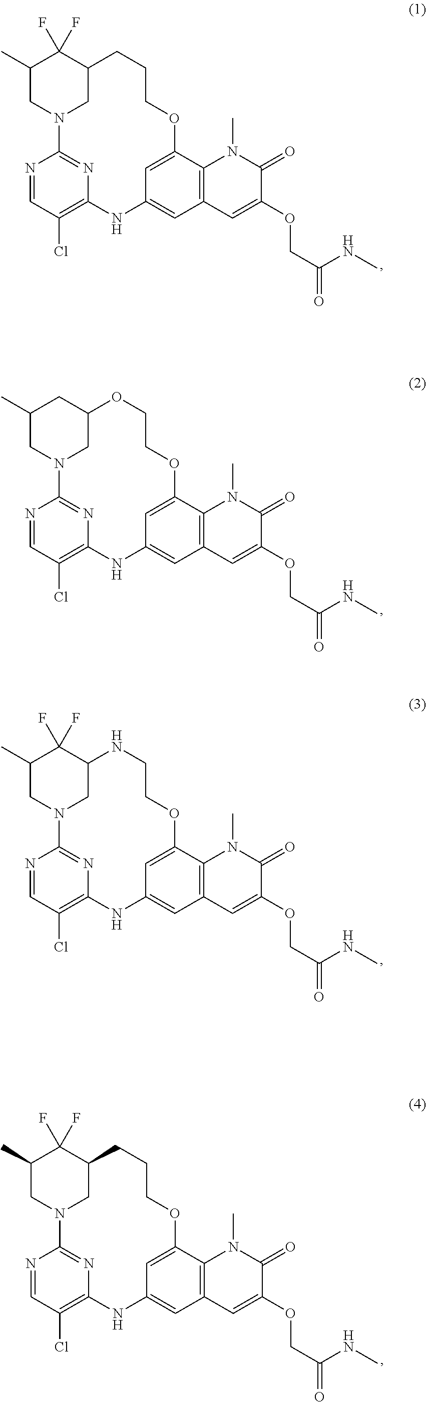

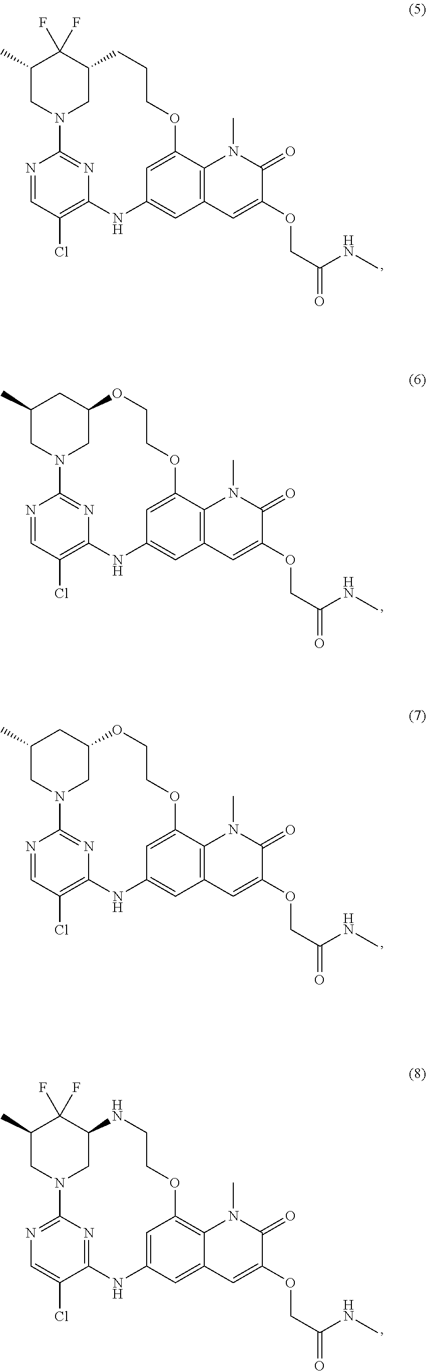

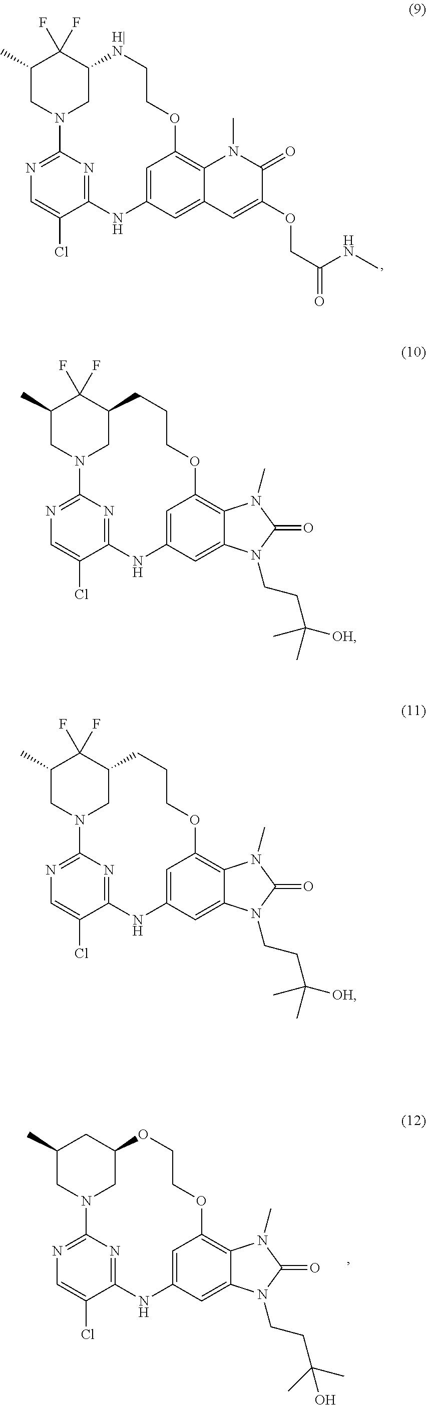

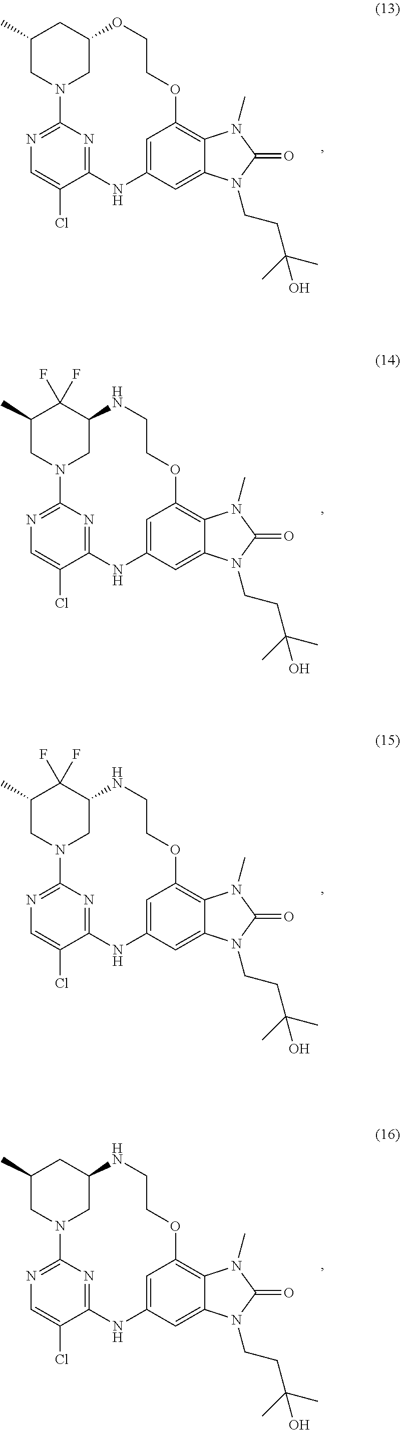

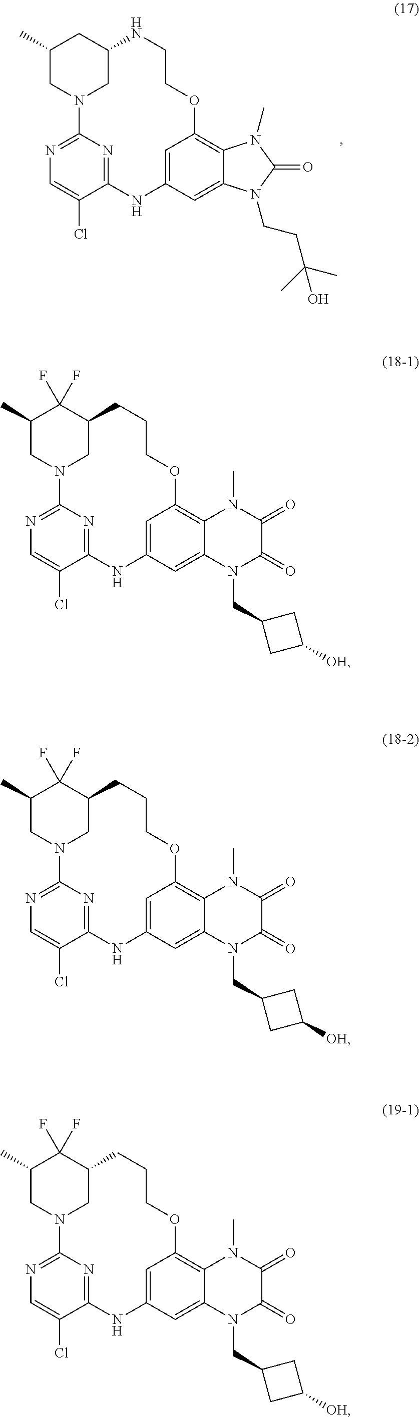

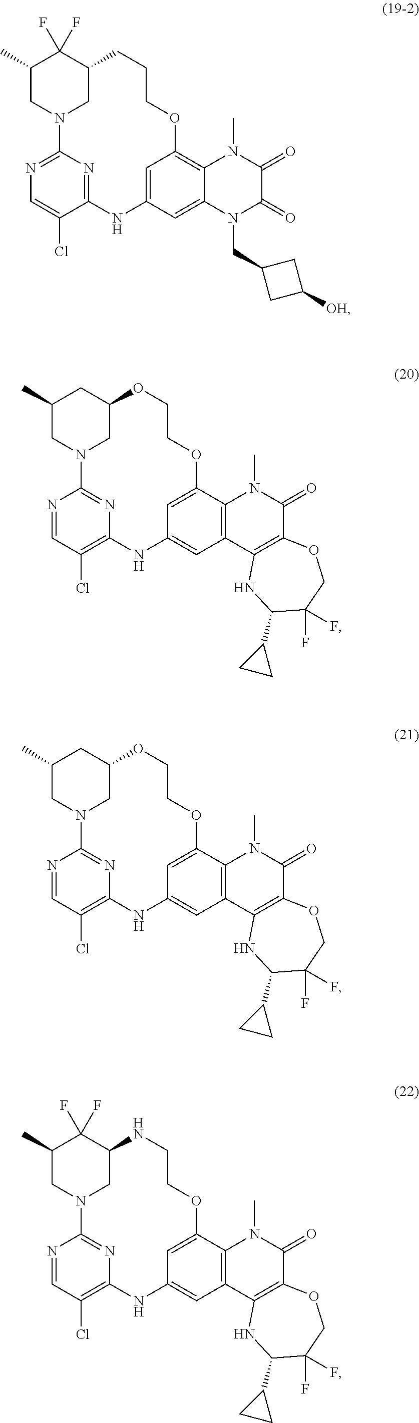 [SMILES: C[C@H]1CN2C[C@@H](CCCOc3cc(cc4c3n(C)c(=O)c(=O)n4C[C@H]3C[C@@H](O)C3)Nc3nc2ncc3Cl)C1(F)F ; C[C@H]1C[C@@H]2CN(C1)c1ncc(Cl)c(n1)Nc1cc(c3c(c1)c1c(c(=O)n3C)OCC(F)(F)[C@H](C3CC3)N1)OCCO2 ; C[C@@H]1C[C@H]2CN(C1)c1ncc(Cl)c(n1)Nc1cc(c3c(c1)c1c(c(=O)n3C)OCC(F)(F)[C@H](C3CC3)N1)OCCO2 ; C[C@@H]1CN2C[C@H](NCCOc3cc(cc4c5c(c(=O)n(C)c34)OCC(F)(F)[C@H](C3CC3)N5)Nc3nc2ncc3Cl)C1(F)F]

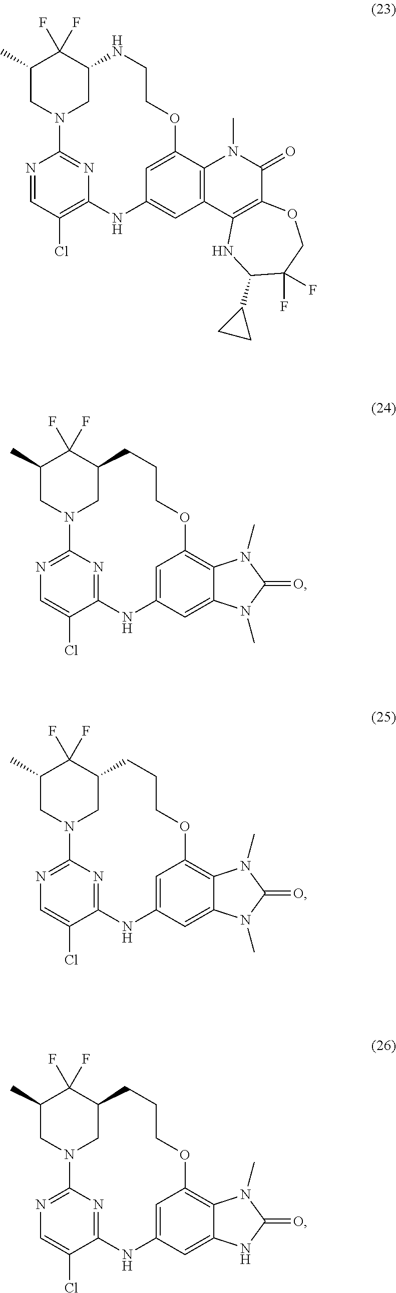 [SMILES: C[C@H]1CN2C[C@@H](NCCOc3cc(cc4c5c(c(=O)n(C)c34)OCC(F)(F)[C@H](C3CC3)N5)Nc3nc2ncc3Cl)C1(F)F ; C[C@@H]1CN2C[C@H](CCCOc3cc(cc4c3n(C)c(=O)n4C)Nc3nc2ncc3Cl)C1(F)F ; C[C@H]1CN2C[C@@H](CCCOc3cc(cc4c3n(C)c(=O)n4C)Nc3nc2ncc3Cl)C1(F)F ; C[C@@H]1CN2C[C@H](CCCOc3cc(cc4[nH]c(=O)n(C)c34)Nc3nc2ncc3Cl)C1(F)F]

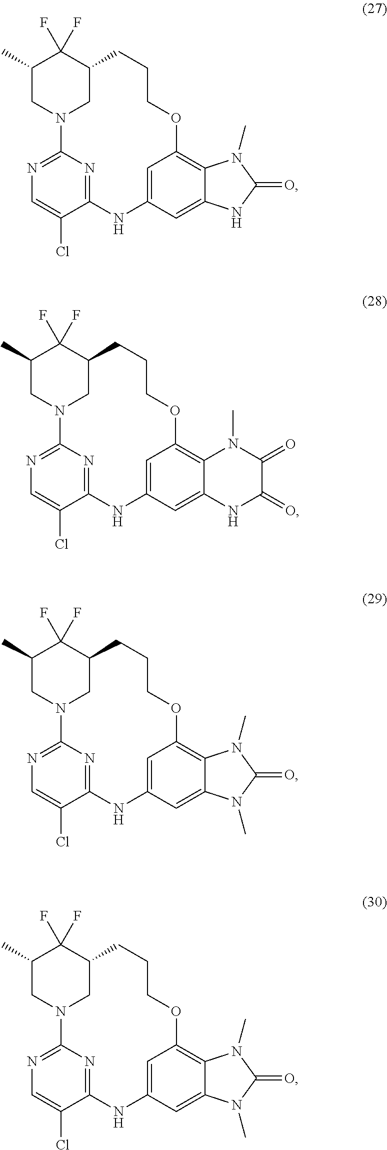 [SMILES: C[C@H]1CN2C[C@@H](CCCOc3cc(cc4[nH]c(=O)n(C)c34)Nc3nc2ncc3Cl)C1(F)F ; C[C@@H]1CN2C[C@H](CCCOc3cc(cc4[nH]c(=O)c(=O)n(C)c34)Nc3nc2ncc3Cl)C1(F)F ; C[C@@H]1CN2C[C@H](CCCOc3cc(cc4c3n(C)c(=O)n4C)Nc3nc2ncc3Cl)C1(F)F ; C[C@H]1CN2C[C@@H](CCCOc3cc(cc4c3n(C)c(=O)n4C)Nc3nc2ncc3Cl)C1(F)F]

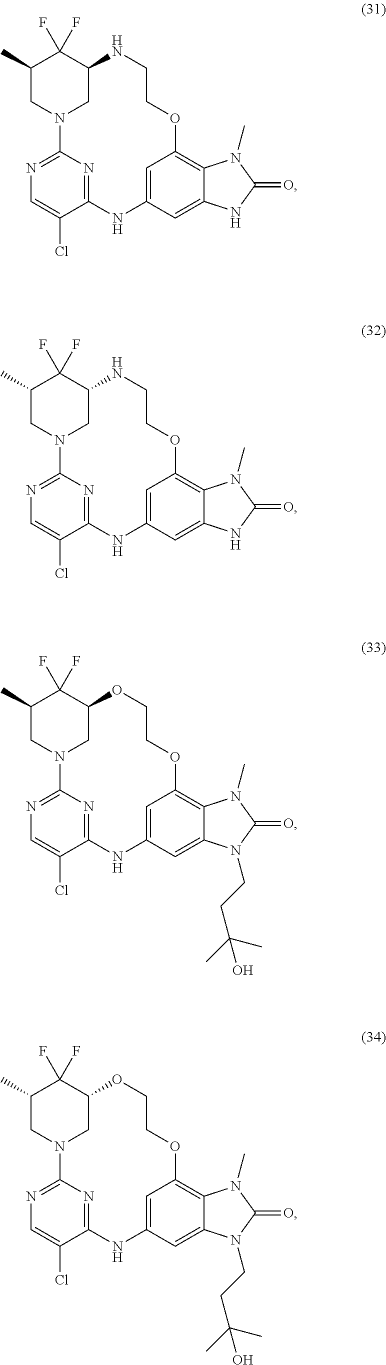 [SMILES: C[C@@H]1CN2C[C@H](NCCOc3cc(cc4[nH]c(=O)n(C)c34)Nc3nc2ncc3Cl)C1(F)F ; C[C@H]1CN2C[C@@H](NCCOc3cc(cc4[nH]c(=O)n(C)c34)Nc3nc2ncc3Cl)C1(F)F ; C[C@@H]1CN2C[C@H](OCCOc3cc(cc4c3n(C)c(=O)n4CCC(C)(C)O)Nc3nc2ncc3Cl)C1(F)F ; C[C@H]1CN2C[C@@H](OCCOc3cc(cc4c3n(C)c(=O)n4CCC(C)(C)O)Nc3nc2ncc3Cl)C1(F)F]

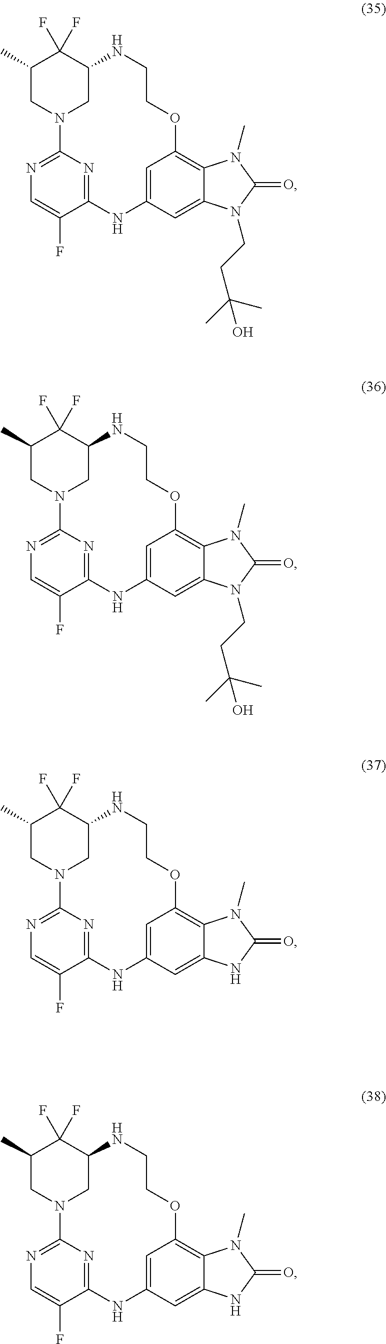 [SMILES: C[C@H]1CN2C[C@@H](NCCOc3cc(cc4c3n(C)c(=O)n4CCC(C)(C)O)Nc3nc2ncc3F)C1(F)F ; C[C@@H]1CN2C[C@H](NCCOc3cc(cc4c3n(C)c(=O)n4CCC(C)(C)O)Nc3nc2ncc3F)C1(F)F ; C[C@H]1CN2C[C@@H](NCCOc3cc(cc4[nH]c(=O)n(C)c34)Nc3nc2ncc3F)C1(F)F ; C[C@@H]1CN2C[C@H](NCCOc3cc(cc4[nH]c(=O)n(C)c34)Nc3nc2ncc3F)C1(F)F]

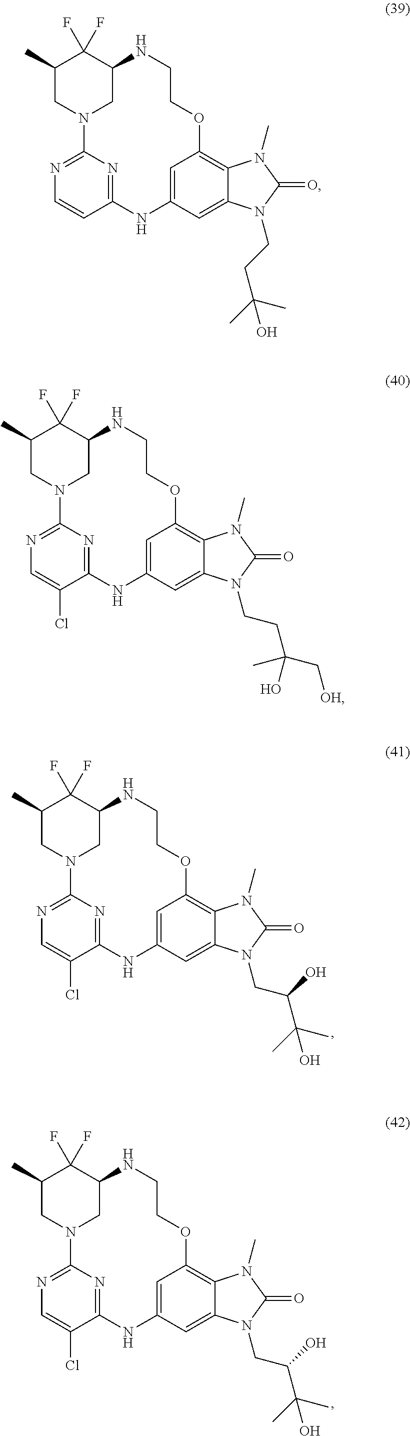

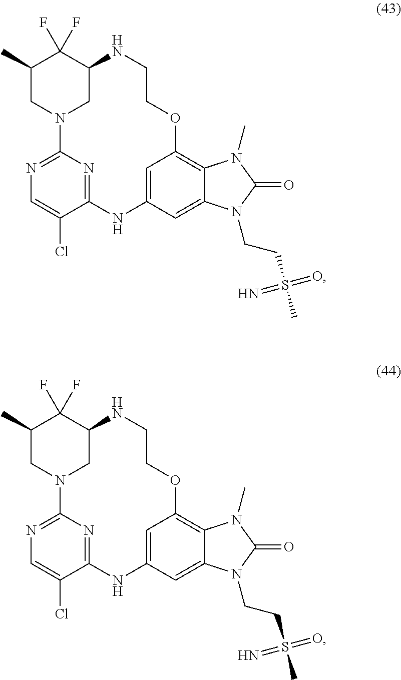 [SMILES: C[C@@H]1CN2C[C@H](NCCOc3cc(cc4c3n(C)c(=O)n4CC[S@](C)(=N)=O)Nc3nc2ncc3Cl)C1(F)F ; C[C@@H]1CN2C[C@H](NCCOc3cc(cc4c3n(C)c(=O)n4CC[S@@](C)(=N)=O)Nc3nc2ncc3Cl)C1(F)F]

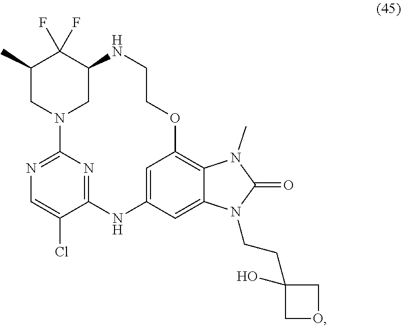 [SMILES: C[C@@H]1CN2C[C@H](NCCOc3cc(cc4c3n(C)c(=O)n4CCC3(O)COC3)Nc3nc2ncc3Cl)C1(F)F]

or a pharmaceutically acceptable salt or stereoisomer thereof.

Compounds of the present disclosure may be in the form of a free acid or free base, or a pharmaceutically acceptable salt. A pharmaceutically acceptable salt of the compounds of this disclosure can be formed, for example, by reaction of an appropriate free base of a compound of the invention and an appropriate pharmaceutically acceptable acid in a suitable solvent under standard conditions well known in the art. See, for example, Gould, P. L., “Salt selection for basic drugs,” International Journal of Pharmaceutics, 33:201-217 (1986); Bastin, R. J., et al., “Salt Selection and Optimization Procedures for Pharmaceutical New Chemical Entities,” Organic Process Research and Development, 4:427-435 (2000); and Berge, S. M., et al., “Pharmaceutical Salts,” Journal of Pharmaceutical Sciences, 66:1-19 (1977).

Compounds of the present disclosure may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present disclosure may be made and used in the form of individual isomers and substantially free of other isomers, or in the form of a mixture of various isomers, e.g., racemic mixtures of stereoisomers.

In some embodiments, the compound of formula (I) is an isotopic derivative in that it has at least one desired isotopic substitution of an atom, at an amount above the natural abundance of the isotope, i.e., enriched. In one embodiment, the compound includes deuterium or multiple deuterium atoms. As used herein, the term “hydrogen”, i.e. H, refers to all isotopes of hydrogen, including protium (1H) and deuterium (2H). As used herein, the term “compound” embraces isotopic derivatives.

Compounds of formula (I) may also be in the form of N-oxides, crystalline forms (also known as polymorphs), co-crystals, active metabolites of the compounds having the same type of activity, prodrugs, tautomers, and unsolvated as well as solvated (e.g., hydrated) forms with pharmaceutically acceptable solvents such as water, ethanol, and the like, of the compounds. As used herein, the term “compound” embraces all these forms.

The compounds of formula (I) may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, 1R spectroscopy, differential scanning calorimetry, powder X-ray diffractogram and/or other known techniques.

In some embodiments, the pharmaceutical composition comprises a co-crystal of a compound of formula (I). The term “co-crystal”, as used herein, refers to a stoichiometric multi-component system comprising a compound of formula (I) and a co-crystal former wherein the compound of formula (I) and the co-crystal former are connected by non-covalent interactions. The term “co-crystal former”, as used herein, refers to compounds which can form intermolecular interactions with a compound of formula (I) and co-crystallize with it. Representative examples of co-crystal formers include benzoic acid, succinic acid, fumaric acid, glutaric acid, trans-cinnamic acid, 2,5-dihydroxybenzoic acid, glycolic acid, trans-2-hexanoic acid, 2-hydroxycaproic acid, lactic acid, sorbic acid, tartaric acid, ferulic acid, suberic acid, picolinic acid, salicylic acid, maleic acid, saccharin, 4,4′-bipyridine p-aminosalicylic acid, nicotinamide, urea, isonicotinamide, methyl-4-hydroxybenzoate, adipic acid, terephthalic acid, resorcinol, pyrogallol, phloroglucinol, hydroxyquinol, isoniazid, theophylline, adenine, theobromine, phenacetin, phenazone, etofylline, and phenobarbital.

### Methods of Synthesis

In another aspect, the present disclosure is directed to a method for making a compound of formula (I), or a pharmaceutically acceptable salt or stereoisomer thereof. Broadly, the compounds of formula (I) or pharmaceutically acceptable salts or stereoisomers thereof may be prepared by any process known to be applicable to the preparation of chemically related compounds. The compounds of formula (I) will be better understood in connection with the synthetic schemes that described in various working examples that illustrate non-limiting methods by which the compounds of formula (I) may be prepared.

### Pharmaceutical Compositions

Another aspect of the present disclosure is directed to a pharmaceutical composition that includes a therapeutically effective amount of a compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present disclosure to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds of formula (I) are formulated for oral or intravenous administration (e.g., systemic intravenous injection).

Accordingly, compounds of formula (I) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of formula (I) may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of formula (I) may be administered in a local rather than systemic manner, for example, via injection of the conjugate directly into an organ, often in a depot preparation or sustained release formulation. In specific embodiments, long-acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The compounds of formula (I) may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of formula (I) may be formulated for topical administration which as used herein, refers to administration intradermally of the formulation to the epidermis. These types of compositions are typically in the form of ointments, pastes, creams, lotions, gels, solutions and sprays.

Representative examples of carriers useful in formulating compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I, and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, III. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a compound of formula (I) or a pharmaceutically acceptable salt or a stereoisomer thereof that is effective in producing the desired therapeutic response in a particular patient suffering from a disease or disorder mediated by aberrant BCL6 activity. The term “therapeutically effective amount” thus includes the amount of the compound or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer) cells, or reduces the amounts of BCL6 in diseased cells.

The total daily dosage of the compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the compound; and like factors well known in the medical arts (see, for example, Goodman and Gilman's, The Pharmacological Basis of Therapeutics, 10th Edition, A. Gilman, J. Hardman and L. Limbird, eds., McGraw-Hill Press, 155-173 (2001)).

### Methods of Use

In some aspects, the present disclosure is directed to treating diseases or disorders that involve (e.g., characterized or mediated by) aberrant (e.g., elevated levels of BCL6 or otherwise functionally abnormal e.g., deregulated BCL6 levels) BCL6 activity relative to a non-pathological state. Such diseases and disorders include cancers and inflammatory diseases and disorders. The methods entail administering a therapeutically effective amount of a compound of formula (I) or a pharmaceutically acceptable salt or stereoisomer thereof, to a subject in need thereof.

The term “subject” (or “patient”) as used herein includes all members of the animal kingdom prone to or suffering from the cancer. In some embodiments, the subject is a mammal, e.g., a human or a non-human mammal. The methods are also applicable to companion animals such as dogs and cats. A subject “in need of” treatment according to the present disclosure may be “suffering from or suspected of suffering from” a specific cancer may have been positively diagnosed or otherwise presents with a sufficient number of risk factors or a sufficient number or combination of signs or symptoms such that a medical professional could diagnose or suspect that the subject is suffering from the cancer. Thus, subjects suffering from a specific disease or disorder, and subjects suspected of suffering from a specific disease or disorder are not necessarily two distinct groups.

In some embodiments, the methods are directed to treating subjects having cancer. The methods embrace treatment of adult tumors/cancers and pediatric tumors/cancers. The cancers may be vascularized, or not yet substantially vascularized, or non-vascularized tumors.

In some embodiments, the cancer is a lymphoid malignancy.

In some embodiments, the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), or cutaneous T-cell lymphoma.

In some embodiments, methods of the present disclosure entail treatment of subjects having cell proliferative diseases or disorders of the hematological system.

In some embodiments, the methods are directed to treating subjects having an inflammatory disease or disorder.

In some embodiments, the inflammatory disease or disorder is inflammatory bowel disease, myocarditis, endometriosis, atherosclerosis, an allergic disease or disorder, or an autoimmune disease or disorder. In some embodiments, the allergic disease or disorder is asthma or pollinosis. In some embodiments, the autoimmune disease is noninfectious meningitis, autoimmune encephalitis, transverse myelitis, or acute disseminated encephalomyelitis.

Compounds of formula (I) may be administered to a cancer patient as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which were unsuccessful or partially successful but who became unresponsive or intolerant to the particular treatment. Therapy may also be given as an adjuvant treatment, i.e., to prevent reoccurrence of cancer in patients with no currently detectable disease or after surgical removal of a tumor. Thus, in some embodiments, the compounds may be administered to a patient who has received another therapy, such as chemotherapy, radioimmunotherapy, surgical therapy, immunotherapy, radiation therapy, targeted therapy or any combination thereof.

The methods of the present disclosure may entail administration of a compound of formula (I) or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day “off” period. In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of 5 days.

### Combination Therapy

The compounds of formula (I) and their pharmaceutically acceptable salts and stereoisomers may be used in combination or concurrently with at least one other active agent, e.g., anti-cancer agent or regimen, in treating cancer. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of formula (I) in combination with one or more additional therapeutics known for use in treating cancer. The dosage of the additional therapeutic may be the same or even lower than known or recommended doses. See, Hardman et al., eds., Goodman & Gilman's the Pharmacological Basis of Basis of Therapeutics, 10th ed., McGraw-Hill, New York, 2001; Physician's Desk Reference 60th ed., 2006. Anti-cancer agents that may be suitable for use in combination with the compounds are known in the art. See, e.g., U.S. Pat. No. 9,101,622 (Section 5.2 thereof) and U.S. Pat. No. 9,345,705 B2 (Columns 12-18 thereof). Representative examples of additional anti-cancer agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy.

In some embodiments, a compound of formula (I) and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

When the active components of the combination are not administered in the same pharmaceutical composition, it is understood that they can be administered in any order to a subject in need thereof. For example, a compound of the present disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional therapeutic, to a subject in need thereof. In various aspects, the therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one example, the (e.g., anticancer) therapeutics are administered within the same office visit. In another example, the combination anticancer therapeutics may be administered at 1 minute to 24 hours apart.

In some embodiments, a compound of formula (I) and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, the compounds of the present disclosure may be used in combination with other anti-cancer agents, examples of which include Etoposide (e.g., lymphomas, and non-lymphocytic leukemia), Vincristine (e.g., leukemia), Daunorubicin (e.g., acute myeloid leukemia (AML), acute lymphocytic leukemia (ALL), chronic myelogenous leukemia (CML), and Kaposi's sarcoma), Rituximab (e.g., non-Hodgkin's lymphoma), Alemtuzumab (e.g., chronic lymphocytic leukemia (CLL), cutaneous T-cell lymphoma (CTCL) and T-cell lymphoma), Bortezomib (e.g., multiple myeloma and mantle cell lymphoma), Pegaspargase (e.g., acute lymphoblastic leukemia), Keytruda® (e.g., Hodgkin lymphoma), and dexamethasone (e.g., acute multiple myeloma).

In some embodiments, the additional anti-cancer agent is an enhancer of zeste homolog 2 (EZH2) inhibitor, examples of which include tazemetostat, GSK126, lirametostat (CPI-1205), CPI-0209, PF-06821497, SHR₂₅₅₄, HH2853, valemetostat (DS3201), MAK-683, and FTX-6058.

These and other aspects of the present disclosure will be further appreciated upon consideration of the following Examples, which are intended to illustrate certain particular embodiments of the disclosure but are not intended to limit its scope, as defined by the claims.

### Examples

### Example 1: Synthesis of 2-(((138, 15R)-2⁵-chloro-1⁴, 1⁴-difluoro-1⁵,4¹-dimethyl-4²-oxo-4¹,4²-dihydro-5-oxa-3-aza-4(6.8)-quinolina-2(2,4)-pyrimidina-1 (1,3)-piperidinacyclooctaphane-43-yl)oxy)-N-methylacetamide (4)

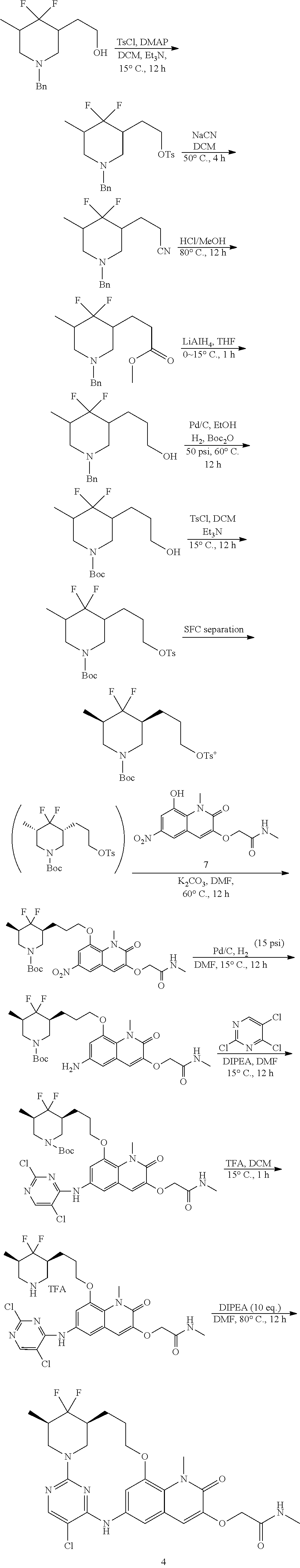

### 2-(1-Benzyl-4,4-difluoro-5-methyl-3-piperidyl)ethyl-4-methylbenzenesulfonate

To a solution of 2-(1-benzyl-4,4-difluoro-5-methyl-3-piperidyl) ethanol (10 g, 37.13 mmol, 1 eq) in DCM (210 mL) were added 4-methylbenzenesulfonyl chloride (8.49 g, 44.56 mmol, 1.2 eq), DMAP (453.60 mg, 3.71 mmol, 0.1 eq) and TEA (7,51 g, 74.26 mmol, 10.34 mL, 2 eq). The mixture was stirred at 15° C. for 12 hr. After addition of water (200 mL), the solution (combined with another batch with the same scale) was extracted with DCM (300 mL×3). The combined organic phase was washed with brine (300 mL), dried over anhydrous Na₂SO₄, filtered and the filtrate was concentrated in vacuo. The residue was purified by flash silica gel chromatography (Silica Flash Column, eluent of 0˜50% ethyl acetate/petroleum ether gradient) to give the title compound as a yellow oil (22 g, 47.27 mmol, 64% yield, 91% purity). ¹H NMR (400 MHz, DMSO-d₆) δ ppm 7.75 (d, J=8.11 Hz, 2H), 7.47 (br d, J=7.87 Hz, 2H), 7.24-7.37 (m, 5H), 3.98-4.13 (m, 2H), 3.37-3.54 (m, 2H), 2.65-2.81 (m, 2H), 2.39 (s, 3H), 1.97-2.03 (m, 1H), 1.76-1.94 (m, 3H), 1.38-1.49 (m, 1H), 1.14-1.22 (m, 1H), 0.84 (d, J=6.68 Hz, 3H).

### 3-(1-Benzyl-4,4-difluoro-5-methyl-3-piperidyl)propanenitrile

To a mixture of 2-(1-benzyl-4,4-difluoro-5-methyl-3-piperidyl)ethyl-4-methyl-benzenesulfonate (22 g, 51.95 mmol, 1 eq) in DMSO (450 mL) was added NaCN(11.55 g, 235.68 mmol, 4.54 eq) under N₂. The mixture was stirred at 50° C. for 4 hr. The reaction mixture was cooled to 15° C., diluted with H₂O (1000 mL), extracted with EtOAc (1000 mL×2), the combined organic phase was washed with brine (1000 mL), dried over anhydrous Na₂SO₄, filtered and concentrated in vacuo. The residue was purified by flash silica gel chromatography (Silica Flash Column, eluent of 0˜50% ethyl acetate/petroleum ether gradient) to give the title compound as a yellow oil (8 g, 25.29 mmol, 49% yield, 88% purity). ¹H NMR (400 MHz, DMSO-d₆) δ ppm 7.21-7.40 (m, 5H), 3.56-3.70 (m, 1H), 3.41-3.52 (m, 1H), 2.90-3.11 (m, 1H), 2.67-2.79 (m, 1H), 2.52-2.63 (m, 2H), 2.02-2.20 (m, 2H), 1.78-1.98 (m, 3H), 1.47 (dq, J=14.38, 7.37 Hz, 1H), 0.88 (d, J=6.79 Hz, 3H)

### Methyl 3-(1-benzyl-4,4-difluoro-5-methyl-3-piperidyl)propanoate

A mixture of 3-(1-benzyl-4,4-difluoro-5-methyl-3-piperidyl)propanenitrile (8 g, 28.74 mmol, 1 eq) in HCl/MeOH (160 mL) was stirred at 80° C. for 12 hr. The mixture was concentrated in vacuo. To the residue was added sat, aq. NaHCO₃ (100 mL) to pH=8, then it was extracted with ethyl acetate (100 mL×2). The combined organic phase was washed with brine (100 mL), dried with anhydrous Na₂SO₄, filtered and the filtrate was concentrated in vacuo. The residue was purified by flash silica gel chromatography (Silica Flash Column, eluent of 0˜50% ethyl acetate/petroleum ether gradient) to give the title compound as a yellow oil (4.9 g, 15.74 mmol, 55% yield). ¹H NMR (400 MHz, CDCl3) δ ppm 7.25-7.38 (m, 5H), 3.66 (s, 3H), 3.45-3.60 (m, 2H), 2.82-2.91 (m, 1H), 2.69-2.80 (m, 1H), 2.39 (t, J=7.70 Hz, 2H), 1.90-2.19 (m, 5H), 1.48-1.61 (m, 1H), 0.97 (d, J=6.72 Hz, 3H).

### 3-(1-Benzyl-4,4-difluoro-5-methyl-3-piperidyl)propan-1-ol

To a mixture of methyl 3-(1-benzyl-4,4-difluoro-5-methyl-3-piperidyl)propanoate (7.07 g, 22.71 mmol, 1 eq) in THF (70 mL) was added LiAlH₄ (1.03 g, 27.25 mmol, 1.2 eq) slowly at 0° C. under N₂. The mixture was stirred at 15° C. for 1 hour. To the mixture was added water (˜1 mL) at 15° C. dropwise until no bubbles occurred. The mixture was dried with anhydrous Na2SO₄ (˜20 g) and stirred at 15° C. for 10 min. Then the mixture was filtered and the filtrate was concentrated in vacuo to give the title compound as a colorless oil (4.9 g, 15.04 mmol, 66% yield, 87% purity). ¹H NMR. (400 MHz, CDCl) δ ppm 7.27-7.19 (m, 5H), 3.56-3.38 (m, 4H), 2.84-2.82 (m, 1H), 2.65-2.62 (m, 1H), 1.96-1.87 (m, 4H), 1.75-1.65 (m, 1H), 1.53-1.49 (m, 3H), 1.20-1.16 (m, 1H), 0.88 (d, J=6.80 Hz, 3H).

### tert-Butyl 4,4-difluoro-3-(3-hydroxypropyl)-5-methyl-piperidine-1-carboxylate

To a solution of 3-(1-benzyl-4,4-difluoro-5-methyl-3-piperidyl)propan-1-ol (4.9 g, 17.29 mmol, 1 eq) in EtOH (50 mL) was added Pd/C (1 g, 10% purity) and Boc₂O (7,55 g, 34.59 mmol, 7.95 mL, 2 eq) under Ar. The suspension was degassed under vacuum and purged with H₂ several times. The mixture was stirred under H₂ (50 psi) at 60° C. for 12 hr. The reaction mixture was filtered and the filtrate was concentrated in vacuo to give the title compound as a gray oil (6.6 g, crude). ¹H NMR (400 MHz, CDCl₃) δ ppm 4.22-3.95 (m, 3H), 3.67-3.63 (m, 2H), 2.57 (s, 2H), 1.86-1.82 (m, 2H), 1.66-1.60 (m, 2H), 1.47-1.45 (m, 9H), 1.29-1.25 (m, 2H), 1.00 (d, J=6.40 Hz, 3H).

### tert-Butyl4,4-difluoro-3-methyl-5-[3-(p-tolylsulfonyloxy)propyl]piperidine-1-carboxylate

To a solution of tert-butyl 4,4-difluoro-3-(3-hydroxypropyl)-5-methyl-piperidine-1-carboxylate (6.6 g, 22.50 mmol, 1 eq) in DCM (70 mL) were added TEA (4.55 g, 45.00 mmol, 6.26 mL, 2 eq) and TsCl (4.29 g, 22.50 mmol, 1 eq) at 15° C. The mixture was stirred at 15° C. for 12 hr. Water (100 mL) was added to the mixture, following by extracting with DCM (100 mL×2). The combined organic phase was washed with brine (120 mL), dried with anhydrous Na₂SO₄, filtered and the filtrate was concentrated in vacuo. The residue was purified by silica gel chromatography (column height: 250 mm, diameter: 100 mm, 100-200 mesh silica gel, petroleum ether/ethyl acetate=10/1, 3/1) to give the title compound as a yellow oil (5.6 g, 12.30 mmol, 55% yield, 98% purity). ¹H NMR (400 MHz, CDCl₃) δ ppm 7.79 (d, J=8.40 Hz, 2H), 7.36 (d, J=8.00 Hz, 2H), 4.13-4.02 (m, 4H), 2.56 (br s, 2H), 2.46 (s, 3H), 1.86-1.83 (m, 1H), 1.75-1.70 (m, 4H), 1.46 (s, 9H), 1.28-1.23 (m, 1H), 1.00 (d, J=6.80 Hz, 3H).

### tert-butyl (3R,5S)-4,4-difluoro-3-methyl-5-[3-(p-tolylsulfonyloxy)propyl]piperidine-1-carboxylate (10C) and tert-butyl (3S,5R)-4,4-difluoro-3-methyl-5-[3-(p-tolylsulfonyloxy)propyl]piperidine-1-carboxylate

tert-Butyl-4,4-difluoro-3-methyl-5-[3-(p-tolylsulfonyloxy)propyl]piperidine-1-carboxylate (3 g, 6.70 mmol, 1 eq) was separated by SFC (column: DAICEL CHIRALPAKIC (250 mm*50 mm, 10 μm); mobile phase: [0.1% NH₃H₂O IPA]; B %: 35%-35%, 4.5 min) to give tert-butyl (3R,5S)-4,4-difluoro-3-methyl-5-[3-(p-tolylsulfonyloxy)propyl]piperidine-1-carboxylate (1.3 g, 2.87 mmol, 43% yield, 99% purity) as a colorless oil and tert-butyl (3S,5R)-4,4-difluoro-3-methyl-5-[3-(p-tolylsulfonyloxy)propyl]piperidine-1-carboxylate (1.3 g, 2.84 mmol, 42% yield, 98% purity) as a colorless oil. ¹H NMR (400 MHz, CDCl₃) δ ppm 7.79 (d, J=8.11 Hz, 2H), 7.35 (br d, J=7.99 Hz, 2H), 3.85-4.19 (m, 4H), 2.35-2.75 (m, 5H), 1.67-1.97 (m, 5H), 1.46 (s, 9H), 1.20-1.32 (m, 1H), 0.96-1.04 (m, 3H). ¹H NMR (400 MHz, CDCl₃) δppm 7.79 (d, J=8.34 Hz, 2H), 7.36 (d, J=7.99 Hz, 2H), 3.88-4.17 (m, 4H), 2.48-2.68 (m, 2H), 2.46 (s, 3H), 1.85 (br s, 1H), 1.63-1.80 (m, 4H), 1.46 (s, 9H), 1.18-1.32 (m, 1H), 1.00 (d, J=6.68 Hz, 3H).

### tert-Butyl (3R,5S)-4,4-difluoro-3-methyl-5-[3-[[1-methyl-3-[2-(methylamino)-2-oxo-ethoxy]-6-nitro-2-oxo-8-quinolyl]oxy]propyl]piperidine-1-carboxylate

To a mixture of 2-[(8-hydroxy-1-methyl-6-nitro-2-oxo-3-quinolyl)oxy]—N-methyl-acetamide (100 mg, 325.46 μmol, 1 eq) and tert-butyl (3R,5S)-4,4-difluoro-3-methyl-5-[3-(p-tolylsulfonyloxy)propyl]piperidine-1-carboxylate (218.48 mg, 488.19 μmol, 1.5 eq) in DMF (1 mL) was added K₂CO₃ (89.96 mg, 650.92 μmol, 2 eq). The reaction mixture was then stirred at 60° C. for 12 hr. The reaction mixture (combined with another batch with 60 mg scale) was treated with H₂O (10 mL) and extracted with EtOAc (10 mL×3), the combined organic phase was washed with brine (20 mL), dried with anhydrous Na₂SO₄, filtered and the filtrate was concentrated in vacuo. The residue was triturated with (petroleum ether/ethyl acetate=1:1, 5 mL) at 15° C. for 30 min to give the title compound as a white solid (200 mg, 83% purity). ¹H NMR (400 MHz, DMSO-d₆) δ ppm 8.22 (br s, 1H), 7.90 (br d, J=2.45 Hz, 1H), 7.77 (br s, 1H), 7.46 (s, 1H), 4.57 (s, 2H), 4.23 (br s, 2H), 4.00-4.15 (m, 1H), 3.90 (s, 4H), 2.56-2.69 (m, 5H), 1.83-2.08 (m, 5H), 1.40 (s, 10H), 0.94 (br d,)=6.48 Hz, 3H). LCMS: [M+H]⁺=583.4.

### tert-Butyl (3S,5R)-3-[3-[[6-amino-1-methyl-3-[2-(methylamino)-2-oxo-ethoxy]-2-oxo-8-quinolyl]oxy]propyl]-4,4-difluoro-5-methyl-piperidine-1-carboxylate

To a solution of tert-butyl (3R,5S)-4,4-difluoro-3-methyl-5-[3-[[1-methyl-3-[2-(methylamino)-2-oxo-ethoxy]-6-nitro-2-oxo-8-quinolyl]oxy]propyl]piperidine-1-carboxylate (150.00 mg, 257.47 μmol, 1 eq) in DMF (3 mL) was added Pd/C (0.1 g, 10% purity) under Ar. The suspension was degassed under vacuum and purged 3 times with H₂. The mixture was stirred under H₂ (15 psi) at 15° C. for 12 hr. The reaction mixture (combined with another batch with 50 mg scale) was filtered and the filtrate was concentrated in vacuo to give the title compound as a yellow oil (160 mg, crude). ¹H NMR (400 MHz, CDCL) δ ppm 7.36-7.46 (m, 1H), 6.76 (s, 1H), 6.39 (d, J=2.27 Hz, 1H), 6.34 (d,/=2.27 Hz, 1H), 4.51 (s, 2H), 4.16-4.35 (m, 1H), 4.01 (br t, J=5.84 Hz, 2H), 3.97 (s, 3H), 3.65-3.84 (m, 1H), 2.89 (br s, 2H), 2.51-2.71 (m, 2H), 1.75-2.08 (m, 6H), 1.47 (s, 9H), 1.37-1.45 (m, 1H), 1.03 (d, J=6.68 Hz, 3H). LCMS: [M+H]⁺=553.2.

### tert-Butyl (3S,5R)-3-[3-[[6-[(2,5-dichloropyrimidin-4-yl)amino]-1-methyl-3-[2-(methylamino)-2-oxo-ethoxy]-2-oxo-8-quinolyl]oxy]propyl]-4,4-difluoro-5-methyl-piperidine-1-carboxylate

To a mixture of tert-butyl (3S,5R)-3-[3-[[6-amino-1-methyl-3-[2-(methylamino)-2-oxo-ethoxy]-2-oxo-8-quinolyl]oxy]propyl]-4,4-difluoro-5-methyl-piperidine-1-carboxylate (100 mg, 180.96 μmol, 1 eq) and 2,4,5-trichloropyrimidine (66.38 mg, 361.92 μmol, 2 eq) in DMF (1 mL) was added DIPEA (58.47 mg, 452.40 μmol, 78.80 μL, 2.5 eq). The reaction mixture was then stirred at 15° C. for 12 hr. The reaction mixture (combined with another batch with 30 mg scale) was treated with H₂O (2 mL), then the suspension was filtered and the solid was collected and dried in vacuo. The residue was purified by column chromatography (SiO₂, petroleum ether/THF =1/1 to 0/1) to give the title compound as a white solid (130 mg, 73% purity). ¹H NMR (400 MHz, DMSO-d₆) δ ppm 9.57 (br s, 1H), 8.39 (br s, 1H), 7.92 (br s, 1H), 7.44 (br d, J=7.63 Hz, 2H), 7.13 (br s, 1H), 4.57 (br s, 2H), 4.09 (br s, 3H), 3.89 (br s, 4H), 2.58-2.73 (m, 5H), 1.81-2.02 (m, 5H), 1.39 (br s, 10H), 0.94 (br d, J=5.75 Hz, 3H). LCMS: [M+H]⁺=699.4.

### 2-[[6-[(2,5-Dichloropyrimidin-4-yl)amino]-8-[3-[(3S,5R)-4,4-difluoro-5-methyl-3-piperidyl]propoxy]-1-methyl-2-oxo-3-quinolyl]oxy]—N-methyl-acetamide

A solution of tert-butyl (3S,5R)-3-[3-[[6-[(2,5-dichloropyrimidin-4-yl)amino]-1-methyl-3-[2˜ (methylamino)-2-oxo-ethoxy]-2-oxo-8-quinolyl]oxy]propyl]-4,4-difluoro-5-methyl-piperidine-1-carboxylate (130 mg, 185.83 μmol, 1 eq) in DCM (4 mL) and TFA (1.0 mL) was stirred at 15° C. for 1 hr. The reaction mixture was concentrated in vacuo to give the title compound as a yellow oil (150 mg, crude, TFA salt). LCMS: [M+H]⁺=599.2.

### 2-(((1³S, 1⁵R)-2⁵-chloro-1⁴,1⁴-difluoro-13,41-dimethyl-4²-oxo-4¹,4²-dihydro-5-oxa-3-aza-4(6,8)-quinolina-2(2,4)-pyrimidina-1 (1,3)-piperidinacyclooctaphane-4³-yl)oxy)-N-methylacetamide (4)

To a solution of 2-[[6-[(2,5-dichloropyrimidin-4-yl)amino]-8-[3-[(3S,5R)-4,4-difluoro-5-methyl-3-piperidyl]propoxy]-1-methyl-2-oxo-3-quinolyl]oxy]—N-methyl-acetamide (150 mg, 210.24 μmol, 1 eq, TFA) in DMF (1.5 mL) was added DIPEA (271.71 mg, 2.10 mmol, 366.19 UL, 10 eq). The reaction mixture was then stirred at 80° C. for 12 hr. The reaction mixture was concentrated in vacuo to give a residue which was purified by prep-HPLC (column: Phenomenex C18 75*30 mm*3 μm, mobile phase: [water (NH₄HCO₃)-ACN], B %: 45%-75%, 8 min) to give the title compound as a white solid (15 mg, 25.84 μmol, 12% yield, 97% purity). ¹H NMR (400 MHz, DMSO-d₆) δ ppm 8.99 (s, 1H), 8.10 (s, 1H), 7.95 (br d, J=4.29 Hz, 1H), 7.86 (d, J=1.55 Hz, 1H), 7.06 (s, 2H), 4.45-4.72 (m, 5H), 4.14-4.24 (m, 1H), 3.84 (s, 3H), 2.74 (br t, J=12.64 Hz, 1H), 2.66 (d, J=4.53 Hz, 3H), 2.53-2.60 (m, 1H), 2.29-2.42 (m, 1H), 1.84-2.02 (m, 2H), 1.59-1.81 (m, 2H), 1.32-1.42 (m, 1H), 0.97 (d, J=6.68 Hz, 3H). LCMS: [M+H]⁺=563.3.

### Example 2: Synthesis of 2-(((1³R,1⁵S)-2⁵-chloro-1⁴, 1⁴ difluoro-1⁵,4¹-dimethyl-4²-oxo-4¹4² dihydro-5-oxa-3-aza-4(6,8)-quinolina-2(2,4)-pyrimidina-1 (1,3)-piperidinacyclooctaphane-4³-yl)oxy)-N-methylacetamide (5)

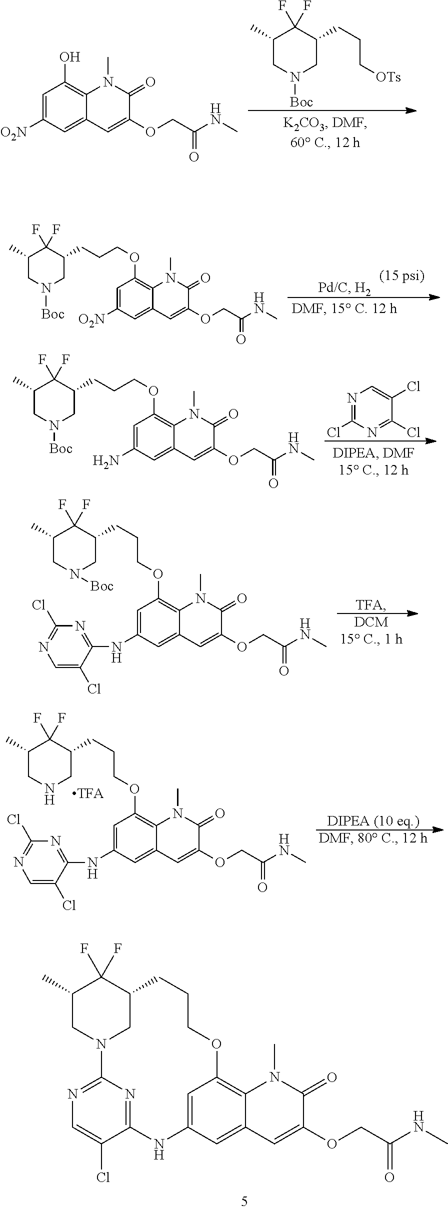

### tert-Butyl (3S,5R)-4,4-difluoro-3-methyl-5-[3-[[1-methyl-3-[2-(methylamino)-2-oxo-ethoxy]-6-nitro-2-oxo-8-quinolyl]oxy]propyl]piperidine-1-carboxylate

To a solution of 2-[(8-hydroxy-1-methyl-6-nitro-2-oxo-3-quinolyl)oxy]—N-methyl-acetamide (120 mg, 390.55 μmol, 1 eq) in DMF (2 mL) was added K₂CO₃ (107.96 mg, 781.10 μmol, 2 and eq) tert-butyl (3S,5R)-4,4-difluoro-3-methyl-5-[3-(ptolylsulfonyloxy)propyl]piperidine-1-carboxylate (262.18 mg, 585.83 μmol, 1.5 eq). The mixture was stirred at 60° C. for 12 hr. The reaction mixture (combined with another batch with 30 mg scale) was cooled to 20° C., and added water (2 mL). The mixture was filtered and the filter cake was washed with water (5 mL), then the filter cake was dried in vacuo. The crude product was triturated with petroleum ether:ethyl acetate=1/1 (˜2 mL) at 20° C. for 5 min. The mixture was filtered and the filter cake was dried in vacuo to give the title compound as a white solid (130 mg).

¹H NMR (400 MHz, DMSO-d₆) δ ppm 8.23 (d, J=2.20 Hz, 1H), 7.93 (br d, J=4.16 Hz, 1H), 7.76 (d, J=2.20 Hz, 1H), 7.46 (s, 1H), 4.57 (s, 2H), 4.23 (br t, J=5.20 Hz, 2H), 3.99-4.17 (m, 1H), 3.90 (s, 4H), 2.66 (d, J=4.52 Hz, 5H), 1.79-2.11 (m, 5H), 1.40 (s, 10H), 0.94 (d, J=6.60 Hz, 3H).

### tert-Butyl (3R,5S)-3-[3-[[6-amino-1-methyl-3-[2-(methylamino) ˜2-oxo-ethoxy]-2-oxo-8-quinolyl]oxy]propyl]-4,4-difluoro-5-methyl-piperidine-1-carboxylate

To a solution of tert-butyl (3S,5R)-4,4-difluoro-3-methyl-5-[3-[[1-methyl-3-[2-(methylamino)-2-oxo-ethoxy]-6-nitro-2-oxo-8-quinolyl]oxy]propyl]piperidine-1-carboxylate (100 mg, 171.65 μmol, 1 eq) in DMF (2 mL) was added Pd/C (40 mg, 10% purity) under Ar. The suspension was degassed under vacuum and purged 3 times with H₂. The mixture was stirred under H₂ (15 psi) at 15° C. for 12 hr. The reaction mixture (combined with another batch with 30 mg scale) was filtered through a pad of Celite® and the filtrate was concentrated in vacuo to give the title compound as a yellow solid (120 mg, crude). ¹H NMR (400 MHz, DMSO-d₆) δ ppm 7.85-7.98 (m, 2H), 6.94 (s, 1H), 6.45 (d, J=2.13 Hz, 1H), 6.26 (d, J=2.25 Hz, 1H), 4.49 (s, 1H), 3.75-3.83 (m, 3H), 3.26-3.33 (m, 4H), 2.89 (s, 4H), 2.73 (s, 4H), 1.79-2.05 (m, 5H), 1.40 (s, 9H), 0.94 (d, J=6.75 Hz, 3H).

### tert-Butyl (3R,5S)-3-[3-[[6-[(2,5-dichloropyrimidin-4-yl)amino]-1-methyl-3-[2-(methylamino)-2-oxo-ethoxy]-2-oxo-8-quinolyl]oxy]propyl]-4,4-difluoro-5-methyl-piperidine-1-carboxylate

To a mixture of tert-butyl (3R,5S)-3-[3-[[6-amino-1-methyl-3-[2-(methylamino)-2-oxo-ethoxy]-2-oxo-8-quinolyl]oxy]propyl]-4,4-difluoro-5-methyl-piperidine-1-carboxylate (100 mg, 180.96 μmol, 1 eq) and 2,4,5-trichloropyrimidine (66.38 mg, 361.92 μmol, 2 eq) in DMF (1 mL) was added DIEA (58.47 mg, 452.40 μmol, 78.80 μL, 2.5 eq). The reaction mixture was then stirred at 15° C. for 12 hr. The reaction mixture (combined with another batch with 20 mg scale) was treated with H₂O) (2 mL). Then the suspension was filtered and the solid was collected and dried in vacuo. The residue was purified by column chromatography (SiO₂, petroleum ether/THF=1/1 to 0/1) to give the title compound as a black solid (110 mg, 94% purity). ¹H NMR (400 MHz, DMSO-d₆) δ ppm 9.58 (s, 1H), 8.40 (s, 1H), 7.93 (br d, J=4.50 Hz, 1H), 7.44 (br d, J=7.63 Hz, 2H), 7.13 (s, 1H), 4.57 (s, 2H), 4.09 (br t, J=5.57 Hz, 2H), 3.89 (s, 4H), 3.30 (s, 2H), 2.55-2.71 (m, 5H), 1.81-2.08 (m, 5H), 1.39 (s, 9H), 0.94 (d, J=6.75 Hz, 3H).

### 2-[[6-[(2,5-Dichloropyrimidin-4-yl)amino]-8-[3-[(3R,5S)-4,4-difluoro-5-methyl-3-piperidyl]propoxy]-1-methyl-2-oxo-3-quinolyl]oxy]—N-methyl-acetamide

A solution of tert-butyl (3R,5S)-3-[3-[[6-[(2,5-dichloropyrimidin-4-yl)amino]-1-methyl-3-[2-(methylamino)-2-oxo-ethoxy]-2-oxo-8-quinolyl]oxy]propyl]-4,4-difluoro-5-methyl-piperidine-1-carboxylate (80 mg, 114.36 μmol, 1 eq) in TFA (800.00 μL) and DCM (2.4 mL) was stirred at 15° C. for 1 hr. The solution was concentrated in vacuo to give the title compound as a yellow oil (80 mg, crude, TFA salt). LCMS: [M+H]⁺=599.3.

### 2-(((1³R, 1⁵S)-2⁵-Chloro-1⁴, 1⁴-difluoro-1⁵,4¹-dimethyl-4²-oxo-4¹,4²-dihydro-5-oxa-3-aza-4(6,8)-quinolina-2(2,4)-pyrimidina-1 (1,3)-piperidinacyclooctaphane-4³-yl)oxy)-N-methylacetamide (5)

A mixture of 2-[[6-[(2,5-dichloropyrimidin-4-yl)amino]-8-[3-[(3R,5S)-4,4-difluoro-5-methyl-3-piperidyl]propoxy]-1-methyl-2-oxo-3-quinolyl]oxy]—N-methyl-acetamide (80 mg, 112.13 μmol, 1 eq, TFA), DIEA (144.92 mg, 1.12 mmol, 195.30 μL, 10 eq) in DMF (1 mL) was stirred at 80° C. for 12 hr. The solution (combined with another batch with 30 mg) was concentrated in vacuo. The residue was purified by prep-HPLC (column: Phenomenex C18 75*30 mm*3 μm; mobile phase: [water (NH₄HCO₃)-ACN]; B %: 30%-60%, 8 min) to give the title compound as a white solid (10 mg, 96% purity). ¹H NMR (400 MHz, DMSO-d₆) δ ppm 8.99 (s, 1H), 8.11 (s, 1H), 7.91-7.98 (m, 1H), 7.86 (d, J=1.83 Hz, 1H), 7.01-7.09 (m, 2H), 4.64-4.72 (m, 1H), 4.54-4.63 (m, 3H), 4.49-4.54 (m, 1H), 4.14-4.24 (m, 1H), 3.85 (s, 3H), 2.74 (br t, J=12.53 Hz, 1H), 2.66 (d, J=4.65 Hz, 3H), 2.55 (br s, 1H), 2.29-2.44 (m, 1H), 1.82-2.05 (m, 2H), 1.59-1.82 (m, 2H), 1.28-1.42 (m, 1H), 0.97 (d, J=6.72 Hz, 3H). LCMS: [M+H]⁺=563.3.

### Example 3: Synthesis of 2-(((1³R, 1⁵S)-2⁵-chloro-1⁵,4¹-dimethyl-4²-oxo-4¹, 4²-dihydro-5,8-dioxa-3-aza-4(6.8)-quinolina-2(2,4)-pyrimidina-1 (1,3)-piperidinacyclooctaphane-4³-yl)oxy)-N-methylacetamide (6)

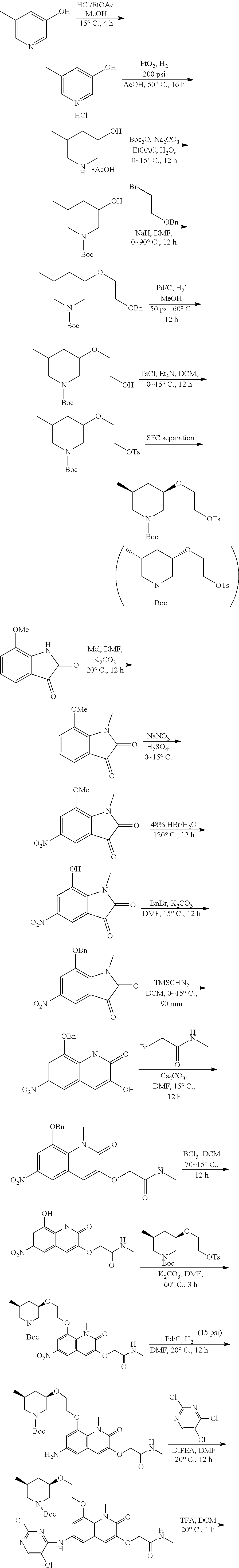

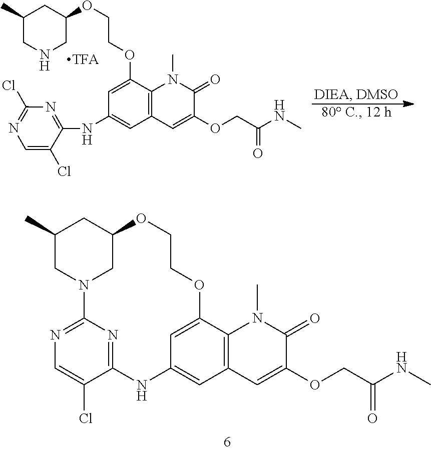

### 5-Methylpyridin-3-ol hydrochloride

To a solution of 5-methylpyridin-3-ol (10 g, 91.64 mmol, 1 eq) in EtOAc (30 mL) and MeOH (10 mL) was added HCl/EtOAc (4 M, 160 mL) in one portion at 15° C. The mixture was stirred at 15° C. for 4 hr. The mixture was filtered, and the filter cake was dried in vacuo to give the title compound as a white solid (26.68 g, crude, HCl salt). ¹H NMR (400 MHz, DMSO-d₆) δ ppm 11.97 (b, 1H), 8.19-8.42 (m, 2H), 7.88 (s, 1H), 2.40 (s, 3H).

### 5-Methylpiperidin-3-ol

To a solution of 5-methylpyridin-3-ol (13 g, 89.29 mmol, 1 eq, HCl) in AcOH (110 mL) was added PtO₂ (1.30 g, 8.93 mmol, 0.1 eq) under Ar. The suspension was degassed under vacuum and purged several times with H₂. The mixture was stirred under H₂ (200 psi) at 50° C. for 16 hr. The mixture was filtered by Celite®, and the filtrate was concentrated in vacuo to give the title compound as a yellow oil (33 g, crude, HOAc). ¹H NMR (400 MHz, DMSO-d₆) δ ppm 8.94-9.58 (m, 2H), 3.67-3.83 (m, 1H), 2.94-3.22 (m, 2H), 2.67 (br t, J=10.01 Hz, 1H), 2.24-2.48 (m, 2H), 1.58-1.85 (m, 2H), 0.96-1.17 (m, 1H), 0.87 (dd, J=8.38, 6.75 Hz, 3H).

### tert-Butyl 3-hydroxy-5-methyl-piperidine-1-carboxylate

To a mixture of 5-methylpiperidin-3-ol (11 g, 62.78 mmol, 1 eq, HOAc) and Na₂CO₃ (13.31 g, 125.55 mmol, 2 eq) in EtOAc (100 mL) and H₂O (100 mL) was added Boc₂O (13.70 g, 62.78 mmol, 14.42 mL, 1 eq) dropwise at 0° C. under N₂. Then the mixture was stirred at 15° C. for 12 hr. The mixture (combined with the other 2 batches with same scale) was extracted with ethyl acetate (200 mL×2). The combined organic phase was washed with brine (500 mL), dried with anhydrous Na₂SO₄, filtered and the filtrate was concentrated in vacuo. The residue was purified by flash silica gel chromatography (Silica Flash Column, eluent of 0˜50% ethyl acetate/petroleum ether gradient) to give the title compound as a white solid (10 g, 46.45 mmol, 25% yield). ¹H NMR (400 MHz, CDCl₃) δ ppm 4.23 (br s, 1H) 3.82-4.09 (m, 1H) 3.62 (br d, J=4.05 Hz, 1H) 2.38 (br t, J=10.67 Hz, 1H) 2.17 (br s, 1H)1.78-2.10 (m, 2H) 1.53-1.72 (m, 1H) 1.45 (s, 9H)1.00 (q, J=11.92 Hz, 1H)0.92 (d, J=6.68 Hz, 3H).

### tert-Butyl 3-(2-benzyloxyethoxy)-5-methyl-piperidine-1-carboxylate

To a solution of tert-butyl-3-hydroxy-5-methyl-piperidine-1-carboxylate (10 g, 46.45 mmol, 1 eq) in DMF (100 mL) was added NaH (3.72 g, 92.90 mmol, 60% purity, 2 eq) in portions at 0° C. under N₂. The mixture was stirred at 0° C. for 30 min, then 2-bromoethoxymethylbenzene (11.99 g, 55.74 mmol, 8.82 mL, 1.2 eq) was added at 0° C., and the mixture was heated to 90° C., and stirred for 11.5 hr. The mixture was quenched by sat, aq. NH₄Cl (200 mL) at 15° C., and the mixture was extracted with ethyl acetate (200 mL×2). The combined organic phase was washed with brine (500 mL), dried with anhydrous Na₂SO₄, filtered and the filtrate was concentrated in vacuo. The residue was purified by flash silica gel chromatography (Silica Flash Column, eluent of 5˜40% ethyl acetate/petroleum ether gradient) to give the title compound as a yellow oil (4.2 g, 9.98 mmol, 44% yield, 83% purity). ¹H NMR (400 MHz, CDCl)8 ppm 7.27-7.38 (m, 5H), 4.58 (s, 2H), 4.22-4.46 (m, 1H), 3.88-4.11 (m, 1H), 3.70 (br d, J=4.63 Hz, 2H), 3.59-3.64 (m, 2H), 3.26-3.36 (m, 1H), 2.40 (br s, 1H), 2.09-2.25 (m, 2H), 1.53-1.64 (m, 1H), 1.46 (s, 9H), 1.01 (q, J=12.09 Hz, 1H), 0.92 (d, J=6.63 Hz, 3H).

### tert-Butyl 3-(2-hydroxyethoxy)-5-methylpiperidine-1-carboxylate

To a solution of tert-butyl 3-(2-benzyloxyethoxy)-5-methyl-piperidine-1-carboxylate (4.2 g, 12.02 mmol, 1 eq) in MeOH (50 mL) was added Pd/C (2 g, 10% purity) under N₂. The suspension was degassed under vacuum and purged several times with H₂. The mixture was stirred under H₂ (50 psi) at 60° C. for 12 hr. The reaction mixture was filtered and the filtrate was concentrated in vacuo to give the title compound as a black oil (3.3 g, 9.29 mmol, 77% yield, 73% purity). ¹H NMR (400 MHz, CDCl₃) δ ppm 4.17-4.43 (m, 1H), 3.86-4.07 (m, 1H), 3.69-3.75 (m, 2H), 3.60-3.66 (m, 2H), 3.26-3.36 (m, 1H), 2.41 (br t,/=11.25 Hz, 1H), 2.04-2.30 (m, 3H), 1.54-1.67 (m, 1H), 1.46 (s, 9H), 0.99 (q, J=11.98 Hz, 1H), 0.92 (d, J=6.60 Hz, 3H).

### tert-Butyl 3-methyl-5-[2-(p-tolylsulfonyloxy)ethoxy]piperidine-1-carboxylate

To a mixture of tert-butyl 3-(2-hydroxyethoxy)-5-methyl-piperidine-1-carboxylate (3.3 g, 12.72 mmol, 1 eq) in DCM (30 mL) was added DMAP (155.45 mg, 1.27 mmol, 0.1 eq) and TEA (1.42 g, 14.00 mmol, 1.95 mL, 1.1 eq). Then TsCl (2.67 g, 14.00 mmol, 1.1 eq) was added into the mixture at 0° C. under N₂. The mixture was stirred at 15° C. for 12 hr. The reaction mixture was poured into water (100 mL) and extracted with ethyl acetate (80 mL×3). The combined organic phase was washed with brine (200 mL), dried with anhydrous Na₂SO₄, filtered and the filtrate was concentrated in vacuo. The residue was purified by column chromatography (SiO₂, petroleum ether/ethyl acetate=1/0 to 3/1) to give the title compound as a yellow oil (3.5 g, 8.32 mmol, 65% yield, 98% purity). ¹H NMR (400 MHz, CDCl) δ ppm 7.80 (d, J=8.34 Hz, 2H)7.34 (d, J=8.11 Hz, 2H)4.07-4.34 (m, 3H) 3.80-4.06 (m, 1H) 3.61-3.73 (m, 2H) 3.12-3.30 (m, 1H) 2.45 (s, 3H) 2.09-2.38 (m, 2H) 1.95-2.04 (m, 1H) 1.48-1.60 (m, 1H) 1.45 (s, 9H)0.80-0.95 (m, 4H).

### tert-Butyl (3S,5R)-3-methyl-5-[2-(p-tolylsulfonyloxy)ethoxy]piperidine-1-carboxylate (8C) and tert-butyl (3R,5S)-3-methyl-5-[2-(p-tolylsulfonyloxy)ethoxy]piperidine-1-carboxylate

tert-Butyl 3-methyl-5-[2-(p-tolylsulfonyloxy)ethoxy]piperidine-1-carboxylate (900 mg, 2.18 mmol, 1 eq) was separated by SFC (column: DAICEL CHIRALPAK IG (250 mm*30 mm, 10 μm); mobile phase: [Neu-ETOH]; B %: 20%-20%, 6 min) to give tert-butyl (3S,5R)-3-methyl-5-[2-(p-tolylsulfonyloxy)ethoxy]piperidine-1-carboxylate (380 mg, 909.74 μmol, 42% yield, 99% purity) as a colorless oil and tert-butyl (3R,5S)-3-methyl-5-[2-(p-tolylsulfonyloxy)ethoxy]piperidine-1-carboxylate (380 mg, 900.55 μmol, 41% yield, 98% purity) as a colorless oil. ¹H NMR (400 MHz, CDCl₃) δ ppm 7.81 (d, J=8.25 Hz, 2H), 7.35 (d, J=8.13 Hz, 2H), 4.14 (t, J=4.82 Hz, 2H), 3.66-3.72 (m, 2H), 3.18-3.26 (m, 1H), 2.46 (s, 4H), 2.10-2.35 (m, 2H), 1.96-2.06 (m, 1H), 1.50-1.61 (m, 2H), 1.46 (s, 9H), 0.83-0.93 (m, 4H). ¹H NMR (400 MHz, CDCl₃) δ ppm 7.81 (d, J=8.25 Hz, 2H), 7.35 (d, J=8.13 Hz, 2H), 4.12-4.17 (m, 2H), 3.66-3.71 (m, 2H), 3.16-3.28 (m, 1H), 2.46 (s, 3H), 2.10-2.32 (m, 2H), 1.97-2.06 (m, 1H), 1.53-1.61 (m, 2H), 1.46 (s, 9H), 0.83-0.94 (m, 5H).

### 7-Methoxy-1-methyl-indoline-2,3-dione

To a solution of 7-methoxyindoline-2,3-dione (50 g, 56.45 mmol, 1 eq) in DMF (600 mL) was added K₂CO₃ (58.5 g, 84.67 mmol, 1.5 eq) in one portion. The reaction mixture was stirred at 20° C. for 30 min. Then Mel (52.08 g, 73.38 mmol, 22.85 mL, 1.3 eq) was added to the reaction mixture. The mixture was stirred at 20° C. for 11.5 hr. The reaction mixture was then poured into water (2000 mL), acidified with 2 N HCl (aq.) slowly until pH=3 and stirred for 45 min. The resulting precipitate was collected by filtration, washed with water (500 mL) and dried under in vacuo to give the title compound as a red solid (50 g, 215.76 mmol, 73% yield). ¹H NMR (400 MHz, DMSO-d₆) δ ppm 7.41 (dd, J=8.17, 0.89 Hz, 1H), 7.14-7.18 (m, 1H), 7.06-7.12 (m, 1H), 3.88 (s, 3H), 3.35 (s, 3H).

### 7-Methoxy-1-methyl-5-nitro-indoline-2,3-dione

To a solution of NaNO₃ (22.23 g, 261.53 mmol, 2 eq) in H₂SO₄ (150 mL) was rapidly added a solution of 7-methoxy-1-methyl-indoline-2,3-dione (25 g, 130.76 mmol, 1 eq) in H₂SO₄ (150 mL) at 0° C. The reaction was stirred at 15° C. for 12 hr. The reaction mixture (combined with another batch with same scale) was then poured into ice/water mixture slowly while the temperature was kept at 0° C. Then the resulting aqueous solution was washed with EtOAc (1 L×3). The combined organic extracts were dried over Na₂SO₄, filtrated and the filtrate was concentrated in vacuo give the title compound as a red solid (60 g, crude). ¹H NMR (400 MHz, DMSO-d₆) δ ppm 8.10 (d, J=1.88 Hz, 1H), 7.90 (d, J=2.13 Hz, 1H), 4.02 (s, 3H), 3.41 (s, 3H)

### 7-Hydroxy-1-methyl-5-nitro-indoline-2,3-dione

A solution of 7-methoxy-1-methyl-5-nitro-indoline-2,3-dione (33 g, 139.72 mmol, 1 eq) in HBr (500 mL, 48% purity) was stirred at 120° C. for 12 hr. The reaction mixture (combined with another batch with same scale) was then poured into ice/water slowly while the temperature was kept at 0° C. Then the resulting aqueous solution was washed with EtOAc (2 L×3). The combined organic were dried over Na₂SO₄, filtrated and the filtrate was concentrated in vacuo. The residue was purified by flash silica gel chromatography (Silica Flash Column, eluent of 10˜50% ethyl acetate/petroleum ether gradient) to give the title compound as a red solid (20 g). ¹H NMR (400 MHz, DMSO-d₆) δ ppm 11.16 (br s, 1H), 7.90 (d, J=2.20 Hz, 1H), 7.75 (d, J=2.20 Hz, 1H), 3.41 (s, 3H).

### 6| 7-Benzyloxy-1-methyl-5-nitro-indoline-2,3-dione

A solution of 7-hydroxy-1-methyl-5-nitro-indoline-2,3-dione (9 g, 40.51 mmol, 1 eq) in DMF (90 mL) was added K₂CO₃ (6.72 g, 48.61 mmol, 1.2 eq) was stirred at 15° C. for 30 min, then BnBr (7.62 g, 44.56 mmol, 5.29 mL, 1.1 eq) was added dropwise and the mixture was stirred at 15° C. for 11.5 hr. To the mixture (combined with another batch with same scale) were added water (200 mL) and brine (200 mL), followed by extracted with ethyl acetate (300 mL×3). The combined organic phase was washed with brine (1000 mL), dried with anhydrous Na₂SO₄, filtered and the filtrate was concentrated in vacuo. The residue was purified by flash silica gel chromatography (Silica Flash Column, eluent of 10˜50% ethyl acetate/petroleum ether gradient) to give the title compound as a red solid (12 g, 23.63 mmol, 29% yield, 61% purity). ¹H NMR. (400 MHz, DMSO-d₆) δ ppm 8.23 (d, J=2.0 Hz, 1H), 7.91 (d, J=2.0 Hz, 1H), 7,55-7,50 (m, 2H), 7.45 (t, J=7.3 Hz, 2H), 7.40-7.36 (m, 1H), 5.40 (s, 2H), 3.39 (s, 3H).

### 8-Benzyloxy-3-hydroxy-1-methyl-6-nitro-quinolin-2-one

A solution of 7-benzyloxy-1-methyl-5-nitro-indoline-2,3-dione (4.2 g, 13.45 mmol, 1 eq) in DCM (80 mL) was added TMSCHN₂ (2 M, 13.45 mL, 2 eq) drop wise at 0° C., then the reaction mixture was stirred at 15° C. for 1.5 hr. The reaction mixture (combined with another batch with the same scale) was quenched with HOAc until no bubbles occurred. Then H₂O (250 mL) was added to the mixture. The resulting mixture was extracted with DCM (300 mL×3). The combined organic phase was washed with brine (500 mL), dried with anhydrous Na₂SO₄, filtered and concentrated in vacuo. The residue was triturated with DCM (20 mL) at 15° C. for 30 min. Then the suspension was filtered and the solid was washed with DCM (8 mL×2) to give the title compound as a brown solid (2.2 g, 6.61 mmol, 25% yield, 98% purity). ¹H NMR (400 MHz, DMSO-d₆) δ ppm 10.03 (s, 1H), 8.19 (d, J=2.26 Hz, 1H), 7.84 (d, J=2.27 Hz, 1H), 7,55 (br d, J=7.15 Hz, 2H), 7.35-7.47 (m, 3H), 7.29 (s, 1H), 5.33 (s, 2H), 3.88 (s, 3H).

### 2-[(8-Benzyloxy-1-methyl-6-nitro-2-oxo-3-quinolyl)oxy]—N-methyl-acetamide

2-Bromo-N-methyl-acetamide (1.35 g, 8.90 mmol, 1.2 eq) and Cs₂CO₃ (4.83 g, 14.83 mmol, 2 eq) was added to a solution of 8-benzyloxy-3-hydroxy-1-methyl-6-nitro-quinolin-2-one (2.42 g, 7.42 mmol, 1 eq) in DMF (70 mL). The reaction mixture was stirred at 15° C. for 12 hr. Then the reaction mixture was treated with H₂O (100 mL) and filtered. The filter cake was collected and dried in vacuo to give the title compound as a pale brown solid (2.5 g, crude). ¹H NMR (400 MHz, DMSO-d₆) δ ppm 8.26 (br s, 1H), 7.92 (br s, 2H), 7,55 (br d, J=6.20 Hz, 2H), 7.41-7.49 (m, 3H), 7.39 (br d, J=5.60 Hz, 1H), 5.29-5.39 (m, 2H), 4.50-4.65 (m, 2H), 3.86 (br s, 3H), 2.60-2.71 (m, 3H).

### 2-[(8-Hydroxy-1-methyl-6-nitro-2-oxo-3-quinolyl)oxy]—N-methyl-acetamide

To a solution of 2-[(8-benzyloxy-1-methyl-6-nitro-2-oxo-3-quinolyl)oxy]—N-methyl-acetamide (2.5 g, 6.29 mmol, 1 eq) in DCM (150 mL) was added BCl (1 M, 12.58 mL, 2 eq) at −70° C. The reaction mixture was then stirred at 15° C. for 12 hr. The reaction mixture (combined with another batch with 50 mg scale) was quenched by a mixture of (CHCl₃: MeOH=10:1, 750 mL) at −70° C. The reaction mixture was then warmed to 15° C., and stirred for 20 min. The mixture was concentrated in vacuo to give the title compound as a yellow solid (1.9 g, 5.44 mmol, 86% yield, 88% purity). ¹H NMR. (400 MHz, DMSO-d₆) δ ppm 11.07 (s, 1H), 8.04 (d, J=2.50 Hz, 1H), 7.90 (br d, J=4.38 Hz, 1H), 7.69 (d, J=2.63 Hz, 1H), 7.40 (s, 1H), 4.56 (s, 2H), 3.94 (s, 3H), 2.66 (d, J=4.75 Hz, 3H).

### tert-Butyl (3S,5R)-3-methyl-5-[2-[[I-methyl-3-[2-(methylamino)-2-oxo-ethoxy]-6-nitro-2-oxo-8-quinolyl]oxy]ethoxy]piperidine-1-carboxylate

To a solution of 2-[(8-hydroxy-1-methyl-6-nitro-2-oxo-3-quinolyl)oxy]—N-methyl-acetamide (250 mg, 813.65 μmol, 1 eq) and tert-butyl (3S,5R)-3-methyl-5-[2-(p-tolylsulfonyloxy)ethoxy]piperidine-1-carboxylate (336.47 mg, 813.65 μmol, 1 eq) in DMF (3 mL) was added K₂CO₃ (281.14 mg, 2.03 mmol, 2.5 eq). The mixture was stirred at 60° C. for 3 hr. The mixture was cooled to 20° C., and water (3 mL) was added which caused a precipitate to form. The mixture was filtered and the filter cake was washed with water (5 mL×2) and then an organic solution (petroleum ether:ethyl acetate=1/1, 10 mL). The filter cake was dried in vacuo to give the title compound as a yellow solid (350 mg, 548.69 μmol, 67% yield, 86% purity). ¹H NMR (400 MHz, DMSO-d₆) δ ppm 8.22 (d,.)=2.25 Hz, 1H), 7.91 (br d,/=2.25 Hz, 1H), 7.80 (d,.)=2.38 Hz, 1H), 7.45 (s, 1H), 4.57 (s, 2H), 4.33 (br t, J=3.94 Hz, 2H), 4.03-4.19 (m, 1H), 3.92-3.95 (m, 3H), 3.73-3.92 (m, 4H), 2.67 (d, J=4.50 Hz, 3H), 2.14-2.44 (m, 2H), 2.09 (br d,)=12.26 Hz, 1H), 1.43-1.56 (m, 1H), 1.38 (s, 9H), 0.89-0.98 (m, 1H), 0.87 (d, J=6.63 Hz, 3H).

### tert-Butyl (3R,5S)-3-[2-[[6-amino-1-methyl-3-[2-(methylamino)-2-oxo-ethoxy]-2-oxo-8-quinolyl]oxy]ethoxy]-5-methyl-piperidine-1-carboxylate

To a solution of tert-butyl (3S,5R)-3-methyl-5-[2-[[1-methyl-3-[2-(methylamino)-2-oxo-ethoxy]-6-nitro-2-oxo-8-quinolyl]oxy]ethoxy]piperidine-1-carboxylate (350 mg, 638.01 μmol, 1 eq) in DMF (5 mL) was added Pd/C (0.1 g, 10% purity) under Ar. The suspension was degassed under vacuum and purged with H₂ several times. The mixture was stirred under H₂ (15 psi) at 20° C. for 12 hr. The mixture was filtered by Celite® and the filter cake was washed with DMF (˜15 mL). The filtrate was concentrated in vacuo to give the title compound as a yellow solid (340 mg, crude). LCMS: [M+H]⁺=519.4.

### tert-Butyl (3R,5S)-3-[2-[[6-[(2,5-dichloropyrimidin-4-yl)amino]-1-methyl-3-[2-(methylamino)-2-oxo-ethoxy]-2-oxo-8-quinolyl]oxy]ethoxy]-5-methyl-piperidine-1-carboxylate

To a solution of tert-butyl (3R,5S)-3-[2-[[6-amino-1-methyl-3-[2-(methylamino)-2-oxo-ethoxy]-2-oxo-8-quinolyl]oxy]ethoxy]-5-methyl-piperidine-1-carboxylate (340 mg, 655.61 μmol, 1 eq) and 2,4,5-trichloropyrimidine (240.51 mg, 1.31 mmol, 2 eq) in DMF (3.5 mL) was added DIPEA (169.46 mg, 1.31 mmol, 228.39 μL, 2 eq). The mixture was stirred at 20° C. for 12 hr. The reaction mixture was purified by flash silica gel chromatography (Silica Flash Column, Eluent of 10˜100% EtOH/Ethyl acetate) to give ˜300 mg of crude product. The crude product was further triturated with (PE/EtOAc=1/1, 3 mL) at 20° C. for 5 min to give the title compound as a yellow solid (200 mg, 283.67 μmol, 43% yield, 94% purity). ¹H NMR (400 MHz, DMSO-d₆) δ ppm 9.58 (s, 1H), 8.39 (s, 1H), 7.94 (br d, J=4.50 Hz, 1H), 7.42 (s, 2H), 7.14 (s, 1H), 4.57 (s, 2H), 4.18 (br t,,J=4.06 Hz, 3H), 3.85-3.95 (m, 5H), 3.79 (dt, J=7.63, 3.69 Hz, 1H), 2.66 (d, J=4.63 Hz, 3H), 2.14-2.46 (m, 2H), 2.07 (br d,/=11.51 Hz, 1H), 1.43-1.57 (m, 1H), 1.38 m 1.34 (m, 10H), 0.93 (q, J=11.84 Hz, 1H), 0.86 (d, J=6.63 Hz, 3H).

### 2-[[6-[(2,5-Dichloropyrimidin-4-yl)amino]-1-methyl-8-[2-[[(3R,5S)-5-methyl-3-piperidyl]oxy]ethoxy]-2-oxo-3-quinolyl]oxy]—N-methyl-acetamide

A solution of tert-butyl (3R,5S)-3-[2-[[6-[(2,5-dichloropyrimidin-4-yl)amino]-1-methyl-3-[2-(methylamino)-2-oxo-ethoxy]-2-oxo-8-quinolyl]oxy]ethoxy]-5-methyl-piperidine-1-carboxylate (200.00 mg, 300.50 μmol, 1 eq) in DCM (2 mL) and TFA (0.5 mL) was stirred at 20° C. for 1 hr. The mixture was concentrated in vacuo to give the title compound as a yellow oil (220 mg, crude, TFA). LCMS: [M+H]⁺=565.2.

### 2-(1³R, 1⁵S)-2⁵-chloro-1⁵,4¹-dimethyl-4²-oxo-4¹, 4²-dihydro-5,8-dioxa-3-aza-4(6,8)-quinolina-2(2,4)-pyrimidina-1 (1,3)-piperidinacyclooctaphane-4³-yl)oxy)-N-methylacetamide (6)

To a solution of 2-[[6-[(2,5-dichloropyrimidin-4-yl)amino]-1-methyl-8-[2-[[(3R,5S)-5-methyl-3-piperidyl]oxy]ethoxy]-2-oxo-3-quinolyl]oxy]—N-methyl-acetamide (220 mg, 323.78 μmol, 1 eq, TFA) in DMSO (9 mL) was added DIPEA (418.46 mg, 3.24 mmol, 563.96 μL, 10 eq). The mixture was stirred at 80° C. for 12 hr. The mixture was filtered and the filtrate was purified directly without workup. The filtrate was purified by p-HPLC (column: Waters Xbridge™ BEH C18 100*30 mm*10 μm; mobile phase: [water (NH₄HCO₃)-ACN]; B %: 40%-60%, 8 min) to give the title compound as a yellow solid (21 mg, 37.48 μmol, 12% yield, 94% purity). ¹H NMR (400 MHz, DMSO-d₆) δ ppm 8.99 (s, 1H), 8.02-8.11 (m, 2H), 7.94 (br d,)=4.05 Hz, 1H), 7.08 (d, J=1.79 Hz, 1H), 7.06 (s, 1H), 4.65-4.77 (m, 1H), 4.50-4.62 (m, 3H), 4.32-4.41 (m, 1H), 4.13-4.26 (m, 1H), 3.68-3.91 (m, 6H), 2.66 (d, J=4.53 Hz, 3H), 2.52-2.59 (m, 2H), 1.98 (br d, J=11.68 Hz, 1H), 1.55-1.70 (m, 1H), 1.09-1.19 (m, 1H), 0.89 (br d, J=6.56 Hz, 3H). LCMS: [M+H]⁺=529.3.

### Example 4: Synthesis of 2-(((1³S,1⁵R)-2⁵-chloro-1⁵,4¹-dimethyl-4²-oxo-41,42 dihydro-5,8-dioxa-3-aza-4(6.8)-quinolina-2(2,4)-pyrimidina-1 (1,3)-piperidinacyclooctaphane-4³-yl)oxy)-N-methylacetamide (7)

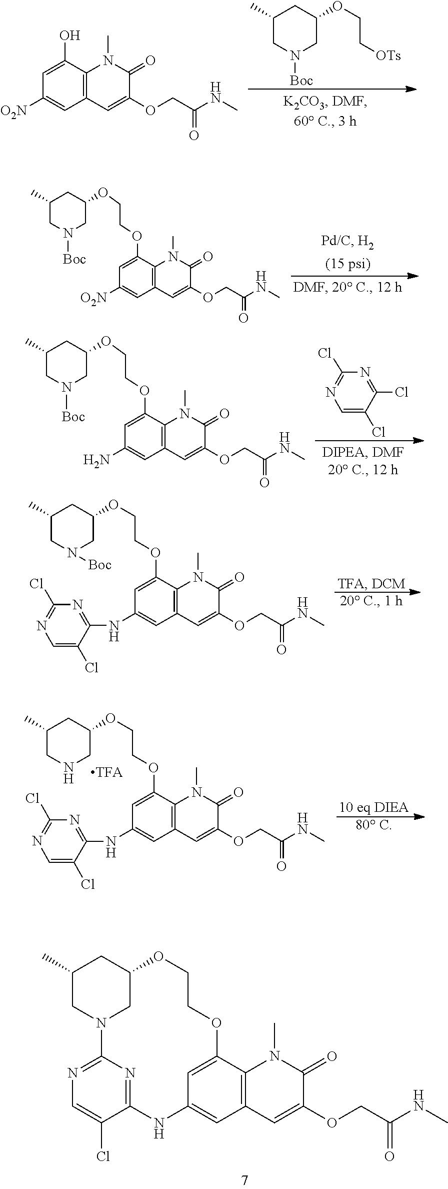

### tert-Butyl (3R,5S)-3-methyl-5-[2-[[1-methyl-3-[2-(methylamino)-2-oxo-ethoxy]-6-nitro-2-oxo-8-quinolyl]oxy]ethoxy]piperidine-1-carboxylate

To a solution of 2-[(8-hydroxy-1-methyl-6-nitro-2-oxo-3-quinolyl)oxy]—N-methyl-acetamide (200 mg, 650.92 μmol, 1 eq) and tert-butyl (3R,5S)-3-methyl-5-[2-(p-tolylsulfonyloxy)ethoxy]piperidine-1-carboxylate (269.17 mg, 650.92 μmol, 1 eq) in DMF (3 mL) was added K₂CO₃ (224.91 mg, 1.63 mmol, 2.5 eq). The mixture was stirred at 60° C. for 3 hr. The mixture (combined with another batch with 50 mg scale) was cooled to 20° C., and ˜3 mL of water was added. A precipitate was occurred. The mixture was filtered and the filter cake was washed with water (5 mL×2) and organic solution (petroleum ether:ethyl acetate=1/1, 10 mL), then the mixture was dried in vacuo to give the title compound as a yellow solid (350 mg, 93.4% purity). ¹H NMR (400 MHz, DMSO-d₆) δ ppm 8.23 (d, J=2.50 Hz, 1H), 7.92 (br d,)=4.25 Hz, 1H), 7.81 (d, J=2.50 Hz, 1H), 7.46 (s, 1H), 4.57 (s, 2H), 4.33 (t, J=4.13 Hz, 2H), 4.01-4.21 (m, 1H), 3.94 (s, 3H), 3.71-3.93 (m, 4H), 2.66 (d, J=4.50 Hz, 3H), 2.15-2.40 (m, 2H), 2.03-2.13 (m, 1H), 1.45-1.57 (m, 1H), 1.38 (s, 9H), 0.89-0.98 (m, 1H), 0.87 (d, J=6.63 Hz, 3H).

### tert-Butyl (3S,5R)-3-[2-[[6-amino-1-methyl-3-[2-(methylamino)-2-oxo-ethoxy]-2-oxo-8-quinolyl]oxy]ethoxy]-5-methyl-piperidine-1-carboxylate

A solution of tert-butyl (3R,5S)-3-methyl-5-[2-[[1-methyl-3-[2-(methylamino)-2-oxo-ethoxy]-6-nitro-2-oxo-8-quinolyl]oxy]ethoxy]piperidine-1-carboxylate (350 mg, 638.01 μmol, 1 eq) in DMF (5 mL) was added Pd/C (0.1 g, 10% purity) under Ar. The suspension was degassed under vacuum and purged with H₂ several times. The mixture was stirred under Hz (15 psi) at 20° C. for 12 hr. The mixture was filtered by Celite® and the filter cake was washed with DMF (˜15 mL), then the filtrate was concentrated in vacuo to give the title compound as a yellow solid (340 mg, crude). LCMS: [M+H]⁺=519.4.

### tert-Butyl (3S,5R)-3-[2-[[6-[(2,5-dichloropyrimidin-4-yl)amino]-1-methyl-3-[2-(methylamino)-2-oxo-ethoxy]-2-oxo-8-quinolyl]oxy]ethoxy]-5-methyl-piperidine-1-carboxylate

To a solution of tert-butyl (3S,5R)-3-[2-[[6-amino-1-methyl-3-[2-(methylamino)-2-oxo-ethoxy]-2-oxo-8-quinolyl]oxy]ethoxy]-5-methyl-piperidine-1-carboxylate (340 mg, 655.61 μmol, 1 eq) and 2,4,5-trichloropyrimidine (240.51 mg, 1.31 mmol, 2 eq) in DMF (3.5 mL) was added DIPEA (169.46 mg, 1.31 mmol, 228.39 μL, 2 eq). The mixture was stirred at 20° C. for 12 hr. The mixture was concentrated in vacuo. The residue was purified by flash silica gel chromatography (Silica Flash Column, Eluent of 10˜100% EtOH/Ethyl acetate) to give ˜300 mg of crude product. The crude product was further triturated with (PE/EtOAc=1/1, 3 mL) at 20° C. for 5 min to give the title compound as a yellow solid (220 mg, 324.93 μmol, 50% yield, 98% purity). ¹H NMR (400 MHz, DMSO-d₆) δ ppm 9.60 (s, 1H), 8.40 (s, 1H), 7.93 (br d, J=4.40 Hz, 1H), 7.42 (br s, 2H), 7.14 (s, 1H), 4.57 (s, 2H), 4.05-4.22 (m, 3H), 3.85-3.97 (m, 5H), 3.72-3.84 (m, 1H), 2.66 (d, J=4.52 Hz, 3H), 2.55-2.59 (m, 2H), 2.16-2.42 (m, 1H), 1.42-1.58 (m, 2H), 1.38 (s, 9H), 0.88-0.98 (m, 1H), 0.82-0.88 (m, 3H).

### 2-[[6-[(2,5-Dichloropyrimidin-4-yl)amino]-1-methyl-8-[2-[[(3S,5R)-5-methyl-3-piperidyl]oxy]ethoxy]-2-oxo-3-quinolyl]oxy]—N-methyl-acetamide

A solution of tert-butyl (3S,5R)-3-[2-[[6-[(2,5-dichloropyrimidin-4-yl)amino]-1-methyl-3-[2-(methylamino)-2-oxo-ethoxy]-2-oxo-8-quinolyl]oxy]ethoxy]-5-methyl-piperidine-1-carboxylate (180 mg, 270.45 μmol, 1 eq) in DCM (2 mL) and TFA (0.5 mL) was stirred at 20° C. for 1 hr. The mixture was concentrated in vacuo to give the title compound as a yellow oil (200 mg, crude, TFA salt). LCMS: [M+H]⁺=565.6.

### 2-(((1³S, 1⁵R)-2⁵-chloro-1⁵,4¹-dimethyl-4²-oxo-4¹,4²-dihydro-5,8-dioxa-3-aza-4(6,8)-quinolina-2(2,4)-pyrimidina-1 (1,3)-piperidinacyclooctaphane-4³-yl)oxy)-N-methylacetamide (7)

To a solution of 2-[[6-[(2,5-dichloropyrimidin-4-yl)amino]-1-methyl-8-[2-[[(3S,5R)-5-methyl-3-piperidyl]oxy]ethoxy]-2-oxo-3-quinolyl]oxy]—N-methyl-acetamide (200 mg, 294.35 μmol, 1 eq, TFA) in DMSO (8 mL) was added DIPEA (380.42 mg, 2.94 mmol, 512.70 μL, 10 eq). The mixture was stirred at 80° C. for 12 hr. The mixture was filtered and the filtrate was purified directly without further workup. The filtrate (combine with another batch at 25 mg) was purified by p-HPLC (column: Waters Xbridge™ BEH C18 100*30 mm*10 μm; mobile phase: [water (NH₄HCO₃)-ACN]; B %: 35%-65%, 8 min) to give the title compound as a yellow solid (25 mg, 95% purity). ¹H NMR (400 MHz, DMSO-d₆) δ ppm 8.66-9.21 (m, 1H), 8.06 (s, 1H), 7.99 (br d, J=1.63 Hz, 1H), 7.88 (s, 1H), 7.05 (s, 1H), 6.91 (br s, 1H), 4.63 (br d, J=10.01 Hz, 2H), 4.54 (s, 2H), 4.36 (br d,/=9.38 Hz, 1H), 4.11-4.24 (m, 1H), 3.84-3.92 (m, 1H), 3.67-3.84 (m, 5H), 2.66 (br d, J=4.13 Hz, 3H), 2.32-2.47 (m, 2H), 1.96 (br d,.)=10.38 Hz, 1H), 1.54-1.67 (m, 1H), 1.10 (q, J=11.51 Hz, 1H), 0.87 (br d, J=6.38 Hz, 3H). LCMS: [M+H]⁺=529.3

### Example 5: Synthesis of (138, 15R)-2⁵-chloro-1⁴,1⁴-difluoro-4-(3-hydroxy-3-methylbutyl)-1⁵,4³-dimethyl-4²,4³-dihydro-4¹H-5-oxa-3-aza-4(6,4)-benzo[d]imidazola-2(2,4)-pyrimidina-1 (1,3)-piperidinacyclooctaphan-4²-one (10)

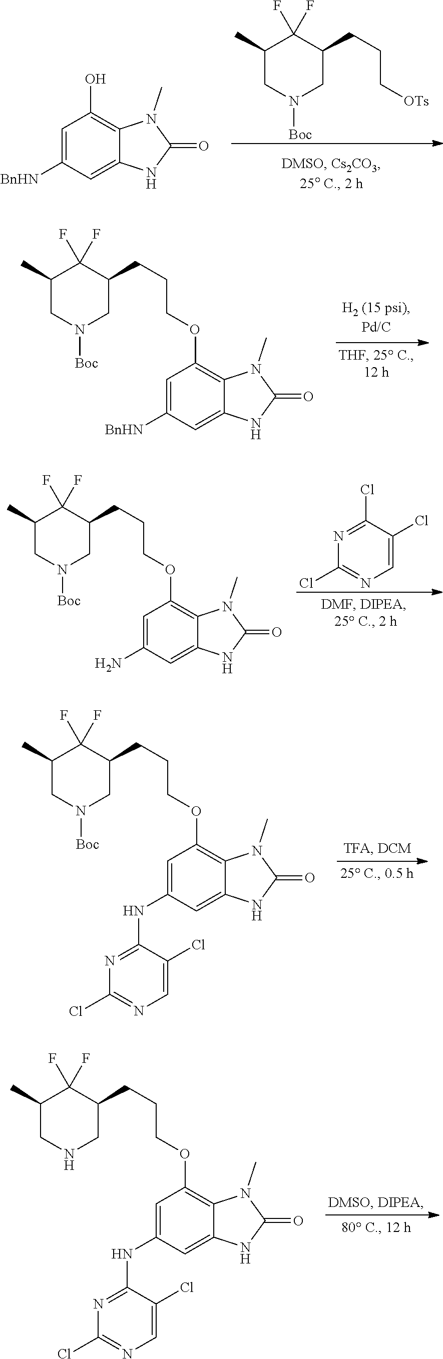

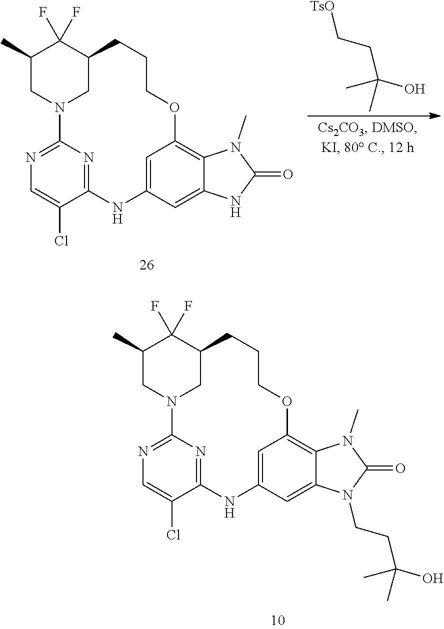 [SMILES: C[C@@H]1CN2C[C@H](CCCOc3cc(cc4[nH]c(=O)n(C)c34)Nc3nc2ncc3Cl)C1(F)F ; Cc1ccc(S(=O)(=O)OCCC(C)(C)O)cc1 ; C[C@@H]1CN2C[C@H](CCCOc3cc(cc4c3n(C)c(=O)n4CCC(C)(C)O)Nc3nc2ncc3Cl)C1(F)F]

### tert-Butyl (3S,5R)-3-(3-((6-(benzylamino)-3-methyl-2-oxo-2,3-dihydro-1H-benzo[d]imidazol-4-yl)oxy)propyl)-4,4-difluoro-5-methylpiperidine-1-carboxylate

To a solution of 5-(benzylamino)-7-hydroxy-1-methyl-1,3-dihydro-2H-benzo[d]imidazol-2-one (660 mg, 2.45 mmol, 1 eq) in DMSO (7 mL) were added tert-butyl (3R,5S)-4,4-difluoro-3-methyl-5-(3-(tosyloxy)propyl)piperidine-1-carboxylate (1.1 g, 2.45 mmol, 1 eq) and Cs₂CO₃ (1.60 g, 4.90 mmol, 2 eq). The reaction mixture was stirred at 25° C. for 2 hr. The reaction mixture (combined with another batch with 500 mg scale) was treated with water (20 mL), then extracted with EtOAc (25 mL×3). The combined organic phase was washed with brine (50 mL), dried with anhydrous Na₂SO₄, filtered and concentrated in vacuo to give a residue which was purified by prep-HPLC (column: Welch Xtimate® C18 180*70 mm*10 μm,mobile phase: [water (NH₄HCO₃)-ACN]; B %: 50%-80%, 15 min) to give the title compound as a white solid (1 g, 1.82 mmol, 66% yield, 99% purity). ¹H NMR (400 MHz, DMSO-d₆) δ=10.35 (br s, 1H), 7.37-7.27 (m, 4H), 7.23-7.18 (m, 1H), 6.03 (d, J=1.6 Hz, 1H), 5,93 (t,/=6.0 Hz, 1H), 5.82 (d,)=1.6 Hz, 1H), 4.22 (d, J=6.0 Hz, 2H), 4.15-3.99 (m, 1H), 3.95 (t, J=5.4 Hz, 3H), 3.33 (s, 3H), 2.68-2.52 (m, 2H), 2.06-1.69 (m, 5H), 1.40 (s, 9H), 1.35-1.26 (m, 1H), 0.94 (d, J=6.4 Hz, 3H)

### ter-Butyl (3S,5R)-3-(3-(6-amino-3-methyl-2-oxo-2,3-dihydro-1H-benzo[d]imidazol-4-yl)oxy)propyl)-4,4-difluoro-5-methylpiperidine-1-carboxylate

To a solution of tert-butyl (3S,5R)-3-(3-((6-(benzylamino)-3-methyl-2-oxo-2,3-dihydro-1H-benzo[d]imidazol-4-yl)oxy)propyl)-4,4-difluoro-5-methylpiperidine-1-carboxylate (1 g, 1.84 mmol, 1 eq) in THF (10 mL) was added Pd/C (300 mg, 10% purity) under Ar. The suspension was degassed under vacuum and purged with Hz several times. The reaction mixture was stirred under H₂ (15 psi) at 25° C. for 12 hr. The reaction mixture was filtered and the filtrate was concentrated in vacuo to give the title compound as a brown solid (800 mg, 1.60 mmol, 87% yield, 91% purity). ¹H NMR (400 MHz, DMSO-d₆)_{6=9.13} (s, 1H), 7.99 (s, 1H), 7.98-7.92 (d, J=4.4 Hz, 2H), 7.80-7.74 (d, J=2.4 Hz, 2H), 7.64-7,58 (m, 1H), 7,57-7,51 (m, 1H), 7.22 (m, 1H), 4.66 (d, J=7.0 Hz, 2H), 4.62-4.56 (m, 2H), 4.55-4.49 (m, 4H), 4.17-4.08 (m, 2H), 3.43 (m, 1H), 2.86-2.72 (m, 2H), 2.67 (d, J=4.8 Hz, 3H), 2.15-1.97 (m, 2H), 0.84 (d, J=6.4 Hz, 6H)

### ter-Butyl (3S,5R)-3-(3-((6-(2,5-dichloropyrimidin-4-yl)amino)-3-methyl-2-oxo-2,3-dihydro-1H-benzo[d]imidazol-4-yl)oxy)propyl)-4,4-difluoro-5-methylpiperidine-1-carboxylate

To a solution of tert-butyl (3S,5R)-3-(3-((6-amino-3-methyl-2-oxo-2,3-dihydro-1H-benzo[d]imidazol-4-yl)oxy)propyl)-4,4-difluoro-5-methylpiperidine-1-carboxylate (500 mg, 1.10 mmol, 1 eq) in DMF (5 mL) were added 2,4,5-trichloropyrimidine (403.56 mg, 2.20 mmol, 2 eq) and DIPEA (355.45 mg, 2.75 mmol, 479.04 μL, 2.5 eq). The reaction mixture was stirred at 25° C. for 2 hr. The reaction mixture was treated with water (8 mL), then the suspension was filtered. The precipate was filtered, and the solid was purified by column chromatography (SiO₂, petroleum ether/ethyl acetate=1/1 to 0/1) to give the title compound as a yellow solid (530 mg, 793.05 μmol, 72% yield, 90% purity). ¹H NMR (400 MHz, DMSO-d₆) δ=10.90 (s, 1H), 9.39 (s, 1H), 8.34 (s, 1H), 6.97 (s, 2H), 4.06 (t,.)=4.8 Hz, 2H), 3.97-3.88 (m, 1H), 3.45 (s, 3H), 2.70-2.54 (m, 2H), 2.02-1.81 (m, 5H), 1.39 (s, 10H), 0,94 (d, J=6.8 Hz, 3H).

### 5-((2,5-Dichloropyrimidin-4-yl)amino)-7-(3-((3S,5R)-4,4-difluoro-5-methylpiperidin-3-yl)propoxy)-1-methyl-1,3-dihydro-2H-benzo[d]imidazol-2-one

A solution of tert-butyl (3S,5R)-3-(3-((6-((2,5-dichloropyrimidin-4-yl)amino)-3-methyl-2-oxo-2,3-dihydro-1H-benzo[d]imidazol-4-yl)oxy)propyl)-4,4-difluoro-5-methylpiperidine-1-carboxylate (500 mg, 831.29 μmol, 1 eq) in DCM (1.5 mL) and TFA (0.5 mL) was stirred at 25° C. for 0.5 hr. The reaction mixture was concentrated in vacuo to give the title compound has a yellow oil (500 mg, crude, TFA). LCMS: [M+H]⁺=501.1.

### (1³S,1³R)-2⁵-chloro-1⁴,1⁴-difluoro-1⁵,4³-dimethyl-4²,4³-dihydro-4 H-5-oxa-3-aza-4(6,4)-benzo[d]imidazola-2(2,4)-pyrimidina-1 (1,3)-piperidinacyclooctaphan-4²-one (26)

To a solution of 5-((2,5-dichloropyrimidin-4-yl)amino)-7-(3-((3S,5R)-4,4-difluoro-5-methylpiperidin-3-yl)propoxy)-1-methyl-1,3-dihydro-2H-benzo[d]imidazol-2-one (500 mg, 812.51 μmol, 1 eq, TFA) in DMSO (30 mL) was added DIPEA (1.05 g, 8.13 mmol, 1.42 mL, 10 eq). The reaction mixture was stirred at 80° C. for 12 hr. The reaction mixture was treated with water (30 mL), then the suspension was filtered to give a residue. The residue was purified by column chromatography (SiO₂, petroleum ether/ethyl acetate=1/1 to 0/1) to give the title compound as a yellow solid (250 mg, 516.25 μmol, 64% yield, 96% purity) as a yellow solid. ¹H NMR (400 MHz, DMSO-d₆) δ=10.80 (s, 1H), 8.75 (s, 1H), 8.06 (s, 1H), 7.49 (s, 1H), 6.63 (s, 1H), 4.70 (d, J=12.6 Hz, 1H), 4.58 (d, J=12.8 Hz, 1H), 4.41 (t, J=11.6 Hz, 1H), 3.42 (s, 3H), 2.76-2.68 (m, 2H), 2.40-2.24 (m, 1H), 2.17 (t, J=6.8 Hz, 1H), 2.04-1.81 (m, 3H), 1.70-1.57 (m, 2H), 0.97 (br d, J=6.0 Hz, 3H). LCMS: [M+H]⁺=465.2. 30 mg of the yellow solid was further purified by prep-HPLC (column: Waters Xbridge BEH C18 100*30 mm*10 μm; mobile phase: [water (NH₄HCO₃)-ACN]; B %: 40%-70%, 8 min) to give the title compound as a white solid (10 mg, 21.30 μmol, 33% yield, 99% purity). ¹H NMR (400 MHz, DMSO-d₆) δ=10.80 (s, 1H), 8.76 (s, 1H), 8.06 (s, 1H), 7.49 (s, 1H), 6.63 (d, J=1.0 Hz, 1H), 4.70 (d, J=13.0 Hz, 1H), 4.59 (d, J=12.5 Hz, 1H), 4.46-4.36 (m, 1H), 4.14-4.00 (m, 1H), 3.42 (s, 3H), 2.73 (br t, J=12.7 Hz, 1H), 2.59-2.53 (m, 1H), 2.39-2.25 (m, 1H), 2.05-1.57 (m, 4H), 1.35 (dt,/=5.2, 12.6 Hz, 1H), 0.97 (d, J=6.7 Hz, 3H). LCMS: [M+H]⁺=465.3.

### (1³S,1⁵R)-2⁵-chloro-1⁴,1⁴-difluoro-4¹-(3-hydroxy-3-methylbutyl)-1⁵,4³-dimethyl-4², 4³-dihydro-4¹H-5-oxa-3-aza-4(6,4)-benzo[d]imidazola-2(2,4)-pyrimidina-1 (1,3)-piperidinacyclooctaphan-4²-one (10)

To a mixture of (1³S,1⁵R)-2⁵-chloro-1⁴,1⁴-difluoro-1⁵,4³-dimethyl-4²,4³-dihydro-41H-5-oxa-3-aza-4(6,4)-benzo[d]imidazola-2(2,4)-pyrimidina-1 (1,3)-piperidinacyclooctaphan-4²-one (150 mg, 322.65 μmol, 1 eq) and (3-hydroxy-3-methyl-butyl) 4-methylbenzenesulfonate (166.70 mg, 645.31 μmol, 2 eq) in DMSO (2 mL) were added KI (26.78 mg, 161.33 μmol, 0.5 eq) and Cs₂CO₃ (210.25 mg, 645.31 μmol, 2 eq). The reaction mixture was stirred at 80° C. for 12 hr. The rection mixture was treated with water and the suspension was filtered. The solid was purified by prep-HPLC (column: Waters Xbridge BEH C18 100*30 mm*10 μm;mobile phase: [water (NH₄HCO₃)-ACN]; B %: 60%-90%, 8 min) to give the title compound as a white solid (30 mg, 53.90 μmol, 17% yield, 99% purity). ¹H NMR (400 MHz, DMSO-d₆) δ=8.84 (s, 1H), 8.07 (s, 1H), 7,54 (s, 1H), 6.76 (s, 1H), 4.70 (br d, J=12.0 Hz, 1H), 4.59 (d, J=12.0 Hz, 1H), 4.49-4.37 (m, 2H), 4.14-4.04 (m, 1H), 3.82 (dd, J=6.0, 9.6 Hz, 2H), 3.46 (s, 3H), 2.79-2.67 (m, 1H), 2.59-2.52 (m, 1H), 2.42-2.23 (m, 1H), 2.01-1.57 (m, 6H), 1.41-1.29 (m, 1H), 1.18 (s, 6H), 0.97 (d, J=6.8 Hz, 3H). LCMS: [M+H]⁺=551.4.

### Example 6: Synthesis of (PRIS)-23-chloro-1⁴, 1⁴-difluoro-4-(3-hydroxy-3-methylbutyl)-1⁵,4³-dimethyl-4²,4³-dihydro-4¹H-5-oxa-3-aza-4(6,4)-benzo[d]imidazola-2(2,4)-pyrimidina-1 (1,3)-piperidinacyclooctaphan-4²-one (11)

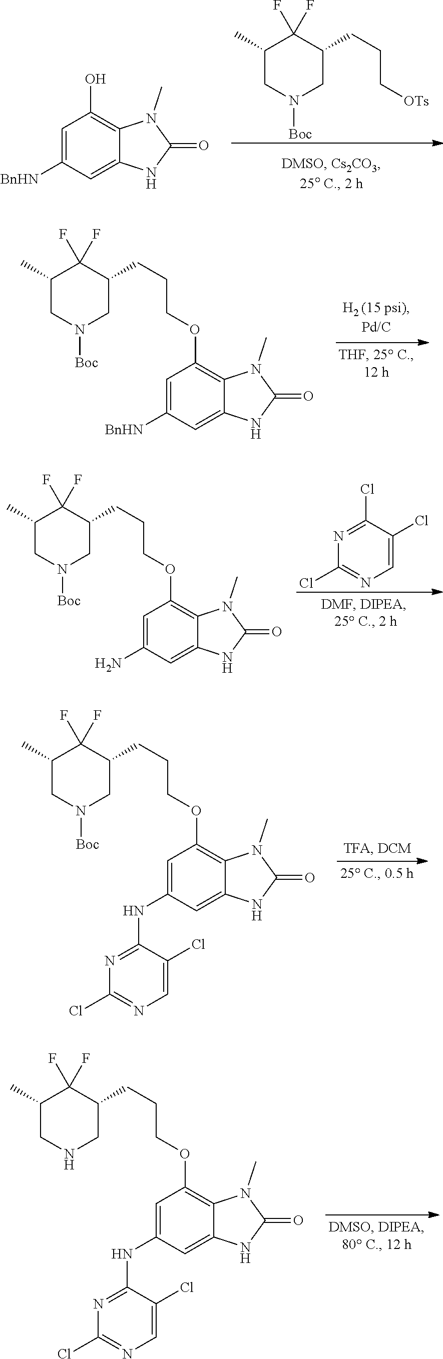

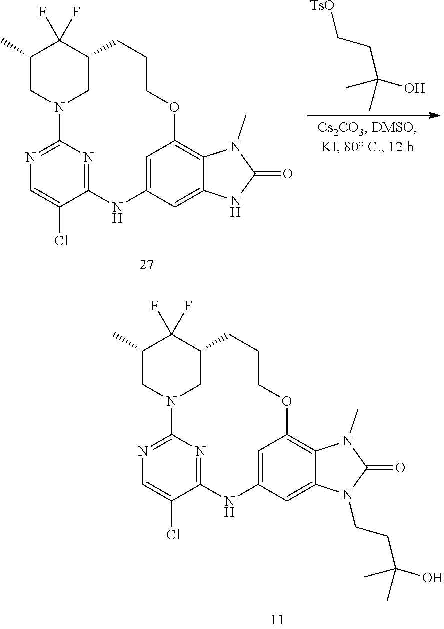 [SMILES: C[C@H]1CN2C[C@@H](CCCOc3cc(cc4[nH]c(=O)n(C)c34)Nc3nc2ncc3Cl)C1(F)F ; Cc1ccc(S(=O)(=O)OCCC(C)(C)O)cc1 ; C[C@H]1CN2C[C@@H](CCCOc3cc(cc4c3n(C)c(=O)n4CCC(C)(C)O)Nc3nc2ncc3Cl)C1(F)F]

### tert-Butyl (3R,5S)-3-(3-(6-(benzylamino)-3-methyl-2-oxo-2,3-dihydro-1H-benzo[d]imidazol-4-yl)oxy)propyl)-4,4-difluoro-5-methylpiperidine-1-carboxylate

To a solution of 5-(benzylamino)-7-methoxy-1-methyl-1,3-dihydro-2H-benzo[d]imidazol-2-one (500 mg, 1.86 mmol, 1 eq) in DMSO (5 mL) were added tert-butyl (3S,5R)-4,4-difluoro-3-methyl-5-(3-(tosyloxy)propyl)piperidine-1-carboxylate (830.93 mg, 1.86 mmol, 1 eq) and Cs₂CO₃ (1.21 g, 3.71 mmol, 2 eq). The reaction mixture was stirred at 25° C. for 2 hr. The reaction mixture (combined with another batch with the same scale) was treated with water (20 mL), then extracted with EtOAc (25 mL×3). The combined organic phase was washed with brine (50 mL), dried with anhydrous Na₂SO₄, filtered and concentrated in vacuo to give a residue which was purified by prep-HPLC (column: Welch Xtimate® C18 180*70 mm #10 μm;mobile phase: [water (NH₄HCO₃)-ACN]; B %: 50%-80%, 15 min) to give the title compound as a white solid (850 mg, 1.51 mmol, 59% yield, 97% purity). ¹H NMR (400 MHz, DMSO-d₆) δ=10.35 (s, 1H), 7.38-7.26 (m, 4H), 7.24-7.17 (m, 1H), 6.03 (d, J=1.6 Hz, 1H), 5.93 (t, J=6.0 Hz, 1H), 5.82 (d, J=1.6 Hz, 1H), 4.21 (d, J=6.0 Hz, 2H), 4.16-3.85 (m, 4H), 3.33 (s, 3H), 2.69-2.53 (m, 2H), 2.04-1.69 (m, 5H), 1.35-1.22 (m, 1H), 0.94 (d, J=6.8 Hz, 3H).

### tert-Butyl (3R,5S)-3-(3-((6-amino-3-methyl-2-oxo-2,3-dihydro-1H-benzo[d]imidazol-4-yl)oxy)propyl)-4,4-difluoro-5-methylpiperidine-1-carboxylate

To a solution of tert-butyl (3R,5S)-3-(3-((6-(benzylamino)-3-methyl-2-oxo-2,3-dihydro-1H-benzo[d]imidazol-4-yl)oxy)propyl)-4,4-difluoro-5-methylpiperidine-1-carboxylate (850.00 mg, 1.56 mmol, 1 eq) in THF (10 mL) was added Pd/C (300 mg, 10% purity) under Ar. The suspension was degassed under vacuum and purged with H₂ several times. The mixture was stirred under H₂ (15 psi) at 25° C. for 12 hr. The reaction mixture was filtered, and the filtrate was concentrated in vacuo to give the title compound as a brown solid (680 mg, crude). ¹H NMR (400 MHz, DMSO-d₆) δ=10.37 (s, 1H), 5.96 (d, J=1.6 Hz, 1H), 5.90 (d, J=1.6 Hz, 1H), 4.74 (s, 2H), 4.18-3.84 (m, 4H), 3.35 (s, 3H), 2.58 (s, 2H), 2.06-1.72 (m, 6H), 1.40 (s, 9H), 0.93 (d, J=6.6 Hz, 3H).

### tert-Butyl (3R,59)-3-(3-(6-((2,5-dichloropyrimidin-4-yl)amino)-3-methyl-2-oxo-2,3 18dihydro-1H-benzo[d]imidazol-4-yl)oxy)propyl)-4,4-difluoro-5-methylpiperidine-1-carboxylate

To a solution of tert-butyl (3R,5S)-3-(3-((6-amino-3-methyl-2-oxo-2,3-dihydro-1H-benzo[d]imidazol-4-yl)oxy)propyl)-4,4-difluoro-5-methylpiperidine-1-carboxylate (500 mg, 1.10 mmol, 1 eq) in DMF (8 mL) were added 2,4,5-trichloropyrimidine (403.56 mg, 2.20 mmol, 2 eq) and DIPEA (355.44 mg, 2.75 mmol, 479.03 μL, 2.5 eq). The reaction mixture was stirred at 25° C. for 2 hr. The reaction mixture was treated with water (8 mL). The suspension was filtered and the precipate was purified by column chromatography (SiO₂, petroleum ether/ethyl acetate=1/1 to 0/1) to give the title compound as a yellow solid (500 mg, 656.72 μmol, 60% yield). ¹H NMR (400 MHz, DMSO-d₆) δ=10.90 (s, 1H), 9.39 (s, 1H), 8.34 (s, 1H), 6.97 (s, 2H), 4.17-4.01 (m, 3H), 3.99-3.85 (m, 1H), 3.45 (s, 3H), 2.66-2.52 (m, 2H), 2.03-1.80 (m, 5H), 1.39 (s, 10H), 0.94 (d, J=6.8 Hz, 3H).

### 5-((2,5-Dichloropyrimidin-4-yl)amino)-7-(3-((3R,5S)-4,4-difluoro-5-methylpiperidin-3-yl)propoxy)-1-methyl-1,3-dihydro-2H-benzo[d]imidazol-2-one

A solution of tert-butyl (3R,5S)-3-(3-((6-((2,5-dichloropyrimidin-4-yl)amino)-3-methyl-2-oxo-2,3-dihydro-1H-benzo[d]imidazol-4-yl)oxy)propyl)-4,4-difluoro-5-methylpiperidine-1-carboxylate (500.00 mg, 831.29 μmol, 1 eq) in DCM (5 mL) and TEA (1 mL) was stirred at 25° C. for 0.5 hr. The reaction mixture was concentrated in vacuo to give the title compound as a yellow oil (500 mg, crude, TFA salt). LCMS: [M+H]⁺=501.2.

(1³R, 1⁵S)-2⁵-chloro-1⁴,1⁴-difluoro-1⁵,4³-dimethyl-4²,4³-dihydro-4¹H-5-oxa-3-aza-4(6,4)-benzo[d]imidazola-2(2,4)-pyrimidina-1 (1,3)-piperidinacyclooctaphan-4²-one (27)

To a solution of S-((2,5-dichloropyrimidin-4-yl)amino)-7-(3-((3R,5S)-4,4-difluoro-5-methylpiperidin-3-yl)propoxy)-1-methyl-1,3-dihydro-2H-benzo[d]imidazol-2-one (500 mg, 812.51 μmol, 1 eq, TFA) in DMSO (20 mL) was added DIPEA (1.05 g, 8.13 mmol, 1.42 mL, 10 eq). The reaction mixture was stirred at 80° C. for 12 hr. The reaction mixture was then treated with water (30 mL). The suspension was filtered to give a residue which was purified by column chromatography (Si(2, petroleum ether/ethyl acetate=1/1 to 0/1) to give the title compound as a yellow solid (250 mg, 510.87 μmol, 63% yield, 95% purity). ¹H NMR (400 MHz, DMSO-d₆) δ=10.80 (s, 1H), 8.75 (s, 1H), 8.05 (s, 1H), 7.56-7.41 (m, 1H), 6.63 (s, 1H), 4.81-4.51 (m, 2H), 4.48-4.33 (m, 1H), 4.17-3.99 (m, 1H), 3.41 (s, 3H), 2.79-2.64 (m, 2H), 2.41-2.14 (m, 2H), 2.05-1.86 (m, 2H), 1.70-1.54 (m, 2H), 0.96 (d,)=6.0 Hz, 3H). LCMS: [M+H]⁺=465.2. 30 mg of the residue was further purified by prep-HPLC (column: Waters Xbridge BEH C18 100*30 mm*10 μm;mobile phase: [water (NH₄HCO₃)-ACN]; B %: 40%-70%, 8 min) to give the title compound as a white solid (10 mg, 99% purity). ¹H NMR (400 MHz, DMSO-d₆) δ=10.81 (s, 1H), 8.77 (s, 1H), 8.06 (s, 1H), 7.49 (d, J=1.2 Hz, 1H), 6.63 (d, J=1.3 Hz, 1H), 4.70 (br d, J=12.8 Hz, 1H), 4.58 (br d,)=11.4 Hz, 1H), 4.48-4.35 (m, 1H), 4.07 (dt, J=7.0, 11.4 Hz, 1H), 3.41 (s, 3H), 2.73 (br t, J=12.6 Hz, 1H), 2.59-2.54 (m, 1H), 2.35-2.25 (m, 1H), 2.03-1.90 (m, 1H), 1.87-1.77 (m, 1H), 1.74-1.58 (m, 2H), 1.35 (dt, J=5.2, 12.8 Hz, 1H), 0.97 (d, J=6.7 Hz, 3H). LCMS: [M+H]⁺=465.3.

### (1³R, 1⁵S)-2⁵-Chloro-1⁴, 1⁴-difluoro-4¹-(3-hydroxy-3-methylbutyl)-1⁵,4³-dimethyl-4²,4³-dihydro-4¹H-5-oxa-3-aza-4(6,4)-benzo[d]imidazola-2(2,4)-pyrimidina-1 (1,3)-piperidinacyclooctaphan-4²-one (11)

To a mixture of (1³R,1⁵S)-2⁵-chloro-1⁴,1⁴-difluoro-1⁵,4³-dimethyl-4²,4³-dihydro-4 H-5-oxa-3-aza-4(6,4)-benzo[d]imidazola-2(2,4)-pyrimidina-1 (1,3)-piperidinacyclooctaphan-4²-one (150 mg, 322.65 μmol, 1 eq) and 3-hydroxy-3-methylbutyl 4-methylbenzenesulfonate (166.70 mg, 645.31 μmol, 2 eq) in DMSO (5 mL) was added KI (26.78 mg, 161.33 μmol, 0.5 eq) and Cs₂CO₃ (210.25 mg, 645.31 μmol, 2 eq). The reaction mixture was stirred at 80° C. for 12 hr under N₂. The rection mixture was treated with water and the suspension was filtered. The solid was purified by prep-HPLC (column: Waters Xbridge™ Prep OBD C18 150*40 mm*10 μm;mobile phase: [water (NH₄HCO₃)-ACN]; B %: 45%-75%, 8 min) to give the title compound as a white solid (65 mg, 115.60 μmol, 36% yield, 98% purity). ¹H NMR (400 MHz, DMSO-d₆) δ=8.84 (s, 1H), 8.07 (s, 1H), 7,53 (s, 1H), 6.76 (s, 1H), 4.69 (d, J=12.4 Hz, 1H), 4.58 (d, J=12.6 Hz, 1H), 4.47-4.37 (m, 2H), 4.13-4.03 (m, 1H), 3.82 (dd, J=6.4, 9.2 Hz, 2H), 3.46 (s, 3H), 2.72 (t,/=12.4 Hz, 1H), 2.57-2.51 (m, 1H), 2.42-2.23 (m, 1H), 2.03-1.89 (m, 1H), 1.86-1.76 (m, 1H), 1.75-1.56 (m, 4H), 1.38-1.29 (m, 1H), 1.18 (s, 6H), 0.96 (d, J=6.4 Hz, 3H). LCMS: [M+H]⁺=551.4.

### Example 7: Synthesis of (1³S,1⁵R)-2⁵-chloro-1⁴,1⁴-difluoro-1⁵,4¹4³-trimethyl-4²,4³-dihydro-4¹H-5-oxa-3-aza-4(6,4)-benzo[d]imidazola-2(2,4)-pyrimidina-1 (1,3)-piperidinacyclooctaphan-4²-one (29)

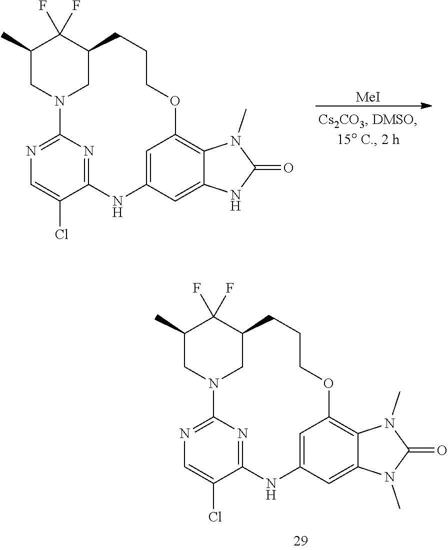 [SMILES: "MeI" ; C[C@@H]1CN2C[C@H](CCCOc3cc(cc4[nH]c(=O)n(C)c34)Nc3nc2ncc3Cl)C1(F)F ; C[C@@H]1CN2C[C@H](CCCOc3cc(cc4c3n(C)c(=O)n4C)Nc3nc2ncc3Cl)C1(F)F]

A mixture of (1³S,1⁵R)-2⁵-chloro-1⁴,1⁴-difluoro-1⁵,4³-dimethyl-4²,4³-dihydro-4¹H-5-oxa-3-aza-4(6,4)-benzo[d]imidazola-2(2,4)-pyrimidina-1 (1,3)-piperidinacyclooctaphan-4²-one (70 mg, 150.57 μmol, 1 eq), Mel (23.51 mg, 165.63 μmol, 10.31 μL, 1.1 eq), and Cs₂CO₃ (73.59 mg, 225.86 μmol, 1.5 eq) in DMSO (1 mL) was stirred at 15° C. for 2 hr. The mixture was filtered and the filtrate was purified by prep-HPLC (column: Waters Xbridge™ Prep OBD C18 150*40 mm*10 μm; mobile phase: [water (NH₄HCO₃)-ACN]; B %: 45%-75%, 8 min) to give the title compound as a white solid (30 mg, 62.01 μmol, 41% yield, 99% purity). ¹H NMR (400 MHz, DMSO-d₆) δ=8.82 (s, 1H), 8.07 (s, 1H), 7,55 (s, 1H), 6.75 (s, 1H), 4.68 (br d, J=12.5 Hz, 1H), 4.58 (br d, J=13.4 Hz, 1H), 4.42 (br t, J=11.8 Hz, 1H), 4.15-4.04 (m, 1H), 3.46 (s, 3H), 3.26 (s, 3H), 2.72 (br t, J=12.6 Hz, 1H), 2.57-2.52 (m, 1H), 2.40-2.24 (m, 1H), 2.02-1.77 (m, 2H), 1.76-1.57 (m, 2H), 1.38-1.26 (m, 1H), 0.96 (d, J=6.7 Hz, 3H), 1.00-0.91 (m, 1H), 0.60-0.35 (m, 1H), LCMS: [M+H]⁺=479.2.

### Example 8: Synthesis of (1³R₁₅₅)-2⁵-chloro-1⁴,1⁴-difluoro-1⁵,4¹4³-trimethyl-4²,4³-dihydro-4¹H-5-oxa-3-aza-4(6,4)-benzo[d]imidazola-2(2,4)-pyrimidina-1 (1,3)-piperidinacyclooctaphan-4²-one (30)

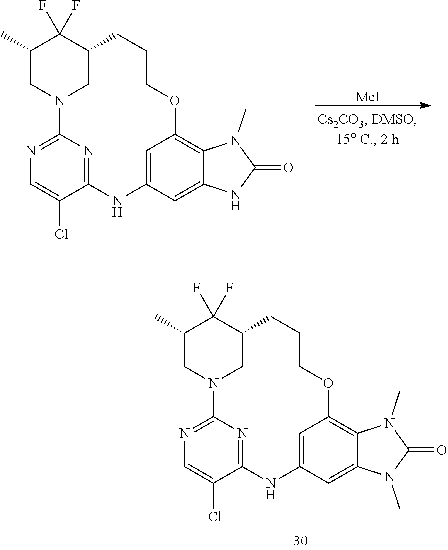 [SMILES: "MeI" ; C[C@H]1CN2C[C@@H](CCCOc3cc(cc4[nH]c(=O)n(C)c34)Nc3nc2ncc3Cl)C1(F)F ; C[C@H]1CN2C[C@@H](CCCOc3cc(cc4c3n(C)c(=O)n4C)Nc3nc2ncc3Cl)C1(F)F]

To a solution of (1³R,1⁵S)-2⁵-chloro-1⁴,1⁴-difluoro-1⁵,4³-dimethyl-4²,4³-dihydro-4 H-5-oxa-3-aza-4(6,4)-benzo[d]imidazola-2(2,4)-pyrimidina-1 (1,3)-piperidinacyclooctaphan-4²-one (30 mg, 64.53 μmol, 1 eq) in DMSO (0.5 mL) were added Cs₂CO₃ (31.54 mg, 96.80 μmol, 1.5 eq) and iodomethane (10.08 mg, 70.98 μmol, 4.42 μL, 1.1 eq). The reaction mixture was stirred at 15° C. for 2 hr. The reaction mixture (combined with 10 mg scale and 30 mg scale) was filtered and the filtrate was concentrated in vacuo to give a residue which was purified by p-HPLC (column: Waters Xbridge™ Prep OBD C18 150*40 mm*10 μm;mobile phase: [water (NH₄HCO₃)-ACN]; B %: 50%-80%, 8 min) to give the title compound as a white solid (20 mg, 41.59 μmol, 99% yield, 99% purity). ¹H NMR (400 MHz, DMSO-d₆) δ=8.82 (s, 1H), 8.07 (s, 1H), 7,55 (d, J=1.1 Hz, 1H), 6.75 (d, J=1.3 Hz, 1H), 4.69 (br d, J=13.4 Hz, 1H), 4.59 (br d, J=13.1 Hz, 1H), 4.43 (br t, J=11.8 Hz, 1H), 4.09 (d, J=7,5, 11.3 Hz, 1H), 3.47 (s, 3H), 3.26 (s, 3H), 2.73 (br t, J=12.6 Hz, 1H), 2.60-2.52 (m, 1H), 2.41-2.25 (m, 1H), 2.01-1.58 (m, 4H), 1.40-1.29 (m, 1H), 0.97 (d, J=6.8 Hz, 3H). LCMS: [M+H]⁺=479.3.

### Example 9: Synthesis of (138, 15R)-23-chloro-1⁴,1⁴-difluoro-4-(3-hydroxy-3-methylbutyl)-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (14)

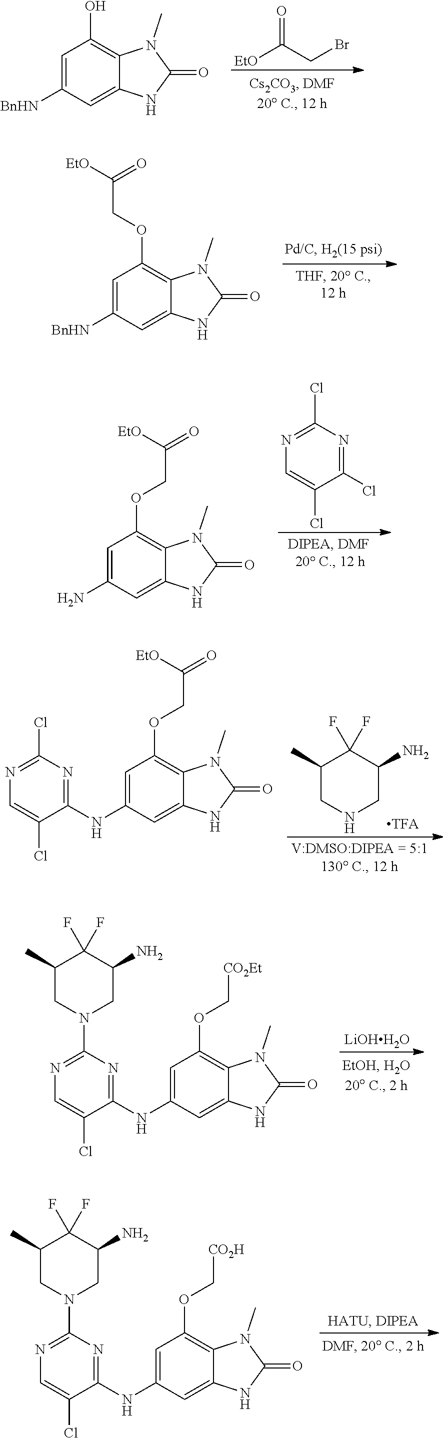

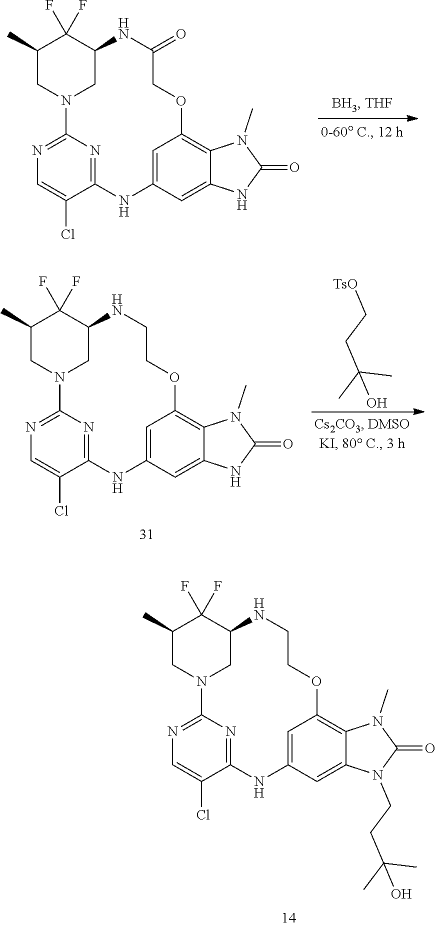 [SMILES: C[C@@H]1CN2C[C@H](NC(=O)COc3cc(cc4[nH]c(=O)n(C)c34)Nc3nc2ncc3Cl)C1(F)F ; C[C@@H]1CN2C[C@H](NCCOc3cc(cc4[nH]c(=O)n(C)c34)Nc3nc2ncc3Cl)C1(F)F ; Cc1ccc(S(=O)(=O)OCCC(C)(C)O)cc1 ; C[C@@H]1CN2C[C@H](NCCOc3cc(cc4c3n(C)c(=O)n4CCC(C)(C)O)Nc3nc2ncc3Cl)C1(F)F]

### Ethyl 2-((6-(benzylamino)-3-methyl-2-oxo-2,3-dihydro-1H-benzo[d]imidazol-4-yl)oxy)acetate

A flask with a mixture of 6-(benzylamino)-4-hydroxy-3-methyl-1H-benzimidazol-2-one (15 g, 55.71 mmol, 1 eq), ethyl 2-bromoacetate (8.35 g, 50.15 mmol, 5.55 mL, 0.9 eq), and Cs₂CO₃ (36.32 g, 111.43 mmol, 2 eq) in DMSO (100 mL) was degassed and purged with N₂3 times. The reaction mixture was stirred at 20° C. for 12 hr under N₂ atmosphere. Water (100 mL) was added to the reaction mixture and the solid was filtered. The filter cake was dried under reduced pressure. The solid was washed with MeOH (50 mL) and then dried under reduced pressure to give the title compound as a white solid (8 g, 22.51 mmol, 40% yield). ¹H NMR (400 MHz, DMSO-d₆) δ ppm 10.41 (s, 1H), 7.35-7.29 (m, 3H), 7.23-7.22 (m, 1H), 5.97-5.94 (m, 2H), 5.88-5.87 (m, 1H), 4.72 (s, 2H), 4.21 (d, J=6.0 Hz, 2H), 4.18-4.13 (m, 2H), 3.38 (s, 3H), 1.21 (t, J=7.2 Hz, 3H).

### Ethyl 2-((6-amino-3-methyl-2-oxo-2,3-dihydro-1H-benzo[d]imidazol-4-yl)oxy)acetate

A flask with a mixture of ethyl 2-((6-(benzylamino)-3-methyl-2-oxo-2,3-dihydro-1H-benzo[d]imidazol-4-yl)oxy)acetate (8 g, 22.52 mmol, 1 eq) and Pd/C (300 mg, 10% purity) in THE (100 mL) was degassed and purged with H_{2 3} times. The reaction mixture was stirred at 20° C. for 12 hr under H₂ atmosphere. The reaction mixture was filtered and the filtrate was concentrated in vacuo to give the title compound as a white solid (4.5 g, 16.96 mmol, 75% yield). LCMS: [M+H]⁻=266.3.

### Ethyl 2-((6-((2,5-dichloropyrimidin-4-yl)amino)-3-methyl-2-oxo-2,3-dihydro-1H-benzo[d]imidazol-4-yl)oxy)acetate

A flask with a mixture of ethyl 2-((6-amino-3-methyl-2-oxo-2,3-dihydro-1H-benzo[d]imidazol-4-yl)oxy)acetate (4.5 g, 16.96 mmol, 1 eq), 2,4,5-trichloropyrimidine (6.22 g, 33.93 mmol, 2 eq), and DIPEA (4.38 g, 33.93 mmol, 5.91 mL, 2 eq) in DMF (30 mL) was degassed and purged with N₂3 times. The reaction mixture was stirred at 20° C. for 12 hr under N₂ atmosphere. Water (30 mL) was added to the reaction mixture and the pink solid was filtered. The filter cake was dried under reduced pressure and then washed with EtOAc (40 mL). The solid was filtered and the filter cake was dried under reduced pressure to give the title compound as a pink solid (4 g, 9.70 mmol, 57% yield). ¹H NMR (400 MHz, DMSO-d₆) δ ppm 10.96 (s, 1H), 9.38 (s, 1H), 8.35 (s, 1H), 7.02 (s, 1H), 6.95 (s, 1H), 4.84 (s, 2H), 4.22-4.16 (m, 2H), 3.51 (s, 3H), 1.22 (t, J=7.2 Hz, 3H).

### Ethyl 2-((6-((2-(3S,5R)-3-amino-4,4-difluoro-5-methylpiperidin-1-yl)-5-chloropy rimidin-4-yl)amino)-3-methyl-2-oxo-2,3-dihydro-1H-benzo[d]imidazol-4-yl)oxy)acetate

A flask with a mixture of ethyl 2-((6-((2,5-dichloropyrimidin-4-yl)amino)-3-methyl-2-oxo-2,3-dihydro-1H-benzo[d]imidazol-4-yl)oxy)acetate (1.5 g, 3.64 mmol, 1 eq) and (3S,5R)-4,4-difluoro-5-methyl-piperidin-3-amine (1.65 g, 6.26 mmol, 1.72 eq, TFA) in DMSO (30 mL) and DIPEA (6 mL) was degassed and purged with N₂3 times. The reaction mixture was stirred at 130° C. for 12 hr under N₂ atmosphere. The reaction mixture was poured into water and filtered. The solid was washed with water to give the title compound as a yellow solid (1.2 g, 2.28 mmol, 63% yield). ¹H NMR (400 MHz, DMSO-d₆) δ ppm 10.90 (s, 1H), 8.63 (s, 1H), 8.04 (s, 1H), 7.05 (s, 1H), 7.02 (s, 1H), 4.83 (s, 2H), 4.58-4.43 (m, 2H), 4.21-4.15 (m, 2H), 3.50 (s, 3H), 2.94-2.86 (m, 1H), 2.67-2.59 (m, 2H), 2.54 (s, 1H), 2.09-1.95 (m, 2H), 1.68 (s, 2H), 1.21 (t, J=7.2 Hz, 3H), 0.97 (d,)=6.8 Hz, 3H).

### 2-(6-(2-((3S,5R)-3-Amino-4,4-difluoro-5-methylpiperidin-1-yl)-5-chloropyrimidin-4-yl)amino)-3-methyl-2-oxo-2,3-dihydro-1H-benzo[d]imidazol-4-yl)oxy)acetic acid

A flask with a mixture of ethyl 2-((6-((2-((3S,5R)-3-amino-4,4-difluoro-5-methylpiperidin-1-yl)-5-chloropyrimidin-4-yl)amino)-3-methyl-2-oxo-2,3-dihydro-1H-benzo[d]imidazol-4-yl)oxy)acetate (1.2 g, 2.28 mmol, 1 eq) and LiOH H₂O (191.49 mg, 4.56 mmol, 2 eq) in EtOH (10 mL) and H₂O (2 mL) was degassed and purged with N₂3 times. The reaction mixture was stirred at 20° C. for 2 hr under N₂ atmosphere. The reaction mixture was concentrated under reduced pressure to give a residue. The residue was adjusted to pH=4 with 1N HCl, filtered, and washed with water to give the title compound as a yellow solid (700 mg, 1.41 mmol, 62% yield). LCMS: [M+H]⁺=498.3.

### (1³S,1′R)-2⁵-chloro-1⁴,1⁴-difluoro-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphane-42,7-dione

A flask with a mixture of 2-((6-((2-((3S,5R)-3-amino-4,4-difluoro-5-methylpiperidin-1-yl)-5-chloropyrimidin-4-yl)amino)-3-methyl-2-oxo-2,3-dihydro-1H-benzo[d]imidazol-4-yl)oxy)acetic acid (700 mg, 1.41 mmol, 1 eq), HATU (534.59 mg, 1.41 mmol, 1 eq), and DIPEA (363.42 mg, 2.81 mmol, 489.78 μL, 2 eq) in DMF (10 mL) was degassed and purged with N₂3 times. The reaction mixture was stirred at 20° C. for 2 hr under N₂ atmosphere. The reaction mixture was poured into water and filtered. The precipitate was washed with water, then with MeOH (10 mL), and filtered to give the title compound as a yellow solid (400 mg, 833.56 μmol, 59% yield). ¹H NMR (400 MHz, DMSO-d₆) δ ppm 10.82 (s, 1H), 8.69 (s, 1H), 8.45 (d, J=8.4 Hz, 1H), 8.00 (s, 1H), 7.08 (s, 1H), 6.57 (s, 1H), 4.95 (d, J=14.8 Hz, 1H), 4.61 (d, J=14.4 Hz, 1H), 4.42-4.27 (m, 2H), 4.03-3.98 (m, 1H), 3.46 (s, 3H), 3.29-3.26 (m, 2H), 2.67-2.54 (m, 1H), 1.09 (d,)=6.8 Hz, 3H). LCMS: [M+H]⁺=480.1.

### (1³S,1⁵R)-2⁵-chloro-1⁴,1⁴-difluoro-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (31)

To a solution of (1³S,1⁵R)-2⁵-chloro-1⁴,1⁴-difluoro-1⁵,4³-dimethyl-4²,4³-dihydro-4¹H₁-5-oxa-3,8-diaza-4(6,4)-benzo[d]imidazola-2(2,4)-pyrimidina-1 (1,3)-piperidinacyclooctaphane-42,7-dione in THF (5 mL) was added BH₃. THF (1 M, 3.33 mL, 4 eq) at 0° C. under N₂. The reaction mixture was stirred at 60° C. for 12 hr. Sat, aq. NH₄CI (40 mL) was added slowly under N₂ and the reaction mixture stirred for 20 min after the addition. The reaction mixture was poured into water (40 mL) and extracted with EtOAc (40 mL×2). The organic phase concentrated in vacuo to give a residue which was purified by column chromatography (SiO₂, petroleum ether/ethyl acetate=1/0 to 0/1) to give the title compound as a light yellow solid (200 mg, 429.29 μmol, 52% yield). ¹H NMR (400 MHz, DMSO-d₆) δ ppm 10.80 (s, 1H), 8.78 (s, 1H), 8.06 (s, 1H), 7,55 (s, 1H), 6.64 (s, 1H), 4.71 (d, J=12.4 Hz, 1H), 4.53-4.39 (m, 2H), 4.07-3.99 (m, 1H), 3.41 (s, 3H), 3.30-3.20 (m, 1H), 2.98-2.92 (m, 2H), 2.73 (t, J=12.6 Hz, 1H), 2.58-2.52 (m, 1H), 2.25-2.15 (m, 1H), 2.05-1.88 (m, 1H), 0.96 (d, J=6.8 Hz, 3H). LCMS: [M+H]⁺=466.3.

### (1³S,1⁵R)-2⁵-chloro-1⁴,1⁴-difluoro-4¹-(3-hydroxy-3-methylbutyl)-1⁵,4³-dimethyl-4²,4³-dihydro-41 H-5-oxa-3,8-diaza-4(6,4)-benzo[d]imidazola-2(2,4)-pyrimidina-1 (1,3)-piperidinacyclooctaphan-4²-one (14)

A flask with a mixture of (1³S,1⁵R)-2⁵-chloro-1⁴,1⁴-difluoro-1⁵,4³-dimethyl-4²,4³-dihydro-4 H-5-oxa-3,8-diaza-4(6,4)-benzo[d]imidazola-2(2,4)-pyrimidina-1 (1,3)-piperidinacyclooctaphan-4²-one (100 mg, 214.65 μmol, 1 eq), (3-hydroxy-3-methyl-butyl) 4-methylbenzenesulfonate (83.18 mg, 321.97 μmol, 1.5 eq), KI (17.82 mg, 107.32 μmol, 0.5 eq), and Cs₂CO₃ (139.87 mg, 429.29 μmol, 2 eq) in DMSO (5 mL) was degassed and purged with N_{2 3} times. The reaction mixture was stirred at 80° C. for 3 hr under N₂ atmosphere. The reaction mixture was concentrated under reduced pressure to give a residue (combined with other 2 batches) which was purified by prep-HPLC (column: Waters Xbridge™ Prep OBD C18 150*40 mm*10 um; mobile phase: [H₂O (10 mM NH₄HCO₃)-ACN]; gradient: 35%-60% B over 8.0 min) to give 80 mg of product. The product (80 mg) was further purified by SFC (column: DAICEL CHIRALPAK AD (250 mm*30 mm*10 μm); mobile phase: [CO₂-MeOH (0.1% NH₃H₂O)]; B %: 46%, isocratic elution mode) to give the title compound as a white solid (47.7 mg, 86.41 μmol). ¹H NMR (400 MHz, DMSO-d₆) δ ppm 8.87 (s, 1H), 8.08 (s, 1H), 7.60 (s, 1H), 6.76 (s, 1H), 4.71 (d, J=12.4 Hz, 1H), 4.52 (d, J=13.6 Hz, 1H), 4.45-4.39 (m, 2H), 4.08-4.01 (m, 1H), 3.84-3.80 (m, 2H), 3.46 (s, 3H), 3.30-3.24 (m, 1H), 2.99-2.92 (m, 2H), 2.74 (t, J=12.8 Hz, 1H), 2.54 (s, 1H), 2.25-2.15 (m, 1H), 2.05-1.88 (m, 1H), 1.73-1.65 (m, 2H), 1.18 (s, 6H), 0.97 (d, J=6.4 Hz, 3H). LCMS: [M+H]⁺=552.3. The stereochemistry of compound 14 was confirmed by crystal structure of BCL6/compound 14.

### Example 10: Synthesis of (1³R,159)-2⁵-chloro-1⁴, 1⁴-difluoro-4¹-(3-hydroxy-3-methylbutyl)-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (15)

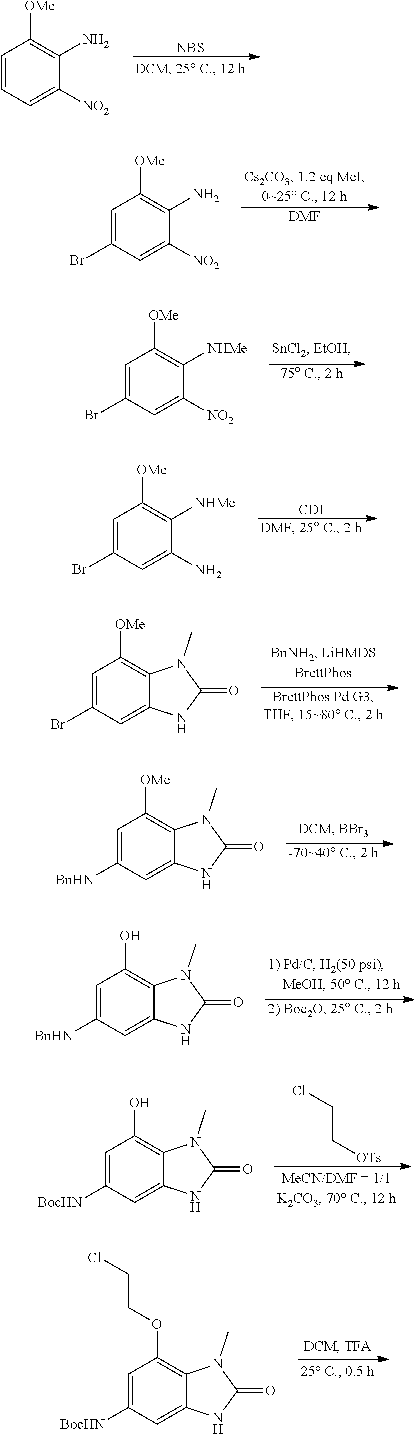

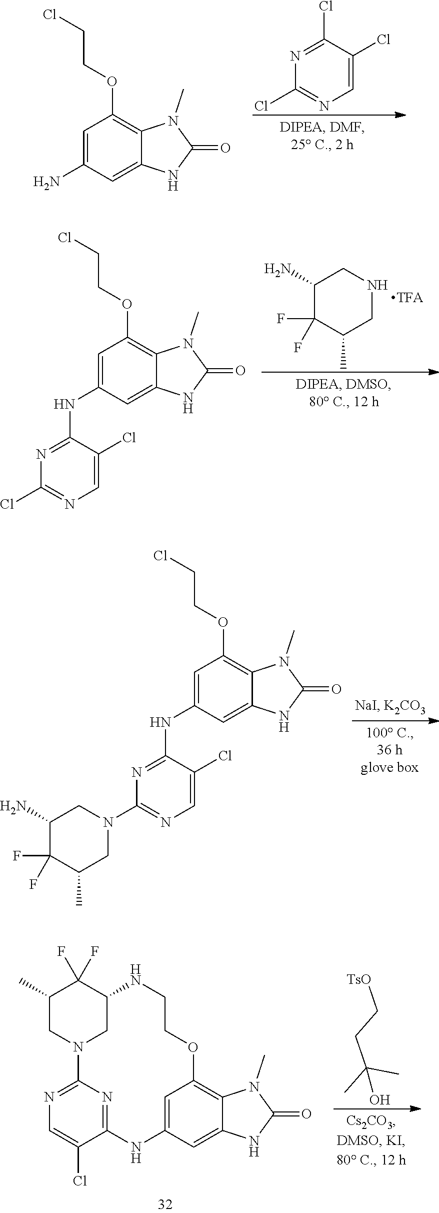

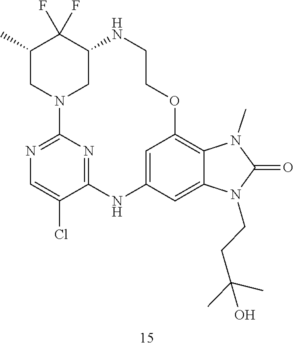 [SMILES: C[C@H]1CN2C[C@@H](NCCOc3cc(cc4c3n(C)c(=O)n4CCC(C)(C)O)Nc3nc2ncc3Cl)C1(F)F]

### 4-Bromo-2-methoxy-6-nitroaniline

To a solution of 2-methoxy-6-nitroaniline (100 g, 594.71 mmol, 100 mL, 1 eq) in DCM (2 L) was added NBS (137.60 g, 773.12 mmol, 1.3 eq). The reaction mixture was stirred at 25° C. for 12 hr. The reaction mixture (combined with other 4 batches with the same scale) was concentrated in vacuo and the residue was triturated with water (1 L) at 25° C. for 30 min and filtered. The filter cake was dried and then triturated with petroleum ether/ethyl acetate=4/1 (500 mL) at 25° C. for 30 min. The solution was filtered and the filter cake was dried to give the title compound as a yellow solid (500 g, 95% purity, 68% yield). ¹H NMR (400 MHz, MeCN-d₃) δ=7.77 (d, J=2.1 Hz, 1H), 7.06 (d, J=2.0 Hz, 1H), 6.64 (br s, 2H), 3.90 (s, 3H).

### 4-Bromo-2-methoxy-N-methyl-6-nitroaniline

A solution of 4-bromo-2-methoxy-6-nitroaniline (50 g, 202.39 mmol, 1 eq) in DMF (2 L) was stirred at 0° C. for 10 min. Cs₂CO₃ (131.89 g, 404.78 mmol, 2 eq) was added and the reaction mixture was stirred at 0° C. for 10 min. Mel (34.47 g, 242.87 mmol, 15.12 mL, 1.2 eq) was added dropwise and the reaction mixture was stirred at 25° C. for 12 hr. The reaction mixture was washed with water (5 L). The red solid was filtered and the filter cake was washed with water (1500 mL×3). Then the solid was dried under reduced pressure to give the title compound as a red solid (375 g, 93% purity, 70% yield). ¹H NMR (400 MHz, CDCl₃) δ=7,59 (d, J=1.6 Hz, 1H), 7.24 (d, J=5.2 Hz, 1H), 7.18 (s, 1H), 3.87 (s, 3H), 2.86 (d, J=5.2 Hz, 3H). LCMS: [M+H]⁺=261.1.

### 4-Bromo-6-methoxy-NI-methylbenzene-1,2-diamine

A mixture of 4-bromo-2-methoxy-N-methyl-6-nitroaniline (75 g, 287.28 mmol, 1 eq) and SnCl₂ (259.30 g, 1.15 mol, 4 eq) in EtOH (1.5 L) was stirred at 75° C. for 2 hr under N₂ atmosphere. The reaction mixture (combined with four other batches of the same scale) was concentrated in vacuo. The residue was treated with water (2 L) and the pH was adjusted to 14 with NaOH. The solid was filtered and the filter cake was washed with EtOAc (4 L). The filtrate was extracted with EtOAc (1 L×3). The combined organic phase was washed with brine (3 L), dried with anhydrous Na₂SO₄, filtered and concentrated in vacuo to give a residue which was purified with column chromatography (SiO₂, petroleum ether/ethyl acetate=50/1 to 2/1) to give the title compound as a colorless oil (260 g, 1.08 mol, 75% yield, 96% purity). ¹H NMR (400 MHz, CDCl₃)8=6.54 (d, J=1.8 Hz, 1H), 6.45 (d, J=2.0 Hz, 1H), 4.11-3.83 (m, 2H), 3.79 (s, 3H), 3.45-2.85 (m, 1H), 2.63 (s, 3H). LCMS: [M+H]=231.0.

### 5-Bromo-7-methoxy-1-methyl-1,3-dihydro-2H-benzo[d]imidazol-2-one

To a solution of 4-bromo-6-methoxy-NI-methylbenzene-1,2-diamine (45 g, 194.73 mmol, 1 eq) in DMF (450 mL) was added CDI (63.15 g, 389.46 mmol, 2 eq). The reaction mixture was stirred at 25° C. for 2 hr. The reaction mixture was diluted with water (500 mL) and then filtered. The filter cake was washed with water (500 mL), followed by EtOAc (500 mL). The solid was dried in vacuo to give the title compound as a white solid (64 g, 221.56 mmol, 57% yield, 89% purity). ¹H NMR (400 MHz, DMSO-d₆) δ=11.95 (br s, 1H), 7.00 (s, 1H), 6.93 (d, J=1.9 Hz, 1H), 3.86 (d, J=1.8 Hz, 3H), 3.63 (d, J=1.9 Hz, 3H).

### 5-(Benzylamino)-7-methoxy-1-methyl-1,3-dihydro-2H-benzo[d]imidazol-2-one

To a mixture of 5-bromo-7-methoxy-1-methyl-1,3-dihydro-2H-benzo[d]imidazol-2-one (32 g, 124.47 mmol, 1 eq) and phenylmethanamine (16.01 g, 149.37 mmol, 16.28 mL, 1.2 eq) in THF (320 mL) was added LiHMDS (1 M, 298.74 mL, 2.4 eq) dropwise at 15° C. The reaction mixture was degassed and purged with N₂ for S min. BrettPhos (2.67 g, 4.98 mmol, 0.04 eq) and BrettPhos Pd G3 (2.26 g, 2.49 mmol, 0.02 eq) were added to the reaction mixture which was stirred at 80° C. for 2 hr under N₂. The reaction mixture (combined with another batch of the same scale) was quenched with sat, aq. NH₄CI (50 mL), filtered, dried with anhydrous Na₂SO₄, and filtered. The filtrate was concentrated in vacuo and the residue was purified with column chromatography (Si(2, petroleum ether/ethyl acetate=50/1 to 2/1) to give 65 g of ˜90% purity product which was triturated with EtOAc (150 mL) at 15° C. for 30 min to give the title compound as a brown solid (58 g, 192.43 mmol, 77% yield, 94% purity). ¹H NMR (400 MHz, DMSO-d₆) δ=10.37 (s, 1H), 7.37-7.29 (m, 4H), 7.24-7.18 (m, 1H), 6.06 (d, J=1.8 Hz, 1H), 5.96 (t, J=6.0 Hz, 1H), 5.83 (d, J=2.0 Hz, 1H), 4.23 (d, J=6.0 Hz, 2H), 3.73 (s, 3H), 3.32 (s, 3H).

### 5-(Benzylamino)-7-hydroxy-1-methyl-1,3-dihydro-2H-benzo[d]imidazol-2-one

To a solution of 5-(benzylamino)-7-methoxy-1-methyl-1,3-dihydro-2H-benzo[d]imidazol-2-one (58 g, 204.71 mmol, 1 eq) in DCM (1.1 L) was added a solution of BBrs (153.86 g, 614.14 mmol, 59.18 mL, 3 eq) in DCM (100 mL) at −70° C. The reaction mixture was warmed to 40° C., and stirred at 40° C. for 2 hr. The reaction mixture was quenched with CH₃Cl: MeOH=10:1 (1.1 L) and then the reaction mixture was concentrated in vacuo to give a residue. The residue was treated with 4N HCl (150 mL) and stirred at 40° C. for 30 min. The resulting mixture was treated with sat, aq. Na₂CO₃ to pH=9 and then filtered. The filter cake was triturated with EtOAc (150 mL) at 15° C. for 30 min to give the title compound as a white solid (44 g, 158.49 mmol, 77% yield, 97% purity). ¹H NMR (400 MHz, DMSO-d₆) δ=10.23 (s, 1H), 9.26 (s, 1H), 7.36-7.27 (m, 4H), 7.23-7.17 (m, 1H), 5.89-5.83 (m, 2H), 5.72 (d, J=2.0 Hz, 1H), 4.17 (d, J=6.0 Hz, 2H), 3.33 (s, 3H). LCMS: [M+H]⁺=270.2.

### tert-Butyl (7-hydroxy-1-methyl-2-oxo-2,3-dihydro-1H-benzo[d]imidazol-5-yl)carbamate

To a solution of 5-(benzylamino)-7-hydroxy-1-methyl-1,3-dihydro-2H-benzo[d]imidazol-2-one (15 g, 55.70 mmol, 1 eq) in MeOH (300 mL) was added Pd/C (5 g, 10% purity) under Ar. The suspension was degassed and purged with H_{2 3} times. The reaction mixture was stirred under H₂ (50 psi) at 50° C. for 12 hr. Boc₂O) (9.74 g, 44.65 mmol, 10.26 mL, 1 eq) was then added and the reaction mixture was stirred at 25° C. for 2 hr. The reaction mixture was filtered and the filtrate was concentrated in vacuo to give a residue which was triturated with ethyl acetate (30 mL) at 15° C. for 10 min to give the title compound as a red solid (12 g). ¹H NMR (400 MHz, DMSO-d₆) δ=10.60 (s, 1H), 9.66 (s, 1H), 9.16 (br s, 1H), 6.77 (s, 2H), 3.45 (s, 3H), 1.52 (s, 9H). LCMS: [M+H]⁺=280.1.

tert-Butyl (7-(2-chloroethoxy)-1-methyl-2-oxo-2,3-dihydro-1H-benzo[d]imidazol-5-yl)carbamate

To a solution of tert-butyl (7-hydroxy-1-methyl-2-oxo-2,3-dihydro-1H-benzo[d]imidazol-5-yl)carbamate (10.00 g, 35.80 mmol, 1 eq) and 2-chloroethyl 4-methylbenzenesulfonate (8.40 g, 35.80 mmol, 1 eq) in ACN(20 mL) and DMF (20 mL) was added K₂CO₃ (12.37 g, 89.51 mmol, 2.5 eq). The reaction mixture was stirred at 70° C. for 12 hr under N₂. The reaction was quenched with water (100 mL) and then extracted with ethyl acetate (80 mL×3). The organic phase was separated and washed with brine (200 mL), dried with anhydrous Na₂SO₄, filtered and concentrated in vacuo. The crude product was triturated with petroleum ether:ethyl acetate=1:1 (20 mL) at 25° C. for 5 min, filtered and the filter cake was dried in vacuo to give the title compound as a white solid (7.8 g, 19.63 mmol, 55% yield, 86% purity). ¹H NMR (400 MHz, DMSO-d₆) δ=10.73 (s, 1H), 9.19 (s, 1H), 6.89 (s, 1H), 6.84 (s, 1H), 4.24 (t, J=4.8 Hz, 2H), 4.00 (t, J=4.8 Hz, 2H), 3.43 (s, 3H), 1.46 (s, 9H).

### 5-Amino-7-(2-chloroethoxy)-1-methyl-1,3-dihydro-2H-benzo[d]imidazol-2-one

A solution of tert-butyl (7-(2-chloroethoxy)-1-methyl-2-oxo-2,3-dihydro-1H-benzo[d]imidazol-5-yl)carbamate (7.8 g, 22.82 mmol, 1 eq) in TFA (16 mL) and DCM (80 mL) was stirred at 25° C. for 30 min. The reaction mixture was concentrated in vacuo to give the title compound as a brown oil (12 g, crude, TFA). LCMS: [M+H]⁺=242.2.

### 7-(2-Chloroethoxy)-5-((2,5-dichloropyrimidin-4-yl)amino)-1-methyl-1,3-dihydro-2H-benzo[d]imidazol-2-one

A mixture of 5-amino-7-(2-chloroethoxy)-1-methyl-1,3-dihydro-2H-benzo[d]imidazol -2-one (9 g, 19.16 mmol, 1 eq, TFA), 2,4,5-trichloropyrimidine (7.03 g, 38.32 mmol, 2 eq), and DIEA (7.43 g, 57.48 mmol, 10.01 mL, 3 eq) in DMF (90 mL) was degassed and purged with N₂3 times. The reaction mixture was stirred at 25° C. for 2 hr under N₂ atmosphere. Water (30 mL) was added and a solid precipitate formed. The mixture was filtered and the filter cake was washed with petroleum ether:ethyl acetate=1:1 (50 mL) and dried in vacuo to give the title compound as a yellow solid (5.7 g, 13.93 mmol, 73% yield, 95% purity). It was used directly for next step without further purification. ¹H NMR (400 MHz, DMSO-d₆) δ=10.94 (s, 1H), 9.40 (s, 1H), 8.34 (s, 1H), 6.98 (br d, J=14.8 Hz, 2H), 4.36-4.28 (m, 2H), 4.07-3.99 (m, 2H), 3.49 (s, 3H). LCMS: [M+H]⁺=388.1.

### 5-((2-((3R,5S)-3-Amino-4,4-difluoro-5-methylpiperidin-1-yl)-5-chloropyrimidin-4-yl)amino)-7-(2-chloroethoxy)-1-methyl-1,3-dihydro-2H-benzo[d]imidazol-2-one

A flask with a mixture of 7-(2-chloroethoxy)-5-((2,5-dichloropyrimidin-4-yl)amino)-1-methyl-1,3-dihydro-2H-benzo[d]imidazol-2-one (1 g, 2.57 mmol, 1 eq), (3R,5S)-4,4-difluoro-5-methylpiperidin-3-amine (1.95 g, 5.15 mmol, 2 eq, TFA), and DIEA (3.33 g, 25.73 mmol, 4.48 mL, 10 eq) in DMSO (10 mL) was degassed and purged with N₂3 times. The reaction mixture was stirred at 80° C. for 12 hr under N₂ atmosphere. The reaction was quenched with water (30 mL) and extracted with ethyl acetate (25 mL×3). The combined organic phase was dried with anhydrous Na₂SO₄, filtered and concentrated in vacuo to give a residue which was purified with column chromatography (SiO₂, petroleum ether:ethyl acetate=1/1 to ethyl acetate: THF=10:1) to give the title compound as a yellow solid (540 mg, 967.46 μmol, 38% yield, 90% purity). ¹H NMR (400 MHz, DMSO-d₆) δ=10.88 (br s, 1H), 8.64 (br s, 1H), 8.04 (s, 1H), 7.12 (br s, 1H), 7.00 (br s, 1H), 4.65-4.43 (m, 2H), 4.35-4.31 (m, 1H), 4.08-3.98 (m, 2H), 3.55-3.42 (m, 3H), 2.97-2.83 (m, 1H), 2.63 (dt, J=7.0, 12.1 Hz, 2H), 2.35-1.86 (m, 4H), 0.98 (br d, J=6.5 Hz, 3H)

### (1³R, 1⁵S)-2⁵-chloro-1⁴,1⁴-difluoro-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (32)

To a solution of 5-((2-((3R,5S)-3-amino-4,4-difluoro-5-methylpiperidin-1-yl)-5-chloropyrimidin-4-yl)amino)-7-(2-chloroethoxy)-1-methyl-1,3-dihydro-2H-benzo[d]imidazol-2-one (180 mg, 358.32 μmol, 1 eq) in anhydrous DMF (30 mL) was added K₂CO₃ (99.04 mg, 716.64 μmol, 2 eq) and NaI (537.10 mg, 3.58 mmol, 10 eq). The reaction mixture was stirred at 100° C. for 36 hr in a glove box. The reaction mixture was concentrated in vacuo and water (50 mL) was added. The mixture was extracted with ethyl acetate (35 mL×3). The combined organic phase was dried with anhydrous Na₂SO₄, filtered and concentrated in vacuo to give a residue which was purified with column chromatography (SiO₂, petroleum ether: THF=1/0 to 1:1) to give the title compound as a white solid (100 mg, 158.84 μmol, 15% yield, 74% purity). ¹H NMR (400 MHz, DMSO-d₆) δ=10.80 (s, 1H), 8.78 (s, 1H), 8.07 (s, 1H), 7,55 (s, 1H), 6.63 (d, J=1.3 Hz, 1H), 4.79-4.66 (m, 1H), 4.58-4.47 (m, 1H), 4.45-4.36 (m, 1H), 4.13-4.01 (m, 1H), 3.41 (s, 3H), 3.05-2.85 (m, 2H), 2.80-2.64 (m, 1H), 2.54 (br s, 1H), 2.23-2.15 (m, 1H), 2.07-1.89 (m, 1H), 0.96 (d, J=6.8 Hz, 3H). LCMS: [M+1]⁺=466.2.

### (1³R, 1⁵S)-2⁵-chloro-1⁴,1⁴-difluoro-4¹-(3-hydroxy-3-methylbutyl)-1⁵,4³-dimethyl-4²,4³-dihydro-41 H-5-oxa-3,8-diaza-4(6,4)-benzo[d]imidazola-2(2,4)-pyrimidina-1 (1,3)-piperidinacyclooctaphan-4²-one (15)

To a solution of (1³R,1⁵S)-2⁵-chloro-1⁴,1⁴-difluoro-4¹-(3-hydroxy-3-methylbutyl)-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (70 mg, 150.25 μmol, 1 eq) and 3-hydroxy-3-methylbutyl 4-methylbenzenesulfonate (77.63 mg, 300.50 μmol, 2 eq) in DMSO (1 mL) was added KI (12.47 mg, 75.13 μmol, 0.5 eq) and Cs₂CO₃ (97.91 mg, 300.50 μmol, 2 eq). The reaction mixture was stirred at 80° C. for 12 hr. The reaction mixture was filtered and the filtrate was purified with prep-HPLC (column: Waters Xbridge™ BEH C18 100*30 mm*10 μm; mobile phase: [water (NH₄HCO₃)-ACN]; B %: 35%-55%, 8 min) to give the title compound as a white solid (19 mg, 33.73 μmol, 22% yield, 98% purity). ¹H NMR (400 MHz, DMSO-d₆) δ=8.88 (s, 1H), 8.08 (s, 1H), 7.60 (s, 1H), 6.76 (d, J=1.0 Hz, 1H), 4.77-4.65 (m, 1H), 4.56-4.38 (m, 3H), 4.10-3.99 (m, 1H), 3.82 (br dd, J=5.9, 10.3 Hz, 2H), 3.46 (s, 3H), 3.28 (br s, 1H), 3.04-2.86 (m, 2H), 2.79-2.64 (m, 1H), 2.54 (br s, 1H), 2.24-2.17 (m, 1H), 2.05-1.87 (m, 1H), 1.69 (br dd, J=4.7, 9.2 Hz, 2H), 1.18 (s, 6H), 0.97 (d, J=6.8 Hz, 3H). LCMS: [M+H]⁺=552.4.

### Example 11: Synthesis of (1³R, 199)-2⁵-chloro-4-(3-hydroxy-3-methylbutyl)-1⁵,4³-dimethyl-4²,4³-dihydro-4¹H-5,8-dioxa-3-aza-4(6,4)-benzo[d]imidazola-2(2,4)-pyrimidina-1 (1,3)-piperidinacyclooctaphan-4²-one (12)

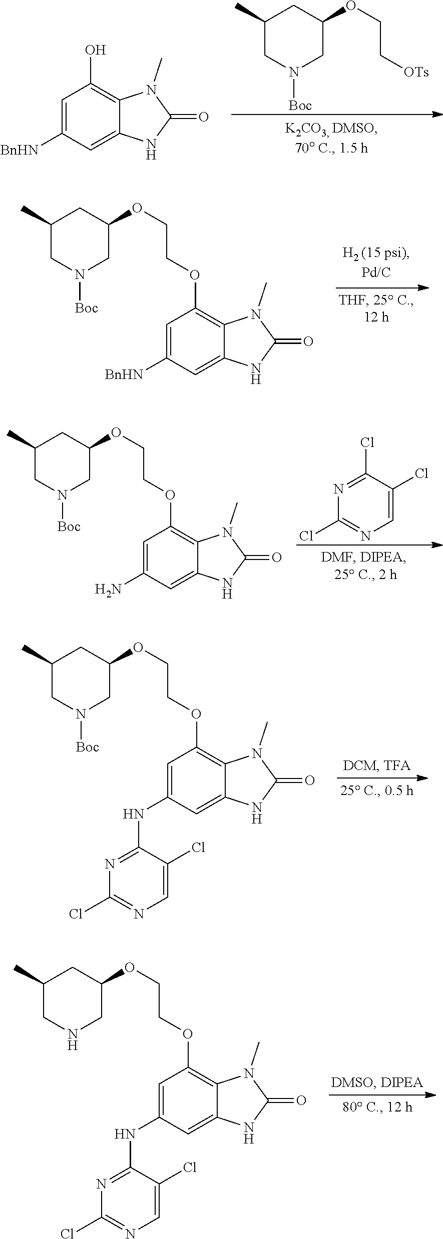

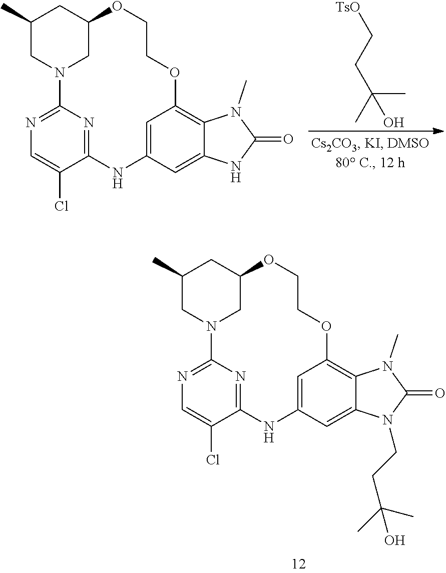 [SMILES: C[C@H]1C[C@@H]2CN(C1)c1ncc(Cl)c(n1)Nc1cc(c3c(c1)[nH]c(=O)n3C)OCCO2 ; Cc1ccc(S(=O)(=O)OCCC(C)(C)O)cc1 ; C[C@H]1C[C@@H]2CN(C1)c1ncc(Cl)c(n1)Nc1cc(c3c(c1)n(CCC(C)(C)O)c(=O)n3C)OCCO2]

tert-Butyl (3R,5S)-3-(2-((6-(benzylamino)-3-methyl-2-oxo-2,3-dihydro-1H-benzo[d]imidazol-4-yl)oxy)ethoxy)-5-methylpiperidine-1-carboxylate

To mixture of 5-(benzylamino)-7-hydroxy-1-methyl-1,3-dihydro-2H-benzo[d]imidazol-2-one (1 g, 3.71 mmol, 1 eq) and tert-butyl (3S,5R)-3-methyl-5-(2-(tosyloxy)ethoxy)piperidine-1-carboxylate (1.54 g, 3.71 mmol, 1 eq) in DMSO (15 mL) was added K₂CO₃ (1.03 g, 7.43 mmol, 2 eq). The reaction mixture was stirred at 70° C. for 1.5 hr under N₂ atmosphere. The reaction mixture was treated with water (20 mL) and extracted with EtOAc (30 mL×3). The combined organic phase was washed with brine (80 mL), dried with anhydrous Na₂SO₄, filtered and concentrated in vacuo to give a residue which was purified by column chromatography (SiO₂, petroleum ether/ethyl acetate=3/1 to 0/1) to give the title compound as a yellow solid (1 g, 1.43 mmol, 37% yield, 73% purity), ¹H NMR (400 MHz, DMSO-d₆) δ=10.40-10.33 (m, 1H), 7.36-7.18 (m, 5H), 6.01 (s, 1H), 5.97-5.90 (m, 1H), 5.84 (d, J=1.8 Hz, 1H), 4.21 (d, J=5.5 Hz, 2H), 4.04-4.00 (m, 2H), 3.85-3.72 (m, 3H), 3.34 (br s, 5H), 2.05 (br d, J=6.1 Hz, 1H), 1.37 (s, 11H), 0.94-0.87 (m, 2H), 0.86 (d, J=6.6 Hz, 3H).

### tert-Butyl (3R,5S)-3-(2-((6-amino-3-methyl-2-oxo-2,3-dihydro-1H-benzo[d]imidazol-4-yl)oxy)ethoxy)-5-methylpiperidine-1-carboxylate

To a solution of tert-butyl (3R,5S)-3-(2-((6-(benzylamino)-3-methyl-2-oxo-2,3-dihydro-1H-benzo[d]imidazol-4-yl)oxy)ethoxy)-5-methylpiperidine-1-carboxylate (1 g, 1.96 mmol, 1 eq) in THF (20 mL) was added Pd/C (400 mg, 10% purity) under Ar. The suspension was degassed under vacuum and purged with H₂ several times. The reaction mixture was stirred under H₂ (15 psi) at 60° C. for 2 hr. The reaction mixture was filtered, and the filtrate was concentrated in vacuo to give the title compound as a black oil (0.9 g, crude). LCMS: [M-100+H]⁺=321.3.

### tert-Butyl (3R,5S)-3-(2-(6-((2,5-dichloropyrimidin-4-yl)amino)-3-methyl-2-oxo-2,3-dihydro-1H-benzo[d]imidazol-4-yl)oxy)ethoxy)-5-methylpiperidine-1-carboxylate

To a solution of tert-butyl (3R,5S)-3-(2-((6-amino-3-methyl-2-oxo-2,3-dihydro-1H-benzo[d]imidazol-4-yl)oxy)ethoxy)-5-methylpiperidine-1-carboxylate (900.00 mg, 2.14 mmol, 1 eq) in DMF (10 mL) were added 2,4,5-trichloropyrimidine (785.16 mg, 4.28 mmol, 2 eq) and DIPEA (691.55 mg, 5.35 mmol, 932.01 AL, 2.5 eq). The reaction mixture was stirred at 25° C. for 2 hr. The reaction mixture was treated with water (25 mL) and the precipitate was filtered. The solid filtered and dried to give the title compound as a white solid (0.7 g, 1.15 mmol, 54% yield, 93% purity). ¹H NMR (400 MHz, DMSO-d₆) δ=10.90 (s, 1H), 9.38 (s, 1H), 8.33 (s, 1H), 6.96 (d,. J=3.2 Hz, 2H), 4.22-4.02 (m, 3H), 3.90-3.72 (m, 3H), 3.47 (s, 3H), 3.29 (s, 1H), 2.47-2.16 (m, 2H), 2.06 (d, J=11.6 Hz, 1H), 1.55-1.44 (m, 1H), 1.37 (s, 9H), 0.99-0.88 (m, 1H), 0.85 (d, J=6.8 Hz, 3H).

### 5-((2,5-Dichloropyrimidin-4-yl)amino)-1-methyl-7˜ (2-(((3R,5S)-5-methylpiperidin-3-yl)oxy)ethoxy)-1,3-dihydro-2H-benzo[d]imidazol-2-one

To a solution of tert-butyl (3R,5S)-3-(2-((6-((2,5-dichloropyrimidin-4-yl)amino)-3-methyl-2-oxo-2,3-dihydro-1H-benzo[d]imidazol-4-yl)oxy)ethoxy)-5-methylpiperidine-1-carboxylate (700.00 mg, 1.23 mmol, 1 eg) in DCM (12 mL) was added TFA (3.23 g, 28.36 mmol, 2.10 mL, 22.99 eq). The reaction mixture was stirred at 25° C. for 30 min. The reaction mixture was concentrated in vacuo to give the title compound as a yellow oil (0.8 g, crude, TFA). LCMS: [M+H]⁺=467.1

### (1³R, 1⁵S)-2⁵-chloro-1⁵,4³-dimethyl-4²,4³-dihydro-4¹H-5,8-dioxa-3-aza-4(6,4)-benzo[d]imidazola-2(2,4)-pyrimidina-1 (1,3)-piperidinacyclooctaphan-4²-one

To a solution of 5-((2,5-dichloropyrimidin-4-yl)amino)-1-methyl-7-(2-(((3R,5S)-5-methylpiperidin-3-yl)oxy)ethoxy)-1,3-dihydro-2H-benzo[d]imidazol-2-one (800 mg, 1.38 mmol, 1 eq, TFA) in DMSO (75 mL) was added DIPEA (1.78 g, 13.76 mmol, 2.40 mL, 10 eq). The reaction mixture was stirred at 80° C. for 12 hr. The reaction was quenched with water (100 mL), filtered, and the solid was purified by column chromatography (SiO₂, petroleum ether/ethyl acetate=2/1 to 0/1) to give the title compound as a white solid (190 mg, 176.38 μmol, 13% yield). LCMS: [M+H]⁺=431.2.

### (1³R, 1⁵S)-2⁵-chloro-4¹-(3-hydroxy-3-methylbutyl)-1⁵,4³-dimethyl-4²,4³-dihydro-4¹H-5,8-dioxa-3-aza-4(6,4)-benzo[d]imidazola-2(2,4)-pyrimidina-1 (1,3)-piperidinacyclooctaphan-4²-one (12)

To a mixture of (1³R,1⁵S)-2⁵-chloro-1⁵,4³-dimethyl-4²,4³-dihydro-4 H-5,8-dioxa-3-aza-4(6,4)-benzo[d]imidazola-2(2,4)-pyrimidina-1 (1,3)-piperidinacyclooctaphan-4²-one (190 mg, 440.95 μmol, 1 eq) and 3-hydroxy-3-methylbutyl 4-methylbenzenesulfonate (227.82 mg, 881.90 μmol, 2 eq) in DMSO (3 mL) were added Cs₂CO₃ (287.34 mg, 881.90 μmol, 2 eq) and KI (36.60 mg, 220.48 μmol, 0.5 eq). The reaction mixture was stirred at 80° C. for 12 hr. The reaction mixture was filtered and residue was purified by prep-HPLC (column: Phenomenex C18 80*40 mm*3 μm;mobile phase: [water (NH₄HCO₃)-ACN]; B %: 25%-55%, 8 min) to give the title compound as a white solid (50 mg, 95.74 μmol, 22% yield, 99% purity). ¹H NMR (400 MHz, DMSO-d₆) δ=8.85 (s, 1H), 8.05 (s, 1H), 7.70 (s, 1H), 6.79 (s, 1H), 4.69-4.58 (m, 2H), 4.45 (s, 1H), 4.43-4.35 (m, 1H), 4.20-4.08 (m, 1H), 3.86-3.68 (m, 5H), 3.45 (s, 3H), 2.48-2.38 (m, 2H), 2.01-1.89 (m, 1H), 1.76-1.55 (m, 3H), 1.18 (s, 6H), 1.15-1.07 (m, 1H), 0.88 (d, J=6.4 Hz, 3H). LCMS: [M+H]⁺=517.4.

### Example 12: Synthesis of (1³S,1⁵R)-2⁵-chloro-4¹-(3-hydroxy-3-methylbutyl)-1⁵,4³-dimethyl-4²,4³-dihydro-4¹H-5,8-dioxa-3-aza-4(6,4)-benzo[d]imidazola-2(2,4)-pyrimidina-1 (1,3)-piperidinacyclooctaphan-4²-one (13)

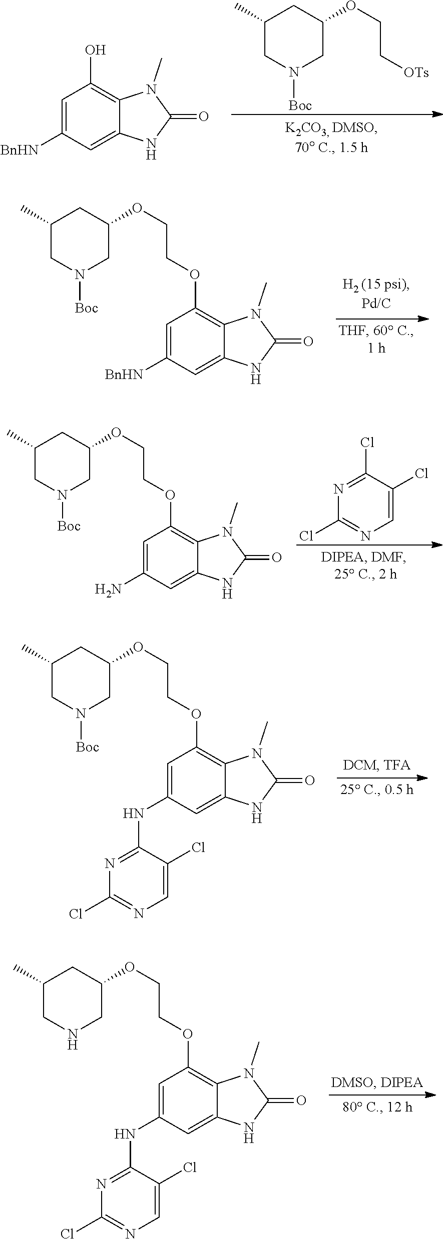

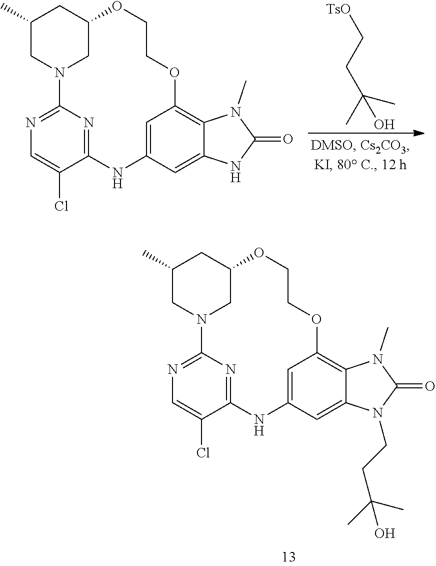 [SMILES: C[C@@H]1C[C@H]2CN(C1)c1ncc(Cl)c(n1)Nc1cc(c3c(c1)[nH]c(=O)n3C)OCCO2 ; Cc1ccc(S(=O)(=O)OCCC(C)(C)O)cc1 ; C[C@@H]1C[C@H]2CN(C1)c1ncc(Cl)c(n1)Nc1cc(c3c(c1)n(CCC(C)(C)O)c(=O)n3C)OCCO2]

### tert-Butyl (3S,5R)-3-(2-((6-(benzylamino)-3-methyl-2-oxo-2,3-dihydro-1H-benzo[d]imidazol-4-yl)oxy)ethoxy)-5-methylpiperidine-1-carboxylate

To a solution of 5-(benzylamino)-7-hydroxy-1-methyl-1,3-dihydro-2H-benzo[d]imidazol-2-one (940 mg, 3.49 mmol, 1 eq) and tert-butyl (3R,5S)-3-methyl-5-(2-(tosyloxy)ethoxy)piperidine-1-carboxylate (1.44 g, 3.49 mmol, 1 eq) in DMSO (10 mL) was added K₂CO₃ (964.86 mg, 6.98 mmol, 2 eq). The reaction mixture was stirred at 70° C. for 1.5 hr. The reaction was quenched with water (30 mL), and then extracted with EtOAc (20 mL×3). The combined organic phase was washed with brine (20 mL), dried with anhydrous Na₂SO₄, filtered and concentrated in vacuo to give a residue which was purified by column chromatography (SiO₂, petroleum ether/ethyl acetate=5/1 to 0/1) to give the title compound as a yellow solid (900 mg, 1.73 mmol, 49% yield). LCMS: [M+H]⁺=511.3.

### tert-Butyl (3S,5R)-3-(2-((6-amino-3-methyl-2-oxo-2,3-dihydro-1H-benzo[d]imidazol-4-yl)oxy)ethoxy)-5-methylpiperidine-1-carboxylate

To a solution of tert-butyl (3S,5R)-3-(2-((6-(benzylamino)-3-methyl-2-oxo-2,3-dihydro-1H-benzo[d]imidazol-4-yl)oxy)ethoxy)-5-methylpiperidine-1-carboxylate (900 mg, 1.76 mmol, 1 eq) in THF (8 mL) was added Pd/C (400 mg, 10% purity) under Ar. The suspension was degassed under vacuum and purged with H₂ several times. The reaction mixture was stirred under H₂ (15 psi) at 60° C. for 1 hour. The reaction mixture was filtered through a pad of Celite® and the filtrate was concentrated in vacuo to give the title compound as a black solid (900 mg, crude). LCMS: [M−100+H]⁺=321.2.

### ter-Butyl (3S,5R)-3-(2-(6-(2,5-dichloropyrimidin-4-yl)amino)-3-methyl-2-oxo-2,3-dihydro-1H-benzo[d]imidazol-4-yl)oxy)ethoxy)-5-methylpiperidine-1-carboxylate

To a solution of tert-butyl (3S,5R)-3-(2-((6-amino-3-methyl-2-oxo-2,3-dihydro-1H-benzo[d]imidazol-4-yl)oxy)ethoxy)-5-methylpiperidine-1-carboxylate (900 mg, 2.14 mmol, 1 eq) and 2,4,5-trichloropyrimidine (785.16 mg, 4.28 mmol, 2 eq) in DMF (9 mL) was added DIEA (553.23 mg, 4.28 mmol, 745.59 μL, 2 eq). The reaction mixture was stirred at 25° C. for 2 h. The reaction was quenched with water (10 mL) and filtered. The filter cake was dried under reduced pressure to give a residue which was triturated with (petroleum ether/ethyl acetate=3/1, 5 mL) at 25° C. for 10 min to give the title compound as a yellow solid (800 mg, 1.38 mmol, 65% yield, 98% purity). ¹H NMR (400 MHz, DMSO-d₆) δ=10.90 (s, 1H), 9.39 (s, 1H), 8.33 (s, 1H), 6.97 (d, J=2.9 Hz, 2H), 4.14 (br t, J=4.4 Hz, 2H), 3.88-3.82 (m, 2H), 3.47 (s, 3H), 2.45-2.16 (m, 3H), 2.06 (br d, J=11.9 Hz, 1H), 1.55-1.44 (m, 2H), 1.37 (s, 11H), 0.85 (d, J=6.5 Hz, 3H).

### 5-((2,5-Dichloropyrimidin-4-yl)amino)-1-methyl-7-(2-(((3S,5R)-5-methylpiperidin-3-yl)oxy)ethoxy)-1,3-dihydro-2H-benzo[d]imidazol-2-one

To a solution of tert-butyl (3S,5R)-3-[2-[[6-[(2,5-dichloropyrimidin-4-yl)amino]-3-methyl-2-oxo-1H-benzimidazol-4-yl]oxy]ethoxy]-5-methyl-piperidine-1-carboxylate (800 mg, 1.41 mmol, 1 eq) in DCM (8 mL) was added TFA (2 mL). The reaction mixture was stirred at 25° C. for 30 min. The reaction mixture was concentrated in vacuo to give the title compound as a yellow oil (800 mg, 1.38 mmol, 98% yield, TFA). LCMS: [M+H]⁺=467.3.

### (1³S,1⁵R)-2⁵-chloro-1⁵,4³-dimethyl-4²,4³-dihydro-4¹H-5,8-dioxa-3-aza-4(6,4)-benzo[d]imidazola-2(2,4)-pyrimidina-1 (1,3)-piperidinacyclooctaphan-4²-one

To a solution of tert-butyl (3S,5R)-3-[2-[[6-[(2,5-dichloropyrimidin-4-yl)amino]-3-methyl-2-oxo-1H-benzimidazol-4-yl]oxy]ethoxy]-5-methyl-piperidine-1-carboxylate (800 mg, 1.38 mmol, 1 eq, TFA) in DMSO (36 mL) was added DIEA (1.78 g, 13.76 mmol, 2.40 mL, 10 eq). The reaction mixture was stirred at 80° C. for 12 h. The reaction mixture was concentrated in vacuo to give a residue which was purified by column chromatography (SiO₂, petroleum ether/THF=5/1 to 0/1) to give the title compound as a yellow solid (120 mg, 161.53 μmol, 12% yield). ¹H NMR (400 MHz, DMSO-d₆) δ=10.83 (s, 1H), 8.77 (s, 1H), 8.03 (s, 1H), 7.65 (d, J=0.9 Hz, 1H), 6.66 (d, J=1.3 Hz, 1H), 4.69-4.55 (m, 2H), 4.42-4.35 (m, 1H), 4.18-4.08 (m, 1H), 3.88-3.66 (m, 3H), 3.40 (s, 3H), 2.47-2.38 (m, 2H), 1.98-1.91 (m, 1H), 1.67-1.54 (m, 1H), 1.12 (br d, J=11.2 Hz, 1H), 0.87 (d, J=6.8 Hz, 3H).

### (1³S,1⁵R)-2⁵-chloro-4¹-(3-hydroxy-3-methylbutyl)-1⁵,4³-dimethyl-4²,4³-dihydro-4¹H-5,8-dioxa-3-aza-4(6,4)-benzo[d]imidazola-2(2,4)-pyrimidina-1 (1,3)-piperidinacyclooctaphan-4²-one (13)

To a solution of (1³S,1⁵R)-2⁵-chloro-1⁵,4³-dimethyl-4²,4³-dihydro-4¹H-5,8-dioxa-3-aza-4(6,4)-benzo[d]imidazola-2(2,4)-pyrimidina-1 (1,3)-piperidinacyclooctaphan-4²-one (90 mg, 208.87 μmol, 1 eq) in DMSO (1 mL) were added KI (17.34 mg, 104.44 μmol, 0.5 eq), Cs₂CO₃ (136.11 mg, 417.74 μmol, 2 eq) and 3-hydroxy-3-methylbutyl 4-methylbenzenesulfonate (107.92 mg, 417.74 μmol, 2 eq). The reaction mixture was stirred at 80° C. for 12 hr, then cooled to 25° C. and purified by prep-HPLC (column: Waters Xbridge™ BEH C18 100*30 mm*10 μm,mobile phase: [water (NH₄HCO₃)-ACN]; B %: 40%-70%, 8 min) to give the title compound as a white solid (20 mg, 38.30 μmol, 18% yield, 96% purity). ¹H NMR (400 MHz, DMSO-d₆) δ=8.85 (s, 1H), 8.05 (s, 1H), 7.70 (s, 1H), 6.79 (d, J=1.1 Hz, 1H), 4.69-4.58 (m, 2H), 4.39 (br dd, J=3.6, 12.8 Hz, 1H), 4.20-4.09 (m, 1H), 3.82 (br dd, J=5.3, 9.8 Hz, 3H), 3.78-3.67 (m, 2H), 3.45 (s, 3H), 2.47-2.36 (m, 2H), 2.01-1.91 (m, 1H), 1.70 (br dd, J=6.3, 10.4 Hz, 2H), 1.65-1.56 (m, 1H), 1.18 (s, 6H), 1.12 (br d, J=11.3 Hz, 1H), 0.88 (d, J=6.5 Hz, 3H). LCMS: [M+H]⁺=517.4.

### Example 13: Synthesis of (1³R,1⁵S)-2⁵-chloro-4¹-(3-hydroxy-3-methylbutyl)-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (16)

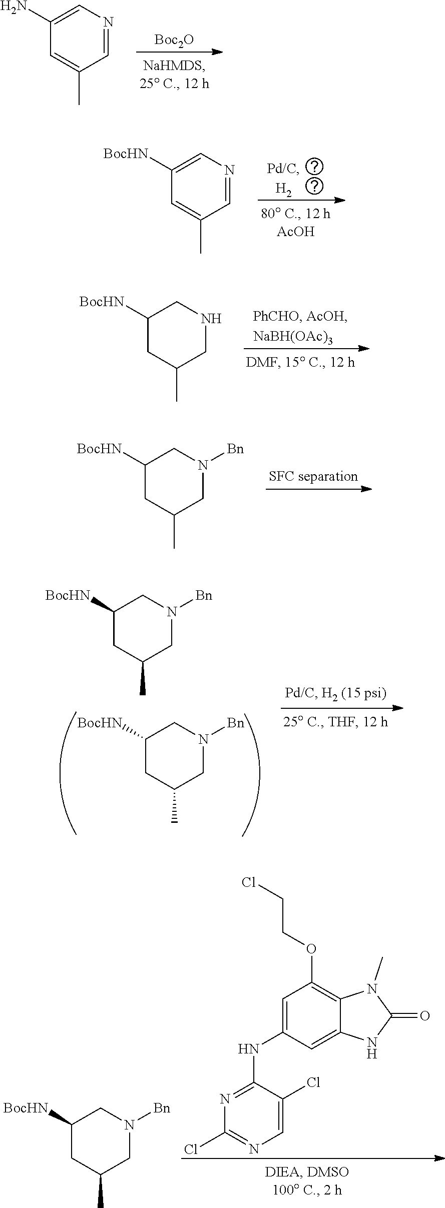

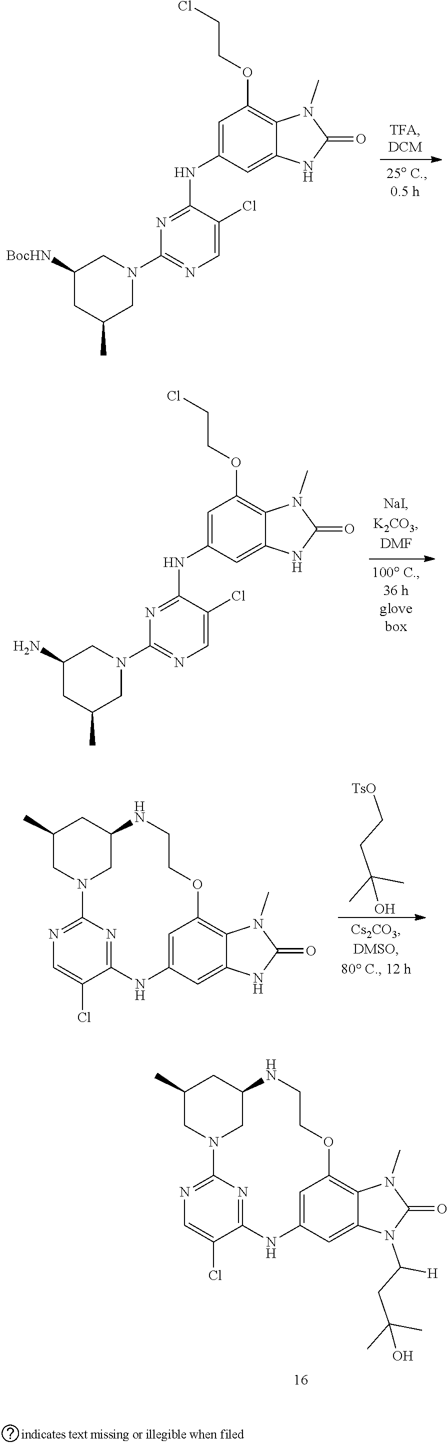 [SMILES: C[C@H]1C[C@@H](NC(=O)OC(C)(C)C)CN(c2ncc(Cl)c(Nc3cc(OCCCl)c4c(c3)[nH]c(=O)n4C)n2)C1 ; C[C@H]1C[C@@H](N)CN(c2ncc(Cl)c(Nc3cc(OCCCl)c4c(c3)[nH]c(=O)n4C)n2)C1 ; C[C@H]1C[C@@H]2CN(C1)c1ncc(Cl)c(n1)Nc1cc(c3c(c1)[nH]c(=O)n3C)OCCN2 ; Cc1ccc(S(=O)(=O)OCCC(C)(C)O)cc1 ; C[C@H]1C[C@@H]2CN(C1)c1ncc(Cl)c(n1)Nc1cc(c3c(c1)n(CCC(C)(C)O)c(=O)n3C)OCCN2]

### tert-Butyl (5-methylpyridin-3-yl)carbamate

To a solution of 5-methylpyridin-3-amine (12.5 g, 115.59 mmol, 1 eq) in THF (100 mL) at 25° C. was added NaHMDS (1 M, 254.30 mL, 2.2 eq) and the reaction mixture was stirred for 30 min. Then Boc₂O) (26.99 g, 123.68 mmol, 28.41 mL, 1.07 eq) in THF (25 mL) was added and the reaction mixture was stirred at 25° C. for 12 hr. The reaction mixture (combined with another batch of the same scale) was quenched with water (50 mL), and treated with 0.2 M HCl (300 mL). The mixture was extracted with EtOAc (500 mL×3). The combined organic phase was washed with brine (500 mL), dried over anhydrous Na₂SO₄, filtered and concentrated in vacuo to give a residue which was purified by silica gel column chromatography (silica gel, petroleum ether/ethyl acetate=5/1 to 0/1) to give the title compound as a white solid (27 g, 128.35 mmol, 56% yield, 99% purity). ¹H NMR (400 MHz, DMSO-d₆) δ ppm 9.49 (s, 1H), 8.39 (s, 1H), 8.01 (s, 1H), 7.74 (s, 1H), 2.24 (s, 3H), 1.47 (s, 9H).

### tert-Butyl (5-methylpiperidin-3-yl)carbamate

To a solution of tert-butyl (5-methylpyridin-3-yl)carbamate (17 g, 81.63 mmol, 1 eq) in AcOH (340 mL) were added Pd/C (14 g, 10% purity) and PtO₂ (1.85 g, 8.16 mmol, 0.1 eq) under Ar. The suspension was degassed under vacuum and purged with H₂ several times. The reaction mixture was stirred under H₂ (3 Mpa) at 80° C. for 12 hr. The reaction mixture was filtered, and the filter cake was washed with MeOH (500 mL×3). The filtrate was concentrated in vacuo to give a residue which was adjusted to pH 8 using sat, aq. NaHCO₃. The mixture was treated with water (400 mL), and then extracted with EtOAc (200 mL×3). The combined organic phase was washed with brine (400 mL), dried with anhydrous Na₂SO₄, filtered and concentrated in vacuo to give a residue which was purified by silica gel column chromatography (silica gel, Ethyl acetate/MeOH =6/1 to 0/1) to give the title compound as a white solid (7,5 g, 35.00 mmol, 43% yield). ¹H NMR (400 MHz, DMSO-d₆) δ=6.64 (br d, J=7.9 Hz, 1H), 3.30-3.15 (m, 1H), 3.05-2.83 (m, 2H), 2.78-2.52 (m, 2H), 2.14-1.97 (m, 1H), 1.89-1.82 (m, 1H), 1.77 (br d, J=12.3 Hz, 1H), 1.69-1.41 (m, 1H), 1.39-1.36 (m, 9H), 0.76 (d, J=6.7 Hz, 3H).

tert-Butyl (1-benzyl-5-methylpiperidin-3-yl)carbamate

A mixture of tert-butyl (5-methylpiperidin-3-yl)carbamate (10 g, 46.66 mmol, 1 eq), benzaldehyde (5.94 g, 56.00 mmol, 5.66 mL, 1.2 eq) and AcOH (1.40 g, 23.33 mmol, 1.33 mL, 0.5 eq) in DMF (250 mL) was stirred at 15° C. for 2 hr. Then sodium triacetoxyborohydride (19.78 g, 93.33 mmol, 2 eq) was added, and the reaction mixture was stirred at 15° C. for 10 hr. The reaction was quenched with water (1 L), then extracted with EtOAc (600 mL×3). The combined organic phase was washed with brine (1 L), dried with anhydrous Na₂SO₄, filtered and the filtrate was concentrated in vacuo to give a residue which was purified by prep-HPLC (column: Agela DuraShell C18 250*70 mm*10 μm; mobile phase: [water (NH₄HCO₃)-ACN]; B %: 50%-75%, 20 min) to give the title compound as a white solid (5 g, 16.26 mmol, 35% yield, 99% purity). ¹H NMR (400 MHz, DMSO-d₆) δ=7.34-7.22 (m, 5H), 6.73 (br d, J=8.1 Hz, 1H), 3.50-3.39 (m, 3H), 2.81 (br d, J=7.4 Hz, 1H), 2.67 (br d, J=9.2 Hz, 1H), 1.73 (br d, J=12.0 Hz, 1H), 1.65-1.49 (m, 2H), 1.39 (br s, 1H), 1.35 (s, 9H), 1.26 (br s, 1H), 0.79 (br d, J=6.7 Hz, 3H).

### tert-Butyl ((3R,5S)-1-benzyl-5-methylpiperidin-3-yl)carbamate and tert-butyl ((3S,5R)-1-benzyl-5-methylpiperidin-3-yl)carbamate

tert-Butyl (1-benzyl-5-methylpiperidin-3-yl)carbamate (5 g, 16.42 mmol, 1 eq) was separated by SFC (column: REGIS (s,s) WHELK-01 (250 mm*50 mm, 10 μm);mobile phase: [Hexane-IPA];B %: 5%-5%, 3.5 min) to give tert-butyl ((3R,5S)-1-benzyl-5-methylpiperidin-3-yl)carbamate as white solid (1.8 g, 5.79 mmol, 98% yield, 98% purity) and tert-butyl ((3S,5R)-1-benzyl-5-methylpiperidin-3-yl)carbamate as a white solid (1.8 g, 5.62 mmol, 95% yield, 95% purity). tert-Butyl ((3R,5S)-1-benzyl-5-methylpiperidin-3-yl)carbamate: ¹H NMR (400 MHz, DMSO-d₆) δ=7.33-7.22 (m, 5H), 6.74 (br d, J=8.2 Hz, 1H), 3.48-3.40 (m, 3H), 2.80 (br d, J=7.9 Hz, 1H), 2.67 (br d, J=9.3 Hz, 1H), 1.73 (br d, J=12.2 Hz, 1H), 1.63-1.50 (m, 2H), 1.44-1.38 (m, 1H), 1.34 (s, 9H), 1.25 (br s, 1H), 0.78 (br d, J=6.7 Hz, 3H). tert-Butyl ((3S,5R)-1-benzyl-5-methylpiperidin-3-yl)carbamate: ¹H NMR (400 MHz, DMSO-d₆) δ=7.34-7.22 (m, 5H), 6.74 (br d, J=8.2 Hz, 1H), 3.44 (br d, J=3.7 Hz, 3H), 2.84-2.76 (m, 1H), 2.67 (br d, J=8.9 Hz, 1H), 1.72 (br d, J=12.0 Hz, 1H), 1.63-1.51 (m, 2H), 1.43-1.38 (m, 1H), 1.34 (s, 9H), 1.25 (br s, 1H), 0.80-0.76 (m, 3H).

### tert-Butyl ((3R,5S)-5-methylpiperidin-3-yl)carbamate

To a solution of tert-butyl ((3S,5R)-1-benzyl-5-methylpiperidin-3-yl)carbamate (1.30 g, 4.27 mmol, 1 eq) in THF (13 mL) was added Pd/C (700 mg, 10% purity) under Ar. The suspension was degassed under vacuum and purged with H₂ several times. The reaction mixture was stirred under H₂ (15 psi) at 25° C. for 12 hr. The reaction mixture was filtered, and the filtrate was concentrated in vacuo to give the title compound as a white solid (0.8 g, crude). ¹H NMR (400 MHz, MeOD-d₄) δ=3.41 (ddd,,J=4.5, 7.1, 11.3 Hz, 1H), 3.11-3.03 (m, 1H), 2.87 (br dd, J=3.5, 12.4 Hz, 1H), 2.14 (t, J=11.6 Hz, 1H), 2.05-1.97 (m, 1H), 1.97-1.90 (m, 1H), 1.68-1.54 (m, 1H), 1.43 (s, 9H), 1.40 (s, 1H), 0.87 (d, J=6.6 Hz, 3H).

tert-Butyl (3R,5S)-1-(5-chloro-4-((7-(2-chloroethoxy)-1-methyl-2-oxo-2,3-dihydro-1H-benzo[d]imidazol-5-yl)amino)pyrimidin-2-yl)-5-methylpiperidin-3-yl)carbamate

To a mixture of 7-(2-chloroethoxy)-5-((2,5-dichloropyrimidin-4-yl)amino)-1-methyl-1,3-dihydro-2H-benzo[d]imidazol-2-one (1.5 g, 3.86 mmol, 1 eq) and tert-butyl ((3R,5S)-5-methylpiperidin-3-yl)carbamate (992.57 mg, 4.63 mmol, 1.2 eq) in DMSO (15 mL) was added DIPEA (997.67 mg, 7.72 mmol, 1.34 mL, 2 eq). The reaction mixture was stirred at 100° C. for 2 hr. The reaction was quenched with water (20 mL) and the solid was filtered. The filter cake was dried under vacuum to give a residue which was purified by column chromatography (SiO₂, petroleum ether/ethyl acetate=2/1 to 0/1) to give the title compound as a yellow solid (1.1 g, 1.84 mmol, 48% yield, 95% purity). LCMS: [M+H]⁺=510.2.

### 5-((2-((3R,5S)-3-Amino-5-methylpiperidin-1-yl)-5-chloropyrimidin-4-yl)amino)-7-(2-chloroethoxy)-1-methyl-1,3-dihydro-2H-benzo[d]imidazol-2-one

A solution of tert-butyl ((3R,5S)-1-(5-chloro-4-((7-(2-chloroethoxy)-1-methyl-2-oxo-2,3-dihydro-1H-benzo[d]imidazol-5-yl)amino)pyrimidin-2-yl)-5-methylpiperidin-3-yl)carbamate (600 mg, 1.06 mmol, 1 eq) in DCM (10 mL) and TFA (2 mL) was stirred at 25° C. for 30 min. The reaction mixture was concentrated in vacuo to give the title compound as a brown oil (600 mg, crude, TEA). LCMS: [M+H]⁺=464.4.

### (1³R, 1⁵S)-23-Chloro-13,43-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one

A mixture of 5-((2-((3R,5S)-3-amino-5-methylpiperidin-1-yl)-5-chloropyrimidin-4-yl)amino)-7-(2-chloroethoxy)-1-methyl-1,3-dihydro-2H-benzo[d]imidazol-2-one (200 mg, 428.85 μmol, 1 eq), K₂CO; (296.35 mg, 2.14 mmol, 5 eq), and NaI (642.82 mg, 4.29 mmol, 10 eq) in DMF (30 mL) in a glove box was stirred at 100° C. for 36 hr. The reaction mixture was concentrated in vacuo and water (50 mL) was added. The mixture was extracted with ethyl acetate (35 mL×3). The combined organic phase was dried with anhydrous Na₂SO₄, filtered and concentrated in vacuo to give a residue which was purified by column chromatography (SiO₂, petroleum ether/THF=4/1 to 1/1) to give the title compound as a yellow solid (320 mg). ¹H NMR. (400 MHz, DMSO-d₆) δ=10.81 (br s, 1H), 8.65 (s, 1H), 8.00 (s, 1H), 7.66 (br s, 1H), 6.87 (s, 2H), 4.80 (br d, J=11.1 Hz, 1H), 4.49 (br d, J=10.8 Hz, 1H), 4.36 (br t, J=10.9 Hz, 1H), 4.07-3.96 (m, 1H), 3.40 (br s, 3H), 3.01 (br t, J=11.1 Hz, 1H), 2.94-2.75 (m, 2H), 2.39 (br t, J=12.1 Hz, 1H), 2.16-2.10 (m, 1H), 1.90 (s, 1H), 1.82 (br d, J=11.9 Hz, 1H), 1.57 (br d, J=3.1 Hz, 1H), 0.85 (br d, J=6.4 Hz, 3H). LCMS: [M+1]⁺=430.2.

(1³R, 1⁵S)-2⁵-Chloro-4¹-(3-hydroxy-3-methylbutyl)-1⁵,4³-dimethyl-4²,4³-dihydro-41 H-5-oxa-3,8-diaza-4(6,4)-benzo[d]imidazola-2(2,4)-pyrimidina-1 (1,3)-piperidinacyclooctaphan-4²-one (16)

A mixture of (1³R,1⁵S)-2⁵-chloro-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (290 mg, 674.57 μmol, 1 eq), 3-hydroxy-3-methylbutyl 4-methylbenzenesulfonate (348.53 mg, 1.35 mmol, 2 eq), KI (55.99 mg, 337.29 μmol, 0.5 eq), and Cs₂CO₃ (439.58 mg, 1.35 mmol, 2 eq) in DMSO (3 mL) was degassed and purged with N₂3 times. The reaction mixture was stirred at 80° C. for 12 hr under N₂ atmosphere. The reaction mixture was filtered and the filtrate was purified by prep-HPLC (column: Waters Xbridge™ BEH C18 100*30 mm*10 μm; mobile phase: [water (NH₄HCO₃)-ACN]; B %: 35%-55%, 8 min) to give the title compound as a white solid (23 mg, 44.12 μmol, 7% yield, 99% purity). ¹H NMR (400 MHz, DMSO-d₆) δ=8.74 (s, 1H), 8.02 (s, 1H), 7.70 (s, 1H), 6.79 (d, J=1.1 Hz, 1H), 4.79 (br d, J=11.6 Hz, 1H), 4.52-4.45 (m, 2H), 4.42-4.34 (m, 1H), 4.02 (dt, J=8.1, 11.1 Hz, 1H), 3.82 (br dd, J=5.2, 9.7 Hz, 2H), 3.45 (s, 3H), 3.05-2.97 (m, 1H), 2.91-2.78 (m, 2H), 2.40 (t, J=12.3 Hz, 1H), 2.15 (br t, J=11.4 Hz, 1H), 1.82 (br d, J=12.5 Hz, 1H), 1.69 (br dd, J=5.1, 9.4 Hz, 2H), 1.63-1.54 (m, 1H), 1.18 (s, 6H), 1.13 (dd, J=2.8, 18.5 Hz, 1H), 1.02-0.92 (m, 1H), 0.86 (d, J=6.6 Hz, 3H). LCMS: [M+H]⁺=516.4.

### Example 14: Synthesis of (135. 15R)-2⁵-chloro-4-(3-hydroxy-3-methylbutyl)-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (17)

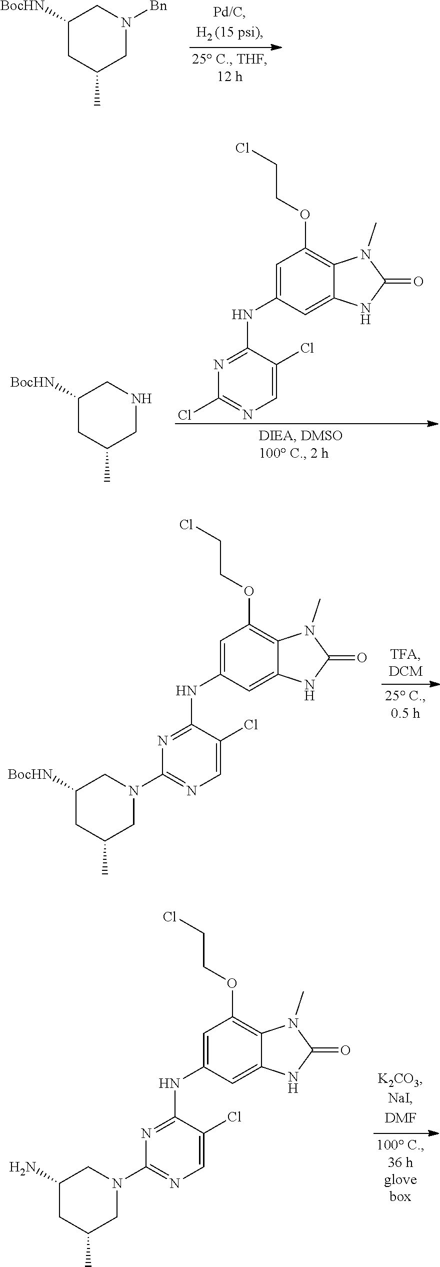 [SMILES: C[C@@H]1C[C@H](NC(=O)OC(C)(C)C)CN(Cc2ccccc2)C1 ; C[C@H]1CNC[C@@H](NC(=O)OC(C)(C)C)C1 ; Cn1c(=O)[nH]c2cc(Nc3nc(Cl)ncc3Cl)cc(OCCCl)c21 ; C[C@@H]1C[C@H](NC(=O)OC(C)(C)C)CN(c2ncc(Cl)c(Nc3cc(OCCCl)c4c(c3)[nH]c(=O)n4C)n2)C1 ; C[C@@H]1C[C@H](N)CN(c2ncc(Cl)c(Nc3cc(OCCCl)c4c(c3)[nH]c(=O)n4C)n2)C1]

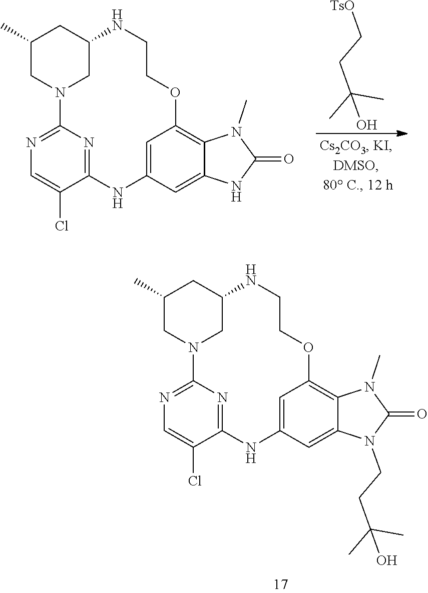 [SMILES: C[C@@H]1C[C@H]2CN(C1)c1ncc(Cl)c(n1)Nc1cc(c3c(c1)[nH]c(=O)n3C)OCCN2 ; Cc1ccc(S(=O)(=O)OCCC(C)(C)O)cc1 ; C[C@@H]1C[C@H]2CN(C1)c1ncc(Cl)c(n1)Nc1cc(c3c(c1)n(CCC(C)(C)O)c(=O)n3C)OCCN2]

### tert-Butyl ((3S,5R)-5-methylpiperidin-3-yl)carbamate

To a solution of tert-butyl N-[(3S,5R)-1-benzyl-5-methyl-3-piperidyl]carbamate (1.30 g, 4.27 mmol, 1 eq) in THF (13 mL) was added Pd/C (700 mg, 10% purity) under Ar. The suspension was degassed under vacuum and purged with H₂ several times. The reaction mixture was stirred under H₂ (15 psi) at 25° C. for 12 hr. The reaction mixture was filtered and the filtrate was concentrated in vacuo to give the title compound as a white solid (0.8 g, crude). ¹H NMR (400 MHz, MeOD-d₄) δ=3.41 (tt, J=4.3, 11.3 Hz, 1H), 3.11-3.03 (m, 1H), 2.87 (br dd, J=3.5, 12.5 Hz, 1H), 2.14 (t, J=11.6 Hz, 1H), 2.05-1.90 (m, 2H), 1.61 (dtd, J=3.8, 7.3, 14.7 Hz, 1H), 1.43 (s, 9H), 1.40 (s, 1H), 0.87 (d, J=6.6 Hz, 3H).

### tert-Butyl ((3S,5R)-1-(5-chloro-4-((7-(2-chloroethoxy)-1-methyl-2-oxo-2,3-dihydro-1H-benzo[d]imidazol-5-yl)amino)pyrimidin-2-yl)-5-methylpiperidin-3-yl)carbamate

To a mixture of 4-(2-chloroethoxy)-6-[(2,5-dichloropyrimidin-4-yl)amino]-3-methyl-1H-benzimidazol-2-one (1.5 g, 3.86 mmol, 1 eq) and tert-butyl N-[(3S,5R)-5-methyl-3-piperidyl]carbamate (992.57 mg, 4.63 mmol, 1.2 eq) in DMSO (20 mL) was added DIPEA (997.64 mg, 7.72 mmol, 1.34 mL, 2 eq). The reaction mixture was stirred at 100° C. for 2 h. The reaction was quenched with water (20 mL) and the solid was filtered and dried. The product was purified by column chromatography (SiO₂, petroleum ether/ethyl acetate=2/1 to 0/1) to give the title compound as a yellow solid (0.95 g, 1.29 mmol, 33% yield). LCMS: [M−56+H]⁺=510.2, [M+1]⁺=566.2.

### 5-((2-((3S,5R)-3-Amino-5-methylpiperidin-1-yl)-5-chloropyrimidin-4-yl)amino)-7-(2-chloroethoxy)-1-methyl-1,3-dihydro-2H-benzo[d]imidazol-2-one

To a solution of tert-butyl N-[(3S,5R)-1-[5-chloro-4-[[7-(2-chloroethoxy)-1-methyl-2-oxo-3H-benzimidazol-5-yl]amino]pyrimidin-2-yl]-5-methyl-3-piperidyl]carbamate (600 mg, 1.06 mmol, 1 eq) in DCM (6 mL) was added TFA (1.5 mL). The reaction mixture was stirred at 25° C. for 30 min. The reaction mixture was filtered and filtrate was concentrated in vacuo to give the title compound as a brown oil (600 mg, crude, TFA). LCMS: [M+H]⁺=466.2.

### (1³S,1⁵R)-2⁵-Chloro-13,43-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one

To a solution of 6-[[2-[(3S,5R)-3-amino-5-methyl-1-piperidyl]-5-chloro-pyrimidin-4-yl]amino]-4-(2-chloroethoxy)-3-methyl-1H-benzimidazol-2-one (200 mg, 344.60 μmol, 1 eq, TFA) in DMF (30 mL) were added K₂CO₃ (238.13 mg, 1.72 mmol, 5 eq) and NaI (516.53 mg, 3.45 mmol, 10 eq) in a glove box. The reaction mixture was stirred at 100° C. for 36 hr under N₂ atmosphere. The reaction mixture was concentrated in vacuo and water (50 mL) was added. The solution was extracted with ethyl acetate (35 mL×3). The combined organic phase was dried with anhydrous Na₂SO₄, filtered and concentrated in vacuo to give a residue which was purified by column chromatography (SiO₂, petroleum ether/ethyl acetate=0/1 to THE/ethyl acetate=2/1) to give the title compound as a yellow solid (250 mg, 430.33 μmol, 42% yield). ¹H NMR (400 MHz, DMSO-d₆) δ=10.80 (s, 1H), 8.65 (s, 1H), 8.00 (s, 1H), 7.66 (s, 1H), 6.69-6.62 (m, 2H), 4.80 (br d, J=11.3 Hz, 1H), 4.51-4.45 (m, 1H), 4.40-4.31 (m, 1H), 4.01 (br d, J=7.6 Hz, 1H), 3.42-3.39 (m, 3H), 3.08-2.94 (m, 1H), 2.92-2.78 (m, 2H), 2.40 (brt, J=12.2 Hz, 1H), 2.15 (br s, 1H), 1.82 (br d, J=12.2 Hz, 1H), 1.63-1.52 (m, 1H), 1.02-0.94 (m, 1H), 0.86 (br d, J=6.4 Hz, 3H). LCMS: [M+H]⁺=430.2.

### (1³S,1⁵R)-2⁵-Chloro-4¹-(3-hydroxy-3-methylbutyl)-1⁵,4³-dimethyl-4²,4³-dihydro-41 H-5-oxa-3,8-diaza-4(6,4)-benzo[d]imidazola-2(2,4)-pyrimidina-1 (1,3)-piperidinacyclooctaphan-4²-one (17)

A mixture of (1³S,1⁵R)-2⁵-chloro-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (200 mg, 465.22 μmol, 1 eq), (3-hydroxy-3-methyl-butyl) 4-methylbenzenesulfonate (240.36 mg, 930.44 μmol, 2 eq), KI (38.61 mg, 232.61 μmol, 0.5 eq), and Cs₂CO₃ (303.16 mg, 930.44 μmol, 2 eq) in DMSO (2 mL) was degassed and purged with N₂3 times. The reaction mixture was stirred at 80° C. for 12 hr under N₂ atmosphere. The reaction mixture was filtered and the filtrate was purified by prep-HPLC (column: Waters Xbridge™ Prep OBD C18 150*40 mm*10 μm;mobile phase: [water (NH₄HCO₃)-ACN]; B %: 30%-60%, 8 min) to give the title compound as a white solid (35 mg, 67.15 μmol, 14% yield, 99% purity). ¹H NMR (400 MHz, DMSO-d₆) δ=8.73 (s, 1H), 8.02 (s, 1H), 7.70 (s, 1H), 6.79 (d, J=1.3 Hz, 1H), 4.80 (br d, J=12.4 Hz, 1H), 4.49 (br dd, J=4.0, 12.6 Hz, 1H), 4.45 (s, 1H), 4.42-4.33 (m, 1H), 4.02 (dt, J=8.0, 11.2 Hz, 1H), 3.86-3.76 (m, 2H), 3.45 (s, 3H), 3.07-2.95 (m, 1H), 2.93-2.76 (m, 2H), 2.40 (br t, J=12.2 Hz, 1H), 2.15 (br t, J=11.4 Hz, 1H), 1.86-1.78 (m, 1H), 1.74-1.65 (m, 2H), 1.63-1.50 (m, 1H), 1.18 (s, 6H), 1.16-1.09 (m, 1H), 0.97 (q, J=11.8 Hz, 1H), 0.86 (d, J=6.6 Hz, 3H). LCMS: [M+H]⁺=516.1.

### Example 15: Synthesis of (138, 15R)-2⁵-chloro-1⁴,1⁴-difluoro-4-(1R,3S)-3-hydroxycyclobutyl)methyl)-1⁵,4⁴-dimethyl-4¹,4², 4³ 4⁴-tetrahydro-5-oxa-3-aza-4(7,5)-quinoxalina-2(2,4)-pyrimidina-1 (1,3)-piperidinacyclooctaphane-4²,4³-dione (18-1) and (1³S,1⁵R)-2⁵-chloro-1⁴, 1⁴-difluoro-4¹-(((1S,3R)-3-hydroxycyclobutyl)methyl)-15.44-dimethyl-4¹,4² 4³,4⁴-tetrahydro-5-oxa-3-aza-4(7,5)-quinoxalina-2(2,4)-pyrimidina-1 (1,3)-piperidinacyclooctaphane-4²,4³-dione (18-2)

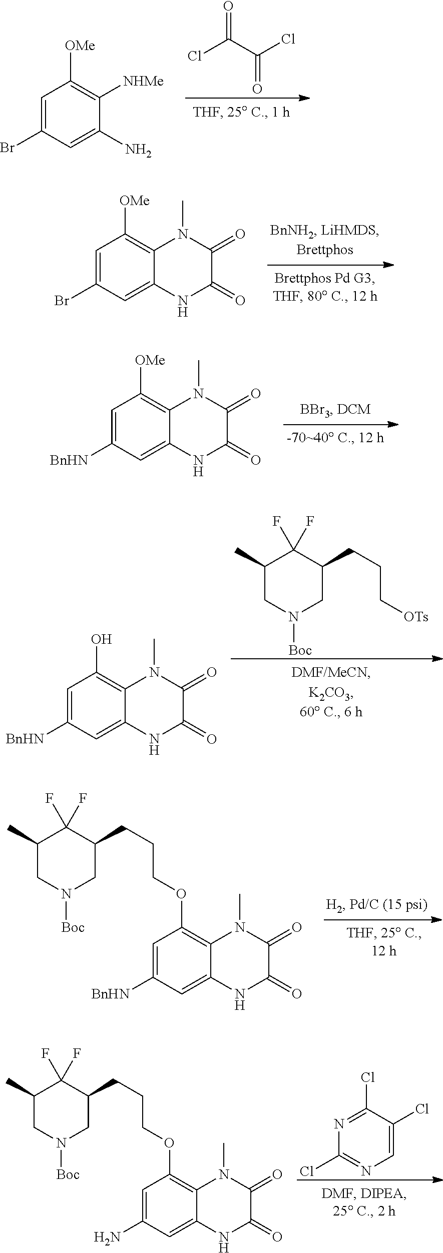

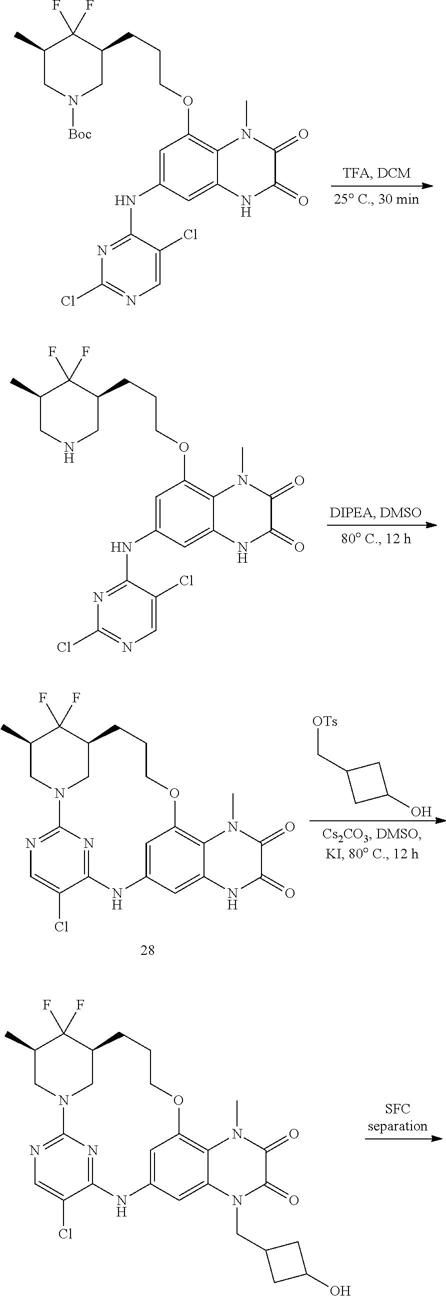 [SMILES: C[C@@H]1CN(C(=O)OC(C)(C)C)C[C@H](CCCOc2cc(Nc3nc(Cl)ncc3Cl)cc3[nH]c(=O)c(=O)n(C)c23)C1(F)F ; C[C@@H]1CNC[C@H](CCCOc2cc(Nc3nc(Cl)ncc3Cl)cc3[nH]c(=O)c(=O)n(C)c23)C1(F)F ; C[C@@H]1CN2C[C@H](CCCOc3cc(cc4[nH]c(=O)c(=O)n(C)c34)Nc3nc2ncc3Cl)C1(F)F ; Cc1ccc(S(=O)(=O)OCC2CC(O)C2)cc1 ; C[C@@H]1CN2C[C@H](CCCOc3cc(cc4c3n(C)c(=O)c(=O)n4CC3CC(O)C3)Nc3nc2ncc3Cl)C1(F)F]

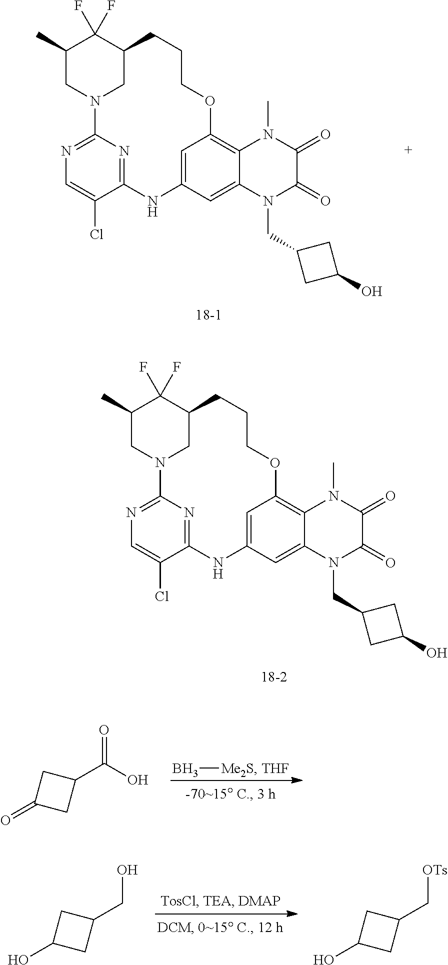

### 6-Bromo-8-methoxy-1-methyl-1,4-dihydroquinoxaline-2,3-dione

To a solution of 4-bromo-6-methoxy-N₁-methylbenzene-1,2-diamine (30 g, 129.82 mmol, 1 eq) in THF (350 mL) was added oxalyl dichloride (32.96 g, 259.64 mmol, 22.73 mL, 2 eq) dropwise at 25° C. The reaction mixture was stirred at 25° C. for 1 hr. The reaction mixture (combined with another batch of the same scale) was filtered and the filter cake was washed with ethyl acetate (300 mL) and dried in vacuo. The residue was triturated with EtOAc (200 mL) at 25° C. for 30 min to give the title compound as a white solid (40 g, 97% purity). 3H NMR (400 MHz, DMSO-d₆) δ=11.95 (s, 1H), 7.02 (d, J=2.0 Hz, 1H), 6.94 (d, J=2.0 Hz, 1H), 3.87 (s, 3H), 3.63 (s, 3H).

### 6-(Benzylamino)-8-methoxy-1-methyl-1,4-dihydroquinoxaline-2,3-dione

To a solution of 6-bromo-8-methoxy-1-methyl-1,4-dihydroquinoxaline-2,3-dione (18 g, 63.14 mmol, 1 eq) and BnNH₂(8.12 g, 75.76 mmol, 8.26 mL, 1.2 eq) in THF (180 mL) was added LiHMDS (1 M, 151.53 mL, 2.4 eq) dropwise at 25° C., and purged with N₂ for 5 min. BrettPhos (1.69 g, 3.16 mmol, 0.04 eq) and BrettPhos Pd G3 (1.43 g, 1.58 mmol, 0.02 eq) was then added at 25° C., and the resulting mixture was stirred at 80° C. for 12 hr under N₂ atmosphere. The reaction mixture (combined with two other batches of the same scale) was quenched with sat, aq. NH₄CI (200 mL) slowly at 0° C. The solution was dried with anhydrous Na₂SO₄, filtered, and the filtrate was concentrated in vacuo to give a residue which was purified by flash silica gel chromatography (Silica Flash Column, eluent of 20˜100% THF/petroleum ether gradient) to give the title compound as a yellow solid (20 g, 94% purity). ¹H NMR (400 MHz, DMSO-d₆) δ=11.62 (br s, 1H), 7.39-7.29 (m, 4H), 7.27-7.19 (m, 1H), 6.45 (t, J=6.0 Hz, 1H), 6.19 (d, J=2.4 Hz, 1H), 5.99 (d, J=2.4 Hz, 1H), 4.23 (d, J=6.0 Hz, 2H), 3.73 (s, 3H), 3.63-3.57 (m, 3H).

### 6-(Benzylamino)-8-hydroxy-1-methyl-1,4-dihydroquinoxaline-2,3-dione

6-(benzylamino)-8-methoxy-1-methyl-1,4-dihydroquinoxaline-2,3-dione (20 g, 64.24 mmol, 1 eq) was dissolved in DCM (400 mL) and cooled to −70° C. BBr₃ (80.47 g, 321.20 mmol, 30.95 mL, 5 eq) in DCM (50 mL) was added dropwise to the reaction mixture under N₂. The reaction mixture was warmed to 40° C., and stirred for 12 hr. The reaction was quenched with a CHCl/McOH solution (10:1, 600 mL) at 0° C., then concentrated in vacuo. The residue was stirred in 4N HCl water solution at 40° C. for 30 min and then filtered. The filter cake was triturated with (EtOAc, 100 mL) at 25° C. for 30 min to give the title compound as a yellow solid (11 g, 34.04 mmol, 53% yield, 92% purity). ¹H NMR (400 MHz, DMSO-d₆) δ=11.59 (br s, 1H), 9.80 (br s, 1H), 7.36-7.16 (m, 5H), 6.15-5.88 (m, 2H), 4.18 (s, 2H), 3.66 (s, 3H).

### tert-Butyl (3S,5R)-3-(3-((7-(benzylamino)-4-methyl-2,3-dioxo-1,2,3,4-tetrahydro quinoxalin-5-yl)oxy)propyl)-4,4-difluoro-5-methylpiperidine-1-carboxylate

To a solution of 6-(benzylamino)-8-hydroxy-1-methyl-1,4-dihydroquinoxaline-2,3-dione (1 g, 3.03 mmol, 90% purity, 1 eq) in ACN(10 mL) and DMF (10 mL) were added K₂CO₃ (836.77 mg, 6.05 mmol, 2 eq) and tert-butyl (3R,5S)-4,4-difluoro-3-methyl-5-(3-(tosyloxy)propyl)piperidine-1-carboxylate (1.35 g, 3.03 mmol, 1 eq). The reaction mixture was stirred at 60° C. for 6 hr. Water (50 mL) was added and the solution (combined with another batch at 1.5 g scale) was extracted with EtOAc (50 mL×3). The combined organic phase was washed with brine (100 mL), dried over anhydrous Na₂SO₄, filtered and concentrated in vacuo to give a residue which was purified by flash silica gel chromatography (Silica Flash Column, eluent of 33˜100% ethyl acetate/petroleum ether gradient) to give the title compound as a yellow solid (1 g). ¹H NMR (400 MHz, DMSO-d₆) δ=11.73-11.60 (m, 1H), 7.27-7.18 (m, 1H), 7.39-7.16 (m, 4H), 6.64 (s, 2H), 6.42 (br t, J=6.0 Hz, 1H), 6.17 (d,)=2.4 Hz, 1H), 5.99 (d, J=2.4 Hz, 1H), 4.23 (br d, J=6.0 Hz, 1H), 4.14-3.99 (m, 1H), 3.98-3.87 (m, 3H), 3.82-3.77 (m, 1H), 3.69-3.56 (m, 3H), 3.31-3.27 (m, 2H), 2.69 (s, 3H), 1.40 (s, 9H), 0.94 (d, J=6.8 Hz, 4H).

### tert-Butyl (3S,5R)-3-[3-[(7-amino-4-methyl-2,3-dioxo-1H-quinoxalin-5-yl)oxy]pro pyl]-4,4-difluoro-5-methyl-piperidine-1-carboxylate

To a solution tert-butyl (3S,5R)-3-(3-((7-(benzylamino)-4-methyl-2,3-dioxo-1,2,3,4-tetrahydroquinoxalin-5-yl)oxy)propyl)-4,4-difluoro-5-methylpiperidine-1-carboxylate (400 mg, 698.52 μmol, 1 eq) in THF (4 mL) was added Pd/C (150 mg, 10% purity) under Ar. The suspension was degassed under vacuum and purged with H_{2 3} times. The reaction mixture was stirred under H₂ (15 psi) at 25° C. for 12 hr. The reaction mixture (combined with another batch of the same scale) was filtered through a pad of Celite® and the filtrate was concentrated in vacuo to give the title compound as a red solid (800 mg, crude). ¹H NMR (400 MHz, DMSO-d₆) δ=11.67 (br d, J=13.6 Hz, 1H), 7.95 (s, 1H), 6.13 (d, J=2.0 Hz, 1H), 5.99 (d, J=2.0 Hz, 1H), 5.21 (s, 1H), 4.20-3.88 (m, 4H), 3.65-3.61 (m, 2H), 2.64-2.53 (m, 1H), 2.22-2.14 (m, 1H), 1.95-1.83 (m, 3H), 1.40 (s, 9H), 1.35 (s, 4H), 0.94 (d, J=6.8 Hz, 3H).

### tert-Butyl (3S,5R)-3-(3-((7-((2,5-dichloropyrimidin-4-yl)amino)-4-methyl-2,3-dioxo-1,2,3,4-tetrahydroquinoxalin-5-yl)oxy)propyl)-4,4-difluoro-5-methylpiperidine-1-carboxylate

To a solution of tert-butyl (3S,5R)-3-(3-((7-amino-4-methyl-2,3-dioxo-1,2,3,4-tetrahydroquinoxalin-5-yl)oxy)propyl)-4,4-difluoro-5-methylpiperidine-1-carboxylate (800 mg, 1.66 mmol, 1 eq) in DMF (8 mL) were added DIPEA (428.55 mg, 3.32 mmol, 577,56 μL, 2 eq) and 2,4,5-trichloropyrimidine (608.22 mg, 3.32 mmol, 2 eq). The reaction mixture was stirred at 25° C. for 2 hr. Water (50 mL) was added and the solution was extracted with EtOAc (50 mL×3). The combined organic phase was washed with brine (50 mL), dried over anhydrous Na₂SO₄, filtered and the filtrate was concentrated in vacuo to give a residue which was purified by flash silica gel chromatography (Silica Flash Column, eluent of 20˜100% THF/petroleum ether gradient) to give the title compound as a gray solid (750 mg, 83% purity). ¹H NMR (400 MHz, DMSO-d₆) δ=12.17-11.94 (m, 1H), 9.73-9.44 (m, 1H), 8.44-8.34 (m, 1H), 7.26-7.10 (m, 2H), 4.18-4.00 (m, 3H), 3.98-3.85 (m, 1H), 3.74-3.63 (m, 3H), 3.32-3.27 (m, 2H), 2.21-2.14 (m, 2H), 1.93-1.84 (m, 4H), 1.43-1.36 (m, 9H), 0.94 (d, J=6.8 Hz, 3H).

### 6-((2,5-Dichloropyrimidin-4-yl)amino)-8-(3-((3S,5R)-4,4-difluoro-5-methylpi peridin-3-yl)propoxy)-1-methyl-1,4-dihydroquinoxaline-2,3-dione

A solution of tert-butyl (3S,5R)-3-(3-((7-((2,5-dichloropyrimidin-4-yl)amino)-4-methyl-2,3-dioxo-1,2,3,4-tetrahydroquinoxalin-5-yl)oxy)propyl)-4,4-difluoro-5-methylpiperidine-1-carboxylate (700 mg, 1.11 mmol, 1 eq) in DCM (10 mL) and TFA (2.5 mL) was stirred at 25° C. for 30 min. The solution was concentrated in vacuo to give the title compound as a yellow solid (700 mg, crude, TFA). LCMS: [M+H]⁺=529.3.

### (1³S,1³R)-2⁵-Chloro-1⁴,1⁴-difluoro-1⁵,4⁴-dimethyl-41, 42, 43,44-tetrahydro-5-oxa-3-aza-4(7,5)-quinoxalina-2(2,4)-pyrimidina-1 (1,3)-piperidinacyclooctaphane-4²,4³-dione (28)

To a solution of 6-((2,5-dichloropyrimidin-4-yl)amino)-8-(3-((3S,5R)-4,4-difluoro-5-methylpiperidin-3-yl)propoxy)-1-methyl-1,4-dihydroquinoxaline-2,3-dione (700 mg, 1.09 mmol, 1 eq, TFA) in DMSO (28 mL) was added DIPEA (1.41 g, 10.88 mmol, 1.90 mL, 10 eq). The reaction mixture was stirred at 80° C. for 12 hr. Water (50 mL) was added and the solution (combined with another 50 mg scale) was extracted with EtOAc (50 mL×3). The combined organic phase was washed with brine (50 mL), dried over anhydrous Na₂SO₄, filtered and the filtrate was concentrated in vacuo to give a residue which was purified by flash silica gel chromatography (Silica Flash Column, eluent of 50˜100% THF/petroleum ether gradient) to give the title compound as yellow solid (250 mg, 77% purity). ¹H NMR (400 MHz, DMSO-d₆) δ=12.15-11.80 (m, 1H), 9.10 (s, 1H), 8.11 (s, 1H), 7.61 (br s, 1H), 6.77 (s, 1H), 4.75-4.54 (m, 2H), 4.47 (br t, J=11.6 Hz, 1H), 4.21-4.10 (m, 1H), 3.67 (s, 3H), 2.74 (br t,/=12.7 Hz, 1H), 2.41-2.28 (m, 1H), 1.99-1.82 (m, 2H), 1.78-1.55 (m, 2H), 1.42-1.30 (m, 2H), 0.97 (br d, J=6.5 Hz, 3H). LCMS: [M+H]⁺=493.2.

### 3-(Hydroxymethyl)cyclobutan-1-ol

To a mixture of 3-oxocyclobutane-1-carboxylic acid (10 g, 87.64 mmol, 1 eq) in THE (500 mL) was added BH₃-Me₂S (10 M, 26.29 mL, 3 eq) in one portion at −70° C. under N₂. The reaction mixture was stirred at 15° C. for 3 hr. The reaction (combined with another two batches of the same scale) was cooled to 0° C. in an ice bath. MeOH (100 mL) was added dropwise to quench the reaction. The solvent was removed under reduced pressure to give the title compound as a yellow oil (26 g, 217.17 mmol, 83% yield, 85% purity). ¹H NMR (400 MHz, CDCl₃) δ=5.51-5. 15 (m, 1H), 4.45-3.96 (m, 1H), 3.84-3.38 (m, 2H), 2.55-1.81 (m, 4H), 1.76-1.49 (m, 1H).

### (3-Hydroxycyclobutyl)methyl 4-methylbenzenesulfonate

To a solution of 3-(hydroxymethyl)cyclobutan-1-ol (26 g, 254.57 mmol, 1 eq) in DCM (300 mL) were added TEA (51.52 g, 509.15 mmol, 70.87 mL, 2 eq) and DMAP (3.11 g, 25.46 mmol, 0.1 eq) at 15° C. TosCl (48.53 g, 254.57 mmol, 1 eq) was then added slowly at 0° C. under N₂ and the reaction mixture was stirred at 15° C. for 12 hr. The reaction mixture was poured into water (400 mL) and the aqueous phase was extracted with DCM (300 mL×2). The combined organic phase was washed with brine (800 mL), dried with anhydrous Na₂SO₄, filtered and the filtrate was concentrated in vacuo to give a residue which was purified by column chromatography (SiO₂, petroleum ether/ethyl acetate=1/0 to 3/1) to give the title compound as a white solid (33 g, 122.80 mmol, 48% yield, 95% purity). ¹H NMR (400 MHz, DMSO-d₆) δ=7.83-7.72 (m, 2H), 7.52-7.41 (m, 2H), 5.18-4.54 (m, 1H), 4.14-3.83 (m, 3H), 2.42 (s, 3H), 2.21-2.11 (m, 1H), 2.01-1.78 (m, 3H), 1.53-1.38 (m, 1H).

### (1³S,1⁵R)-2⁵-Chloro-1⁴,1⁴-difluoro-4¹-((3-hydroxycyclobutyl)methyl)-1⁵,4⁴-dimethyl-41, 42, 43,44-tetrahydro-5-oxa-3-aza-4(7,5)-quinoxalina-2(2,4)-pyrimidina-1 (1,3)-piperidinacyclooctaphane-4²,4³-dione

To a solution of (1³S,1⁵R)-2⁵-chloro-1⁴,1⁴-difluoro-1⁵,4⁴-dimethyl-4¹,4²,4³,4⁴-tetrahydro-5-oxa-3-aza-4(7,5)-quinoxalina-2(2,4)-pyrimidina-1 (1,3)-piperidinacy clooctaphane-4²,4³-dione (150 mg, 304.32 μmol, 1 eq) in DMSO (2 mL) were added (3-hydroxycyclobutyl)methyl 4-methylbenzenesulfonate (156.00 mg, 608.64 μmol, 2 eq), Cs₂CO₃ (198.31 mg, 608.64 μmol, 2 eq) and KI (25.26 mg, 152.16 μmol, 0.5 eq). The reaction mixture was stirred at 80° C. for 12 hr under N₂. The reaction mixture (combined with another batch at 100 mg scale) was filtered and the filtrate was concentrated in vacuo to give a residue which was purified by prep-HPLC (column: Phenomenex C18 80*40 mm*3 μm; mobile phase: [water (NH₄HCO₃)-ACN]; B %: 35%-65%, 8 min) to give the title compound as a white solid (60 mg, 97% purity). LCMS: [M+H]⁺=577.3.

### (1³S,1⁵R)-2⁵-Chloro-1⁴,1⁴-difluoro-4¹-(((1r,3S)-3-hydroxycyclobutyl)methyl)-1⁵,4⁴-dimethyl-4¹,4²,4³,4⁴-tetrahydro-5-oxa-3-aza-4(7,5)-quinoxalina-2(2,4)-pyrimidina-1 (1,3)-piperidinacyclooctaphane-4²,4³-dione (18-1) and (1⁵S, 1R)-2⁵-chloro-1⁴,1⁴-difluoro-4¹-((1s,3R)-3-hydroxycyclobutyl)methyl)-1⁵, 4⁴-dimethyl-4¹,4²,4³,4⁴-tetrahydro-5-oxa-3-aza-4(7,5)-quinoxalina-2(2,4)-pyrimidina-1 (1,3)-piperidinacyclooctaphane-4²,4³-dione (18-2)

(1³S,1⁵R)-2⁵-Chloro-1⁴,1⁴-difluoro-4¹-((3-hydroxycyclobutyl)methyl)-1⁵,4⁴-dimethyl-4¹,4²,4³,4⁴-tetrahydro-5-oxa-3-aza-4(7,5)-quinoxalina-2(2,4)-pyrimidina-1 (1,3)-piperidinacyclooctaphane-4²,4³-dione (combined with another 20 mg scale) was purified by SFC (column: DAICEL CHIRALPAK IC (250 mm*30 mm, 10 μm); mobile phase: [ACN/EtOH (0.1% NH₃H₂O)]; B %: 70%-70%, 13 min) to give 18-1 as a white solid (7 mg, 98% purity) and 18-2 as a white solid (20 mg, 97.8% purity).

18-1: ¹H NMR (400 MHz, DMSO-d₆) δ=9.01 (s, 1H), 8.15 (s, 1H), 7.77 (d, J=1.4 Hz, 1H), 7.05 (d, J=1.4 Hz, 1H), 5.04 (d, J=6.0 Hz, 1H), 4.70 (br d, J=14.0 Hz, 1H), 4.65-4.56 (m, 1H), 4.51-4.41 (m, 1H), 4.40-4.33 (m, 1H), 4.30-4.14 (m, 2H), 4.09 (br dd, J=8.4, 14.1 Hz, 1H), 3.63 (s, 3H), 2.80-2.72 (m, 1H), 2.65-2.55 (m, 1H), 2.44-2.31 (m, 1H), 2.05-1.98 (m, 2H), 1.95-1.86 (m, 3H), 1.83-1.70 (m, 1H), 1.64 (br t, J=13.2 Hz, 1H), 1.39-1.31 (m, 3H), 0.98 (d, J=6.6 Hz, 3H). LCMS: [M+H]⁺=577.4.

18-2: ¹H NMR (400 MHz, DMSO-d₆) δ=9.00 (s, 1H), 8.15 (s, 1H), 7.77 (d, J=1.4 Hz, 1H), 7.05 (d, J=1.4 Hz, 1H), 5.01 (d, J=6.8 Hz, 1H), 4.75-4.55 (m, 2H), 4.53-4.37 (m, 1H), 4.28-4.11 (m, 2H), 4.03 (br dd, J=6.6, 13.9 Hz, 1H), 3.90-3.77 (m, 1H), 3.63 (s, 3H), 2.76 (br t, J=12.8 Hz, 1H), 2.64-2.5S (m, 1H), 2.45-2.31 (m, 1H), 2.30-2.22 (m, 2H), 2.20-2.12 (m, 1H), 2.05-1.85 (m, 2H), 1.83-1.58 (m, 4H), 1.37 (dt, J=5.4, 12.5 Hz, 1H), 0.98 (d, J=6.8 Hz, 3H). LCMS: [M+H]⁺=577.4.

### Example 16: Synthesis of (1³R, 1⁵S)-2⁵-chloro-1⁴,1⁴-difluoro-4-(1R,3R)-3-hydroxycyclobutyl)methyl)-1⁵,4⁴-dimethyl-4¹,4²,4³,4⁴-tetrahydro-5-oxa-3-aza-4(7,5)-quinoxalina-2(2,4)-pyrimidina-1 (1,3)-piperidinacyclooctaphane-4²,4³-dione (19-1) and (1³R,1⁵S)-2⁵-chloro-1⁴, 1⁴-difluoro-4¹-(((15,3S)-3-hydroxycyclobutyl)methyl)-1⁵,4⁴-dimethyl-4¹,4²,4³,4⁴-tetrahydro-5-oxa-3-aza-4(7,5)-quinoxalina-2(2,4)-pyrimidina-1 (1,3)-piperidinacyclooctaphane-4²,4³-dione (19-2)

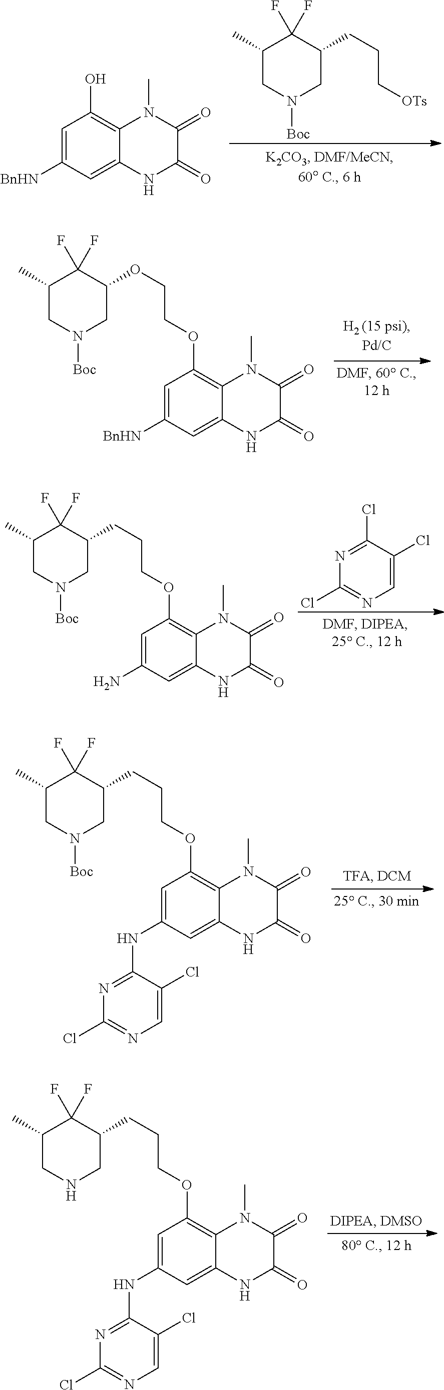

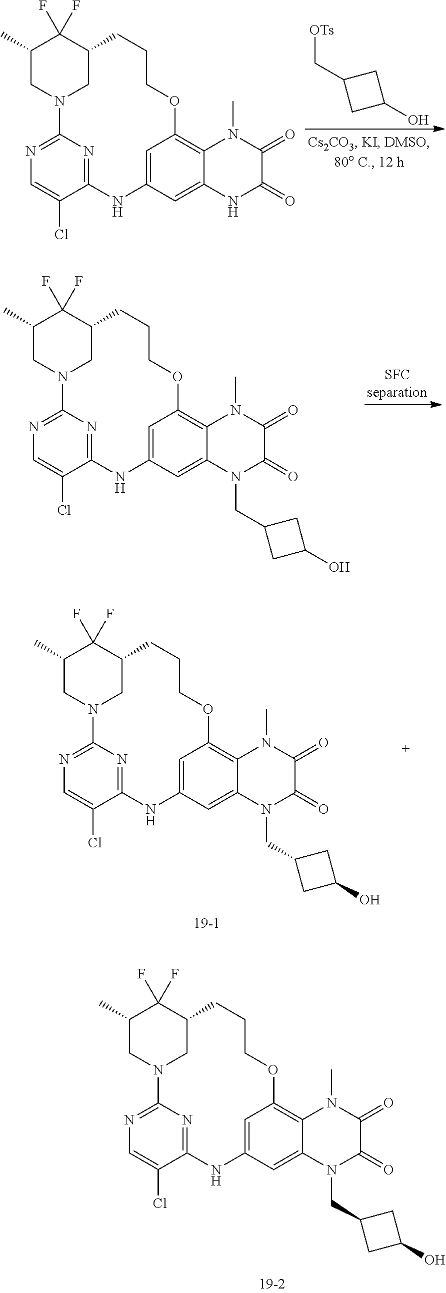 [SMILES: C[C@H]1CN2C[C@@H](CCCOc3cc(cc4[nH]c(=O)c(=O)n(C)c34)Nc3nc2ncc3Cl)C1(F)F ; Cc1ccc(S(=O)(=O)OCC2CC(O)C2)cc1 ; C[C@H]1CN2C[C@@H](CCCOc3cc(cc4c3n(C)c(=O)c(=O)n4CC3CC(O)C3)Nc3nc2ncc3Cl)C1(F)F ; C[C@H]1CN2C[C@@H](CCCOc3cc(cc4c3n(C)c(=O)c(=O)n4C[C@H]3C[C@H](O)C3)Nc3nc2ncc3Cl)C1(F)F ; C[C@H]1CN2C[C@@H](CCCOc3cc(cc4c3n(C)c(=O)c(=O)n4C[C@H]3C[C@@H](O)C3)Nc3nc2ncc3Cl)C1(F)F]

### tert-Butyl (3R,5S)-3-(3˜ ((7-(benzylamino)-4-methyl-2,3-dioxo-1,2,3,4-tetrahydro quinoxalin-5-yl)oxy)propyl)-4,4-difluoro-5-methylpiperidine-1-carboxylate

To a solution of 6-(benzylamino)-8-hydroxy-1-methyl-1,4-dihydroquinoxaline-2,3-dione (2.5 g, 8.41 mmol, 1 eq) and tert-butyl (3S,5R)-4,4-difluoro-3-methyl-5-[3-(p-tolylsulfonyloxy)propyl]piperidine-1-carboxylate (3.76 g, 8.41 mmol, 1 eq) in ACN(25 mL) and DMF (25 mL) was added K₂CO₃ (2.91 g, 21.02 mmol, 2.5 eq). The reaction mixture was stirred at 60° C. for 6 hr. The reaction was quenched with water (40 mL) and extracted with EtOAc (30 mL×3). The combined organic phase was washed with brine (40 mL), dried with anhydrous Na₂SO₄, filtered and concentrated in vacuo to give a residue which was purified by column chromatography (SiO₂, petroleum ether/ethyl acetate/MeOH=3/I/O to 0/3/1) to give the title compound as a yellow solid (1.5 g, 2.28 mmol, 27% yield, 87% purity). ¹H NMR (400 MHz, DMSO-d₆) δ=11.71-11.52 (m, 1H), 7.42-7.18 (m, 5H), 6.42 (br t,/=5.9 Hz, 1H), 6.17 (d, J=1.4 Hz, 1H), 5.99 (s, 1H), 4.22 (br d, J=5.9 Hz, 1H), 4.13-4.01 (m, 1H), 3.98-3.88 (m, 3H), 3.61 (s, 2H), 2.68-2.55 (m, 2H), 2.03-1.74 (m, 6H), 1.40 (s, 9H), 1.33-1.19 (m, 2H), 0.94 (br d, J=6.6 Hz, 3H).

### tert-Butyl (3R,5S)-3-(3-((7-amino-4-methyl-2,3-dioxo-1,2,3,4-tetrahydroquinoxalin-5-yl)oxy)propyl)-4,4-difluoro-5-methylpiperidine-1-carboxylate

To a solution of tert-butyl (3R,5S)-3-(3-((7-(benzylamino)-4-methyl-2,3-dioxo-1,2,3,4-tetrahydroquinoxalin-5-yl)oxy)propyl)-4,4-difluoro-5-methylpiperidine-1-carboxylate (1.3 g, 2.27 mmol, 1 eq) in DMF (15 mL) was added Pd/C (600 mg, 10% purity) under Ar. The suspension was degassed under vacuum and purged 3 times with H₂. The reaction mixture was stirred under H₂ (15 psi) at 60° C. for 12 hr. The reaction mixture (combined with another batch at 200 mg scale) was filtered and the filtrate was concentrated in vacuo to give the title compound as a black oil (1,5 g). LCMS: [M−56+H]⁺=427.2.

### tert-Butyl (3R,5S)-3-(3-((7-((2,5-dichloropyrimidin-4-yl)amino)-4-methyl-2,3-dioxo-1,2,3,4-tetrahydroquinoxalin-5-yl)oxy)propyl)-4,4-difluoro-5-methylpiperidine-1-carboxylate

To a solution of tert-butyl (3R,5S)-3-(3-((7-amino-4-methyl-2,3-dioxo-1,2,3,4-tetrahydroquinoxalin-5-yl)oxy)propyl)-4,4-difluoro-5-methylpiperidine-1-carboxylate (1.5 g, 3.11 mmol, 1 eq) and 2,4,5-trichloropyrimidine (1.14 g, 6.22 mmol, 2 eq) in DMF (16 mL) was added DIPEA (803.55 mg, 6.22 mmol, 1.08 mL, 2 eq). The reaction mixture was stirred at 25° C. for 12 hr. The reaction was quenched with water (30 mL) and then extracted with EtOAc (20 mL×3). The combined organic phase was washed with brine (30 mL), dried with anhydrous Na₂SO₄, filtered and concentrated in vacuo to give a residue which was purified by column chromatography (SiO₂, petroleum ether/ethyl acetate/MeOH=4/I/O to 0/0/1) to give the title compound as a yellow solid (1.1 g, 1.26 mmol, 40% yield, 72% purity). LCMS: [M−56+H]⁺=573.1.

### 6-(2,5-Dichloropyrimidin-4-yl)amino)-8-(3-(3R,5S)-4,4-difluoro-5-methylpiperidin-3-yl)propoxy)-1-methyl-1,4-dihydroquinoxaline-2,3-dione

A solution of tert-butyl (3R,5S)-3-[3-[[7-[(2,5-dichloropyrimidin-4-yl)amino]-4-methyl-2,3-dioxo-1H-quinoxalin-5-yl]oxy]propyl]-4,4-difluoro-5-methyl-piperidine-1-carboxylate (1 g, 1.59 mmol, 1 eq) in DCM (10 mL) and TFA (2.5 mL) was stirred at 25° C. for 30 min. The reaction mixture was concentrated in vacuo to give the title compound as a yellow oil (1 g, crude, TFA). LCMS: [M+H]⁺=529.2.

### (1³R, 1⁵S)-2⁵-Chloro-1⁴,1⁴-difluoro-1⁵,4⁴-dimethyl-4¹,4²,4³,4⁴-tetrahydro-5-oxa-3-aza-4(7,5)-quinoxalina-2(2,4)-pyrimidina-1 (1,3)-piperidinacyclooctaphane-4²,4³-dione

To a solution of 6-((2,5-dichloropyrimidin-4-yl)amino)-8-(3-((3R,5S)-4,4-difluoro-5-methylpiperidin-3-yl)propoxy)-1-methyl-1,4-dihydroquinoxaline-2,3-dione (1 g, 1.55 mmol, 1 eq, TFA) in DMSO (40 mL) was added DIEA (2.01 g, 15.54 mmol, 2.71 mL, 10 eq). The reaction mixture was stirred at 80° C. for 12 hr. The reaction (combined with another batch at 100 mg scale) was quenched with water (60 mL) and extracted with EtOAc (30 mL×3). The combined organic phase was washed with brine (60 mL), dried with anhydrous Na₂SO₄, filtered and the filtrate was concentrated in vacuo to give a residue which was purified by column chromatography (SiO₂, Ethyl acetate/MeOH=1/0 to 0/1) to give the title compound as a yellow solid (380 mg). ¹H NMR (400 MHz, DMSO-d₆) δ=12.25-11.79 (m, 1H), 9.08 (s, 1H), 8.10 (s, 1H), 7.60 (s, 1H), 6.76 (d, J=1.9 Hz, 1H), 4.73-4.64 (m, 1H), 4.59 (br d, J=14.1 Hz, 1H), 4.46 (br t, J=11.8 Hz, 1H), 4.20-4.08 (m, 1H), 3.67 (s, 3H), 2.79-2.69 (m, 1H), 2.61-2.52 (m, 2H), 2.43-2.29 (m, 1H), 2.03-1.81 (m, 2H), 1.80-1.68 (m, 1H), 1.67-1.57 (m, 1H), 1.43-1.30 (m, 1H), 0.97 (d, J=6.6 Hz, 3H).

### (1³R, 1⁵S)-2⁵-Chloro-1⁴, 1⁴-difluoro-4¹-((3-hydroxycyclobutyl)methyl)-1⁵,4⁴-dimethyl-4¹,4²,4³,4⁴-tetrahydro-5-oxa-3-aza-4(7,5)-quinoxalina-2(2,4)-pyrimidina-1 (1,3)-piperidinacyclooctaphane-4²,4³-dione

To a solution of (1³R,1⁵S)-2⁵-chloro-1⁴,1⁴-difluoro-1⁵,4⁴-dimethyl-4¹,4²,4³,4⁴-tetrahydro-5-oxa-3-aza-4(7,5)-quinoxalina-2(2,4)-pyrimidina-1 (1,3)-piperidinacyclooctaphane-4²,4³-dione (260 mg, 527.48 μmol, 1 eq) and (3-hydroxycyclobutyl)methyl 4-methylbenzenesulfonate (270.41 mg, 1.05 mmol, 2 eq) in DMSO (2.6 mL) were added Cs₂CO₃ (343.73 mg, 1.05 mmol, 2 eq) and KI (43.78 mg, 263.74 μmol, 0.5 eq). The reaction mixture was stirred at 80° C. for 12 hr. The reaction was quenched with water (20 mL) and extracted with EtOAc (10 mL×3). The combined organic phase was washed with brine (20 mL), dried with anhydrous Na₂SO₄, filtered and concentrated in vacuo to give a residue which was purified by prep-HPLC (column: Waters Xbridge™ BEH C18 100*30 mm*10 μm; mobile phase: [water (NH₄HCO₃)-ACN]; B %: 45%-65%, 8 min) to give the title compound as a white solid (60 mg, 102.94 μmol, 20% yield, 99% purity). ¹H NMR (400 MHz, DMSO-d₆) δ=9.05-8.95 (m, 1H), 8.15 (s, 1H), 7.77 (d, J=1.3 Hz, 1H), 7.05 (s, 1H), 5.05-4.97 (m, 1H), 4.70 (br d, J=12.5 Hz, 1H), 4.64-4.56 (m, 1H), 4.46 (br t, J=11.4 Hz, 1H), 4.27-4.15 (m, 2H), 4.03 (br dd, J=6.6, 13.9 Hz, 1H), 3.88-3.79 (m, 1H), 3.63 (s, 3H), 2.76 (brt, J=12.8 Hz, 1H), 2.64-2.54 (m, 1H), 2.44-2.34 (m, 1H), 2.30-2.11 (m, 3H), 1.90 (br dd, J=5.6, 7.4 Hz, 2H), 1.82-1.59 (m, 4H), 1.44-1.31 (m, 1H), 0.98 (d, J=6.6 Hz, 3H).

### (1³R, 1⁵S)-2⁵-Chloro-1⁴, 1⁴-difluoro-4¹-(((1r, 3R)-3-hydroxycyclobutyl)methyl)-1⁵, 4⁴-dimethyl-4¹,4²,4³,4⁴-tetrahydro-5-oxa-3-aza-4(7,5)-quinoxalina-2(2,4)-pyrimidina-1 (1,3)-piperidinacyclooctaphane-4²,4³-dione (19-1) and (1³R, 1⁵S)-2⁵-chloro-1⁴,1⁴-difluoro-4¹-(((1s,3S)-3-hydroxycyclobutyl)methyl)-1²,4⁴-dimethyl-4²,4²,4³,4⁴-tetrahydro-5-oxa-3-aza-4(7,5)-quinoxalina-2(2,4)-pyrimidina-1 (1,3)-piperidinacyclooctaphane-4²,4³-dione (19-2)

(1³R,1⁵S)-2⁵-Chloro-1⁴,1⁴-difluoro-4¹-((3-hydroxycyclobutyl)methyl)-1⁵,4⁴-dimethyl-4¹,4²,4³,4⁴-tetrahydro-5-oxa-3-aza-4(7,5)-quinoxalina-2(2,4)-pyrimidina-1 (1,3)-piperidinacyclooctaphane-4²,4³-dione (90 mg) was separated by SFC (column: DAICEL CHIRALCEL OD (250 mm*30 mm, 10 μm);mobile phase: [0.1% NH₃H₂O IPA];B %: 50%-50%, 17 min) to give 19-1 as a white solid (7 mg, 12% yield, 99% purity) and 19-2 as a white solid (15 mg, 25% yield, 99% purity).

19-1: ¹H NMR (400 MHz, DMSO-d₆) δ=9.01 (s, 1H), 8.15 (s, 1H), 7.77 (s, 1H), 7.05 (s, 1H), 5.04 (br d, J=5.8 Hz, 1H), 4.70 (br d, J=12.6 Hz, 1H), 4.60 (br d, J=12.5 Hz, 1H), 4.46 (br t, J=12.1 Hz, 1H), 4.40-4.31 (m, 1H), 4.30-4.14 (m, 2H), 4.13-4.01 (m, 1H), 3.63 (s, 3H), 2.83-2.65 (m, 2H), 2.63-2.55 (m, 1H), 2.44-2.30 (m, 1H), 2.09-1.96 (m, 3H), 1.89 (br d, J=5. 6 Hz, 3H), 1.81-1.57 (m, 2H), 1.44-1.31 (m, 1H), 0.98 (br d, J=6.6 Hz, 3H). LCMS: [M+H]⁻=577.4.

19-2: ¹H NMR (400 MHz, DMSO-d₆) δ=9.00 (s, 1H), 8.15 (s, 1H), 7.77 (br s, 1H), 7.05 (br s, 1H), 5.01 (br d, J=6.8 Hz, 1H), 4.70 (br d, J=12.3 Hz, 1H), 4.60 (br d, J=12.6 Hz, 1H), 4.45 (br t. J=11.6 Hz, 1H), 4.26-4.13 (m, 2H), 4.08-3.98 (m, 1H), 4.03 (br dd, J=6.4, 13.6 Hz, 1H), 3.83 (br dd, J=7.3, 14.3 Hz, 1H), 3.63 (s, 3H), 2.76 (br t, J=12.5 Hz, 1H), 2.63-2.55 (m, 1H), 2.43-2.31 (m, 1H), 2.30-2.21 (m, 2H), 2.20-2.11 (m, 1H), 2.04-1.86 (m, 2H), 1.83-1.59 (m, 4H), 1.44-1.31 (m, 1H), 0.98 (br d, J=6.4 Hz, 3H). LCMS: [M+H]⁺=577.4.

### Example 17: Synthesis of (1³S,1⁵R)-2⁵-chloro-1⁴, 1⁴-difluoro-4-(3-hydroxy-3-methylbutyl)-1⁵,4³-dimethyl-4²,4³-dihydro-4 H-5,8-dioxa-3-aza-4(6,4)-benzo[d]imidazola-2(2,4)-pyrimidina-1 (1,3)-piperidinacyclooctaphan-4²-one (33)

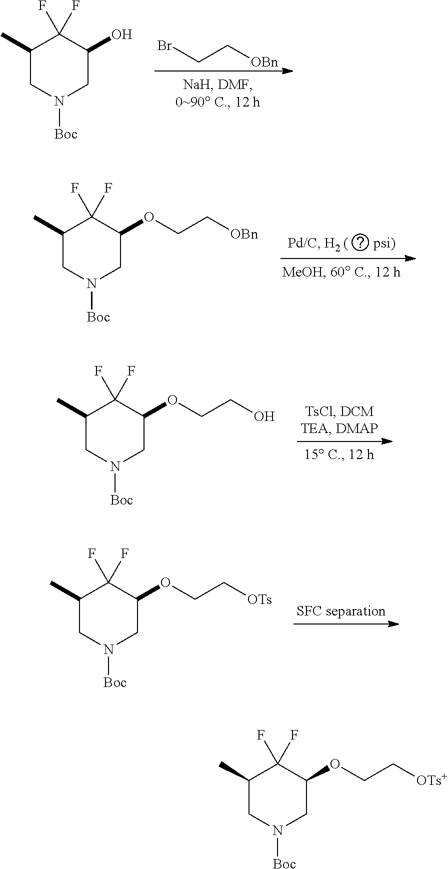

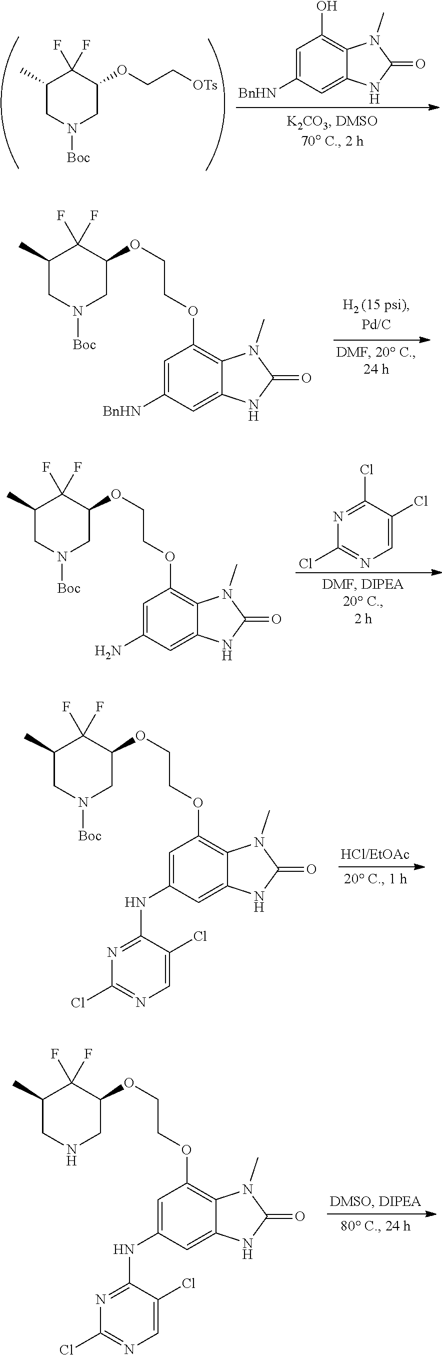

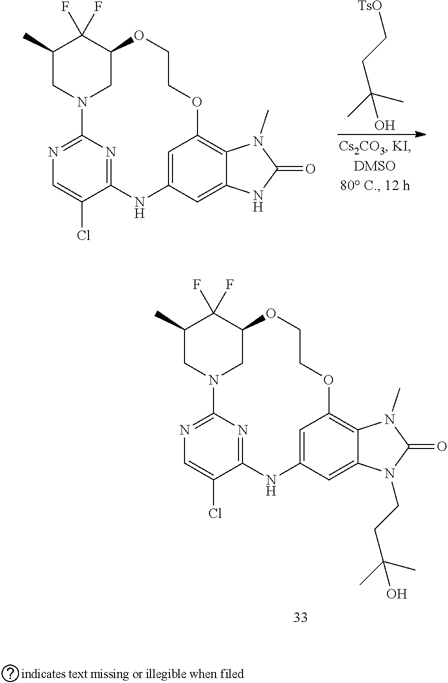 [SMILES: C[C@@H]1CN2C[C@H](OCCOc3cc(cc4[nH]c(=O)n(C)c34)Nc3nc2ncc3Cl)C1(F)F ; Cc1ccc(S(=O)(=O)OCCC(C)(C)O)cc1 ; C[C@@H]1CN2C[C@H](OCCOc3cc(cc4c3n(C)c(=O)n4CCC(C)(C)O)Nc3nc2ncc3Cl)C1(F)F]

### tert-Butyl (3S,5R)-3-(2-(benzyloxy)ethoxy)-4,4-difluoro-5-methylpiperidine-1-carboxylate

To a solution of tert-butyl 4,4-difluoro-3-hydroxy-5-methyl-piperidine-1-carboxylate (13.00 g, 51.74 mmol, 1 eq) in DMF (100 mL) was added NaH (4.14 g, 103.47 mmol, 60% purity, 2 eq) at 0° C. The reaction mixture was stirred at 0° C. for 30 minutes and then 2-bromoethoxymethylbenzene (13.35 g, 62.08 mmol, 9.82 mL, 1.2 eq) was added. The reaction mixture was heated to 90° C., and stirred for 12 hr under N₂. The reaction mixture was quenched slowly with sat. NH₄Cl (100 mL) and then extracted with ethyl acetate (150 mL×2). The combined organic phase was washed with brine (200 mL), dried with anhydrous Na₂SO₄, filtered and concentrated in vacuo. The residue was purified by flash silica gel chromatography (ISCO®, SepaFlash® Silica Flash Column, eluent of 5˜50% ethyl acetate/petroleum ether gradient) to give the title compound as a colorless oil (5.7 g, 11.53 mmol, 22% yield, 78% purity). ¹H NMR (400 MHz, CDCl₃) δ=7.38-7.28 (m, 5H), 4.63-4.55 (m, 2H), 4.35-4.12 (m, 1H), 4.03-3.80 (m, 3H), 3.71-3.62 (m, 2H), 3.60-3.49 (m, 1H), 2.93 (t, J=10.4 Hz, 1H), 2.70 (s, 1H), 2.07-1.88 (m, 1H), 1.46 (s, 9H), 1.07 (d, J=6.8 Hz, 3H).

### tert-Butyl (3S,5R)-4,4-difluoro-3-(2-hydroxyethoxy)-5-methylpiperidine-1-carboxylate

To a solution of tert-butyl 3-(2-benzyloxyethoxy)-4,4-difluoro-5-methyl-piperidine-1-carboxylate (5.70 g, 14.79 mmol, 1 eq) in MeOH (60 mL) was added Pd/C (2 g, 10% purity) under Ar. The suspension was degassed under vacuum and purged with H₂ several times. The reaction mixture was stirred under H₂ (50 psi) at 60° C. for 12 hr. The reaction mixture was filtered and concentrated in vacuo to give the title compound as a colorless oil (4.3 g, crude). ¹H NMR (400 MHz, CDCl₃) δ=4.20-3.63 (m, 6H), 3.58-3.42 (m, 1H), 3.23-2.63 (m, 2H), 2.55-2.10 (m, 1H), 2.07-1.88 (m, 1H), 1.47 (s, 9H), 1.08 (d, J=6.8 Hz, 3H).

### tert-Butyl (3R,5S)-4,4-difluoro-3-methyl-5-(2-(tosyloxy)ethoxy)piperidine-1-carboxylate

To a solution of tert-butyl 4,4-difluoro-3-(2-hydroxyethoxy)-5-methyl-piperidine-1-carboxylate (4.3 g, 14.56 mmol, 1 eq) in DCM (40 mL) was added DMAP (177.88 mg, 1.46 mmol, 0.1 eq) and TEA (2.95 g, 29.12 mmol, 4.05 mL, 2 eq). 4-methylbenzenesulfonyl chloride (4.16 g, 21.84 mmol, 1.5 eq) was then added at 15° C. The reaction mixture was stirred at 15° C. for 12 hr. The reaction mixture was treated with water (50 mL), then extracted with EtOAc (50 mL×3). The combined organic phase was washed with brine (80 mL), dried with anhydrous Na₂SO₄, filtered and concentrated in vacuo. The residue was purified by column chromatography (SiO₂, petroleum ether/ethyl acetate=8/1 to 1/1) to give the title compound as a colorless oil (5 g, 11.01 mmol, 76% yield, 99% purity). ¹H NMR (400 MHz, CDCl₃) δ=7.81 (d, J=8.4 Hz, 2H), 7.35 (d, J=8.0 Hz, 2H), 4.19-4.05 (m, 3H), 4.00-3.78 (m, 3H), 3.50-3.36 (m, 1H), 2.85 (s, 1H), 2.67 (s, 1H), 2.45 (s, 3H), 2.02-1.84 (m, 1H), 1.47 (s, 9H), 1.03 (d,)=6.8 Hz, 3H).

### tert-Butyl (3R,5S)-4,4-difluoro-3-methyl-5-(2-(tosyloxy)ethoxy)piperidine-1-carboxylate and tert-butyl (3S,5R)-4,4-difluoro-3-methyl-5-(2-(tosyloxy)ethoxy)piperidine-1-carboxylate

The cis-mixture of tert-butyl (3R,5S)-4,4-difluoro-3-methyl-5-(2-(tosyloxy)ethoxy)piperidine-1-carboxylate was separated by SFC (column: DAICEL CHIRALPAK AD (250 mm*50 mm, 10 μm);mobile phase: [CO₂-EtOH (0.1% NH₃H₂O)]; B %: 15%, isocratic elution mode) to give tert-butyl (3R,5S)-4,4-difluoro-3-methyl-5-[2-(p-tolylsulfonyloxy)ethoxy]piperidine-1-carboxylate as a white solid (2.2 g, 4.85 mmol, 44% yield, 99% purity); ¹H NMR (400 MHz, CDCL) δ=7.81 (d, J=8.2 Hz, 2H), 7.35 (d, J=8.2 Hz, 2H), 4.19-4.06 (m, 3H), 3.99-3.79 (m, 3H), 3.49-3.35 (m, 1H), 2.85 (s, 1H), 2.68 (s, 1H), 2.45 (s, 3H), 2.03-1.84 (m, 1H), 1.47 (s, 9H), 1.03 (d, J=6.8 Hz, 3H); LCMS: [M−Boc+H]⁺=350.2; and tert-butyl (3S,5R)-4,4-difluoro-3-methyl-5-[2-(p-tolylsulfonyloxy)ethoxy]piperidine-1-carboxylate as a white solid (2.2 g, 4.85 mmol, 44% yield, 99% purity); ¹H NMR (400 MHz, CDCl₃) δ=7.73 (d, J=8.4 Hz, 2H), 7.28 (d, J=8.0 Hz, 2H), 4.11-3.99 (m, 3H), 3.92-3.72 (m, 3H), 3.40-3.28 (m, 1H), 2.77 (t, J=9.9 Hz, 1H), 2.69-2.52 (m, 1H), 2.38 (s, 3H), 1.95-1.79 (m, 1H), 1.40 (s, 9H), 0.96 (d, J=6.8 Hz, 3H); LCMS: [M−Boc+H]⁺=350.2.

### tert-Butyl (3S,5R) ˜3-(2-((6-(benzylamino)-3-methyl-2-oxo-2,3-dihydro-1H-benzo[d]imidazol-4-yl)oxy)ethoxy)-4,4-difluoro-5-methylpiperidine-1-carboxylate

A mixture of 6-(benzylamino)-4-hydroxy-3-methyl-1H-benzimidazol-2-one (800 mg, 2.97 mmol, 1 eq), tert-butyl (3R,5S)-4,4-difluoro-3-methyl-5-[2-(p-tolylsulfonyloxy) ethoxy]piperidine-1-carboxylate (1.34 g, 2.97 mmol, 1 eq), and K₂CO₃ (821.13 mg, 5.94 mmol, 2 eq) in DMSO (10 mL) was degassed and purged with N₂ three times. The reaction mixture was stirred at 70° C. for 2 hr under N₂. The reaction mixture was poured into water and filtered. The filter cake was washed with water and dried in vacuo to give the title compound as a yellow solid (800 mg, 1.46 mmol, 49% yield). ¹H NMR (400 MHz, DMSO-d₆) δ=10.36 (s, 1H), 7.40-7.15 (m, 5H), 6.03 (s, 1H), 5.93 (t, J=6.0 Hz, 1H), 5.84 (s, 1H), 4.21 (d, J=5.6 Hz, 2H), 4.10-3.85 (m, 5H), 3.85-3.70 (m, 2H), 3.32 (s, 3H), 2.93-2.64 (m, 2H), 2.10-1.95 (m, 1H), 1.37 (s, 9H), 0.96 (d, J=6.8 Hz, 3H).

### tert-Butyl (3S,5R)-3-(2-((6-amino-3-methyl-2-oxo-2,3-dihydro-1H-benzo[d]imidazol-4-yl)oxy)ethoxy)-4,4-difluoro-5-methylpiperidine-1-carboxylate

To a solution of tert-butyl (3S,5R)-3-(2-((6-(benzylamino)-3-methyl-2-oxo-2,3-dihydro-1H-benzo[d]imidazol-4-yl)oxy)ethoxy)-4,4-difluoro-5-methylpiperidine-1-carboxylate (800 mg, 1.46 mmol, 1 eq) in DMF (10 mL) was added Pd/C (400.00 mg, 375.87 μmol, 10% purity) under Ar. The suspension was degassed under vacuum and purged with H₂ several times. The reaction mixture was stirred under H₂ (15 psi) at 20° C. for 24 hr. The reaction mixture was filtered, and then concentrated under reduced pressure to give the title compound as a yellow solid (600 mg, crude). ¹H NMR (400 MHz, DMSO-d₆) δ=10.36 (s, 1H), 5.96 (d, J=1.2 Hz, 1H), 5.91 (d, J=1.6 Hz, 1H), 4.76 (s, 2H), 4.13-3.90 (m, 5H), 3.85-3.67 (m, 2H), 3.35 (s, 3H), 2.97-2.61 (m, 2H), 2.15-1.96 (m, 1H), 1.37 (s, 9H), 0.96 (d, J=6.8 Hz, 3H).

### tert-Butyl (3S,5R)-3-(2-((6-((2,5-dichloropyrimidin-4-yl)amino)-3-methyl-2-oxo-2,3-dihydro-1H-benzo[d]imidazol-4-yl)oxy)ethoxy)-4,4-difluoro-5-methylpiperidine-1-carboxylate

A mixture of tert-butyl (3S,5R)-3-(2-((6-amino-3-methyl-2-oxo-2,3-dihydro-1H-benzo[d]imidazol-4-yl)oxy)ethoxy)-4,4-difluoro-5-methylpiperidine-1-carboxylate (600 mg, 1.31 mmol, 1 eq), 2,4,5-trichloropyrimidine (337,53 mg, 1.84 mmol, 1.4 eq), and DIPEA (339.75 mg, 2.63 mmol, 457.89 μL, 2 eq) in DMF (8 mL) was degassed and purged with N₂ three times. The reaction mixture was stirred at 20° C. for 2 hr under N₂ atmosphere. The reaction mixture was poured into water and filtered. The filter cake was washed with water and dried in vacuo to give the title compound as a yellow solid (600 mg, 994.29 μmol, 76% yield). ¹H NMR (400 MHz, DMSO-d₆) δ=10.89 (s, 1H), 9.38 (s, 1H), 8.34 (s, 1H), 7.00-6.94 (m, 2H), 4.23-4.13 (m, 2H), 4.07-3.95 (m, 3H), 3.83-3.70 (m, 2H), 3.47 (s, 3H), 2.97-2.83 (m, 1H), 2.79-2.64 (m, 1H), 2.15-1.96 (m, 1H), 1.37 (s, 9H), 0.96 (d, J=6.8 Hz, 3H).

### 5-((2,5-Dichloropyrimidin-4-yl)amino)-7-(2-(((3S,5R)-4,4-difluoro-5-methylpiperidin-3-yl)oxy)ethoxy)-1-methyl-1,3-dihydro-2H-benzo[d]imidazol-2-one

A solution of tert-butyl (3S,5R)-3-(2-((6-((2,5-dichloropyrimidin-4-yl)amino)-3-methyl-2-oxo-2,3-dihydro-1H-benzo[d]imidazol-4-yl)oxy)ethoxy)-4,4-difluoro-5-methylpiperidine-1-carboxylate (800 mg, 1.33 mmol, 1 eq) in HCl/EtOAc (10 mL) was stirred at 20° C. for 1 hr under N₂ atmosphere. The reaction mixture was concentrated in vacuo and the residue was dissolved in MeOH (50 mL) and basic resin was added to pH>7. The solution was filtered and the filtrate was concentrated in vacuo to give the title compound as a yellow solid (600 mg, crude). LCMS: [M+H]⁺=503.2.

### (1³S,1⁵R)-2⁵-Chloro-1⁴,1⁴-difluoro-1⁵,4³-dimethyl-4²,4³-dihydro-4 H-5,8-dioxa-3-aza-4(6,4)-benzo[d]imidazola-2(2,4)-pyrimidina-1 (1,3)-piperidinacyclooctaphan-4²-one

A mixture of 5-((2,5-dichloropyrimidin-4-yl)amino)-7-(2-(((3S,5R)-4,4-difluoro-5-methylpiperidin-3-yl)oxy)ethoxy)-1-methyl-1,3-dihydro-2H-benzo[d]imidazol-2-one (400 mg, 794.71 μmol, 1 eq) and DIPEA (1.03 g, 7.95 mmol, 1.38 mL, 10 eq) in DMSO (40 mL) was degassed and purged with N₂ three times. The reaction mixture was stirred at 80° C. for 24 hr under N₂ atmosphere. The reaction mixture was concentrated in vacuo and the residue was purified by column chromatography (SiO₂, petroleum ether/ethyl acetate=1/0 to 0/1) to give the title compound as a yellow solid (120 mg, 257.03 μmol, 32% yield, 70% purity). ¹H NMR (400 MHz, DMSO-d₆) δ=10.83 (s, 1H), 8.91 (s, 1H), 8.08 (s, 1H), 7.65 (s, 1H), 6.64 (s, 1H), 4.75-4.64 (m, 1H), 4.55-4.30 (m, 2H), 4.24-4.10 (m, 2H), 3.88 (t, J=6.4 Hz, 2H), 3.41 (s, 3H), 3.00-2.75 (m, 2H), 2.10-1.94 (m, 1H), 0.97 (d, J=6.8 Hz, 3H).

### (1³S,1⁵R)-2⁵-Chloro-1⁴, 1⁴-difluoro-4¹-(3-hydroxy-3-methylbutyl)-1⁵,4³-dimethyl-4²,4³-dihydro-4¹H-5,8-dioxa-3-aza-4(6,4)-benzo[d]imidazola-2(2,4)-pyrimidina-1 (1,3)-piperidinacyclooctaphan-4²-one (33)

A mixture of (138,15R)-2⁵-chloro-1⁴,1⁴-difluoro-1⁵,4³-dimethyl-4²,4³-dihydro-4¹H-5,8-dioxa-3-aza-4(6,4)-benzo[d]imidazola-2(2,4)-pyrimidina-1 (1,3)-piperidinacyclooctaphan-4²-one (120 mg, 257.03 μmol, 1 eq), (3-hydroxy-3-methyl-butyl) 4-methylbenzenesulfonate (99.60 mg, 385.55 μmol, 1.5 eq), KI (21.33 mg, 128.52 μmol, 0.5 eq) and Cs₂CO₃ (167.49 mg, 514.07 μmol, 2 eq) in DMSO (3 mL) was degassed and purged with N₂ three times. The reaction mixture was stirred at 80° C. for 12 hr under N₂ atmosphere. The reaction mixture was concentrated in vacuo and the residue was purified by p-HPLC (column: Waters™ Xbridge BEH C18 100*30 mm*10 μm; mobile phase: [H₂O (10 mM NH₄HCO₃)-ACN]; gradient: 35%-65% B over 8.0 min) to give the title compound as a white solid (50 mg, 98.9% purity). ¹H NMR (400 MHz, DMSO-d₆) δ=9.01 (s, 1H), 8.09 (s, 1H), 7.70 (s, 1H), 6.76 (s, 1H), 4.76-4.64 (m, 1H), 4.55-4.30 (m, 3H), 4.25-4.10 (m, 2H), 3.93-3.76 (m, 4H), 3.46 (s, 3H), 2.88 (s, 2H), 2.07-1.93 (m, 1H), 1.75-1.63 (m, 2H), 1.18 (s, 6H), 0.97 (d, J=6.8 Hz, 3H). LCMS: [M+H]⁺=553.1.

### Example 18: Synthesis of (1³R₁₉₈)-2⁵-chloro-1⁴, 1⁴-difluoro-4-(3-hydroxy-3-methylbutyl)-1⁵,4³-dimethyl-4²,4³-dihydro-4¹H-5,8-dioxa-3-aza-4(6,4)-benzo[d]imidazola-2(2,4)-pyrimidina-1 (1,3)-piperidinacyclooctaphan-4²-one (34)

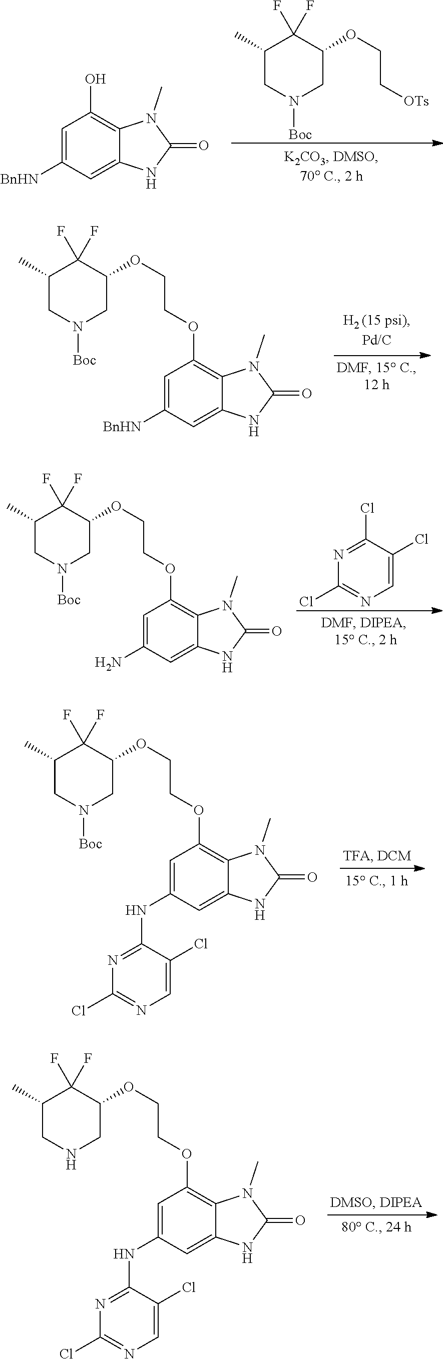

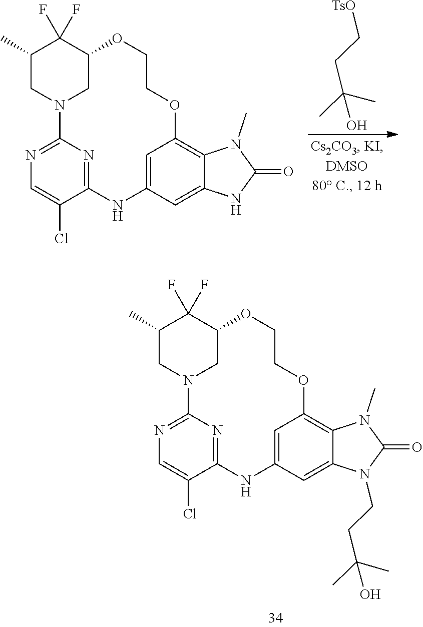 [SMILES: C[C@H]1CN2C[C@@H](OCCOc3cc(cc4[nH]c(=O)n(C)c34)Nc3nc2ncc3Cl)C1(F)F ; Cc1ccc(S(=O)(=O)OCCC(C)(C)O)cc1 ; C[C@H]1CN2C[C@@H](OCCOc3cc(cc4c3n(C)c(=O)n4CCC(C)(C)O)Nc3nc2ncc3Cl)C1(F)F]

### tert-Butyl (3R,5S)-3-(2-((6-(benzylamino)-3-methyl-2-oxo-2,3-dihydro-1H-benzo[(d]imidazol-4-yl)oxy)ethoxy)-4,4-difluoro-5-methylpiperidine-1-carboxylate

To a solution of 5-(benzylamino)-7-hydroxy-1-methyl-1,3-dihydro-2H-benzo[d]imidazol-2-one (800 mg, 2.97 mmol, 1 eq) and tert-butyl (3S,5R)-4,4-difluoro-3-methyl-5˜ (2-(tosyloxy)ethoxy)piperidine-1-carboxylate (1.27 g, 2.82 mmol, 0.95 eq) in DMSO (10 mL) was added K₂CO₃ (821.13 mg, 5.94 mmol, 2 eq). The reaction mixture was stirred at 70° C. for 2 hr under N₂. The reaction mixture was poured into water (20 mL) and filtered. The filter cake was washed with PE/EtOAc (15 mL, 2:1). The solid was dried in vacuo to give the title compound as a white solid (600 mg, 933.03 μmol, 31% yield, 85% purity). ¹H NMR (400 MHz, DMSO-d₆) δ=10.36 (s, 1H), 7.38-7.27 (m, 4H), 7.24-7.18 (m, 1H), 6.03 (d, J=1.5 Hz, 1H), 5.93 (t, J=6.0 Hz, 1H), 5.83 (d, J=1.5 Hz, 1H), 4.22 (d, J=6.0 Hz, 2H), 4.09-3.89 (m, 5H), 3.83-3.64 (m, 2H), 3.34 (s, 3H), 2.94-2.61 (m, 2H), 2.12-1.98 (m, 1H), 1.37 (br s, 9H), 0.96 (d, J=6.7 Hz, 3H).

### tert-Butyl (3R,5S)-3-(2-((6-amino-3-methyl-2-oxo-2,3-dihydro-1H-benzo[d]imidazol-4-yl)oxy)ethoxy)-4,4-difluoro-5-methylpiperidine-1-carboxylate

To a solution of tert-butyl (3R,5S)-3-(2-((6-(benzylamino)-3-methyl-2-oxo-2,3-dihydro-1H-benzo[d]imidazol-4-yl)oxy)ethoxy)-4,4-difluoro-5-methylpiperidine-1-carboxylate (600 mg, 1.10 mmol, 1 eq) in DMF (12 mL) was added Pd/C (0.1 g, 10% purity) under Ar. The suspension was degassed under vacuum and purged with H₂ several times. The reaction mixture was stirred at 15° C. for 12 hr under H₂ (15 psi). The reaction mixture was filtered, and the filtrate was concentrated in vacuo to give the title compound as a brown solid (500 mg, 985.80 μmol, 90% yield, 90% purity). LCMS: [M+H]⁺=457.4.

### tert-Butyl (3R,5S)-3-(2-((6-((2,5-dichloropyrimidin-4-yl)amino)-3-methyl-2-oxo-2,3-dihydro-1H-benzo[d]imidazol-4-yl)oxy)ethoxy)-4,4-difluoro-5-methylpiperidine-1-carboxylate

To a solution of tert-butyl (3R,5S)-3-(2-((6-amino-3-methyl-2-oxo-2,3-dihydro-1H-benzo[d]imidazol-4-yl)oxy)ethoxy)-4,4-difluoro-5-methylpiperidine-1-carboxylate (500 mg, 1.10 mmol, 1 eq) in DMF (5 mL) was added DIEA (283.13 mg, 2.19 mmol, 381.57 μL, 2 eq) and 2,4,5-trichloropyrimidine (401.82 mg, 2.19 mmol, 2 eq). The reaction mixture was stirred at 15° C. for 2 hr. Water (10 mL) was then added and the reaction mixture was filtered. The filter cake was washed by EtOAc (10 mL). The solid was concentrated in vacuo to give the title compound as a gray solid (550 mg, 902.32 μmol, 82% yield, 99% purity). ¹H NMR (400 MHz, DMSO-d₆) δ=10.90 (s, 1H), 9.39 (s, 1H), 8.34 (s, 1H), 7.02-6.91 (m, 2H), 4.21-4.12 (m, 2H), 4.07-3.93 (m, 3H), 3.82-3.69 (m, 2H), 3.47 (s, 3H), 2.97-2.88 (m, 1H), 2.72-2.64 (m, 1H), 2.13-2.00 (m, 1H), 1.43-1.32 (m, 9H), 0.96 (d, J=6.8 Hz, 3H).

### 5-((2,5-Dichloropyrimidin-4-yl)amino)-7-(2-(((3R,5S)-4,4-difluoro-5-methylpiperidin-3-yl)oxy)ethoxy)-1-methyl-1,3-dihydro-2H-benzo[d]imidazol-2-one

A mixture of tert-butyl (3R,5S)-3-(2-((6-((2,5-dichloropyrimidin-4-yl)amino)-3-methyl-2-oxo-2,3-dihydro-1H-benzo[d]imidazol-4-yl)oxy)ethoxy)-4,4-difluoro-5-methylpiperidine-1-carboxylate (550 mg, 911.43 μmol, 1 eq) in DCM (11 mL) and TFA (2.75 mL) was stirred at 15° C. for 30 minutes under N₂ atmosphere. The reaction mixture was concentrated in vacuo and the residue was dissolved in MeOH (10 mL). Basic resin was then added until pH=8. The mixture was filtered and the filtrate was concentrated in vacuo to give the title compound as a yellow solid (400 mg, 667,56 μmol, 73% yield, 84% purity). LCMS: [M+H]⁺=503.2.

### (1³R, 1⁵S)-2⁵-Chloro-1⁴,1⁴-difluoro-1⁵,4³-dimethyl-4²,4³-dihydro-4 H-5,8-dioxa-3-aza-4(6,4)-benzo[d]imidazola-2(2,4)-pyrimidina-1 (1,3)-piperidinacyclooctaphan-4²-one

To a solution of 5-((2,5-dichloropyrimidin-4-yl)amino)-7-(2-(((3R,5S)-4,4-difluoro-5-methylpiperidin-3-yl)oxy)ethoxy)-1-methyl-1,3-dihydro-2H-benzo[d]imidazol-2-one (400 mg, 794.71 μmol, 1 eq) in DMSO (16 mL) was added DIPEA (2.05 g, 15.89 mmol, 2.77 mL, 20 eq). The reaction mixture was stirred at 80° C. for 24 hr. Water (100 mL) was added and the mixture was extracted with ethyl acetate (50 mL×2). The combined organic phase was washed with brine (100 mL), dried with anhydrous Na₂SO₄, and filtered. The filtrate was concentrated in vacuo and the residue was purified by silica gel chromatography (silica gel, petroleum ether/EtOAc=4/1, 0/1) to give the title compound as a yellow solid (120 mg, 241.61 μmol, 30% yield). ¹H NMR (400 MHz, DMSO-d₆) δ=10.81 (s, 1H), 8.90 (s, 1H), 8.08 (s, 1H), 7.65 (d, J=1.3 Hz, 1H), 6.64 (d, J=1.4 Hz, 1H), 4.68 (td, J=6.4, 12.4 Hz, 1H), 4.51-4.29 (m, 2H), 4.14 (td, J=6.7, 12.9 Hz, 2H), 3.88 (br t, J=6.8 Hz, 2H), 3.41 (s, 3H), 2.98-2.81 (m, 2H), 2.12-1.93 (m, 1H), 0.97 (d, J=6.8 Hz, 3H).

### (1³R, 1⁵S)-2⁵-Chloro-1⁴,1⁴-difluoro-4¹-(3-hydroxy-3-methylbutyl)-1⁵,4³-dimethyl-4²,4³-dihydro-4 H-5,8-dioxa-3-aza-4(6,4)-benzo[d]imidazola-2(2,4)-pyrimidina-1 (1,3)-piperidinacyclooctaphan-4²-one (34)

To a solution of (1³R,1⁵S)-2⁵-chloro-1⁴,1⁴-difluoro-1⁵,4³-dimethyl-4²,4³-dihydro-4 H-5,8-dioxa-3-aza-4(6,4)-benzo[d]imidazola-2(2,4)-pyrimidina-1 (1,3)-piperidinacyclooctaphan-4²-one (90 mg, 192.77 μmol, 1 eq) and (3-hydroxy-3-methyl-butyl) 4-methylbenzenesulfonate (74.70 mg, 289.16 μmol, 1.5 eq) in DMSO (4 mL) was added Cs₂CO₃ (125.62 mg, 385.55 μmol, 2 eq) and KI (16.00 mg, 96.39 μmol, 0.5 eq). The reaction mixture was stirred at 80° C. for 12 hr. The reaction mixture (combined with another batch with 30 mg scale) was concentrated in vacuo and the residue was purified by prep-HPLC (column: Waters™ Xbridge BEH C18 100*30 mm 5 μm; mobile phase: [H₂O (10 mM NH₄HCO₃)-ACN];gradient: 33%-59% B over 8.0 min) to give the title compound as a white solid (25 mg, 99% purity). ¹H NMR (400 MHz, DMSO-d₆) δ=9.00 (s, 1H), 8.09 (s, 1H), 7.69 (s, 1H), 6.76 (s, 1H), 4.76-4.67 (m, 1H), 4.52-4.28 (m, 3H), 4.22-4.11 (m, 2H), 3.95-3.78 (m, 4H), 3.46 (s, 3H), 2.89 (br s, 2H), 2.09-1.95 (m, 1H), 1.70 (br dd, J=6.1, 10.0 Hz, 2H), 1.18 (s, 6H), 0.97 (br d, J=6.8 Hz, 3H). LCMS: [M+H]⁺=552.21.

### Example 19: Synthesis of (1³R,19S)-1⁴,1⁴,2⁵-trifluoro-4¹-(3-hydroxy-3-methylbutyl)-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (35) and (1³S,1⁵R)-1⁴,1⁴,2⁵-trifluoro-4¹-(3-hydroxy-3-methylbutyl)-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (36)

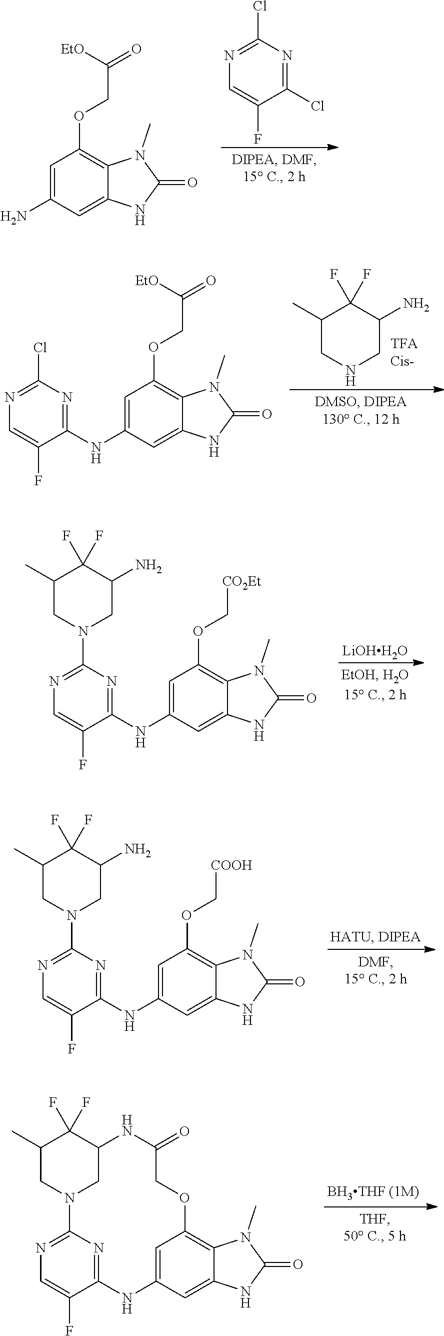

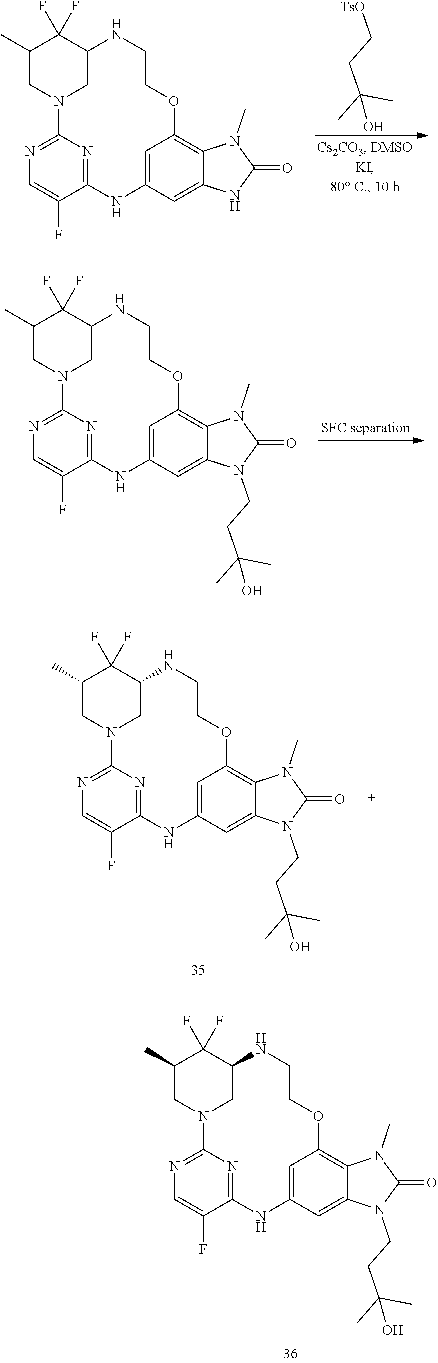 [SMILES: CC1CN2CC(NCCOc3cc(cc4[nH]c(=O)n(C)c34)Nc3nc2ncc3F)C1(F)F ; Cc1ccc(S(=O)(=O)OCCC(C)(C)O)cc1 ; CC1CN2CC(NCCOc3cc(cc4c3n(C)c(=O)n4CCC(C)(C)O)Nc3nc2ncc3F)C1(F)F ; C[C@H]1CN2C[C@@H](NCCOc3cc(cc4c3n(C)c(=O)n4CCC(C)(C)O)Nc3nc2ncc3F)C1(F)F ; C[C@@H]1CN2C[C@H](NCCOc3cc(cc4c3n(C)c(=O)n4CCC(C)(C)O)Nc3nc2ncc3F)C1(F)F]

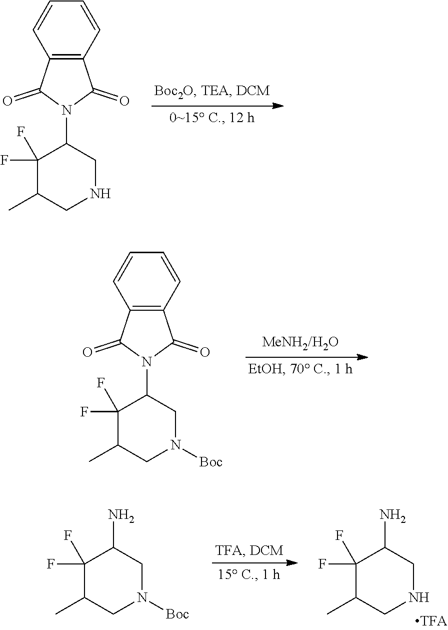

### tert-Butyl 3-(1,3-dioxoisoindolin-2-yl)-4,4-difluoro-5-methylpiperidine-1-carboxylate

To a solution of 2-(4,4-difluoro-5-methylpiperidin-3-yl) isoindoline-1,3-dione (10 g, 35.68 mmol, 1 eq) in DCM (20 mL) was added Boc₂O (7.79 g, 35.68 mmol, 8.20 mL, 1 eq) and TEA (7.22 g, 71.36 mmol, 9.93 mL, 2 eq) at 0° C. The reaction mixture was stirred at 15° C. for 12 hr. Water (100 mL) was added and the solution was extracted with DCM (100 mL×3). The combined organic phase was washed with brine (100 mL), dried over anhydrous Na₂SO₄, filtered and the filtrate was concentrated in vacuo. The residue was purified by flash silica gel chromatography (Silica Flash Column, Eluent of 0˜50% ethyl acetate/petroleum ether gradient) to give the title compound as a yellow oil (9 g, 23.19 mmol, 88% yield, 98% purity). ¹H NMR (400 MHz, CDCl₃) δ=7.93-7.86 (m, 2H), 7.79-7.73 (m, 2H), 4.57-4.37 (m, 1H), 4.36-4.00 (m, 3H), 2.97-2.66 (m, 1H), 2.32-2.08 (m, 1H), 1.48 (s, 9H), 1.08 (d,/=6.7 Hz, 3H).

tert-Butyl 3-amino-4,4-difluoro-5-methylpiperidine-1-carboxylate

To a solution of tert-butyl 3-(1,3-dioxoisoindolin-2-yl)-4,4-difluoro-5-methylpiperidine-1-carboxylate (9.00 g, 23.66 mmol, 1 eq) in EtOH (90 mL) was added MeNH2 (90 mL, 40% purity). The reaction mixture was stirred at 70° C. for 1 hr. The reaction mixture was concentrated in vacuo to give the title compound as a yellow oil (5.6 g, 22.37 mmol, 95% yield). ¹H NMR (400 MHz, CDCl₃) δ=4.40-3.82 (m, 2H), 3.00-2.83 (m, 1H), 2.59 (br d, J=9.3 Hz, 2H), 2.09-1.85 (m, 1H), 1.46 (s, 9H), 1.41-1.27 (m, 2H), 1.05 (d, J=6.8 Hz, 3H)

4,4-Difluoro-5-methylpiperidin-3-amine

To a solution of tert-butyl 3-amino-4,4-difluoro-5-methylpiperidine-1-carboxylate (5.6 g, 22.37 mmol, 1 eq) in DCM (30 mL) was added TFA (15 mL). The reaction mixture was stirred at 15° C. for 1 hr. The reaction mixture was concentrated in vacuo to give the title compound as a yellow solid (8 g, 21.15 mmol, 95% yield, 2TFA). ¹H NMR (400 MHz, DMSO-d₆) δ=9.38 (br s, 4H), 4.14-3.93 (m, 1H), 3.66 (br d, J=12.5 Hz, 1H), 3.46 (br d, J=12.9 Hz, 1H), 3.13 (t,)=12.5 Hz, 1H), 2.86 (t, J=12.9 Hz, 1H), 2.58-2.50 (m, 1H), 1.04 (d, J=6.8 Hz, 3H).

Ethyl 2-(6-((2-chloro-5-fluoropyrimidin-4-y)amino)-3-methyl-2-oxo-2,3-dihydro-1H-benzo[d]imidazol-4-yl)oxy)acetate

To a solution of ethyl 2-((6-amino-3-methyl-2-oxo-2,3-dihydro-1H-benzo[d]imidazol-4-yl)oxy)acetate (9 g, 33.93 mmol, 1 eq) and 2,4-dichloro-5-fluoro-pyrimidine (11.33 g, 67.86 mmol, 2 eq) in DMF (72 mL) was added DIPEA (8.77 g, 67.86 mmol, 11.82 mL, 2 eq). The reaction mixture was stirred at 15° C. for 2 hr under N₂. Water (100 mL) was added and the mixture was filtered and the filter cake was washed with EtOAc (100 mL) and dried in vacuo to give the title compound as a white solid (10 g, 23.25 mmol, 69% yield). ¹H NMR (400 MHz, DMSO-d₆)=10.94 (s, 1H), 9.84 (s, 1H), 8.27 (d, J=3.3 Hz, 1H), 7.18 (s, 1H), 7.01 (d, J=1.0 Hz, 1H), 4.82 (s, 2H), 4.19 (q, J=7.2 Hz, 2H), 3.49 (s, 3H), 1.21 (t, J=7.2 Hz, 3H).

Ethyl 2-(6-((2-(3-amino-4,4-difluoro-5-methylpiperidin-1-yl)-5-fluoropyrimidin-4-yl)amino)-3-methyl-2-oxo-2,3-dihydro-1H-benzo[d]imidazol-4-yl)oxy)acetate

A solution of ethyl 2-((6-((2-chloro-5-fluoropyrimidin-4-yl)amino)-3-methyl-2-oxo-2,3-dihydro-1H-benzo[d]imidazol-4-yl)oxy)acetate (4 g, 10.11 mmol, 1 eq) and 4,4-difluoro-5-methyl-piperidin-3-amine (6.12 g, 16.17 mmol, 1.6 eq, TFA) in DMSO (80 mL) and DIPEA (16 mL) was stirred at 130° C. for 12 hr under N₂. The reaction mixture (combined with another batch with 1 g scale) was poured into water (200 mL) and filtered. The filter cake was washed with water (100 mL) and dried in vacuo. The residue was triturated PE/EtOAc (20 mL, 3:1) at 15° C. for 30 minutes. The mixture was filtered and the filter cake was dried in vacuo to give the title compound as a yellow solid (4.3 g, 66% yield). ¹H NMR (400 MHz, DMSO-d₆) δ=10.91 (s, 1H), 9.17 (s, 1H), 7.99 (d, J=3.5 Hz, 1H), 7.11 (d, J=7.6 Hz, 2H), 4.83 (s, 2H), 4.62-4.46 (m, 2H), 4.18 (q, J=7.1 Hz, 2H), 3.53-3.45 (m, 3H), 2.97-2.82 (m, 1H), 2.71-2.61 (m, 2H), 2.08-1.93 (m, 1H), 1.88-1.52 (m, 2H), 1.24-1.17 (m, 3H), 0.99 (br d, J=6.6 Hz, 3H).

2-((6-((2-(3-Amino-4,4-difluoro-5-methylpiperidin-1-yl) ˜5-fluoropyrimidin-4-yl)amino)-3-methyl-2-oxo-2,3-dihydro-1H-benzo[d]imidazol-4-yl)oxy)acetic acid

To a solution of ethyl 2-((6-((2-(3-amino-4,4-difluoro-5-methylpiperidin-1-yl)-5-fluoropyrimidin-4-yl)amino)-3-methyl-2-oxo-2,3-dihydro-1H-benzo[d]imidazol-4-yl)oxy)acetate (3.3 g, 6.48 mmol, 1 eq) in EtOH (30 mL) and H₂O (6 mL) was added LiOH. H₂O (543.61 mg, 12.95 mmol, 2 eq). The reaction mixture was stirred at 15° C. for 2 hr. The reaction mixture (combined with another batch with 1 g scale) was concentrated in vacuo and water (20 mL) was added. The solution was acidified with 1 N HCl until pH=4. The mixture was filtered and the filter cake was dried in vacuo to give the title compound as a yellow solid (2.8 g, 68% yield). ¹H NMR (400 MHz, DMSO-d₆) δ=10.78 (s, 1H), 9.08 (s, 1H), 7.96 (d, J=3.5 Hz, 1H), 6.77 (s, 1H), 6.53 (br s, 1H), 4.84-4.75 (m, 1H), 4.63 (br d, J=10.6 Hz, 1H), 4.44 (d, J=15.7 Hz, 1H), 4.24 (br d, J=15.7 Hz, 1H), 3.76-3.62 (m, 1H), 3.47 (s, 3H), 2.66-2.53 (m, 2H), 2.30-2.12 (m, 1H), 1.02 (d, J=6.7 Hz, 3H).

1⁴,1⁴,2⁵-Trifluoro-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphane-42,7-dione

To a solution of 2-[[6-[[2-(3-amino-4,4-difluoro-5-methyl-1-piperidyl)-5-fluoro-pyrimidin-4-yl]amino]-3-methyl-2-oxo-1H-benzimidazol-4-yl]oxy]acetic acid (2 g, 4.15 mmol, 1 eq) in DMF (28 mL) was added HATU (1.58 g, 4.15 mmol, 1 eq) and DIPEA (1.07 g, 8.31 mmol, 1.45 mL, 2 eq). The reaction mixture was stirred at 15° C. for 2 hr under N₂. The reaction mixture (combined with another batch with 0.8 g scale) was poured into water (100 mL), filtered, and washed with water (50 mL). The filter cake was dried in vacuo and the residue was triturated with MeOH (10 mL) at 15° C. for 10 minutes. The mixture was filtered, and the filter cake was dried in vacuo to give the title compound as a yellow solid (2 g, 74% yield). ¹H NMR (400 MHz, DMSO-d₆) δ=10.78 (s, 1H), 9.18 (s, 1H), 8.51 (br d, J=8.1 Hz, 1H), 7.95 (br d, J=3.5 Hz, 1H), 7.19 (s, 1H), 6.49 (s, 1H), 4.92 (br d, J=14.8 Hz, 1H), 4.63 (br d, J=14.7 Hz, 2H), 4.45 (br d, J=14.3 Hz, 1H), 4.32-4.22 (m, 1H), 3.99-3.87 (m, 1H), 3.46 (s, 3H), 3.31-3.23 (m, 1H), 3.17 (d, J=4.8 Hz, 1H), 1.09 (br d, J=6.8 Hz, 3H).

### 1⁴,1⁴,2⁵-Trifluoro-1⁵,4³-dimethyl-4², 4³-dihydro-41 H-5-oxa-3,8-diaza-4(6,4)-benzo[d]imidazola-2(2,4)-pyrimidina-1 (1,3)-piperidinacyclooctaphan-4²-one

To a solution of 1⁴,1⁴,2⁵-trifluoro-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphane-42,7-dione (1.4 g, 3.02 mmol, 1 eq) in THF (27 mL) was added BH₃ THF (1 M, 12.08 mL, 4 eq) at 0° C. under N₂. The reaction mixture was stirred at 50° C. for 5 hr. The reaction mixture was quenched with sat. NH₄Cl (50 mL) slowly under N₂. After stirring at 15° C. for 30 minutes, the mixture was extracted with ethyl acetate (40 mL×3). The organic layer was dried over Na₂SO₄, filtered and the filtrate was concentrated in vacuo and the residue (combined with another batch with 600 mg scale) was triturated with EtOAc (5 mL) and filtered. The filtrate was dried in vacuo to give the title compound as a yellow solid (870 mg, 44% yield). ¹H NMR (400 MHz, DMSO-d₆) δ=10.77 (br d, J=3.5 Hz, 1H), 9.30 (br s, 1H), 8.03 (br t, J=3.8 Hz, 1H), 7.69 (br s, 1H), 6.61-6.52 (m, 1H), 4.76-4.23 (m, 3H), 4.08 (br d, J=8.7 Hz, 1H), 3.41 (br d, J=3.9 Hz, 3H), 3.25 (br dd, J=4.9, 8.5 Hz, 1H), 2.98 (br d,)=1.5 Hz, 2H), 2.74 (br t, J=11.1 Hz, 1H), 2.62-2.53 (m, 1H), 2.32-2.08 (m, 1H), 2.03-1.86 (m, 1H), 0.96 (br d, J=6.0 Hz, 3H).

### 1⁴,1⁴,2⁵-Trifluoro-4¹-(3-hydroxy-3-methylbutyl)-1³, 4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one

To a solution of 1⁴,1⁴,2⁵-trifluoro-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (290 mg, 645.26 μmol, 1 eq) and (3-hydroxy-3-methyl-butyl) 4-methylbenzenesulfonate (250.04 mg, 967.90 μmol, 1.5 eq) in DMSO (5 mL) was added Cs₂CO₃ (420.48 mg, 1.29 mmol, 2 eq) and KI (53.56 mg, 322.63 μmol, 0.5 eq). The reaction mixture was stirred at 80° C. for 10 hr under N₂. The reaction mixture was concentrated in vacuo and the residue was purified by prep-HPLC (column: Waters™ Xbridge BEH C18 250*50 mm*10 μm; mobile phase: [H₂O (10 mM NH₄HCO₃)-ACN];gradient: 30%-60% B over 10 min) to give the title compound as a white solid (350 mg, 640.45 μmol, 33% yield). ¹H NMR (400 MHz, DMSO-d₆) δ=9.40 (s, 1H), 8.04 (d, J=3.7 Hz, 1H), 7.74 (s, 1H), 6.67 (d, J=1.2 Hz, 1H), 4.75-4.66 (m, 1H), 4.55-4.46 (m, 2H), 4.40-4.25 (m, 1H), 4.15-4.02 (m, 1H), 3.81 (br dd, J=6.1, 10.1 Hz, 2H), 3.46 (s, 3H), 3.31-3.19 (m, 1H), 3.03-2.93 (m, 2H), 2.74 (br t, J=12.6 Hz, 1H), 2.61-2.51 (m, 1H), 2.28-2.19 (m, 1H), 2.04-1.85 (m, 1H), 1.68 (br dd, J=5.1, 9.7 Hz, 2H), 1.18 (s, 6H), 0.96 (d, J=6.7 Hz, 3H).

### (1³R, 1⁵S)-1⁴,1⁴,2⁵-Trifluoro-4¹-(3-hydroxy-3-methylbutyl)-1⁵,4³-dimethyl-4²,4³-dihydro-4¹H-5-oxa-3,8-diaza-4(6,4)-benzo[d]imidazola-2(2,4)-pyrimidina-1 (1,3)-piperidinacyclooctaphan-4²-one (35) and (1³S, 15R)-1⁴,1⁴,2⁵-trifluoro-4¹-(3-hydroxy-3-methylbutyl)-13,43-dimethyl-4², 4³-dihydro-4H-5-oxa-3,8-diaza-4(6,4)-benzo[d]imidazola-2(2,4)-pyrimidina-1 (1,3)-piperidinacyclooctaphan-4²-one (36)

1⁴,1⁴,2⁵-trifluoro-4¹-(3-hydroxy-3-methylbutyl)-1⁵,4³-dimethyl-4²,4³-dihydro-4 H-5-oxa-3,8-diaza-4(6,4)-benzo[d]imidazola-2(2,4)-pyrimidina-1 (1,3)-piperidinacyclooctaphan-4²-one (350 mg, 653.52 μmol, 1 eq) was separated by SFC (column: DAICEL CHIRALPAK AD (250 mm*30 mm, 10 μm); mobile phase: [CO₂-MeOH (0.1% NH: H₂O)]; B %: 40%, isocratic elution mode) to give compound 35 as a white solid (120 mg, 224.06 μmol, 34% yield) and compound 36 as a white solid (125 mg, 233.40 μmol, 36% yield). Compound 35: ¹H NMR (400 MHz, DMSO-d₆) δ=9.39 (s, 1H), 8.04 (d, J=3.8 Hz, 1H), 7.74 (s, 1H), 6.67 (s, 1H), 4.69 (br d, J=12.9 Hz, 1H), 4.54-4.44 (m, 2H), 4.38-4.29 (m, 1H), 4.13-4.03 (m, 1H), 3.81 (br dd, J=6.6, 9.7 Hz, 2H), 3.46 (s, 3H), 3.30-3.19 (m, 1H), 3.04-2.92 (m, 2H), 2.74 (br t,)=12.6 Hz, 1H), 2.59-2.53 (m, 1H), 2.26-2.18 (m, 1H), 2.05-1.86 (m, 1H), 1.68 (br dd, J=5.2, 9.7 Hz, 2H), 1.18 (s, 6H), 0.96 (d, J=6.6 Hz, 3H). LCMS: [M+H]⁺=536.2. Compound 36: ¹H NMR (400 MHz, DMSO-d₆) δ=9.40 (s, 1H), 8.04 (d, J=3.6 Hz, 1H), 7.74 (s, 1H), 6.67 (d, J=1.0 Hz, 1H), 4.69 (br d, J=13.0 Hz, 1H), 4.54-4.44 (m, 2H), 4.39-4.29 (m, 1H), 4.14-4.03 (m, 1H), 3.81 (br dd, J=6.4, 9.9 Hz, 2H), 3.46 (s, 3H), 3.29-3.19 (m, 1H), 3.04-2.89 (m, 2H), 2.80-2.69 (m, 1H), 2.59-2.53 (m, 1H), 2.25-2.19 (m, 1H), 2.03-1.87 (m, 1H), 1.68 (br dd, J=4.9, 9.6 Hz, 2H), 1.18 (s, 6H), 0.96 (d,,/=6.8 Hz, 3H). LCMS: [M+H]⁺=536.2.

### Example 20: Degradation Activity

### HiBiT Protocol

DC₅₀ (concentration to reach 50% degradation) values were determined from a cellular degradation assay (HiBiT, Promega™) in Su-DHL-4 cells (Table 1). Endogenous BCL6 was tagged with the 11-amino acid SmBiT through CRISPR/Cas9 gene editing and single cell clone selection. After 24 hr of compound treatment, cells were lysed and incubated with LgBiT protein to reconstitute intact nanoluciferase. Substrate was then added and relative luciferase units were measured. Degradation levels for each treatment were taken as a percentage compared to the control, 100% DMSO (Prism).

TABLE 1
Degradation activity of compounds

| | DC₅₀ |
|---|---|
| Compound | (nM) |
| 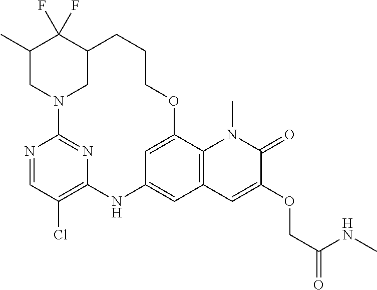 | 1.9 |
| (1) | |
| 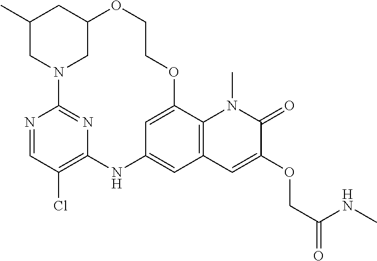 | 0.34 |
| (2) | |
| 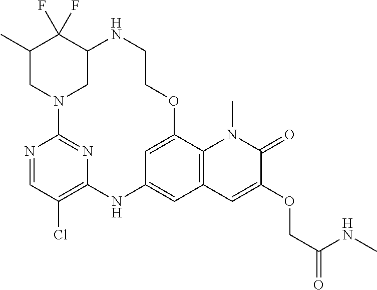 | 0.36 |
| (3) | |
| 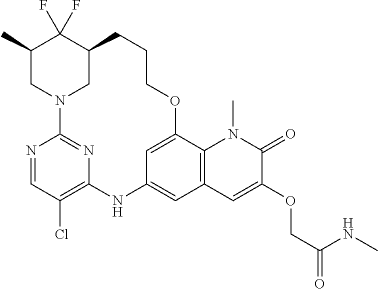 | 0.68 |
| (4), | |
| 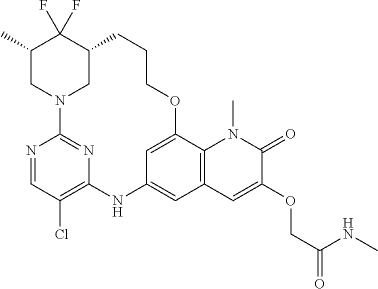 | 150 |
| (5), | |
| 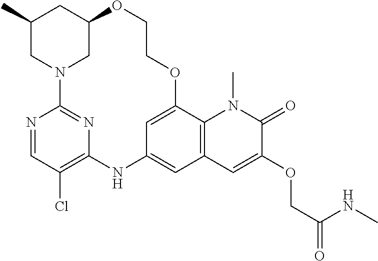 | 0.61 |
| (6), | |
| 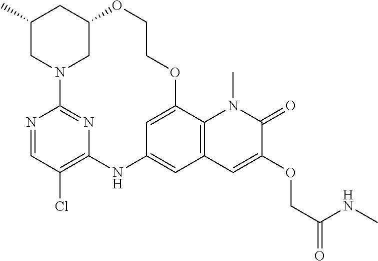 | 121 |
| (7), | |
| 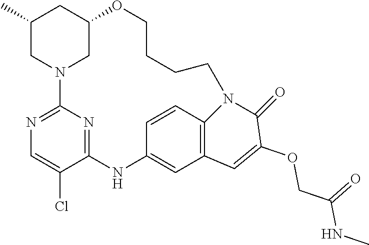 | >400 |
| (Compound A) | |
| 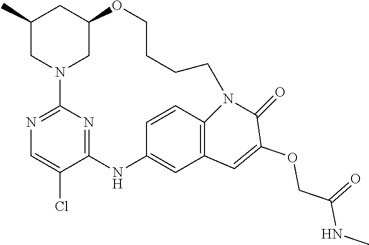 | >400 |
| (Compound B) | |
| 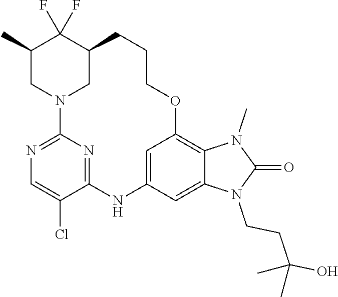 [SMILES: C[C@@H]1CN2C[C@H](CCCOc3cc(cc4c3n(C)c(=O)n4CCC(C)(C)O)Nc3nc2ncc3Cl)C1(F)F] | 11 |
| (10) | |
| 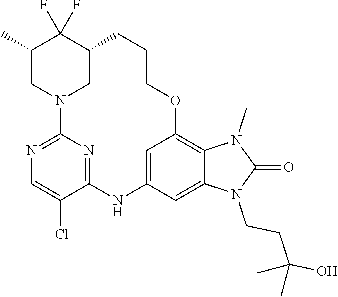 [SMILES: C[C@H]1CN2C[C@@H](CCCOc3cc(cc4c3n(C)c(=O)n4CCC(C)(C)O)Nc3nc2ncc3Cl)C1(F)F] | >400 |
| (11) | |
| 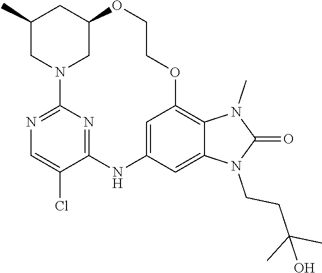 [SMILES: C[C@H]1C[C@@H]2CN(C1)c1ncc(Cl)c(n1)Nc1cc(c3c(c1)n(CCC(C)(C)O)c(=O)n3C)OCCO2] | 2 |
| (12) | |
| 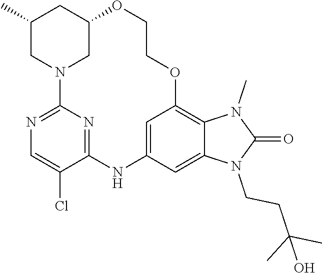 [SMILES: C[C@@H]1C[C@H]2CN(C1)c1ncc(Cl)c(n1)Nc1cc(c3c(c1)n(CCC(C)(C)O)c(=O)n3C)OCCO2] | >400 |
| (13) | |
| 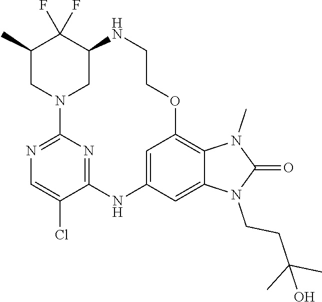 [SMILES: C[C@@H]1CN2C[C@H](NCCOc3cc(cc4c3n(C)c(=O)n4CCC(C)(C)O)Nc3nc2ncc3Cl)C1(F)F] | 2.9 |
| (14) | |
| 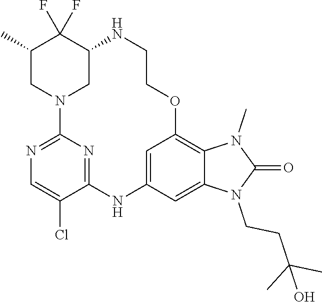 [SMILES: C[C@H]1CN2C[C@@H](NCCOc3cc(cc4c3n(C)c(=O)n4CCC(C)(C)O)Nc3nc2ncc3Cl)C1(F)F] | 82 |
| (15) | |
| 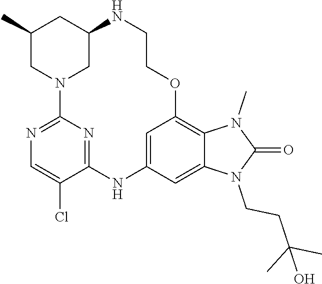 [SMILES: C[C@H]1C[C@@H]2CN(C1)c1ncc(Cl)c(n1)Nc1cc(c3c(c1)n(CCC(C)(C)O)c(=O)n3C)OCCN2] | 2.4 |
| (16) | |
| 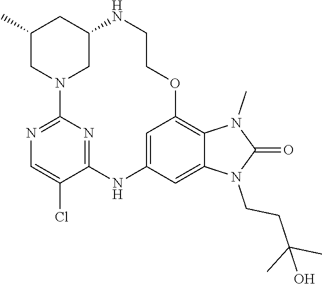 [SMILES: C[C@@H]1C[C@H]2CN(C1)c1ncc(Cl)c(n1)Nc1cc(c3c(c1)n(CCC(C)(C)O)c(=O)n3C)OCCN2] | 33 |
| (17) | |
| 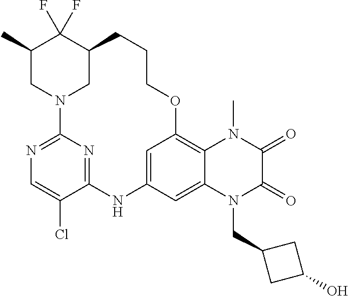 [SMILES: C[C@@H]1CN2C[C@H](CCCOc3cc(cc4c3n(C)c(=O)c(=O)n4C[C@H]3C[C@H](O)C3)Nc3nc2ncc3Cl)C1(F)F] | 31 |
| (18-1) | |
| 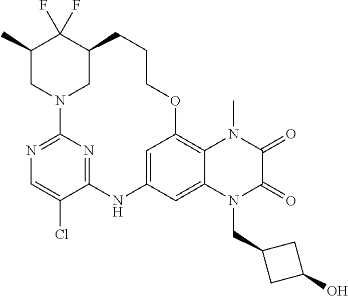 [SMILES: C[C@@H]1CN2C[C@H](CCCOc3cc(cc4c3n(C)c(=O)c(=O)n4C[C@H]3C[C@@H](O)C3)Nc3nc2ncc3Cl)C1(F)F] | 4.4 |
| (18-2) | |
| 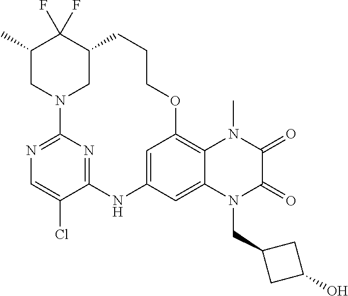 [SMILES: C[C@H]1CN2C[C@@H](CCCOc3cc(cc4c3n(C)c(=O)c(=O)n4C[C@H]3C[C@H](O)C3)Nc3nc2ncc3Cl)C1(F)F] | >400 |
| (19-1) | |
| 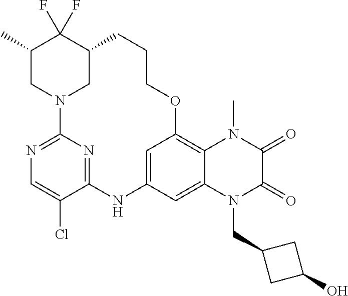 [SMILES: C[C@H]1CN2C[C@@H](CCCOc3cc(cc4c3n(C)c(=O)c(=O)n4C[C@H]3C[C@@H](O)C3)Nc3nc2ncc3Cl)C1(F)F] | >400 |
| (19-2) | |
| 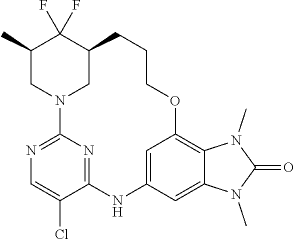 [SMILES: C[C@@H]1CN2C[C@H](CCCOc3cc(cc4c3n(C)c(=O)n4C)Nc3nc2ncc3Cl)C1(F)F] | 76 |
| (24) | |
| 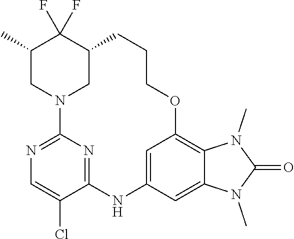 [SMILES: C[C@H]1CN2C[C@@H](CCCOc3cc(cc4c3n(C)c(=O)n4C)Nc3nc2ncc3Cl)C1(F)F] | >400 |
| (25) | |
| 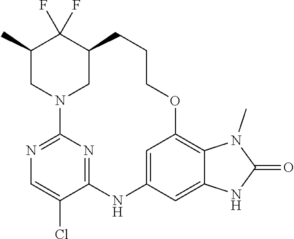 [SMILES: C[C@@H]1CN2C[C@H](CCCOc3cc(cc4[nH]c(=O)n(C)c34)Nc3nc2ncc3Cl)C1(F)F] | 72 |
| (26) | |
| 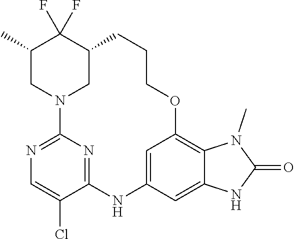 [SMILES: C[C@H]1CN2C[C@@H](CCCOc3cc(cc4[nH]c(=O)n(C)c34)Nc3nc2ncc3Cl)C1(F)F] | >400 |
| (27) | |
| 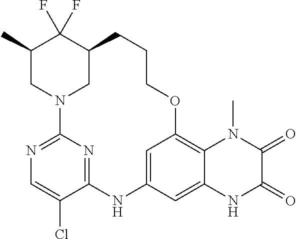 [SMILES: C[C@@H]1CN2C[C@H](CCCOc3cc(cc4[nH]c(=O)c(=O)n(C)c34)Nc3nc2ncc3Cl)C1(F)F] | 22 |
| (28) | |
| 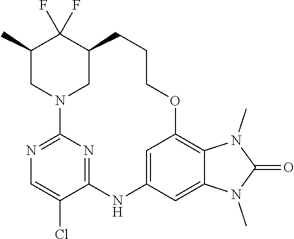 [SMILES: C[C@@H]1CN2C[C@H](CCCOc3cc(cc4c3n(C)c(=O)n4C)Nc3nc2ncc3Cl)C1(F)F] | 76 |
| (29) | |
| 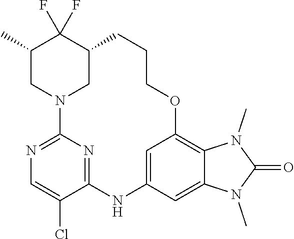 [SMILES: C[C@H]1CN2C[C@@H](CCCOc3cc(cc4c3n(C)c(=O)n4C)Nc3nc2ncc3Cl)C1(F)F] | >400 |
| (30) | |
| 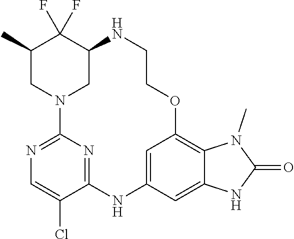 [SMILES: C[C@@H]1CN2C[C@H](NCCOc3cc(cc4[nH]c(=O)n(C)c34)Nc3nc2ncc3Cl)C1(F)F] | 7.5 |
| (31) | |
| 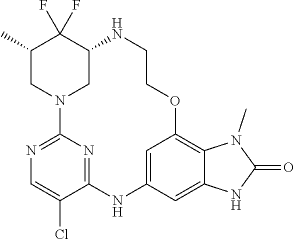 [SMILES: C[C@H]1CN2C[C@@H](NCCOc3cc(cc4[nH]c(=O)n(C)c34)Nc3nc2ncc3Cl)C1(F)F] | >400 |
| (32) | |
| 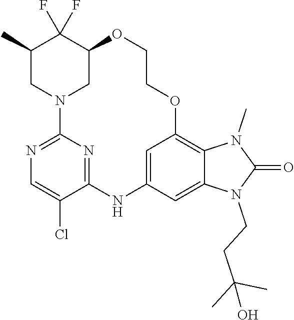 [SMILES: C[C@@H]1CN2C[C@H](OCCOc3cc(cc4c3n(C)c(=O)n4CCC(C)(C)O)Nc3nc2ncc3Cl)C1(F)F] | 1.9 |
| (33) | |
| 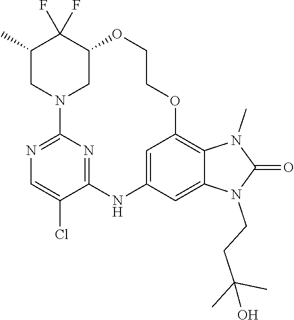 [SMILES: C[C@H]1CN2C[C@@H](OCCOc3cc(cc4c3n(C)c(=O)n4CCC(C)(C)O)Nc3nc2ncc3Cl)C1(F)F] | >400 |
| (34) | |
| 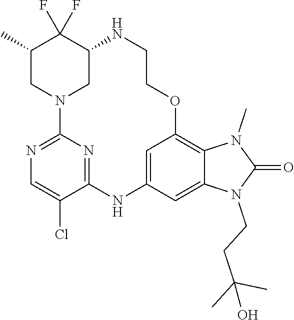 [SMILES: C[C@H]1CN2C[C@@H](NCCOc3cc(cc4c3n(C)c(=O)n4CCC(C)(C)O)Nc3nc2ncc3Cl)C1(F)F] | >400 |
| (35) | |
| 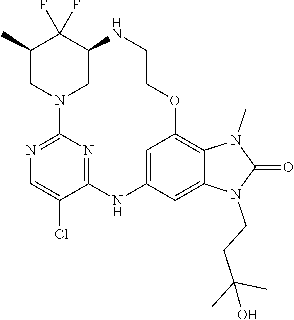 [SMILES: C[C@@H]1CN2C[C@H](NCCOc3cc(cc4c3n(C)c(=O)n4CCC(C)(C)O)Nc3nc2ncc3Cl)C1(F)F] | 1.5 |
| (36) | |
| 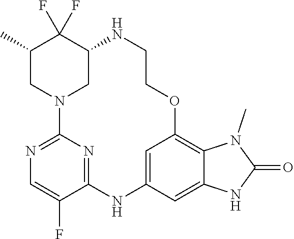 [SMILES: C[C@H]1CN2C[C@@H](NCCOc3cc(cc4[nH]c(=O)n(C)c34)Nc3nc2ncc3F)C1(F)F] | >400 |
| (37) | |
| 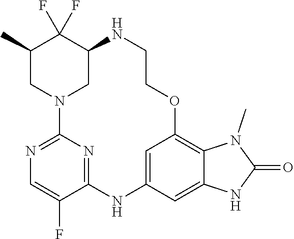 [SMILES: C[C@@H]1CN2C[C@H](NCCOc3cc(cc4[nH]c(=O)n(C)c34)Nc3nc2ncc3F)C1(F)F] | 14.2 |
| (38) | |
| 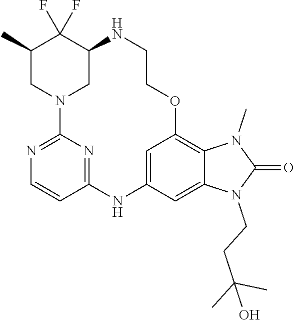 [SMILES: C[C@@H]1CN2C[C@H](NCCOc3cc(cc4c3n(C)c(=O)n4CCC(C)(C)O)Nc3ccnc2n3)C1(F)F] | 96.7 |
| (39) | |
| 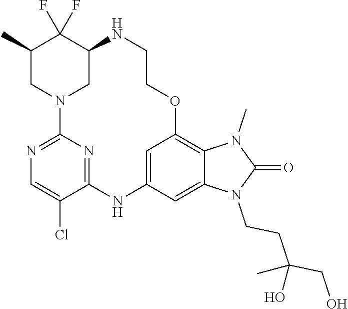 [SMILES: C[C@@H]1CN2C[C@H](NCCOc3cc(cc4c3n(C)c(=O)n4CCC(C)(O)CO)Nc3nc2ncc3Cl)C1(F)F] | 3.7 |
| (40) | |
| 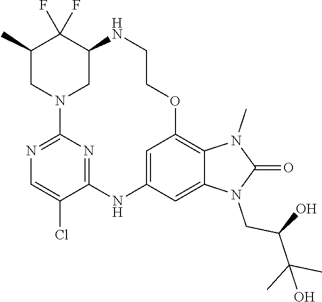 [SMILES: C[C@@H]1CN2C[C@H](NCCOc3cc(cc4c3n(C)c(=O)n4C[C@@H](O)C(C)(C)O)Nc3nc2ncc3Cl)C1(F)F] | 1.0 |
| (41) | |
| 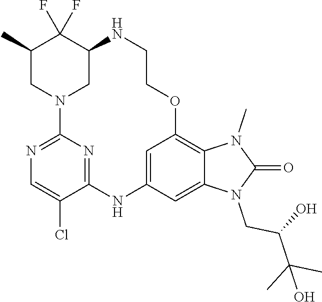 [SMILES: C[C@@H]1CN2C[C@H](NCCOc3cc(cc4c3n(C)c(=O)n4C[C@H](O)C(C)(C)O)Nc3nc2ncc3Cl)C1(F)F] | 8.5 |
| (42) | |
| 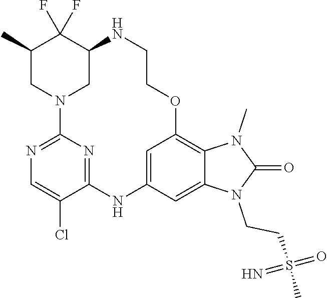 [SMILES: C[C@@H]1CN2C[C@H](NCCOc3cc(cc4c3n(C)c(=O)n4CC[S@](C)(=N)=O)Nc3nc2ncc3Cl)C1(F)F] | 11.3 |
| (43) | |
| 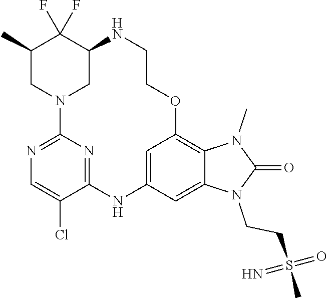 [SMILES: C[C@@H]1CN2C[C@H](NCCOc3cc(cc4c3n(C)c(=O)n4CC[S@@](C)(=N)=O)Nc3nc2ncc3Cl)C1(F)F] | 18.9 |
| (44) | |
| 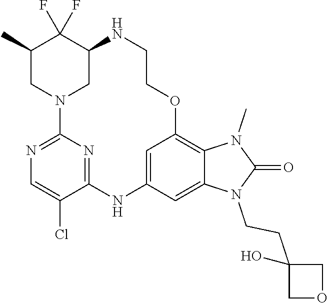 [SMILES: C[C@@H]1CN2C[C@H](NCCOc3cc(cc4c3n(C)c(=O)n4CCC3(O)COC3)Nc3nc2ncc3Cl)C1(F)F] | 8.7 |
| (45) | |

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A compound having a structure represented by formula I: 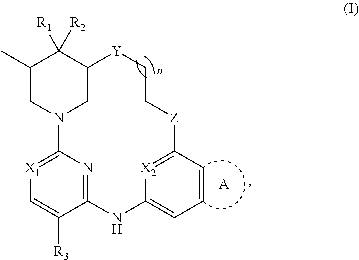
or a pharmaceutically acceptable salt or stereoisomer thereof,
wherein:
X₁ is N, CH, CCl, CF, or CCN;
X₂ is N or CR₄;
R₄ is H, (C₁-C₄)alkyl, halo, OH, NH₂, (C₁-C₄)alkoxy, (C₁-C₄)haloalkyl, (C₁-C₄)haloalkoxy, (C₂-C₄)alkenyl, (C₂-C₄)alkynyl, NO₂, CN, NH(C₁-C₄)alkyl, or N(C₁-C₄ alkyl)₂;
R₁ is H, OH, or halo;
R₂ is H, OH, or halo;
R₃ is H, halo, or CN;
Y is CH₂, NH, or O;
Z is CH₂, NH, S, or O;
n is 0, 1, or 2;
A is 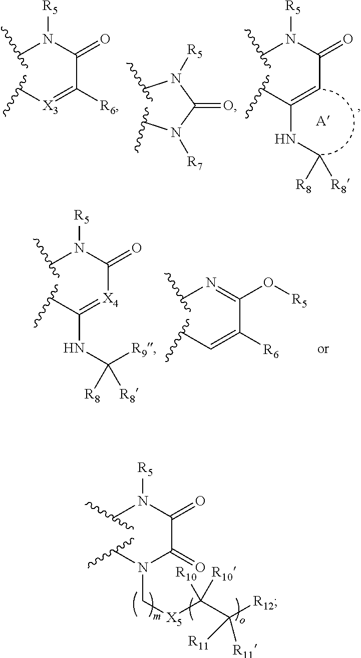
X₃ is CH or N;
R₅ is H, (C₁-C₆)alkyl, (C₁-C₆) hydroxyalkyl, (C₁-C₆)aminoalkyl, (C₃-C₆)carbocyclyl, 4- to 7-membered heterocyclyl, (C₁-C₆)alkyl-(C₃-C₇)carbocyclyl, or (C₃-C₇)carbocyclyl-4- to 7-membered heterocyclyl; wherein said alkyl, carbocyclyl, or heterocyclyl is further optionally substituted by one or more, identical or different R₁₃ groups, wherein R₁₃ is (C₁-C₆)alkyl, (C₁-C₆)alkoxy, halo, NH₂, OH, (C₁-C₆)haloalkyl, NH(C₁-C₆)alkyl, or N((C₁-C₆)alkyl)₂, (C₃-C₆)carbocyclyl, 4- to 7-membered heterocyclyl, or
R₅ is -L-Y₁-Z₁, wherein
L is absent or (C₁-C₅)alkylene optionally substituted by one or more substituents selected from (C₁-C₂)alkyl and oxo;
Y₁is absent, O, S, S(O), S(O)₂, NR″, C(O), C(O)O, OC(O), C(O)N(R″), N(R″)C(O), N(R″)C(O)N(R″), N(R″)C(O)O, OC(O)N(R″), S(O)₂N(R″), or N(R″)S(O)₂;
each R′ is independently H or (C₁-C₄)alkyl;
Z₁is H, (C₁-C₆)alkyl, (C₂-C₆)alkenyl, (C₂-C₆)alkynyl, (C₃-C₁₀)carbocyclyl, or 3- to 10-membered heterocyclyl, wherein Z₁ is optionally substituted by one or more substituents independently selected from (C₁-C₄)alkyl, halo, (C₁-C₄)haloalkyl, (C₁-C₄)haloalkoxy, NH₂, (C₁-C₄)aminoalkyl, CN, OH, carboxy, carbamoyl, sulphamoyl, mercapto, ureido, NR^{r}R^{s}, OR^{r}, C(O)R″, C(O)OR″, OC(O)R″, C(O)NR^{r}R₅, N(R″)C(O)R″, S(O)₀₋₂R″, S(O)₂NR^{r}R^{s}, N(R″)SO₂R″, Si(R^{r})(R^{s})R^{t} and (CH₂)₁₋₃NR^{r}R^{s}; wherein R″, R₅, and R^{t} are each independently H, (C₁-C₆)alkyl, or (C₃-C₆)cycloalkyl; or R′ and R^{s} together with the nitrogen atom to which they are attached form a 4- to 9-membered heterocyclyl which is optionally substituted by one or more substituents selected from (C₁-C₄)alkyl, halo, (C₁-C₄)haloalkyl, (C₁-C₄)haloalkoxy, (C₁-C₄)alkoxy, (C₁-C₄)alkylamino, NH₂, CN, and OH;
R₆ is -L₁CR₁₄R₁₅R₁₆, or —CH═CH-R₁₆, wherein
L₁ is absent, O, S, (C₁-C₄)alkylene, —O-(C₁-C₄)alkylene, or -5-(C₁-C₄)alkylene;
R₁₄ is H or (C₁-C₄)alkyl;
R₁₅ is H or (C₁-C₄)alkyl, or
R₁₄ and R₁₅ together with the carbon atom to which they are attached form a (C₃-C₅)carbocyclyl, 4- to 7-membered heterocyclyl, or C═O;
R₁₆ is (C₁-C₆)alkyl, —NR₁₇R₁₈, —OR₁₇, —C(O)R₁₇, —C(O)OR₁₇, —N(R₁₈)C(O)R₁₇, —C(O)NR₁₇R₁₈, —S(O)(C₁-C₆)alkyl, —S(O)₂ (C₁-C₆)alkyl, —P(O)-(C₁-C₆ alkyl)₂, —C(NH)NH₂, -(C₁-C₄)alkyl-NR₁₈C(O)R₁₇, or 4- to 7-membered heterocyclyl, wherein
R₁₇ is H, 3- to 6-membered heterocyclyl, or (C₁-C₄)alkyl optionally substituted by one or more, identical or different groups selected from OH, C₁, F, CF₃, N(C₁-C₄ alkyl)₂, (C₃-C₆)carbocyclyl, 3- to 6-membered heterocyclyl, (C₂-C₄)alkenyl, and (C₂-C₄)alkynyl;
R₁₈ is H or (C₁-C₄)alkyl;
R₇ is H, methyl, —(CH₂)₁₋₃W₁W₂, or 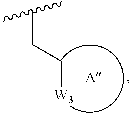
wherein
W₁ is CR₁₉R₁₉′ or C(O);
R₁₉ and R₁₉′ are independently H, (C₁-C₂)alkyl, F, OH, CN, NO₂, (C₁-C₂)alkoxy, (C₁-C₂)haloalkyl, (C₁-C₂)haloalkoxy, NH₂, NH(C₁-C₂)alkyl, or N(C₁-C₂ alkyl)₂, or
R₁₉ and R₁₉′ together with the carbon atom to which they are attached form C(O), (C₃-C₆)carbocyclyl or 3- to 6-membered heterocyclyl, which is optionally substituted by one or more substituents independently selected from (C₁-C₂)alkyl, halo, (C₁-C₂)haloalkyl, (C₁-C₂)haloalkoxy, (C₁-C₂)alkoxy, (C₁-C₂)alkylamino, NH₂, CN, and OH;
W₂ is CN, OH, 5- or 6-membered heteroaryl, phenyl, C(O)-(C₁-C₂)alkyl, S(O)₂-(C₁-C₂)alkyl, S(O)(NH)-(C₁-C₂)alkyl, C(O)OCH₃, C(O)NHCH₃, CR₂₀R₂₁R₂₂, NH₂, NH(C₁-C₂)alkyl, or N(C₁-C₂ alkyl)₂, wherein
R₂₀ is H, (C₁-C₂)alkyl, F, Cl, Br, OH, NH₂, CN, NO₂, (C₁-C₂)alkoxy, (C₁-C₂)haloalkyl, or (C₁-C₂)haloalkoxy;
R₂₁ is H, (C₁-C₂)alkyl, F, Cl, Br, OH, CN, NO₂, (C₁-C₂)alkoxy, (C₁-C₂)haloalkyl, (C₁-C₂)haloalkoxy, or -Y₂-L₂-Z₂, wherein
Y₂ is absent, O, S, S(O), S(O)₂, NR″, C(O), C(O)O, OC(O), C(O)N(R″), N(R″)C(O), S(O)₂N(R″), or N(R″)SO₂;
L₂ is absent or (C₁-C₂)alkylene;
Z₂ is H, (C₁-C₆)alkyl, (C₂-C₄)alkenyl, (C₂-C₄)alkynyl, phenyl, (C₃-C₆)carbocyclyl, or 4- to 6-membered heterocyclyl, wherein Z₂ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, NH₂, CN, OH, C(O)R″, C(O)OR″, OC(O)R″, C(O)NR^{r}R″, and N(R″)C(O)R″, wherein each R′ is independently H or (C₁-C₄)alkyl;
or R₂₀ and R₂₁ together with the carbon atom to which they are attached form (C₃-C₆)carbocyclyl or 3- to 6-membered heterocyclyl, optionally substituted by one or more substituents selected from (C₁-C₂)alkyl, halo, (C₁-C₂)haloalkyl, (C₁-C₂)haloalkoxy, (C₁-C₂)alkoxy, (C₁-C₂)alkylamino, NH₂, CN, and OH;
R₂₂ is (C₁-C₂)alkyl, —C(O)OR″, OR″, —C(O)NR″, NR″R″, phenyl, or 5-membered
heteroaryl, wherein each R″ is independently H or (C₁-C₂)alkyl;
A″ is (C₄-C₆)carbocyclyl or 4- to 6-membered heterocyclyl, optionally substituted with one or more substituents independently selected from (C₁-C₂)alkyl, halo, OH, oxo, CN, and (C₁-C₂)alkoxy;
W₃ is NR₂₃ or CR₂₄R₂₄′, wherein
R₂₃ is H, (C₁-C₂)alkyl, (C₁-C₄)haloalkyl, (C₁-C₄) hydroxyalkyl, —C(O)CH₃, or —C(O)O-(C₁-C₄)alkyl;
R₂₄ and R₂₄′ are independently H, (C₁-C₂)alkyl, cyclopropyl, F, Cl, Br, OH, NH₂, CN, NO₂, (C₁-C₂)haloalkyl, (C₁-C₂)haloalkoxy, (C₁-C₂)alkoxy, —C(O)OR″, NR″R″, phenyl, or 5-membered heteroaryl;
R₈ is H, (C₁-C₄)alkyl, (C₃-C₆)cycloalkyl, (C₁-C₄)haloalkyl, or CN, wherein said alkyl or cycloalkyl is optionally substituted by one or more substituents selected from (C₁-C₄)alkyl, (C₃-C₆)cycloalkyl, OH, (C₁-C₂)alkoxy, NH₂, NH(C₁-C₂)alkyl, N((C₁-C₂)alkyl)₂, (C₁-C₂)aminoalkyl, and halo;
R₈′ is H, (C₁-C₄)alkyl, CN, (C₁-C₄)haloalkyl, or -Y₃-L₃-Z₃, wherein
Y₃ is absent, C(O)O, or C(O)N(R″);
L₃ is absent or (C₁-C₂)alkylene;
Z₃ is H, (C₁-C₆)alkyl, phenyl, (C₃-C₆)cycloalkyl, or 4- to 6-membered heterocyclyl, wherein Z₃ is optionally substituted by one or more substituents independently selected from (C₁-C₂)alkyl, halo, (C₁-C₂)haloalkyl, (C₁-C₂)haloalkoxy, (C₁-C₂)alkoxy, NH₂, NO₂, CN, and OH, or R₈ and R₈′, together with the carbon atom to which they are attached, form a (C₄-C₆)carbocyclyl, or 4- to 6-membered heterocyclyl;
A′ is a 6- or 7-membered heterocyclyl, which in addition to R₈ and R₈′, is optionally further substituted by one more substituents independently selected from oxo, (C₁-C₂)alkyl, cyclopropyl, spiro-cyclopropyl, halo, (C₁-C₂)haloalkyl, (C₁-C₂)haloalkoxy, (C₁-C₂)alkoxy, NH₂, CN, and OH;
X₄ is CR₂₅ or N, wherein
R₂₅ is H, F, C₁, or methyl;
R₉ is H, (C₁-C₂)alkyl, (C₃-C₄)cycloalkyl, (C₁-C₂)haloalkyl, CN, (C₂-C₄)alkenyl, or (C₂-C₄)alkynyl;
R₉′ is (C₁-C₄)alkyl, CN, (C₁-C₄)haloalkyl, or -Y₄-L₄-Z₄, wherein
Y₄ is absent, C(O), C(O)O, OC(O), C(O)N(R″), or S(O)₂N(R″);
L₄ is absent or (C₁-C₂)alkylene optionally substituted by one or more substituents selected from (C₁-C₂)alkyl and oxo;
Z₄ is H, (C₁-C₆)alkyl, (C₂-C₄)alkenyl, (C₂-C₄)alkynyl, phenyl, (C₃-C₆)carbocyclyl, (C₃-C₆)cycloalkenyl, or 4- to 6-membered heterocyclyl, wherein Z₄ is optionally substituted by one or more substituents independently selected from oxo, (C₁-C₄)alkyl, (C₃-C₆)cycloalkyl, halo, (C₁-C₄)haloalkyl, (C₁-C₄)haloalkoxy, (C₁-C₄)alkoxy, (C₁-C₄)alkylamino, NH₂, NO₂, CN, OH, C(O)R^{u}, C(O)OR^{u}, OC(O)R^{u}, C(O)NR^{u}R^{u}, and N(R^{u})C(O)R^{u}, wherein each R₁₁ is independently H, (C₁-C₄)alkyl, (C₃-C₆)cycloalkyl, or
Z₄ is -Q-L₅-W₄, wherein
Q is absent, O, NH, or N(C₁-C₂)alkyl;
L₅ is absent or (C₁-C₂)alkylene optionally substituted by one or more substituents selected from oxo and (C₁-C₂)alkyl;
W₄ is (C₁-C₄)alkyl, phenyl, (C₃-C₆)cycloalkyl, (C₃-C₆)cycloalkenyl, or 5- or 6-membered heterocyclyl, wherein W₄ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, NH₂, NO₂, CN, or OH; or R₉ and R₉′, together with the carbon atom to which they are attached, for a (C₃-C₁₀)carbocyclyl or a 4- to 10-membered heterocyclyl, which is optionally substituted by one or more substituents independently selected from oxo, (C₁-C₂)alkyl, halo, (C₁-C₂)haloalkyl, (C₁-C₂)haloalkoxy, (C₁-C₂)alkoxy, (C₁-C₂)alkylamino, NH₂, NO₂, CN, or OH; or the (C₃-C₁₀)carbocyclyl or 4- to 10-membered heterocyclyl is optionally fused to a 5- or 6-membered heteroaryl or phenyl ring, and the 5- or 6-membered heteroaryl or phenyl ring is optionally substituted by (C₁-C₂)alkyl, halo, (C₁-C₂)haloalkyl, (C₁-C₂)haloalkoxy, (C₁-C₂)alkoxy, (C₁-C₂)alkylamino, NH₂, NO₂, CN, or OH;
R₉″ is H, (C₁-C₄)alkyl, (C₁-C₂)haloalkyl, (C₁-C₂)alkoxy, (C₁-C₂)haloalkoxy, CN, NO₂, acetylenyl, phenyl, or 5- or 6-membered heteroaryl, wherein said alkyl, phenyl, or heteroaryl is optionally substituted by one or more substituents independently selected from halo, OH, and NH₂;
X₅ is a absent, (C₃-C₆)carbocyclyl, (C₃-C₆)carbocyclyl (C═O), or SO₂;
R₁₀ and R₁₀′ are each independently H, (C₁-C₃)alkyl, (C₁-C₃) hydroxyalkyl, (C₁-C₃)aminoalkyl, or R₁₀ and R₁₀′, together with the same carbon atom to which they are attached, form (C₃-C₆)carbocyclyl or 4- to 6-membered heterocyclyl, wherein said alkyl, hydroxyalkyl, aminoalkyl, carbocyclyl, or heterocyclyl is optionally substituted by one or more, identical or different R₁₀ₐ groups, wherein R₁₀ₐ is (C₁-C₆)alkyl, (C₁-C₆)alkoxy, (C₁-C₆)alkyl-(C₁-C₃)alkoxy, halo, NH₂, OH, (C₁-C₆)haloalkyl, NH-(C₁-C₆)alkyl, N((C₁-C₆)alkyl)₂, (C₃-C₆)carbocyclyl, 4- to 6-membered heterocyclyl, or
R₁₀′ and R₁₁′, together with the carbon atoms to which they are attached, form (C₃-C₆)carbocyclyl or 4- to 6-membered heterocyclyl;
R₁₁ and R₁₁′ are each independently H, (C₁-C₃)alkyl, (C₁-C₃) hydroxyalkyl, (C₁-C₃)aminoalkyl, or
R₁₁ and R₁₁′, together with the same carbon atom to which they are attached, form C—O, (C₃-C₆)carbocyclyl, or 4- to 6-membered heterocyclyl, wherein said carbocyclyl or heterocyclyl is optionally substituted by one or more, identical or different R₁₁ₐ groups, wherein each R₁₁ₐ is independently (C₁-C₆)alkyl, (C₁-C₆)alkoxy, (C₁-C₆)alkyl-(C₁-C₃)alkoxy, halo, NH₂, OH, (C₁-C₆)haloalkyl, NH-(C₁-C₆)alkyl, N((C₁-C₆)alkyl)₂, (C₃-C₆)carbocyclyl, or 4- to 6-membered heterocyclyl, or wherein two R₁₁ₐ groups, together with the same carbon atom to which they are attached, form C—O;
R₁₂ is H, OH, NH-(C₁-C₆)alkyl, NH-(C₁-C₆) hydroxyalkyl, NH-(C₁-C₆)aminoalkyl, NH-(C₃-C₆)carbocyclyl, NH-4- to 6-membered heterocyclyl, NH-(C₁-C₆)alkyl-(C₃-C₆)carbocyclyl, or NH-(C₁-C₆)alkyl-4- to 6-membered heterocyclyl, wherein said alkyl, hydroxyalkyl, aminoalkyl, carbocyclyl, or heterocyclyl is further optionally substituted by one or more, identical or different R₁₂ₐ groups, wherein each R₁₂ₐ is independently (C₁-C₆)alkyl, (C₁-C₆)alkoxy, (C₁-C₆)alkyl-(C₁-C₃)alkoxy, halo, NH₂, OH, (C₁-C₆)haloalkyl, NH-(C₁-C₆)alkyl, N((C₁-C₆)alkyl)₂, (C₃-C₆)carbocyclyl, or 4- to 6-membered heterocyclyl;
m is 0, 1, or 2; and
o is 0 or 1.

2. The compound of claim 1, wherein Z is O or CH₂.

3. (canceled)

4. The compound of claim 1, wherein Y is CH₂.

5. The compound of claim 1, wherein Y is O.

6. The compound of claim 1, wherein Y is NH.

7. The compound of claim 1, wherein n is 0.

8. The compound of claim 1, wherein n is 1.

9. The compound of claim 1, wherein n is 2.

10. The compound of claim 1, wherein R₁ and R₂ are each H.

11. The compound of claim 1, wherein R₁ and R₂ are each halo.

12. The compound of claim 11, wherein R₁ and R₂ are each F.

13. The compound of claim 1, wherein X₁ is N.

14. The compound of claim 1, wherein R₃ is halo.

15. The compound of claim 14, wherein R₃ is C₁ or F.

16. The compound of claim 1, wherein X₂ is CH.

17. The compound of claim 1, wherein A is 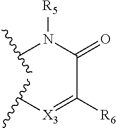
and the compound of formula (I) has the structure of formula I-1, 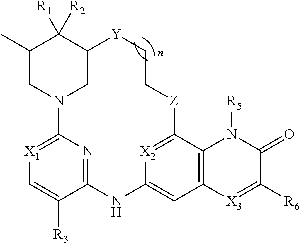
(I-1), or a pharmaceutically acceptable salt or stereoisomer thereof.

18. The compound of claim 17, wherein R₅ is methyl or X₃ is CH.

19. (canceled)

20. The compound of claim 17, wherein R₆ is -L₁CR₁₄R₁₅R₁₆.

21. The compound of claim 20, wherein L₁ is —O-(C₁)alkylene.

22. The compound of claim 20, wherein R₁₄ and R₁₅ together with the same carbon atom to which they are attached form C═O.

23. The compound of claim 20, wherein R₁₆ is methyl, OH, NH₂, or NHMe.

24. The compound of claim 17, which has the structure of formula I-1a, 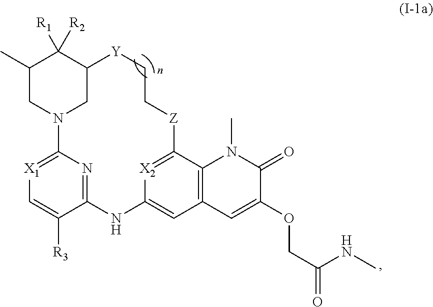
or a pharmaceutically acceptable salt or stereoisomer thereof.

25. The compound of claim 1, wherein A is 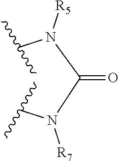
and the compound of formula (I) has the structure of formula I-2, 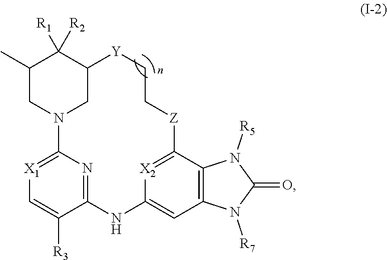
or a pharmaceutically acceptable salt or stereoisomer thereof.

26. The compound of claim 25, wherein R₅ is methyl or R₂₁s H or methyl.

27. The compound of claim 25, wherein R₂₁s-(CH₂)₂W₁W₂.

28. The compound of claim 27, wherein W₁ is CR₁₉R_{19′.}

29. The compound of claim 28, wherein R₁₉ and R₁₉′ are both methyl.

30. The compound of claim 27, wherein W₂ is CN, OH, or NH₂.

31. (canceled)

32. The compound of claim 25, which has the structure of formula I-2a, I-2b, or I-2c: 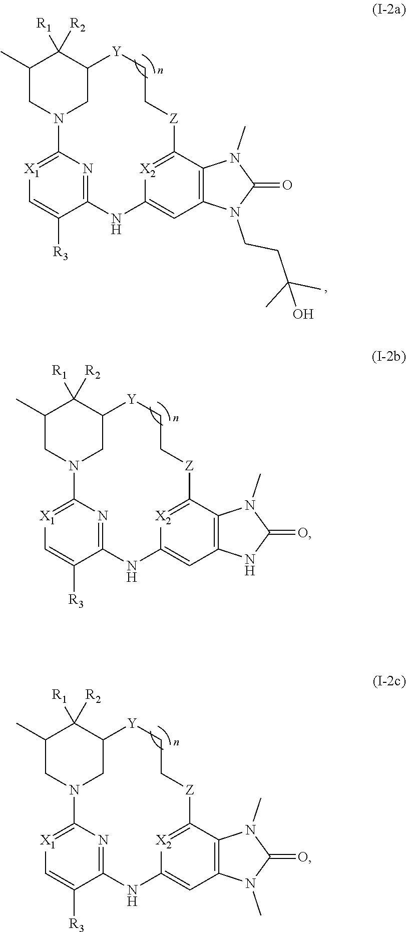
or a pharmaceutically acceptable salt or stereoisomer thereof.

33. The compound of claim 1, wherein A is 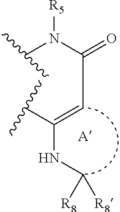
and the compound of formula (I) has the structure of formula I-3, 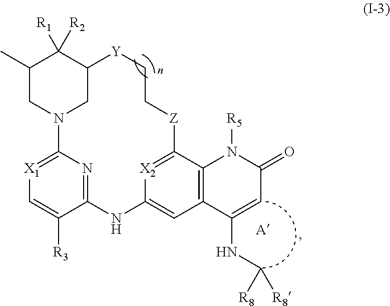
or a pharmaceutically acceptable salt or stereoisomer thereof.

34. The compound of claim 33, wherein R₅ is methyl or R₈ is (C₃-C₆)cycloalkyl and R₈′ is H.

35. (canceled)

36. The compound of claim 33, wherein A′ is a 7-membered heterocyclyl, wherein the heterocyclyl contains 2 heteroatoms selected from N and O, and which in addition to R₈ and R₈′, is optionally further substituted by one more substituents independently selected from oxo, (C₁-C₂)alkyl, cyclopropyl, spiro-cyclopropyl, halo, (C₁-C₂)haloalkyl, (C₁-C₂)alkoxy, NH₂, CN, and OH.

37. The compound of claim 33, which has the structure of formula I-3a, 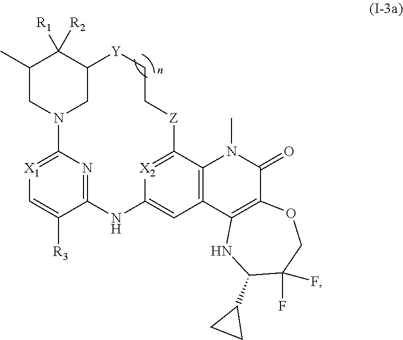
or a pharmaceutically acceptable salt or stereoisomer thereof.

38. The compound of claim 1, wherein A is 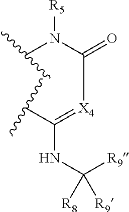
and the compound of formula (I) has the structure of formula I-4, 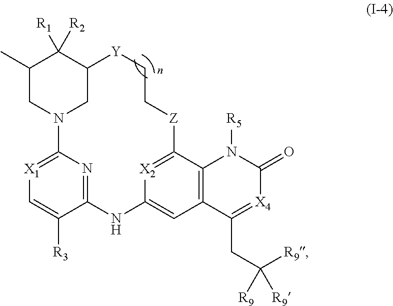
or a pharmaceutically acceptable salt or stereoisomer thereof.

39. The compound of claim 1, wherein A is 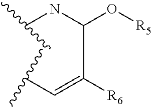
and the compound of formula (I) has the structure of formula I-5, 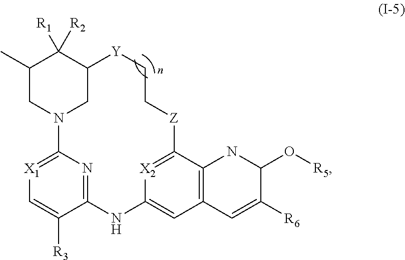
or a pharmaceutically acceptable salt or stereoisomer thereof.

40. The compound of claim 1, wherein A is 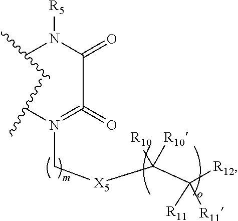
and the compound of formula (I) has the structure of formula I-6, 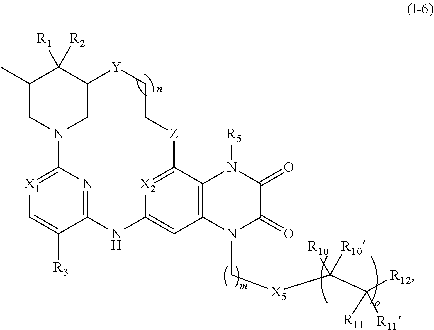
or a pharmaceutically acceptable salt or stereoisomer thereof.

41. The compound of claim 40, wherein R₅ is methyl or X₅ is absent.

42. The compound of claim 40, wherein m is 1.

43. The compound of claim 40, wherein m is 0.

44. (canceled)

45. The compound of claim 40, wherein o is 1.

46. The compound of claim 45, wherein R₁₀ and R₁₁ are each H and R₁₀′ and R₁₁′, together with the carbon atoms to which they are attached, form cyclobutyl.

47. The compound of claim 40, wherein R₁₂ is OH.

48. The compound of claim 40, wherein o is 0.

49. The compound of claim 48, wherein R₁₂ is H.

50. The compound of claim 40, which has the structure of formula I-6a or I-6b, 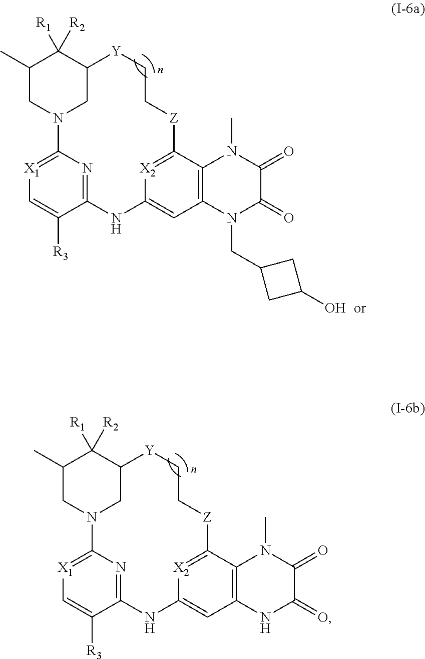
or a pharmaceutically acceptable salt or stereoisomer thereof.

51. The compound of claim 1, which is: 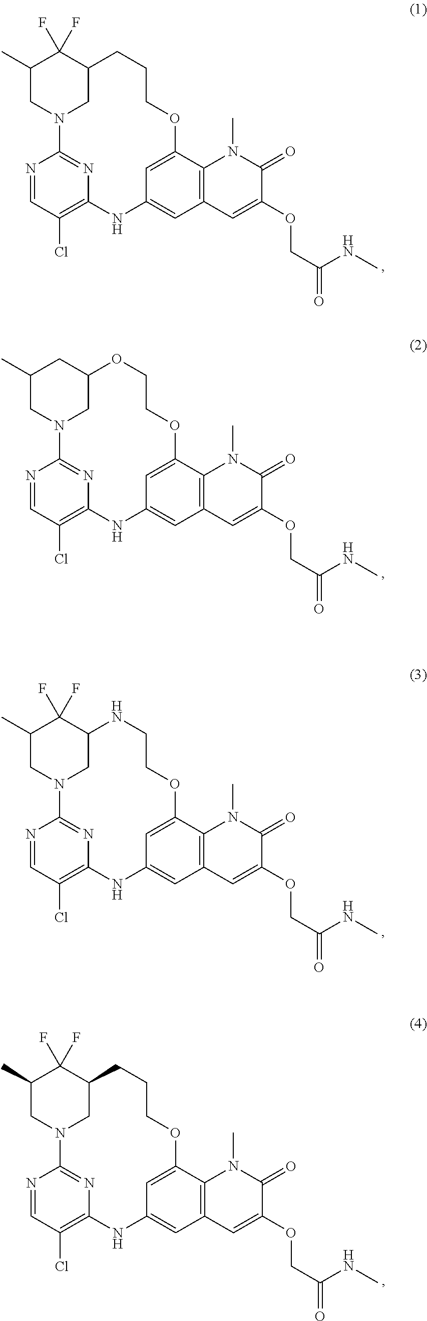 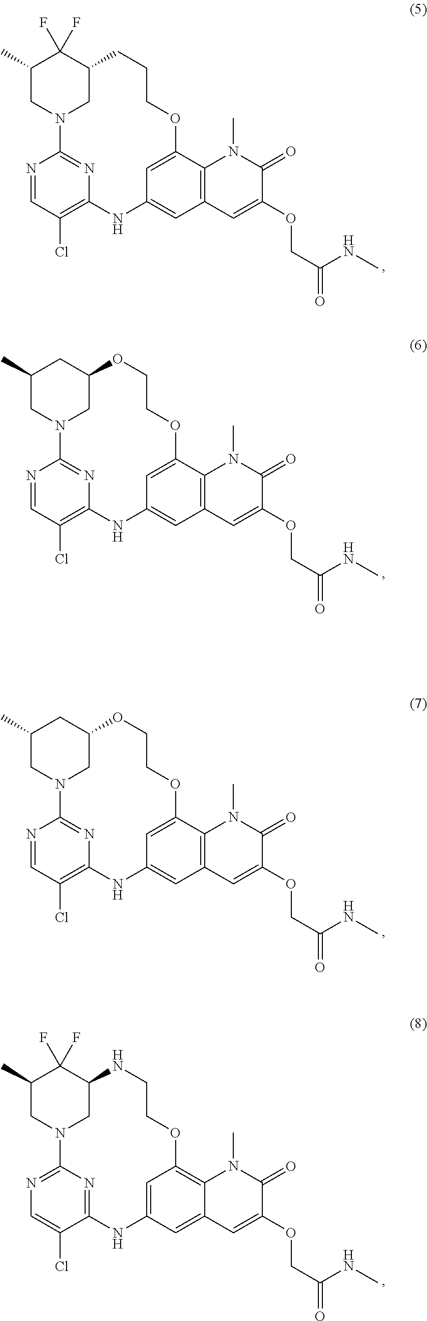 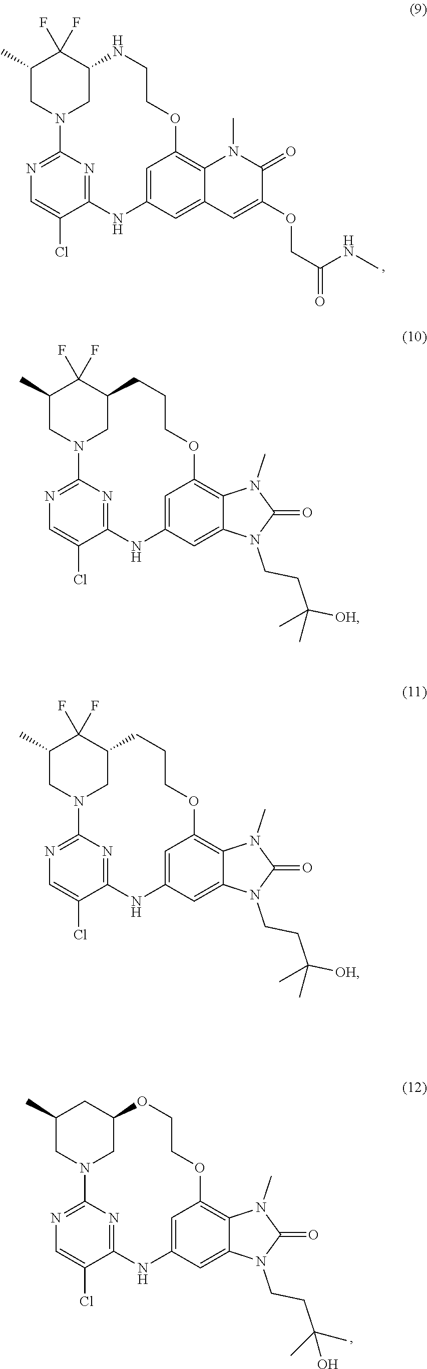 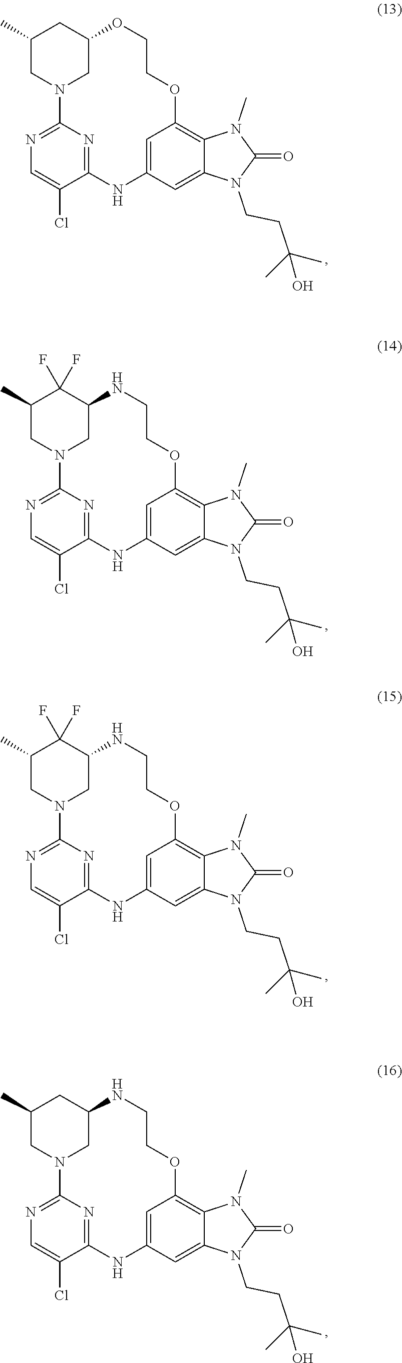 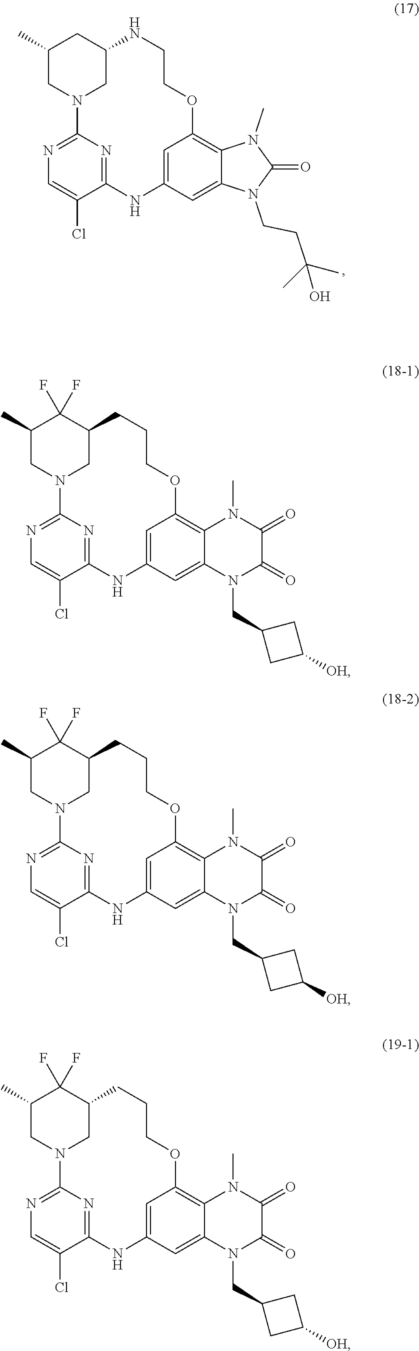 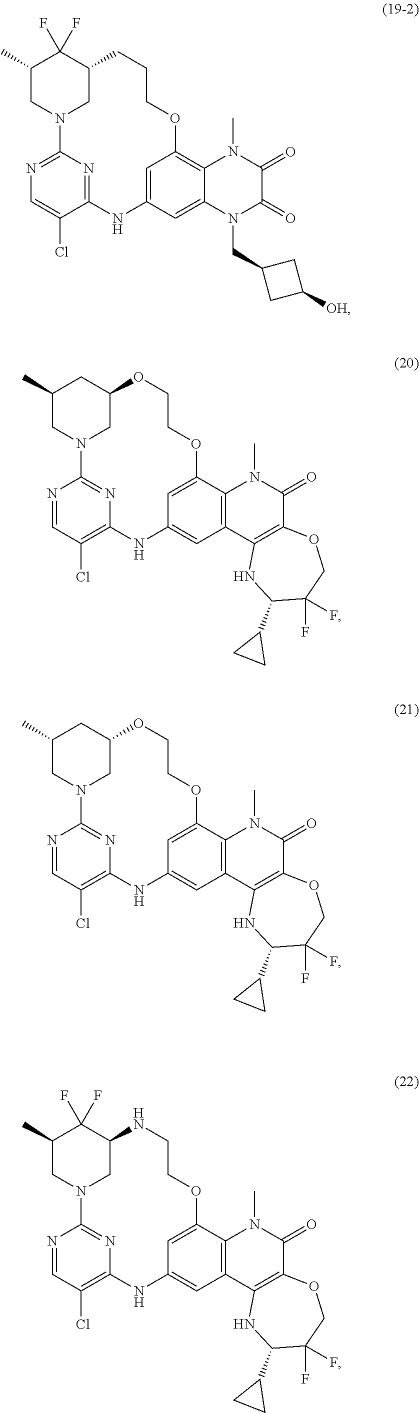 [SMILES: C[C@H]1CN2C[C@@H](CCCOc3cc(cc4c3n(C)c(=O)c(=O)n4C[C@H]3C[C@@H](O)C3)Nc3nc2ncc3Cl)C1(F)F ; C[C@H]1C[C@@H]2CN(C1)c1ncc(Cl)c(n1)Nc1cc(c3c(c1)c1c(c(=O)n3C)OCC(F)(F)[C@H](C3CC3)N1)OCCO2 ; C[C@@H]1C[C@H]2CN(C1)c1ncc(Cl)c(n1)Nc1cc(c3c(c1)c1c(c(=O)n3C)OCC(F)(F)[C@H](C3CC3)N1)OCCO2 ; C[C@@H]1CN2C[C@H](NCCOc3cc(cc4c5c(c(=O)n(C)c34)OCC(F)(F)[C@H](C3CC3)N5)Nc3nc2ncc3Cl)C1(F)F] 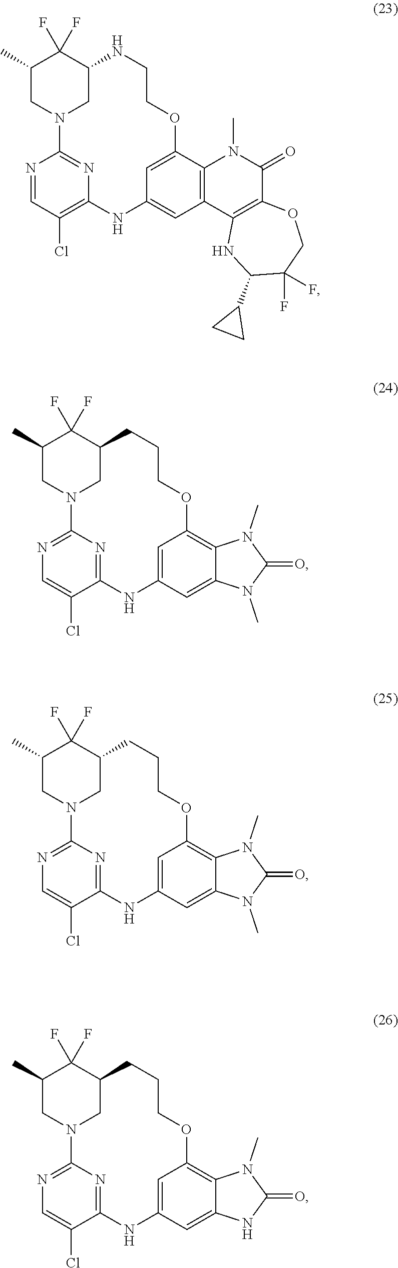 [SMILES: C[C@H]1CN2C[C@@H](NCCOc3cc(cc4c5c(c(=O)n(C)c34)OCC(F)(F)[C@H](C3CC3)N5)Nc3nc2ncc3Cl)C1(F)F ; C[C@@H]1CN2C[C@H](CCCOc3cc(cc4c3n(C)c(=O)n4C)Nc3nc2ncc3Cl)C1(F)F ; C[C@H]1CN2C[C@@H](CCCOc3cc(cc4c3n(C)c(=O)n4C)Nc3nc2ncc3Cl)C1(F)F ; C[C@@H]1CN2C[C@H](CCCOc3cc(cc4[nH]c(=O)n(C)c34)Nc3nc2ncc3Cl)C1(F)F] 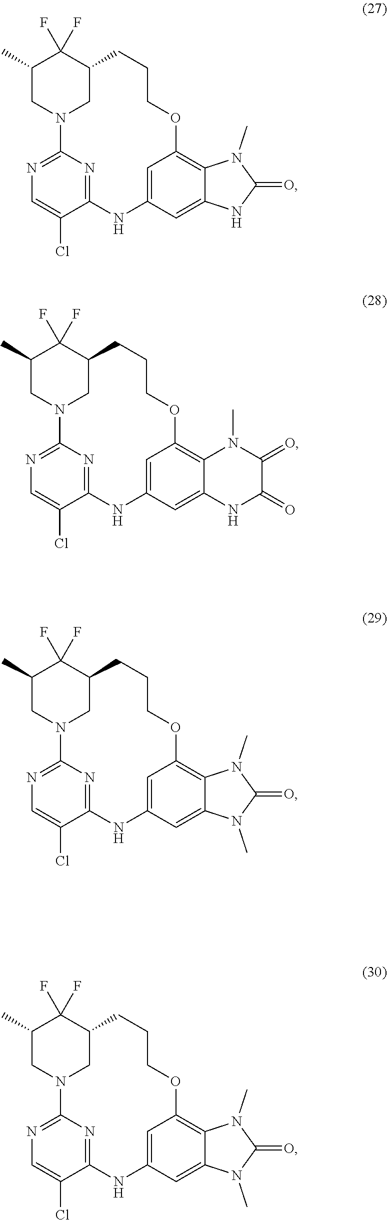 [SMILES: C[C@H]1CN2C[C@@H](CCCOc3cc(cc4[nH]c(=O)n(C)c34)Nc3nc2ncc3Cl)C1(F)F ; C[C@@H]1CN2C[C@H](CCCOc3cc(cc4[nH]c(=O)c(=O)n(C)c34)Nc3nc2ncc3Cl)C1(F)F ; C[C@@H]1CN2C[C@H](CCCOc3cc(cc4c3n(C)c(=O)n4C)Nc3nc2ncc3Cl)C1(F)F ; C[C@H]1CN2C[C@@H](CCCOc3cc(cc4c3n(C)c(=O)n4C)Nc3nc2ncc3Cl)C1(F)F] 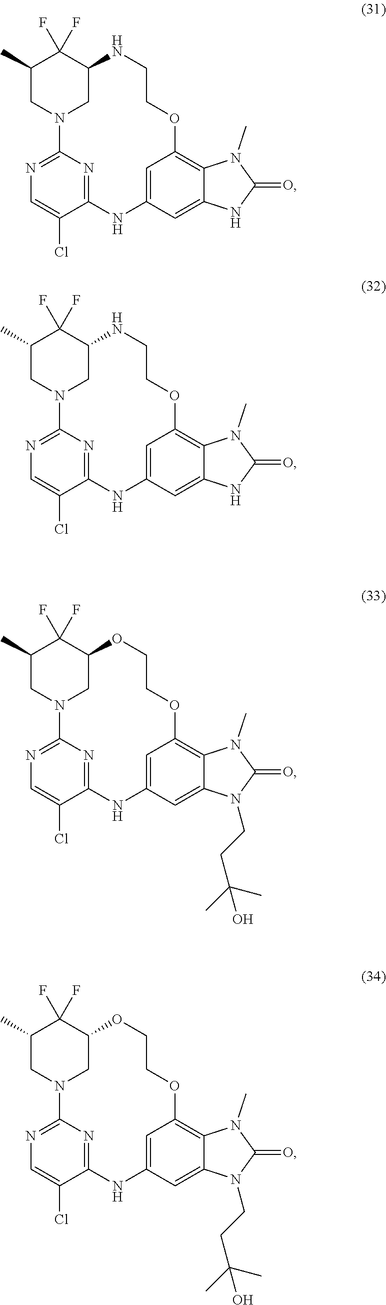 [SMILES: C[C@@H]1CN2C[C@H](NCCOc3cc(cc4[nH]c(=O)n(C)c34)Nc3nc2ncc3Cl)C1(F)F ; C[C@H]1CN2C[C@@H](NCCOc3cc(cc4[nH]c(=O)n(C)c34)Nc3nc2ncc3Cl)C1(F)F ; C[C@@H]1CN2C[C@H](OCCOc3cc(cc4c3n(C)c(=O)n4CCC(C)(C)O)Nc3nc2ncc3Cl)C1(F)F ; C[C@H]1CN2C[C@@H](OCCOc3cc(cc4c3n(C)c(=O)n4CCC(C)(C)O)Nc3nc2ncc3Cl)C1(F)F] 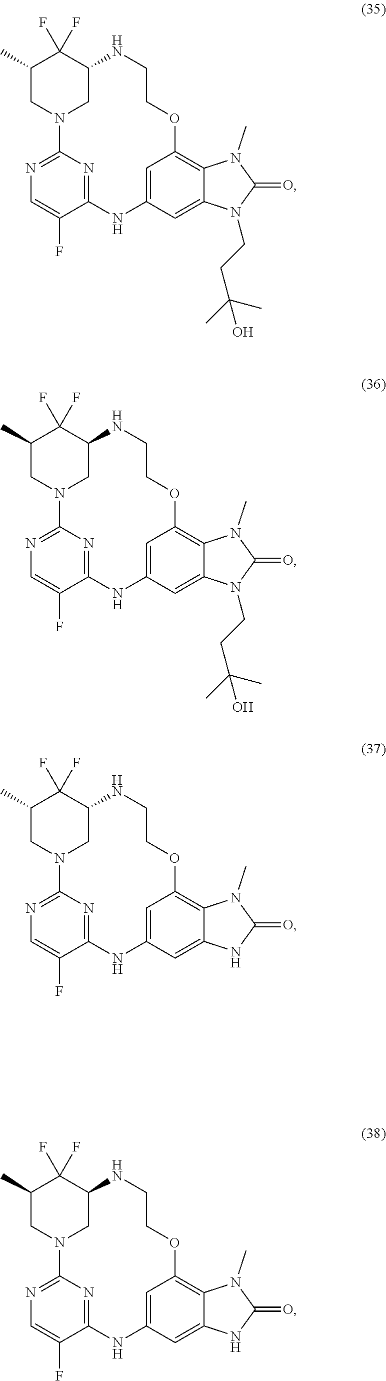 [SMILES: C[C@H]1CN2C[C@@H](NCCOc3cc(cc4c3n(C)c(=O)n4CCC(C)(C)O)Nc3nc2ncc3F)C1(F)F ; C[C@@H]1CN2C[C@H](NCCOc3cc(cc4c3n(C)c(=O)n4CCC(C)(C)O)Nc3nc2ncc3F)C1(F)F ; C[C@H]1CN2C[C@@H](NCCOc3cc(cc4[nH]c(=O)n(C)c34)Nc3nc2ncc3F)C1(F)F ; C[C@@H]1CN2C[C@H](NCCOc3cc(cc4[nH]c(=O)n(C)c34)Nc3nc2ncc3F)C1(F)F] 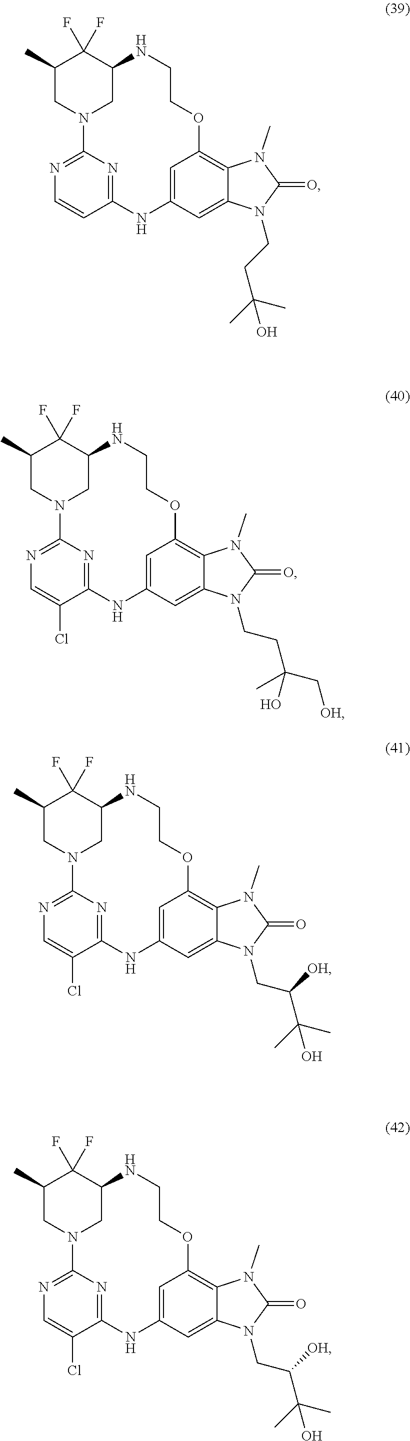 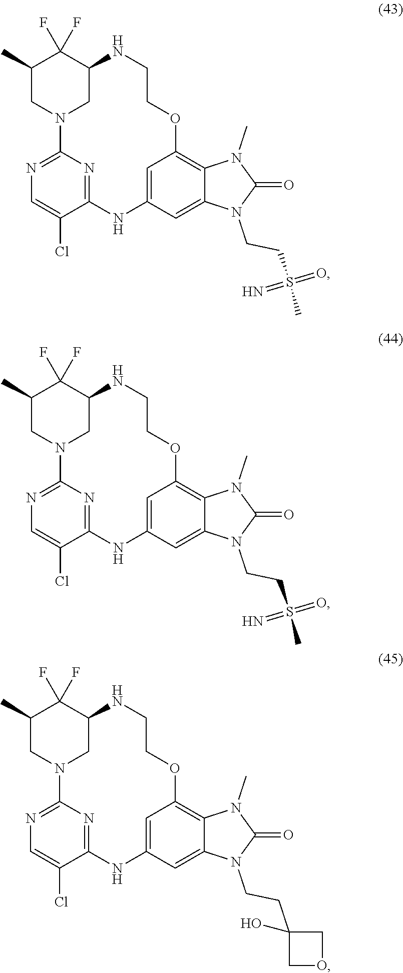 [SMILES: C[C@@H]1CN2C[C@H](NCCOc3cc(cc4c3n(C)c(=O)n4CC[S@](C)(=N)=O)Nc3nc2ncc3Cl)C1(F)F ; C[C@@H]1CN2C[C@H](NCCOc3cc(cc4c3n(C)c(=O)n4CC[S@@](C)(=N)=O)Nc3nc2ncc3Cl)C1(F)F ; C[C@@H]1CN2C[C@H](NCCOc3cc(cc4c3n(C)c(=O)n4CCC3(O)COC3)Nc3nc2ncc3Cl)C1(F)F]
or a pharmaceutically acceptable salt or stereoisomer thereof.

52. A pharmaceutical composition, comprising a therapeutically effective amount of the compound or pharmaceutically acceptable salt or stereoisomer of claim 1, and a pharmaceutically acceptable carrier.

53. A method of treating a disease or disorder that involves aberrant B-cell lymphoma 6 (BCL6) activity, comprising administering to a subject in need thereof a therapeutically effective amount of the compound or a pharmaceutically acceptable salt or stereoisomer thereof of claim 1, or the pharmaceutical composition of claim 52.

54. The method of claim 53, wherein the disease or disorder is cancer or an inflammatory disease.

55. The method of claim 54, wherein the cancer is a lymphoid malignancy.

56. The method of claim 55, wherein the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), or cutaneous T-cell lymphoma.

57. The method of claim 55, further comprising administering an additional anti-cancer agent.

58. The method of claim 57, wherein the additional anti-cancer agent is an enhancer of zeste homolog 2 (EZH2) inhibitor.

59. (canceled)

60. The method of claim 54, wherein the inflammatory disease is inflammatory bowel disease, myocarditis, endometriosis, atherosclerosis, an allergic disease, or an autoimmune disease.

61. The method of claim 60, wherein the allergic disease is asthma or pollinosis.

62. The method of claim 60, wherein the autoimmune disease is noninfectious meningitis, autoimmune encephalitis, transverse myelitis, or acute disseminated encephalomyelitis.
